(19) United States
(12) Patent Application Publication
(10) Pub. No.: US 2024/0150737 A1
(43) Pub. Date: May 9, 2024

(54) SOLUBLE ALKALINE PHOSPHATASE CONSTRUCTS AND EXPRESSION VECTORS INCLUDING A POLYNUCLEOTIDE ENCODING FOR SOLUBLE ALKALINE PHOSPHATASE CONSTRUCTS

(71) Applicants: RAMPART BIOSCIENCE, INC., Tucson, AZ (US); THE REGENTS OF THE UNIVERSITY OF CALIFORNIA, Oakland, CA (US)

(72) Inventors: JEFFREY S. BARTLETT, Columbus, OH (US); ANJIE ZHEN, Los Angeles, CA (US); MING YAN, Encino, CA (US); LOUIS RANDALL BRETON, Tucson, AZ (US); Irvin S.Y. CHEN, Palos Verdes Estates, CA (US)

(21) Appl. No.: 18/114,528

(22) Filed: Feb. 27, 2023

Related U.S. Application Data
(63) Continuation of application No. PCT/US21/48943, filed on Sep. 2, 2021.
(60) Provisional application No. 63/074,418, filed on Sep. 3, 2020.

(51) Int. Cl. C12N 9/16; C12N 15/86

(52) U.S. Cl. CPC C12N 9/16; C12N 15/86; C07K 2319/30; C12N 2740/15043; C12Y 301/03001

(57) ABSTRACT

The present disclosure is directed to polypeptides, such as polypeptides comprising an alkaline phosphatase and a bone-targeting moiety. Also disclosed are expression vectors, such as lentiviral expression vectors, including a nucleotide sequence encoding a polypeptide. Also disclosed are methods of treating hypophosphatasia or treating, mitigating, or preventing one or more symptoms of hypophosphatasia by administering to a subject in need of treatment thereof the disclosed polypeptides or host cells transduced to express any of the disclosed polypeptides.

### CROSS-REFERENCE TO RELATED APPLICATIONS

The present application is a continuation of International Application No. PCT/US2021/048943 filed on Sep. 2, 2021, which application claims the benefit of the filing date of U.S. Provisional Patent Application No. 63/074,418 filed on Sep. 3, 2020, the disclosure of which is hereby incorporated by reference herein in its entirety.

### FIELD OF THE DISCLOSURE

The present disclosure relates to polypeptides and, in particular, polypeptides which target bone tissue. Also disclosed are expression vectors, such as lentiviral expression vectors, including one or more nucleotide sequences encoding a polypeptide. In some embodiments, the polypeptides are useful for treating hypophosphatasia or for treating, mitigating, or preventing one or more symptoms of hypophosphatasia in a subject in need of treatment thereof.

### SEQUENCE LISTING

The contents of the electronic sequence listing (RPT-003WO.xml; Size: 637,881 bytes; and Date of Creation: Feb. 20, 2023) is herein incorporated by reference in its entirety.

### BACKGROUND OF THE DISCLOSURE

Abnormalities in the levels of calcification and ossification lead to a spectrum of diseases, such as general arterial calcification of infancy (GACI), idiopathic infantile arterial calcification (IIAC), pseudoxanthoma elasticum (PXE), ossification of posterior longitudinal ligament (OPLL), medial wall vascular calcification (MWVC), autosomal recessive hypophosphatemia rickets type-2 (ARHR2), end state renal disease (ESRD), chronic kidney disease-bone/mineral disorder (CKD-MBD), X-linked hypophosphatemia (XLH), age related osteopenia, calcific uremic arteriolopathy (CUA) and hypophosphatemic rickets.

Hypophosphatasia (HPP) is a rare, heritable skeletal disease with an incidence of 1 per 100,000 births for the most severe forms of the disease. The disorder results from loss-of-function mutations in the gene encoding tissue-nonspecific alkaline phosphatase (TNSALP). HPP patients present a remarkable range of symptoms, from teeth loss or osteomalacia (rickets) to almost complete absence of bone mineralization in utero. Many patients with HPP present the characteristics of skeletal deformities, short stature, muscle and bone pain, impaired mobility, and premature loss of teeth. Perinatal-onset or infantile-onset HPP can also be characterized by the presence of rachitic chest deformity, vitamin B6-dependent seizures, and failure to thrive. In particular, HPP presenting at less than six months of age is often lethal due to respiratory insufficiency, with a low survival rate at one year of age.

### BRIEF SUMMARY OF THE DISCLOSURE

A first aspect of the present disclosure is a polypeptide comprising Formula (I):

[A]ᵥ-[B]-[C]_{w}-[R]_{q}-([D]ₓ-[E]_{y})_{z}, (I)
wherein
A comprises an amino acid sequence encoding a secretion signal peptide;
B comprises an amino acid encoding an alkaline phosphatase;
C comprises an amino acid sequence encoding a GPI anchor;
R is -(Mₒ(Fc)Nₚ)-, where M and N each independently include between 1 and 6 amino acids, where Fc is a Fc domain, and o and p are each independently 0, 1, or 2;
D comprises an amino acid sequence having between 4 and 6 amino acids, or is F(G)ₜF, where each F is the same amino acid, G is an amino acid sequence having 3, 4, or 5 amino acids, and t is an integer ranging from 2-5;
E comprises an amino acid sequence having between 1 and 8 amino acids;
q is 0 or 1;
v is 0 or 1;
w is 0 or 1;
x is 0 or an integer ranging from 1 to 6;
y is 0 or an integer ranging from 1 to 16; and
z is 0 or an integer ranging from 1 to 6.

In some embodiments, the polypeptide is catalytically competent to allow formation of hydroxyapatite crystals, such as in bone. In some embodiments, the polypeptide is capable of catalyzing the cleavage of inorganic pyrophosphate.

In some embodiments, q is zero. In some embodiments, q is zero and x is one or more.

In some embodiments, B comprises an amino acid sequence having at least 95% identity to SEQ ID NO: 11. In some embodiments, B comprises an amino acid sequence having at least 99% identity to SEQ ID NO: 11. In some embodiments, B comprises the amino acid sequence of SEQ ID NO: 11.

In some embodiments, the group [A]ᵥ-[B]-[C]_{w} comprises an amino acid sequence having at least 90% sequence identity to SEQ ID NO: 10. In some embodiments, the group [A], [B]-[C]_{w} comprises an amino acid sequence having at least 95% sequence identity to SEQ ID NO: 10. In some embodiments, the group [A]ᵥ-[B]-[C]_{w} comprises an amino acid sequence having at least 96% sequence identity to SEQ ID NO: 10. In some embodiments, [A]ᵥ-[B]-[C]_{w} comprises an amino acid sequence having at least 97% sequence identity to SEQ ID NO: 10. In some embodiments, the group [A]ᵥ-[B]-[C]_{w} comprises an amino acid sequence having at least 98% sequence identity to SEQ ID NO: 10. In some embodiments, the group [A]ᵥ-[B]-[C]_{w} comprises an amino acid sequence having at least 99% sequence identity to SEQ ID NO: 10. In some embodiments, the group [A]ᵥ-[B]-[C]_{w} comprises an amino acid sequence having SEQ ID NO: 10.

In some embodiments, A comprises an amino acid sequence having at least 95% identity to any one of SEQ ID NOS: 12 and 33-43. In some embodiments, A comprises an amino acid sequence having any one of SEQ ID NOS: 12 and 33-43. In some embodiments, v is 1 and w is 0. In some embodiments, z is 0. In some embodiments, x is 0. In some embodiments, the polypeptide has at least 95% sequence identity to any one of SEQ ID NO: 2. In some embodiments, the polypeptide comprises SEQ ID NO: 2.

In some embodiments, E comprises at most two amino acids. In some embodiments, E comprises 1 amino acid. In some embodiments, E comprises aspartic acid. In some embodiments, E comprises aspartic acid and y ranges from between 8 and 12. In some embodiments, E comprises aspartic acid, y ranges from between 8 and 12, and v is 1 and w is 0. In some embodiments, E comprises aspartic acid, y ranges from between 8 and 12, and v is 0. In some embodiments, E comprises aspartic acid, y ranges from between 8 and 12, and y is 10. In some embodiments, the polypeptide has at least 95% sequence identity to SEQ ID NO: 3. In some embodiments, the polypeptide comprises SEQ ID NO: 3.

In some embodiments, D comprises at most 5 amino acids (e.g., D may be -G-G-G-G-S-, as described herein). In some embodiments, at least three contiguous amino acids of the at most 5 amino acids are the same. In some embodiments, the at least three contiguous amino acids are each glycine. In some embodiments, the at least three contiguous amino acids are each glycine, and x is an integer ranging from 1 to 3. In some embodiments, the at least three contiguous amino acids are each glycine, and x is 2. In some embodiments, the at least three contiguous amino acids are each glycine, and y is an integer ranging from between 4 and 8. In some embodiments, the at least three contiguous amino acids are each glycine, and y is between 5 and 7. In some embodiments, y is 6.

In some embodiments, E comprises 3 amino acids. In some embodiments, at least 2 contiguous amino acids of the three amino acids are the same. In some embodiments, two contiguous amino acids of the 3 amino acids are each serine. In some embodiments, E comprises -asp-ser-ser-. In some embodiments, the polypeptide of Formula (I) has at least 95% sequence identity to SEQ ID NO: 6. In some embodiments, the polypeptide of Formula (I) comprises SEQ ID NO: 6.

In some embodiments, E comprises 1 or 2 amino acids. In some embodiments, E comprises 1 amino acid. In some embodiments, E comprises a single aspartic acid. In some embodiments, E is aspartic acid and y is between 8 and 12. In some embodiments, E is aspartic acid and y is 10. In some embodiments, the polypeptide of Formula (I) has at least 95% sequence identity to SEQ ID NO: 4. In some embodiments, the polypeptide of Formula (I) has at least 97% sequence identity to SEQ ID NO: 4. In some embodiments, the polypeptide of Formula (I) has at least 99% sequence identity to SEQ ID NO: 4. In some embodiments, the polypeptide of Formula (I) comprises SEQ ID NO: 4.

In some embodiments, E comprises between 2 and 8 amino acids. In some embodiments, E comprises between 2 and 6 amino acids. In some embodiments, E comprises between 2 and 4 amino acids. In some embodiments, E comprises 3 amino acids. In some embodiments, at least 2 contiguous amino acids of the 3 amino acids of the group E are the same. In some embodiments, y ranges from 4 to 8. In some embodiments, y ranges from 5 to 7. In some embodiments, y is 6. In some embodiments, E comprises -asp-ser-ser-. In some embodiments, v is 1 and w is 0.

In some embodiments, the polypeptide of Formula (I) has at least 80% sequence identity to SEQ ID NO: 5. In some embodiments, the polypeptide of Formula (I) has at least 85% sequence identity to SEQ ID NO: 5. In some embodiments, the polypeptide of Formula (I) has at least 90% sequence identity to SEQ ID NO: 5. In some embodiments, the polypeptide of Formula (I) has at least 95% sequence identity to SEQ ID NO: 5. In some embodiments, the polypeptide of Formula (I) has at least 96% sequence identity to SEQ ID NO: 5. In some embodiments, the polypeptide of Formula (I) has at least 97% sequence identity to SEQ ID NO: 5. In some embodiments, the polypeptide of Formula (I) has at least 98% sequence identity to SEQ ID NO: 5. In some embodiments, the polypeptide of Formula (I) has at least 99% sequence identity to SEQ ID NO: 5. In some embodiments, polypeptide of Formula (I) comprises SEQ ID NO: 5.

In some embodiments, the polypeptide of Formula (I) has at least 80% sequence identity to any one of SEQ ID NOS: 44-54, 68, 105, and 116-125. In some embodiments, the polypeptide of Formula (I) has at least 85% sequence identity to any one of SEQ ID NOS: 44-54, 105, and 116-125. In some embodiments, the polypeptide of Formula (I) has at least 90% sequence identity to any one of SEQ ID NOS: 44-54, 68, 105, and 116-125. In some embodiments, the polypeptide of Formula (I) has at least 95% sequence identity to any one of SEQ ID NOS: 44-54, 68, 105, and 116-125. In some embodiments, the polypeptide of Formula (I) has at least 96% sequence identity to any one of SEQ ID NOS: 44-54, 68, 105, and 116-125. In some embodiments, the polypeptide of Formula (I) has at least 97% sequence identity to any one of SEQ ID NOS: SEQ ID NOS: 44-54, 68, 105, and 116-125. In some embodiments, the polypeptide of Formula (I) has at least 98% sequence identity to any one of SEQ ID NOS: 44-54, 105, and 116-125. In some embodiments, the polypeptide of Formula (I) has at least 99% sequence identity to any one of SEQ ID NOS: SEQ ID NOS: 44-54, 68, 105, and 116-125. In some embodiments, polypeptide of Formula (I) comprises any one of SEQ ID SEQ ID NOS: 44-54, 68, 105, and 116-125.

In some embodiments, the amino acid sequence encoding the GPI anchor has at least 95% sequence identity to SEQ ID NO: 13. In some embodiments, the amino acid sequence encoding the GPI anchor has SEQ ID NO: 14. In some embodiments, the polypeptide of Formula (I) has at least 95% sequence identity to SEQ ID NO: 8. In some embodiments, the polypeptide of Formula (I) has at least 97% sequence identity to SEQ ID NO: 8. In some embodiments, the polypeptide of Formula (I) has at least 99% sequence identity to SEQ ID NO: 8. In some embodiments, the polypeptide comprises SEQ ID NO: 8.

In some embodiments, v is 1, w is 1, and the amino acid sequence encoding the GPI anchor has at least 95% sequence identity to SEQ ID NO: 13. In some embodiments, v is 1, w is 1, and the amino acid sequence encoding the GPI anchor has at least 97% sequence identity to SEQ ID NO: 13. In some embodiments, v is 1, w is 1, and the amino acid sequence encoding the GPI anchor has at least 99% sequence identity to SEQ ID NO: 13. In some embodiments, v is 1, w is 1, and the amino acid sequence encoding the GPI anchor has SEQ ID NO: 14. In some embodiments, v is 1, w is 1 and the polypeptide of Formula (I) has at least 95% sequence identity to SEQ ID NO: 7. In some embodiments, v is 1, w is 1 and polypeptide of Formula (I) comprises SEQ ID NO: 7.

A second aspect of the present disclosure is a lentiviral vector including a nucleic acid sequence encoding a polypeptide having Formula (I):

[A]ᵥ-[B]-[C]_{w}-[R]_{q}-([D]ₓ-[E]_{y})_{z}, (I)
wherein
A comprises an amino acid sequence encoding a secretion signal peptide;
B comprises an amino acid encoding an alkaline phosphatase;
C comprises an amino acid sequence encoding a GPI anchor;
R is -(Mₒ(Fc)Nₚ)-, where M and N each independently include between 1 and 6 amino acids, where Fc is a Fc domain, and o and p are each independently 0, 1, or 2;
D comprises an amino acid sequence having between 4 and 6 amino acids, or is F(G)ₜF, where each F is the same amino acid, G is an amino acid sequence having 3, 4, or 5 amino acids, and t is an integer ranging from 2-5;
E comprises an amino acid sequence having between 1 and 8 amino acids;
q is 0 or 1;
v is 0 or 1;
w is 0 or 1;
x is 0 or an integer ranging from 1 to 6;
y is 0 or an integer ranging from 1 to 16; and
z is 0 or an integer ranging from 1 to 6.

Specific examples of polypeptides having Formula (I) are recited herein and described above with regard to the first aspect of the present disclosure. In some embodiments, the polypeptide does not comprise the amino acid sequence of SEQ ID NO: 1 or does not comprise the amino acid sequence encoded by the nucleotide sequence of SEQ ID NO: 107.

In some embodiments, the nucleic acid sequence encoding the polypeptide is operably linked to a promoter. In some embodiments, the promoter is selected from the group consisting of EF1A, MND, CD11b, CD68LPP, EF1a1, EFS, and UbC. In some embodiments, the lentiviral vector further comprises a UCOE promoter element. In some embodiments, the promoter has at least 95% identity to any one of SEQ ID NOS: 66, 67, 96, 99, 100, 101, and 126. In some embodiments, the promoter has at least 96% identity to any one of SEQ ID NOS: 66, 67, 96, 99, 100, 101, and 126. In some embodiments, the promoter has at least 97% identity to any one of SEQ ID NOS: 66, 67, 96, 99, 100, 101, and 126. In some embodiments, the promoter has at least 95% identity to any one of SEQ ID NOS: 66, 67, 96, 99, 100, 101, and 126. In some embodiments, the promoter has at least 98% identity to any one of SEQ ID NOS: 66, 67, 96, 99, 100, 101, and 126. In some embodiments, the promoter has at least 99% identity to any one of SEQ ID NOS: 66, 67, 96, 99, 100, 101, and 126. In some embodiments, the promoter comprises any one of SEQ ID NOS: 66, 67, 96, 99, 100, 101, and 126. In some embodiments, the lentiviral vector further comprises an insulator. In some embodiments, the lentiviral vector further comprises one or more Matrix Attachment Regions. In some embodiments, the lentiviral vector further comprises a WPRE element. In some embodiments, the lentiviral vector does not include a WPRE element.

In some embodiments, the lentiviral vector comprises a nucleotide sequence having at least 80% sequence identity to any one of SEQ ID NOS: 16-26, 55-65, 74, 81, 82, 84-95, 97, and 98. In some embodiments, the lentiviral vector comprises a nucleotide sequence having at least 85% sequence identity to any one of SEQ ID NOS: 16-26, 55-65, 74, 81, 82, 84-95, 97, and 98. In some embodiments, the lentiviral vector comprises a nucleotide sequence having at least 90% sequence identity to any one of SEQ ID NOS: 16-26, 55-65, 74, 81, 82, 84-95, 97, and 98. In some embodiments, the lentiviral vector comprises a nucleotide sequence having at least 95% sequence identity to any one of SEQ ID NOS: 16-26, 55-65, 74, 81, 82, 84-95, 97, and 98. In some embodiments, the lentiviral vector comprises a nucleotide sequence having at least 96% sequence identity to any one of SEQ ID NOS: 16-26, 55-65, 74, 81, 82, 84-95, 97, and 98. In some embodiments, the lentiviral vector comprises a nucleotide sequence having at least 97% sequence identity to any one of SEQ ID NOS: 16-26, 55-65, 74, 81, 82, 84-95, 97, and 98. In some embodiments, the lentiviral vector comprises a nucleotide sequence having at least 98% sequence identity to any one of SEQ ID NOS: 16-26, 55-65, 74, 81, 82, 84-95, 97, and 98. In some embodiments, the lentiviral vector comprises a nucleotide sequence having at least 99% sequence identity to any one of SEQ ID NOS: 16-26, 55-65, 74, 81, 82, 84-95, 97, and 98. In some embodiments, the lentiviral viral vector comprises a nucleotide sequence having any one of SEQ ID NOS: 16-26, 55-65, 74, 81, 82, 84-95, 97, and 98.

A third aspect of the present disclosure is a population of host cells transduced with an expression vector, wherein the expression vector includes a nucleic acid sequence encoding a polypeptide having Formula (I):

[A]ᵥ-[B]-[C]_{w}-[R]_{q}-([D]ₓ-[E]_{y})_{z}, (I)
wherein
A comprises an amino acid sequence encoding a secretion signal peptide;
B comprises an amino acid encoding an alkaline phosphatase;
C comprises an amino acid sequence encoding a GPI anchor;
R is -(Mₒ(Fc)Nₚ)-, where M and N each independently include between 1 and 6 amino acids, where Fc is a Fc domain, and o and p are each independently 0, 1, or 2;
D comprises an amino acid sequence having between 4 and 6 amino acids, or is F(G)ₜF, where each F is the same amino acid, G is an amino acid sequence having 3, 4, or 5 amino acids, and t is an integer ranging from 2-5;
E comprises an amino acid sequence having between 1 and 8 amino acids;
q is 0 or 1;
v is 0 or 1;
w is 0 or 1;
x is 0 or an integer ranging from 1 to 6;
y is 0 or an integer ranging from 1 to 16; and
z is 0 or an integer ranging from 1 to 6.

Specific examples of polypeptides having Formula (I) are recited herein and described above with regard to the first aspect of the present disclosure. In some embodiments, the polypeptide does not comprise the amino acid sequence of SEQ ID NO: 1 or does not comprise the amino acid sequence encoded by the nucleotide sequence of SEQ ID NO: 107.

In some embodiments, the expression vector is a retroviral vector. In some embodiments, the expression vector is a lentiviral vector. In some embodiments, the lentiviral vector comprises a nucleotide sequence having at least 80% sequence identity to any one of SEQ ID NOS: 15-26, 55-65, 74, 81, 82, 84-95, 97, and 98. In some embodiments, the lentiviral vector comprises a nucleotide sequence having at least 85% sequence identity to any one of SEQ ID NOS: 15-26, 55-65, 74, 81, 82, 84-95, 97, and 98. In some embodiments, the lentiviral vector comprises a nucleotide sequence having at least 90% sequence identity to any one of SEQ ID NOS: 15-26, 55-65, 74, 81, 82, 84-95, 97, and 98. In some embodiments, the lentiviral vector comprises a nucleotide sequence having at least 95% sequence identity to any one of SEQ ID NOS: 15-26, 55-65, 74, 81, 82, 84-95, 97, and 98. In some embodiments, the lentiviral vector comprises a nucleotide sequence having at least 96% sequence identity to any one of SEQ ID NOS: 15-26, 55-65, 74, 81, 82, 84-95, 97, and 98. In some embodiments, the lentiviral vector comprises a nucleotide sequence having at least 97% sequence identity to any one of SEQ ID NOS: 15-26, 55-65, 74, 81, 82, 84-95, 97, and 98. In some embodiments, the lentiviral vector comprises a nucleotide sequence having at least 98% sequence identity to any one of SEQ ID NOS: 15-26, 55-65, 74, 81, 82, 84-95, 97, and 98. In some embodiments, the lentiviral vector comprises a nucleotide sequence having at least 99% sequence identity to any one of SEQ ID NOS: 15-26, 55-65, 74, 81, 82, 84-95, 97, and 98. In some embodiments, the lentiviral viral vector comprises a nucleotide sequence having any one of SEQ ID NOS: 15-26, 55-65, 74, 81, 82, 84-95, 97, and 98.

In some embodiments, the host cells are hematopoietic stem cells. In some embodiments, the host cells are mesenchymal cells. In some embodiments, the host cells are bone marrow cells. In some embodiments, the host cells are hepatocytes. In some embodiments, the host cells are endothelial cells. In some embodiments, the host cells are transduced ex vivo. In some embodiments, the host cells are transduced in vivo.

In some embodiments, the transduced host cells express a polypeptide having Formula (I). In some embodiments, the transduced host cells may be administered to a mammalian subject in need of treatment thereof.

A fourth aspect of the present disclosure is a method of transducing a population of host cells comprising: obtaining a population of host cells and contacting the obtained population of host cells with an expression vector including a nucleic acid sequence encoding a polypeptide having Formula (I):

[A]ᵥ-[B]-[C]_{w}-[R]_{q}-([D]ₓ-[E]_{y})_{z}, (I)
wherein
A comprises an amino acid sequence encoding a secretion signal peptide;
B comprises an amino acid encoding an alkaline phosphatase;
C comprises an amino acid sequence encoding a GPI anchor;
R is -(Mₒ(Fc)Nₚ)-, where M and N each independently include between 1 and 6 amino acids, where Fc is a Fc domain, and o and p are each independently 0, 1, or 2;
D comprises an amino acid sequence having between 4 and 6 amino acids, or is F(G)ₜF, where each F is the same amino acid, G is an amino acid sequence having 3, 4, or 5 amino acids, and t is an integer ranging from 2-5;
E comprises an amino acid sequence having between 1 and 8 amino acids;
q is 0 or 1;
v is 0 or 1;
w is 0 or 1;
x is 0 or an integer ranging from 1 to 6;
y is 0 or an integer ranging from 1 to 16; and
z is 0 or an integer ranging from 1 to 6.

In some embodiments, the transduction occurs ex vivo.

In some embodiments, the transduction occurs in vivo.

Specific examples of polypeptides having Formula (I) are recited herein and described above with regard to the first aspect of the present disclosure. In some embodiments, the polypeptide does not comprise the amino acid sequence of SEQ ID NO: 1 or does not comprise the amino acid sequence encoded by the nucleotide sequence of SEQ ID NO: 107.

In some embodiments, the expression vector is a retroviral vector. In some embodiments, the expression vector is a lentiviral vector. In some embodiments, the lentiviral vector comprises a nucleotide sequence having at least 80% sequence identity to any one of SEQ ID NOS: 15-26, 55-65, 74, 81, 82, 84-95, 97, and 98. In some embodiments, the lentiviral vector comprises a nucleotide sequence having at least 85% sequence identity to any one of SEQ ID NOS: 15-26, 55-65, 74, 81, 82, 84-95, 97, and 98. In some embodiments, the lentiviral vector comprises a nucleotide sequence having at least 90% sequence identity to any one of SEQ ID NOS: 15-26, 55-65, 74, 81, 82, 84-95, 97, and 98. In some embodiments, the lentiviral vector comprises a nucleotide sequence having at least 95% sequence identity to any one of SEQ ID NOS: 15-26, 55-65, 74, 81, 82, 84-95, 97, and 98. In some embodiments, the lentiviral vector comprises a nucleotide sequence having at least 96% sequence identity to any one of SEQ ID NOS: 15-26, 55-65, 74, 81, 82, 84-95, 97, and 98. In some embodiments, the lentiviral vector comprises a nucleotide sequence having at least 97% sequence identity to any one of SEQ ID NOS: 15-26, 55-65, 74, 81, 82, 84-95, 97, and 98. In some embodiments, the lentiviral vector comprises a nucleotide sequence having at least 98% sequence identity to any one of SEQ ID NOS: 15-26, 55-65, 74, 81, 82, 84-95, 97, and 98. In some embodiments, the lentiviral vector comprises a nucleotide sequence having at least 99% sequence identity to any one of SEQ ID NOS: 15-26, 55-65, 74, 81, 82, 84-95, 97, and 98. In some embodiments, the lentiviral viral vector comprises a nucleotide sequence having any one of SEQ ID NOS: 15-26, 55-65, 74, 81, 82, 84-95, 97, and 98.

An fifth aspect of the present disclosure is a pharmaceutical composition comprising a population of modified host cells, wherein the population of modified host cells express a polypeptide having Formula (I):

[A]ᵥ-[B]-[C]_{w}-[R]_{q}-([D]ₓ-[E]_{y})_{z}, (I)
wherein
A comprises an amino acid sequence encoding a secretion signal peptide;
B comprises an amino acid encoding an alkaline phosphatase;
C comprises an amino acid sequence encoding a GPI anchor;
R is -(Mₒ(Fc)Nₚ)-, where M and N each independently include between 1 and 6 amino acids, where Fc is a Fc domain, and o and p are each independently 0, 1, or 2;
D comprises an amino acid sequence having between 4 and 6 amino acids, or is F(G)ₜF, where each F is the same amino acid, G is an amino acid sequence having 3, 4, or 5 amino acids, and t is an integer ranging from 2-5;
E comprises an amino acid sequence having between 1 and 8 amino acids;
q is 0 or 1;
v is 0 or 1;
w is 0 or 1;
x is 0 or an integer ranging from 1 to 6;
y is 0 or an integer ranging from 1 to 16; and
z is 0 or an integer ranging from 1 to 6.

Specific examples of polypeptides having Formula (I) are recited herein and described above with regard to the first aspect of the present disclosure. In some embodiments, the polypeptide does not comprise the amino acid sequence of SEQ ID NO: 1 or does not comprise the amino acid sequence encoded by the nucleotide sequence of SEQ ID NO: 107. In some embodiments, the host cells are transduced ex vivo. In some embodiments, the host cells are transduced in vivo.

A sixth aspect of the present disclosure is a method of treating a mammalian subject comprising administering a pharmaceutically effective amount of a population of modified host cells to the mammalian subject, wherein the population of modified host cells express a polypeptide having Formula (I):

[A]ᵥ-[B]-[C]_{w}-[R]_{q}-([D]ₓ-[E]_{y})_{z}, (I)
wherein
A comprises an amino acid sequence encoding a secretion signal peptide;
B comprises an amino acid encoding an alkaline phosphatase;
C comprises an amino acid sequence encoding a GPI anchor;
R is -(Mₒ(Fc)Nₚ)-, where M and N each independently include between 1 and 6 amino acids, where Fc is a Fc domain, and o and p are each independently 0, 1, or 2;
D comprises an amino acid sequence having between 4 and 6 amino acids, or is F(G)ₜF, where each F is the same amino acid, G is an amino acid sequence having 3, 4, or 5 amino acids, and t is an integer ranging from 2-5;
E comprises an amino acid sequence having between 1 and 8 amino acids;
q is 0 or 1;
v is 0 or 1;
w is 0 or 1;
x is 0 or an integer ranging from 1 to 6;
y is 0 or an integer ranging from 1 to 16; and
z is 0 or an integer ranging from 1 to 6.

Specific examples of polypeptides having Formula (I) are recited herein and described above with regard to the first aspect of the present disclosure. In some embodiments, the polypeptide does not comprise the amino acid sequence of SEQ ID NO: 1 or does not comprise the amino acid sequence encoded by the nucleotide sequence of SEQ ID NO: 107.

In some embodiments, the mammalian subject was previously treated with, will be treated with, or is concurrently being treated with a polypeptide having an amino acid sequence having SEQ ID NO: 1.

In some embodiments, q is 0. In some embodiments, q is 0 and wherein E comprises between 2 and 4 amino acids. In some embodiments, q is 0 and wherein E comprises three amino acids. In some embodiments, q is 0 and wherein E comprises -D-S-S-. In some embodiments, q is 0, E comprises -D-S-S-, and x is 0.

In some embodiments, q is 1. In some embodiments, q is 1 and wherein E comprises between 2 and 4 amino acids. In some embodiments, q is 1 and wherein E comprises three amino acids. In some embodiments, q is 1 and wherein E comprises -D-S-S-. In some embodiments, q is 1, E comprises -D-S-S-, and x is 0.

In some embodiments, q is 0. In some embodiments, q is 0 and [D]ₓ is [-G-G-G-G-S-]₂. In some embodiments, q is 0, [D]ₓ is [-G-G-G-G-S-]₂, and [E]y is [D]₁₀. In some embodiments, q is 0, [D]ₓ is [-G-G-G-G-S-]₂, and [E]y is [D]₆.

In some embodiments, q is 1. In some embodiments, q is 1 and [D]ₓ is [-G-G-G-G-S-]₂. In some embodiments, q is 1, [D]ₓ is [-G-G-G-G-S-]₂, and [E]y is [D]₁₀. In some embodiments, q is 1, [D]ₓ is [-G-G-G-G-S-]₂, and [E]y is [D]₆.

A seventh aspect of the present disclosure is a polypeptide comprising Formula (IA):

[A]ᵥ-[B]-[C]_{w}-[R]_{q}-([D]ₓ-[E]_{y})_{z}, (IA)
wherein
A comprises an amino acid sequence encoding a secretion signal peptide;
B comprises an amino acid encoding an alkaline phosphatase;
C comprises an amino acid sequence encoding a GPI anchor;
R is -(Mₒ(Fc)Nₚ)-, where M and N each independently include between 1 and 6 amino acids, where Fc is a Fc domain, and o and p are each independently 0, 1, or 2;
D comprises an amino acid sequence having between 4 and 6 amino acids, or is F(G)ₜF, where each F is the same amino acid, G is an amino acid sequence having 3, 4, or 5 amino acids, and t is an integer ranging from 2-5;
E comprises an amino acid sequence having between 1 and 8 amino acids;
q is 0 or 1;
v is 0 or 1;
w is 0 or 1;
x is 0 or an integer ranging from 1 to 6;
y is 0 or an integer ranging from 1 to 16; and
z is 0 or an integer ranging from 1 to 6;
provided that the polypeptide of Formula (IA) does not have the amino acid sequence of SEQ ID NO: 1 or does not comprise the amino acid sequence encoded by the nucleotide sequence of SEQ ID NO: 107.

In some embodiments, the polypeptide is catalytically competent to allow formation of hydroxyapatite crystals, such as in bone. In some embodiments, the polypeptide is capable of catalyzing the cleavage of inorganic pyrophosphate.

In some embodiments, B comprises at least 99% sequence identity to SEQ ID NO: 11. In some embodiments, B comprises SEQ ID NO: 11.

In some embodiments, [A]ᵥ-[B]-[C]_{w} comprises an amino acid sequence having at least 97% sequence identity to SEQ ID NO: 10. In some embodiments, [A]ᵥ-[B]-[C]_{w} comprises SEQ ID NO: 10.

In some embodiments, A comprises an amino acid sequence having at least 99% identity to any one of SEQ ID NOS: 12 and 33-43. In some embodiments, A comprises an amino acid sequence having any one of SEQ ID NOS: 12 and 33-43.

In some embodiments, R comprises at least 99% identity to that of SEQ ID NO: 9. In some embodiments, R comprises SEQ ID NO: 9.

In some embodiments, o, p, and q are each 1. In some embodiments, M comprises 2 amino acids; and N comprises two amino acids; and M and N are different. In some embodiments, M is -L-K-. In some embodiments, N is -D-I-. In some embodiments, E comprises between 2 and 4 amino acids. In some embodiments, E comprises 3 amino acids. In some embodiments, at least 2 contiguous amino acids of the 3 amino acids are the same. In some embodiments, E comprises -D-S-S-.

In some embodiments, q is 0.

In some embodiments, q is 1. In some embodiments, x is 0. In some embodiments, z is 1. In some embodiments, y ranges from 4-8. In some embodiments, y is 6.

In some embodiments, [E]y is [-D-S-S-]₆.

In some embodiments, [E]y is [-D-S-S-]₆ and q is 0. In some embodiments, [E]_{y} is [-D-S-S-]₆, q is 0 and x is 0.

In some embodiments, [E]_{y} is [-D-S-S-]₆ and q is 1. In some embodiments, [E]_{y} is [-D-S-S-]₆, q is 1, and x is 0. In some embodiments, [E]_{y} is [-D-S-S-]₆, q is 1, and Fc comprises at least 97% identity to SEQ ID NO: 130. In some embodiments, [E]_{y} is [-D-S-S-]₆, q is 1, and Fc comprises at least 99% identity to SEQ ID NO: 130. In some embodiments, [E]_{y} is [-D-S-S-]₆, q is 1, and Fc comprises SEQ ID NO: 130.

In some embodiments, q is 1 and R comprises at least 97% identity to SEQ ID NO: 9. In some embodiments, q is 1 and R comprises at least 99% identity to SEQ ID NO: 9. In some embodiments, q is 1 and R comprises SEQ ID NO: 9.

In some embodiments, [D]ₓ is [-G-G-G-G-S-]₂. In some embodiments, [E]_{y} is [D]₁₀. In some embodiments, [E]_{y} is [D]₆. In some embodiments, [R] is -[L-K]-Fc-[D-I]-, and Fc comprises at least 97% identity to SEQ ID NO: 130. In some embodiments, [R] is -[L-K]-Fc-[D-I]-, and Fc comprises at least 98% identity to SEQ ID NO: 130. In some embodiments, [R] is -[L-K]-Fc-[D-I]-, and Fc comprises at least 99% identity to SEQ ID NO: 130. In some embodiments, R comprises SEQ ID NO: 9.

In some embodiments, the polypeptide has an amino acid sequence having at least 90% identity to any one of 2-8, 44-54, 68, 75, 105, and 116-125. In some embodiments, the polypeptide has an amino acid sequence having at least 95% identity to any one of 2-8, 44-54, 68, 75, 105, and 116-125. In some embodiments, the polypeptide has an amino acid sequence having at least 96% identity to any one of 2-8, 44-54, 68, 75, 105, and 116-125. In some embodiments, the polypeptide has an amino acid sequence having at least 97% identity to any one of 2-8, 44-54, 68, 75, 105, and 116-125. In some embodiments, the polypeptide has an amino acid sequence having at least 98% identity to any one of 2-8, 44-54, 68, 75, 105, and 116-125. In some embodiments, the polypeptide has an amino acid sequence having at least 99% identity to any one of 2-8, 44-54, 68, 75, 105, and 116-125. In some embodiments, the polypeptide comprises an amino acid sequence having any one of 2-8, 44-54, 68, 75, 105, and 116-125.

In some embodiments, the polypeptide is encoded by a nucleotide sequence having at least 90% identity to any one of SEQ ID NOS: 106, 108-115, and 131. In some embodiments, the polypeptide is encoded by a nucleotide sequence having at least 95% identity to any one of SEQ ID NOS: 106, 108-115, and 131. In some embodiments, the polypeptide is encoded by a nucleotide sequence having at least 96% identity to any one of SEQ ID NOS: 106, 108-115, and 131. In some embodiments, the polypeptide is encoded by a nucleotide sequence having at least 97% identity to any one of SEQ ID NOS: 106, 108-115, and 131. In some embodiments, the polypeptide is encoded by a nucleotide sequence having at least 98% identity to any one of SEQ ID NOS: 106, 108-115, and 131. In some embodiments, the polypeptide is encoded by a nucleotide sequence having at least 99% identity to any one of SEQ ID NOS: 106, 108-115, and 131. In some embodiments, the polypeptide is encoded by a nucleotide sequence having any one of SEQ ID NOS: 106, 108-115, and 131.

An eighth aspect of the present disclosure is a lentiviral vector including a nucleotide sequence encoding a polypeptide having Formula (IA) (such as any of the polypeptides having Formula (IA) described herein). In some embodiments, the nucleotide sequence encoding the polypeptide having Formula (IA) is operably linked to a promoter. In some embodiments, the promoter is selected from EF1A, MND, CD11b, CD68LP, EF1a1, EFS, and UbC. In some embodiments, the promoter has at least 95% identity to any one of SEQ ID NOS: 66, 67, 96, 99, 100, 101, and 126. In some embodiments, the promoter has at least 96% identity to any one of SEQ ID NOS: 66, 67, 96, 99, 100, 101, and 126. In some embodiments, the promoter has at least 97% identity to any one of SEQ ID NOS: 66, 67, 96, 99, 100, 101, and 126. In some embodiments, the promoter has at least 95% identity to any one of SEQ ID NOS: 66, 67, 96, 99, 100, 101, and 126. In some embodiments, the promoter has at least 98% identity to any one of SEQ ID NOS: 66, 67, 96, 99, 100, 101, and 126. In some embodiments, the promoter has at least 99% identity to any one of SEQ ID NOS: 66, 67, 96, 99, 100, 101, and 126. In some embodiments, the promoter comprises any one of SEQ ID NOS: 66, 67, 96, 99, 100, 101, and 126.

In some embodiments, the lentiviral vector further comprises a UCOE promoter element. In some embodiments, the lentiviral vector further comprises an insulator. In some embodiments, the lentiviral vector further comprises one or more Scaffold/Matrix Attachment Regions. In some embodiments, the lentiviral vector further comprises a WPRE element. In some embodiments, the lentiviral vector does not include a nucleotide sequence encoding a WPRE element. In some embodiments, the lentiviral vector comprises a nucleotide sequence having at least 95% sequence identity to any one of SEQ ID NOS: 16-26, 55-65, 74, 81, 82, 84-95, 97, and 98. In some embodiments, the lentiviral vector comprises a nucleotide sequence having at least 96% sequence identity to any one of SEQ ID NOS: 16-26, 55-65, 74, 81, 82, 84-95, 97, and 98. In some embodiments, the lentiviral vector comprises a nucleotide sequence having at least 97% sequence identity to any one of SEQ ID NOS: 16-26, 55-65, 74, 81, 82, 84-95, 97, and 98. In some embodiments, the lentiviral vector comprises a nucleotide sequence having at least 98% sequence identity to any one of SEQ ID NOS: 16-26, 55-65, 74, 81, 82, 84-95, 97, and 98. In some embodiments, the lentiviral vector comprises a nucleotide sequence having at least 99% sequence identity to any one of SEQ ID NOS: 16-26, 55-65, 74, 81, 82, 84-95, 97, and 98. In some embodiments, the lentiviral vector comprises a nucleotide sequence having any one of SEQ ID NOS: 16-26, 55-65, 74, 81, 82, 84-95, 97, and 98.

A ninth aspect of the present disclosure is a population of host cells transduced with an expression vector encoding a polypeptide having Formula (IA), e.g., a retroviral vector, a lentiviral vector, etc. In some embodiments, the nucleotide sequence encoding the polypeptide having Formula (IA) is operably linked to a promoter. In some embodiments, the population of transduced host cells expresses a polypeptide having Formula (IA). In some embodiments, the promoter is selected from EF1A, MND, CD11b, CD68Lp, EF1a1, EFS, and UbC. In some embodiments, the lentiviral vector further comprises a UCOE promoter element. In some embodiments, the lentiviral vector further comprises an insulator. In some embodiments, the lentiviral vector further comprises one or more Scaffold/Matrix Attachment Regions. In some embodiments, the lentiviral vector further comprises a WPRE element. In some embodiments, the lentiviral vector does not include a nucleotide sequence encoding a WPRE element. In some embodiments, the host cells are autologous. In some embodiments, the host cells are allogeneic. In some embodiments, the host cells are transduced ex vivo. In some embodiments, the host cells are transduced in vivo.

A tenth aspect of the present disclosure is a method of treating a mammalian subject, e.g., a human subject, comprising administering a population of transduced host cells to the mammalian subject, the population of transduced host cells expressing a polypeptide having Formula (IA). Examples of polypeptides having Formula (IA) are described herein. In some embodiments, the polypeptide of Formula (IA) has an amino acid sequence having at least 97% identity to any one of 2-8, 44-54, 68, 75, 105, and 116-125. In some embodiments, the polypeptide of Formula (IA) has an amino acid sequence having at least 99% identity to any one of 2-8, 44-54, 68, 75, 105, and 116-125. In some embodiments, the polypeptide of Formula (IA) comprises an amino acid sequence having any one of 2-8, 44-54, 68, 75, 105, and 116-125. In some embodiments, the polypeptide of Formula (IA) is encoded by a nucleotide sequence having at least 95% identity to any one of SEQ ID NOS: 106, 108-115, and 131. In some embodiments, the polypeptide of Formula (IA) is encoded by a nucleotide sequence having at least 97% identity to any one of SEQ ID NOS: 106, 108-115, and 131. In some embodiments, the polypeptide of Formula (IA) is encoded by a nucleotide sequence having at least 99% identity to any one of SEQ ID NOS: 106, 108-115, and 131. In some embodiments, the polypeptide of Formula (IA) is encoded by a nucleotide sequence having any one of SEQ ID NOS: 106, 108-115, and 131.

In some embodiments, the host cells are autologous. In some embodiments, the host cells are allogeneic. In some embodiments, the mammalian subject was previously treated with, will be treated with, or is concurrently being treated with a polypeptide having an amino acid sequence having SEQ ID NO: 1.

An eleventh aspect of the present disclosure is a method of treating hypophosphatasia in a mammalian subject comprising administering a therapeutically effective amount of the population of transduced host cells to the mammalian subject, the population of transduced host cells expressing a polypeptide having Formula (IA) to the mammalian subject. Examples of polypeptides having Formula (IA) are described herein. In some embodiments, the polypeptide of Formula (IA) has an amino acid sequence having at least 97% identity to any one of 2-8, 44-54, 68, 75, 105, and 116-125. In some embodiments, the polypeptide of Formula (IA) has an amino acid sequence having at least 99% identity to any one of 2-8, 44-54, 68, 75, 105, and 116-125. In some embodiments, the polypeptide of Formula (IA) comprises an amino acid sequence having any one of 2-8, 44-54, 68, 75, 105, and 116-125. In some embodiments, the polypeptide of Formula (IA) is encoded by a nucleotide sequence having at least 95% identity to any one of SEQ ID NOS: 106, 108-115, and 131. In some embodiments, the polypeptide of Formula (IA) is encoded by a nucleotide sequence having at least 97% identity to any one of SEQ ID NOS: 106, 108-115, and 131. In some embodiments, the polypeptide of Formula (IA) is encoded by a nucleotide sequence having at least 99% identity to any one of SEQ ID NOS: 106, 108-115, and 131. In some embodiments, the polypeptide of Formula (IA) is encoded by a nucleotide sequence having any one of SEQ ID NOS: 106, 108-115, and 131.

In some embodiments, the host cells are autologous. In some embodiments, the host cells are allogeneic. In some embodiments, the mammalian subject was previously treated with, will be treated with, or is concurrently being treated with a polypeptide having an amino acid sequence having SEQ ID NO: 1.

A twelfth aspect of the present disclosure is a method of treating, mitigating, or preventing a symptom of hypophosphatasia in a mammalian subject comprising administering a therapeutically effective amount of the population of transduced host cells to the mammalian subject, the population of transduced host cells expressing a polypeptide having Formula (IA) to the mammalian subject. Examples of polypeptides having Formula (IA) are described herein. In some embodiments, the polypeptide of Formula (IA) has an amino acid sequence having at least 97% identity to any one of 2-8, 44-54, 68, 75, 105, and 116-125. In some embodiments, the polypeptide of Formula (IA) has an amino acid sequence having at least 99% identity to any one of 2-8, 44-54, 68, 75, 105, and 116-125. In some embodiments, the polypeptide of Formula (IA) comprises an amino acid sequence having any one of 2-8, 44-54, 68, 75, 105, and 116-125. In some embodiments, the polypeptide of Formula (IA) is encoded by a nucleotide sequence having at least 95% identity to any one of SEQ ID NOS: 106, 108-115, and 131. In some embodiments, the polypeptide of Formula (IA) is encoded by a nucleotide sequence having at least 97% identity to any one of SEQ ID NOS: 106, 108-115, and 131. In some embodiments, the polypeptide of Formula (IA) is encoded by a nucleotide sequence having at least 99% identity to any one of SEQ ID NOS: 106, 108-115, and 131. In some embodiments, the polypeptide of Formula (IA) is encoded by a nucleotide sequence having any one of SEQ ID NOS: 106, 108-115, and 131.

In some embodiments, the host cells are autologous. In some embodiments, the host cells are allogeneic. In some embodiments, the mammalian subject was previously treated with, will be treated with, or is concurrently being treated with a polypeptide having an amino acid sequence having SEQ ID NO: 1.

A thirteenth aspect of the present disclosure is a polypeptide comprising Formula (VB):

([A]-[B])-[R]_{q}-([E]_{y}) (VB)
wherein
A comprises an amino acid sequence encoding a secretion signal peptide;
B comprises an amino acid encoding an alkaline phosphatase;
R is -(Mₒ(Fc)Nₚ)-, where M and N each independently include between 1 and 6 amino acids, where Fc is a Fc domain, and o and p are each independently 0, 1, or 2;
q is 0 or 1;
E comprises an amino acid sequence having between 2 and 4 amino acids; and
y is integer ranging from 1 to 16,
provided that when q is 1 and when [E] comprises only aspartic acid, then the polypeptide of Formula (VB) does not terminate in an amino acid sequence having ten to sixteen contiguous aspartic acid residues.

In some embodiments, the polypeptide is catalytically competent to allow formation of hydroxyapatite crystals, such as in bone. In some embodiments, the polypeptide is capable of catalyzing the cleavage of inorganic pyrophosphate.

In some embodiments, y is an integer ranging from 4-8. In some embodiments, y is 6.

In some embodiments, q is 0.

In some embodiments, q is 1. In some embodiments, M comprises 2 amino acids; and wherein N comprises two amino acids; and wherein M and N are different. In some embodiments, M is -L-K-. In some embodiments, N is -D-I-.

In some embodiments, E comprises between 2 and 4 amino acids. In some embodiments, E comprises 3 amino acids. In some embodiments, at least 2 contiguous amino acids of the 3 amino acids are the same. In some embodiments, E comprises -D-S-S-.

In some embodiments, B comprises at least 99% sequence identity to SEQ ID NO: 11. In some embodiments, B comprises SEQ ID NO: 11. In some embodiments, [A]ᵥ-[B]-[C]_{w} comprises an amino acid sequence having at least 99% sequence identity to SEQ ID NO: 10. In some embodiments, [A]ᵥ-[B]-[C]_{w} comprises SEQ ID NO: 10.

In some embodiments, A comprises an amino acid sequence having at least 99% identity to any one of SEQ ID NOS: 12 and 33-43. In some embodiments, A comprises an amino acid sequence having any one of SEQ ID NOS: 12 and 33-43.

In some embodiments, Fc comprises at least 97% identity to that of SEQ ID NO: 130. In some embodiments, Fc comprises at least 98% identity to that of SEQ ID NO: 130. In some embodiments, Fc comprises at least 99% identity to that of SEQ ID NO: 130. In some embodiments, Fc comprises SEQ ID NO: 130. In some embodiments, R comprises at least 97% identity to SEQ ID NO: 9. In some embodiments, R comprises at least 98% identity to SEQ ID NO: 9. In some embodiments, R comprises at least 99% identity to SEQ ID NO: 9. In some embodiments, R comprises SEQ ID NO: 9.

A fourteenth aspect of the present disclosure is a lentiviral vector including a nucleotide sequence encoding a polypeptide having Formula (VB). Examples of polypeptides having Formula (VB) are described herein. In some embodiments, the polypeptide of Formula (VB) has an amino acid sequence having at least 90% identity to any one of 5, 44-54, 68, 75, 105, and 116-125. In some embodiments, the polypeptide of Formula (VB) has an amino acid sequence having at least 95% identity to any one of 5, 44-54, 68, 75, 105, and 116-125. In some embodiments, the polypeptide of Formula (VB) has an amino acid sequence having at least 96% identity to any one of 5, 44-54, 68, 75, 105, and 116-125. In some embodiments, the polypeptide of Formula (VB) has an amino acid sequence having at least 97% identity to any one of 5, 44-54, 68, 75, 105, and 116-125. In some embodiments, the polypeptide of Formula (VB) has an amino acid sequence having at least 98% identity to any one of 5, 44-54, 68, 75, 105, and 116-125. In some embodiments, the polypeptide of Formula (VB) has an amino acid sequence having at least 99% identity to any one of 5, 44-54, 68, 75, 105, and 116-125. In some embodiments, the polypeptide of Formula (VB) comprises an amino acid sequence having any one of 5, 44-54, 68, 75, 105, and 116-125.

In some embodiments, the polypeptide having Formula (VB) is encoded by a nucleotide sequence having at least 90% identity to any one of SEQ ID NOS: 111, 115, and 131. In some embodiments, the polypeptide having Formula (VB) is encoded by a nucleotide sequence having at least 95% identity to any one of SEQ ID NOS: 111, 115, and 131. In some embodiments, the polypeptide having Formula (VB) is encoded by a nucleotide sequence having at least 96% identity to any one of SEQ ID NOS: 111, 115, and 131. In some embodiments, the polypeptide having Formula (VB) is encoded by a nucleotide sequence having at least 97% identity to any one of SEQ ID NOS: 111, 115, and 131. In some embodiments, the polypeptide having Formula (VB) is encoded by a nucleotide sequence having at least 98% identity to any one of SEQ ID NOS: 111, 115, and 131. In some embodiments, the polypeptide having Formula (VB) is encoded by a nucleotide sequence having at least 99% identity to any one of SEQ ID NOS: 111, 115, and 131. In some embodiments, the polypeptide having Formula (VB) is encoded by a nucleotide sequence having any one of SEQ ID NOS: 111, 115, and 131.

In some embodiments, the nucleotide sequence encoding the polypeptide having Formula (VB) is operably linked to a promoter. In some embodiments, the promoter is selected from EF1A, MND, CD11b, CD68Lp, EF1a1, EFS, and UbC. In some embodiments, the lentiviral vector further comprises a UCOE promoter element. In some embodiments, the promoter has at least 95% identity to any one of SEQ ID NOS: 66, 67, 96, 99, 100, 101, and 126. In some embodiments, the promoter has at least 96% identity to any one of SEQ ID NOS: 66, 67, 96, 99, 100, 101, and 126. In some embodiments, the promoter has at least 97% identity to any one of SEQ ID NOS: 66, 67, 96, 99, 100, 101, and 126. In some embodiments, the promoter has at least 95% identity to any one of SEQ ID NOS: 66, 67, 96, 99, 100, 101, and 126. In some embodiments, the promoter has at least 98% identity to any one of SEQ ID NOS: 66, 67, 96, 99, 100, 101, and 126. In some embodiments, the promoter has at least 99% identity to any one of SEQ ID NOS: 66, 67, 96, 99, 100, 101, and 126. In some embodiments, the promoter comprises any one of SEQ ID NOS: 66, 67, 96, 99, 100, 101, and 126.

In some embodiments, the lentiviral vector further comprises an insulator. In some embodiments, the lentiviral vector further comprises one or more Scaffold/Matrix Attachment Regions. In some embodiments, the lentiviral vector further comprises a WPRE element. In some embodiments, the lentiviral vector further comprises a WPRE element.

A fifteenth aspect of the present disclosure is a method of treating a mammalian subject, e.g., a human subject, comprising administering a population of transduced host cells to the mammalian subject, the population of transduced host cells expressing a polypeptide having Formula (VB). Examples of polypeptides having Formula (VB) are described herein. In some embodiments, the polypeptide of Formula (VB) has an amino acid sequence having at least 97% identity to any one of 5, 44-54, 68, 75, 105, and 116-125. In some embodiments, the polypeptide of Formula (VB) has an amino acid sequence having at least 99% identity to any one of 5, 44-54, 68, 75, 105, and 116-125. In some embodiments, the polypeptide of Formula (VB) comprises an amino acid sequence having any one of 5, 44-54, 68, 75, 105, and 116-125. In some embodiments, the host cells are autologous. In some embodiments, the host cells are allogeneic. In some embodiments, the mammalian subject was previously treated with, will be treated with, or is concurrently being treated with a polypeptide having an amino acid sequence having SEQ ID NO: 1.

An sixteenth aspect of the present disclosure is a method of treating hypophosphatasia in a mammalian subject comprising administering a therapeutically effective amount of the population of transduced host cells to the mammalian subject, the population of transduced host cells expressing a polypeptide having Formula (VB) to the mammalian subject. Examples of polypeptides having Formula (VB) are described herein. In some embodiments, the polypeptide of Formula (VB) has an amino acid sequence having at least 97% identity to any one of 5, 44-54, 68, 75, 105, and 116-125. In some embodiments, the polypeptide of Formula (VB) has an amino acid sequence having at least 99% identity to any one of 5, 44-54, 68, 75, 105, and 116-125. In some embodiments, the polypeptide of Formula (VB) comprises an amino acid sequence having any one of 5, 44-54, 68, 75, 105, and 116-125. In some embodiments, the host cells are autologous. In some embodiments, the host cells are allogeneic. In some embodiments, the mammalian subject was previously treated with, will be treated with, or is concurrently being treated with a polypeptide having an amino acid sequence having SEQ ID NO: 1.

A seventeenth aspect of the present disclosure is a method of treating, mitigating, or preventing a symptom of hypophosphatasia in a mammalian subject comprising administering a therapeutically effective amount of the population of transduced host cells to the mammalian subject, the population of transduced host cells expressing a polypeptide having Formula (VB) to the mammalian subject. Examples of polypeptides having Formula (VB) are described herein. In some embodiments, the polypeptide of Formula (VB) has an amino acid sequence having at least 97% identity to any one of 5, 44-54, 68, 75, 105, and 116-125. In some embodiments, the polypeptide of Formula (VB) has an amino acid sequence having at least 99% identity to any one of 5, 44-54, 68, 75, 105, and 116-125. In some embodiments, the polypeptide of Formula (VB) comprises an amino acid sequence having any one of 5, 44-54, 68, 75, 105, and 116-125. In some embodiments, the host cells are autologous. In some embodiments, the host cells are allogeneic. In some embodiments, the mammalian subject was previously treated with, will be treated with, or is concurrently being treated with a polypeptide having an amino acid sequence having SEQ ID NO: 1.

An eighteenth aspect of the present disclosure is a method of treating a condition or disease related to a bone defect characterized by a lack of or an insufficient amount of functional alkaline phosphatase comprising: administering a therapeutically effective amount of transduced host cells expressing a polypeptide having any one of Formulas (I), (IA), (IB), (II), (III), (IV), (VA), and (VB) (such as described herein). In some embodiments, the expressed polypeptide has an amino acid sequence having at least 97% identity to any one of 2-8, 44-54, 68, 75, 105, and 116-125. In some embodiments, the expressed polypeptide has an amino acid sequence having at least 99% identity to any one of 2-8, 44-54, 68, 75, 105, and 116-125. In some embodiments, the expressed polypeptide comprises an amino acid sequence having any one of 2-8, 44-54, 68, 75, 105, and 116-125. In some embodiments, the expressed polypeptide is encoded by a nucleotide sequence having at least 95% identity to any one of SEQ ID NOS: 106, 108-115, and 131. In some embodiments, the expressed polypeptide is encoded by a nucleotide sequence having at least 97% identity to any one of SEQ ID NOS: 106, 108-115, and 131. In some embodiments, the expressed polypeptide is encoded by a nucleotide sequence having at least 99% identity to any one of SEQ ID NOS: 106, 108-115, and 131. In some embodiments, the expressed polypeptide is encoded by a nucleotide sequence having any one of SEQ ID NOS: 106, 108-115, and 131.

In some embodiments, the host cells are transduced with an expression vector, e.g., a retroviral expression vector or a lentiviral expression vector, wherein the expression vector comprises a nucleotide encoding a polypeptide having any one of Formulas (I), (IA), (IB), (II), (III), (IV), (VA), and (VB) (such as described herein). In some embodiments, the nucleotide sequence encoding the polypeptide having any one of Formulas (I), (IA), (IB), (II), (III), (IV), (VA), and (VB) is operably linked to a promoter. In some embodiments, the promoter is selected from EF1A, MND, CD11b, CD68Lp, EF1a1, EFS, and UbC. In some embodiments, the lentiviral vector further comprises a UCOE promoter element. In some embodiments, the lentiviral vector further comprises an insulator. In some embodiments, the lentiviral vector further comprises one or more Scaffold/Matrix Attachment Regions. In some embodiments, the lentiviral vector further comprises a WPRE element. In some embodiments, the lentiviral vector further comprises a WPRE element. In some embodiments, the transduction of the host cells occurs ex vivo. In some embodiments, the transduction of the host cells occurs in vivo. In some embodiments, the host cells are autologous. In some embodiments, the host cells are allogeneic. In some embodiments, the mammalian subject was previously treated with, will be treated with, or is concurrently being treated with a polypeptide having an amino acid sequence having SEQ ID NO: 1.

A nineteenth aspect of the present disclosure is a polypeptide comprising Formula (I):

[A]ᵥ-[B]-[C]_{w}-[R]_{q}-([D]ₓ-[E]_{y})_{z}, (IB)
wherein
A comprises an amino acid sequence encoding a secretion signal peptide;
B comprises an amino acid encoding an alkaline phosphatase;
C comprises an amino acid sequence encoding a GPI anchor;
R is -(Mₒ(Fc)Nₚ)-, where M and N each independently include between 1 and 6 amino acids, where Fc is a Fc domain, and o and p are each independently 0, 1, or 2;
D comprises an amino acid sequence having between 4 and 6 amino acids, or is F(G)ₜF, where each F is the same amino acid, G is an amino acid sequence having 3, 4, or 5 amino acids, and t is an integer ranging from 2-5;
E comprises an amino acid sequence having between 1 and 8 amino acids;
q is 0 or 1;
v is 0 or 1;
w is 0 or 1;
x is 0 or an integer ranging from 1 to 6;
y is 0 or an integer ranging from 1 to 16; and
z is 0 or an integer ranging from 1 to 6;
provided that when v is 1, w is 0, q is 1, o is 1, p is 1, N is the diamino acid -D-I-, M is the diamino acid -L-K-, [B] comprises SEQ ID NO: 11, Fc comprises SEQ ID NO: 130, and x is 0, then [E]_{y} does not comprise between ten and sixteen contiguous aspartic acid residues.

In some embodiments, E comprises between 2 and 4 amino acids. In some embodiments, E comprises 3 amino acids. In some embodiments, at least 2 contiguous amino acids of the 3 amino acids are the same. In some embodiments, E comprises -D-S-S-.

In some embodiments, q is 0.

In some embodiments, q is 1.

In some embodiments, x is 0. In some embodiments, z is 1. In some embodiments, y ranges from 4-8. In some embodiments, y is 6.

In some embodiments, [E]_{y} is [-D-S-S-]₆.

In some embodiments, q is 0 and x is 0.

In some embodiments, q is 1 and [R] is -[L-K]-Fc-[D-I]-, and wherein Fc comprises at least 97% identity to SEQ ID NO: 130. In some embodiments, q is 1 and [R] is -[L-K]-Fc-[D-I]-, and wherein Fc comprises at least 98% identity to SEQ ID NO: 130. In some embodiments, q is 1 and [R] is -[L-K]-Fc-[D-I]-, and wherein Fc comprises at least 99% identity to SEQ ID NO: 130. In some embodiments, [R] comprises SEQ ID NO: 9.

In some embodiments, the polypeptide has an amino acid sequence having at least 90% identity to any one of 2-8, 44-54, 68, 75, 105, and 116-125. In some embodiments, the polypeptide has an amino acid sequence having at least 95% identity to any one of 2-8, 44-54, 68, 75, 105, and 116-125. In some embodiments, the polypeptide has an amino acid sequence having at least 96% identity to any one of 2-8, 44-54, 68, 75, 105, and 116-125. In some embodiments, the polypeptide has an amino acid sequence having at least 97% identity to any one of 2-8, 44-54, 68, 75, 105, and 116-125. In some embodiments, the polypeptide has an amino acid sequence having at least 98% identity to any one of 2-8, 44-54, 68, 75, 105, and 116-125. In some embodiments, the polypeptide has an amino acid sequence having at least 99% identity to any one of 2-8, 44-54, 68, 75, 105, and 116-125. In some embodiments, the polypeptide comprises an amino acid sequence having any one of 2-8, 44-54, 68, 75, 105, and 116-125.

In some embodiments, the polypeptide is encoded by a nucleotide sequence having at least 90% identity to any one of SEQ ID NOS: 106, 108-115, and 131. In some embodiments, the polypeptide is encoded by a nucleotide sequence having at least 95% identity to any one of SEQ ID NOS: 106, 108-115, and 131. In some embodiments, the polypeptide is encoded by a nucleotide sequence having at least 96% identity to any one of SEQ ID NOS: 106, 108-115, and 131. In some embodiments, the polypeptide is encoded by a nucleotide sequence having at least 97% identity to any one of SEQ ID NOS: 106, 108-115, and 131. In some embodiments, the polypeptide is encoded by a nucleotide sequence having at least 98% identity to any one of SEQ ID NOS: 106, 108-115, and 131. In some embodiments, the polypeptide is encoded by a nucleotide sequence having at least 99% identity to any one of SEQ ID NOS: 106, 108-115, and 131. In some embodiments, the polypeptide is encoded by a nucleotide sequence having any one of SEQ ID NOS: 106, 108-115, and 131.

A twentieth aspect of the present disclosure is an expression vector comprising a nucleic acid sequence encoding a polypeptide having at least 90% identity to any one of SEQ ID NOS: 1-8, 44-54, 68, 75, 105, and 116-125. In some embodiments, the polypeptide has at least 91% identity to any one of SEQ ID NOS: 1-8, 44-54, 68, 75, 105, and 116-125. In some embodiments, the polypeptide has at least 92% identity to any one of SEQ ID NOS: 1-8, 44-54, 68, 75, 105, and 116-125. In some embodiments, the polypeptide has at least 93% identity to any one of SEQ ID NOS: 1-8, 44-54, 68, 75, 105, and 116-125. In some embodiments, the polypeptide has at least 94% identity to any one of SEQ ID NOS: 1-8, 44-54, 68, 75, 105, and 116-125. In some embodiments, the polypeptide has at least 95% identity to any one of SEQ ID NOS: 1-8, 44-54, 68, 75, 105, and 116-125. In some embodiments, the polypeptide has at least 96% identity to any one of SEQ ID NOS: 1-8, 44-54, 68, 75, 105, and 116-125. In some embodiments, the polypeptide has at least 97% identity to any one of SEQ ID NOS: 1-8, 44-54, 68, 75, 105, and 116-125. In some embodiments, the polypeptide has at least 98% identity to any one of SEQ ID NOS: 1-8, 44-54, 68, 75, 105, and 116-125. In some embodiments, the polypeptide has at least 99% identity to any one of SEQ ID NOS: 1-8, 44-54, 68, 75, 105, and 116-125. In some embodiments, the polypeptide has any one of SEQ ID NOS: 1-8, 44-54, 68, 75, 105, and 116-125. In some embodiments, the expression vector is a retroviral vector. In some embodiments, the retroviral vector is a lentiviral vector. In some embodiments, the expression vector is an AAV vector.

A twenty-first aspect of the present disclosure is an isolated nucleotide sequence having at least 90% identity to any one of SEQ ID NOS: 111, 115, and 131 (e.g., 91% identity, 92% identity, 93% identity, 94% identity, 95% identity, 96% identity, 97% identity, 98% identity, 99% identity, 100% identity).

A twenty-second aspect of the present disclosure is an isolated nucleotide sequence comprising at least first and second nucleotide sequences, wherein the first nucleotide sequence comprises at least 90% identity to SEQ ID NO: 115 (e.g., 91% identity, 92% identity, 93% identity, 94% identity, 95% identity, 96% identity, 97% identity, 98% identity, 99% identity, 100% identity); and wherein the second nucleotide sequence encodes for a signal peptide. In some embodiments, the signal peptide has at least 95% identity (e.g., 96% identity, 97% identity, 98% identity, 99% identity, 100% identity) to any one of SEQ ID NOS: 12 and 33-43.

A twenty-third aspect of the present disclosure is a lentiviral vector comprising: (i) a first nucleotide sequence having at least 90% identity to SEQ ID NO: 115 (e.g., 95% identity, 97% identity, 99% identity, 100% identity); and (ii) a second nucleotide sequence encoding a signal peptide. In some embodiments, the signal peptide comprises an amino acid sequence having at least 95% identity (e.g., 96% identity, 97% identity, 98% identity, 99% identity, 100% identity) to any one of SEQ ID NOS: 12 and 33-43. In some embodiments, the lentiviral vector further comprises a third nucleotide sequence encoding a promoter, such as a promoter operably linked to the first nucleotide sequence. In some embodiments, the promoter is selected from the group consisting of EF1A, MND, CD11b, CD68Lp, EF1a1, EFS, and UbC. In some embodiments, the promoter has at least 95% identity to any one of SEQ ID NOS: 66, 67, 96, 99, 100, 101, and 126. In some embodiments, the promoter has at least 96% identity to any one of SEQ ID NOS: 66, 67, 96, 99, 100, 101, and 126. In some embodiments, the promoter has at least 97% identity to any one of SEQ ID NOS: 66, 67, 96, 99, 100, 101, and 126. In some embodiments, the promoter has at least 95% identity to any one of SEQ ID NOS: 66, 67, 96, 99, 100, 101, and 126. In some embodiments, the promoter has at least 98% identity to any one of SEQ ID NOS: 66, 67, 96, 99, 100, 101, and 126. In some embodiments, the promoter has at least 99% identity to any one of SEQ ID NOS: 66, 67, 96, 99, 100, 101, and 126. In some embodiments, the promoter comprises any one of SEQ ID NOS: 66, 67, 96, 99, 100, 101, and 126.

A twenty-fourth aspect of the present disclosure is a population of host cells transduced with a lentiviral vector, wherein the lentiviral vector comprises a first nucleotide sequence having at least 90% identity to SEQ ID NO: 115 (e.g., 95% identity, 97% identity, 99% identity, 100% identity); and a second nucleotide sequence encoding a signal peptide. In some embodiments, the signal peptide comprises an amino acid sequence having at least 95% identity (e.g., 96% identity, 97% identity, 98% identity, 99% identity, 100% identity) to any one of SEQ ID NOS: 12 and 33-43. In some embodiments, the lentiviral vector further comprises a third nucleotide sequence encoding a promoter. In some embodiments, the promoter is selected from the group consisting of EF1A, MND, CD11b, CD68Lp, EF1a1, EFS, and UbC. In some embodiments, the host cells are transduced ex vivo. In some embodiments, the host cells are transduced in vivo.

A twenty-fifth aspect of the present disclosure is an isolated nucleotide sequence comprising at least 97% identity (e.g., 98% identity, 99% identity, 100% identity) to that of SEQ ID NO: 111.

A twenty-sixth aspect of the present disclosure is an amino acid sequence having at least 80% identity (e.g., 90% identity, 95% identity, 97% identity, 99% identity, 100% identity) to SEQ ID NO: 75.

A twenty-seventh aspect of the present disclosure is a polypeptide comprising at least first and second portions, wherein the first portion comprises an amino acid sequence having at least 95% identity (e.g., 96% identity, 97% identity, 98% identity, 99% identity, 100% identity) to any one of SEQ ID NOS: 4-8 and 44-54; and wherein the second portion comprises an amino acid sequence encoding an Fc domain. In some embodiments, the amino acid sequence encoding the Fc domain comprises at least 95% identity (e.g., 96% identity, 97% identity, 98% identity, 99% identity, 100% identity) to SEQ ID NO: 130.

A twenty-eighth aspect of the present disclosure is an isolated nucleotide sequence comprising at least 90% identity to SEQ ID NO: 72. In some embodiments, the isolated nucleotide sequence comprises at least 95% identity to SEQ ID NO: 72. In some embodiments, the isolated nucleotide sequence comprises at least 96% identity to SEQ ID NO: 72. In some embodiments, the isolated nucleotide sequence comprises at least 97% identity to SEQ ID NO: 72. In some embodiments, the isolated nucleotide sequence comprises at least 98% identity to SEQ ID NO: 72. In some embodiments, the isolated nucleotide sequence comprises at least 99% identity to SEQ ID NO: 72.

A twenty-ninth aspect of the present disclosure is an isolated nucleotide sequence comprising SEQ ID NO: 72.

A thirtieth aspect of the present disclosure is a polypeptide comprising an alkaline phosphatase coupled to a bone-targeting moiety. In some embodiments, a nucleotide sequence of the bone-targeting moiety comprises at least 90% identity (e.g., 91% identity, 92% identity, 93% identity, 94% identity, 955 identity, 96% identity, 97% identity, 98% identity, 99% identity, 100% identity) to SEQ ID NO: 72. In some embodiments, the alkaline phosphatase comprises an amino acid sequence having at least 90% identity (e.g., 91% identity, 92% identity, 93% identity, 94% identity, 955 identity, 96% identity, 97% identity, 98% identity, 99% identity, 100% identity) to SEQ ID NO: 11. In some embodiments, the alkaline phosphatase is coupled to the bone-targeting sequence via a peptide linker. In some embodiments, the peptide linker comprises (GGGGS)ₙ, wherein n ranges from 1 to 10. In some embodiments, is ranges from 2-8. In some embodiments, n is 2. In some embodiments, the polypeptide further comprises an Fc domain. In some embodiments, the Fc domain comprises at least 95% identity (e.g., 96% identity, 97% identity, 98% identity, 99% identity, 100% identity) to SEQ ID NO: 130.

A thirty-first aspect of the present disclosure is a lentiviral vector comprising a first nucleotide sequence encoding an alkaline phosphatase coupled to a bone-targeting moiety, wherein the first nucleotide sequence is operably linked to a promoter. In some embodiments, a nucleotide sequence of the bone-targeting moiety comprises at least 90% identity (e.g., 91% identity, 92% identity, 93% identity, 94% identity, 955 identity, 96% identity, 97% identity, 98% identity, 99% identity, 100% identity) to SEQ ID NO: 72. In some embodiments, the alkaline phosphatase comprises an amino acid sequence having at least 90% identity (e.g., 91% identity, 92% identity, 93% identity, 94% identity, 955 identity, 96% identity, 97% identity, 98% identity, 99% identity, 100% identity) to SEQ ID NO: 11. In some embodiments, the promoter is selected from the group consisting of EF1A, MND, CD11b, CD68Lp, EF1a1, EFS, and UbC. In some embodiments, the promoter has at least 95% identity to any one of SEQ ID NOS: 66, 67, 96, 99, 100, 101, and 126. In some embodiments, the promoter has at least 96% identity to any one of SEQ ID NOS: 66, 67, 96, 99, 100, 101, and 126. In some embodiments, the promoter has at least 97% identity to any one of SEQ ID NOS: 66, 67, 96, 99, 100, 101, and 126. In some embodiments, the promoter has at least 95% identity to any one of SEQ ID NOS: 66, 67, 96, 99, 100, 101, and 126. In some embodiments, the promoter has at least 98% identity to any one of SEQ ID NOS: 66, 67, 96, 99, 100, 101, and 126. In some embodiments, the promoter has at least 99% identity to any one of SEQ ID NOS: 66, 67, 96, 99, 100, 101, and 126. In some embodiments, the promoter comprises any one of SEQ ID NOS: 66, 67, 96, 99, 100, 101, and 126.

A thirty-second aspect of the present disclosure is a host cell transduced with a lentiviral vector comprising a first nucleotide sequence encoding an alkaline phosphatase coupled to a bone-targeting moiety, wherein the first nucleotide sequence is operably linked to a promoter. In some embodiments, a nucleotide sequence of the bone-targeting moiety comprises at least 90% identity (e.g., 91% identity, 92% identity, 93% identity, 94% identity, 955 identity, 96% identity, 97% identity, 98% identity, 99% identity, 100% identity) to SEQ ID NO: 72. In some embodiments, the alkaline phosphatase comprises at least 90% identity (e.g., 91% identity, 92% identity, 93% identity, 94% identity, 955 identity, 96% identity, 97% identity, 98% identity, 99% identity, 100% identity) to SEQ ID NO: 11. In some embodiments, the promoter is selected from the group consisting of EF1A, MND, CD11b, CD68Lp, EF1a1, EFS, and UbC. In some embodiments, the host cells are transduced ex vivo. In some embodiments, the host cells are transduced in vivo.

A thirty-third aspect of the present disclosure is a pharmaceutical composition comprising a polypeptide, wherein the polypeptide comprises an alkaline phosphatase coupled to a bone-targeting moiety. In some embodiments, a nucleotide sequence of the bone-targeting moiety comprises at least 90% identity (e.g., 91% identity, 92% identity, 93% identity, 94% identity, 955 identity, 96% identity, 97% identity, 98% identity, 99% identity, 100% identity) to SEQ ID NO: 72. In some embodiments, the alkaline phosphatase comprises at least 90% identity (e.g., 91% identity, 92% identity, 93% identity, 94% identity, 955 identity, 96% identity, 97% identity, 98% identity, 99% identity, 100% identity) to SEQ ID NO: 11. In some embodiments, the pharmaceutical composition is used to treat hypophosphatasia in a mammal in need of treatment thereof. In some embodiments, the pharmaceutical composition is used to mitigate or prevent a symptom of hypophosphatasia in a mammal in need of treatment thereof.

### BRIEF DESCRIPTION OF THE FIGURES

For a general understanding of the features of the disclosure, reference is made to the drawings. In the drawings, like reference numerals have been used throughout to identify identical elements.

FIG. 1 sets forth a vector map of a lentiviral vector comprising a nucleic acid sequence encoding an alkaline phosphatase, which polypeptide includes a secretion signal peptide, but does not include a GPI anchor. The polypeptide, in some embodiments, further includes a linker, an Fc domain, and a peptide having ten aspartic acid residues.

FIG. 2 sets forth a vector map of a lentiviral vector comprising a nucleic acid sequence encoding a polypeptide (SEQ ID NO: 2).

FIG. 3 sets forth a vector map of a lentiviral vector comprising a nucleic acid sequence encoding a polypeptide (SEQ ID NO: 3).

FIG. 4 sets forth a vector map of a lentiviral vector comprising a nucleic acid sequence encoding a polypeptide (SEQ ID NO: 4).

FIG. 5 sets forth a vector map of a lentiviral vector comprising a nucleic acid sequence encoding a polypeptide (SEQ ID NO: 5).

FIG. 6 sets forth a vector map of a lentiviral vector comprising a nucleic acid sequence encoding a polypeptide (SEQ ID NO: 6).

FIG. 7 sets forth a vector map of a lentiviral vector comprising a nucleic acid sequence encoding a polypeptide (SEQ ID NO: 7).

FIG. 8 sets forth a vector map of a lentiviral vector comprising a nucleic acid sequence encoding a polypeptide (SEQ ID NO: 8).

FIG. 9 sets forth a vector map of a lentiviral vector comprising a nucleic acid sequence encoding a polypeptide including a tissue non-specific alkaline phosphatase, and which polypeptide includes a secretion signal peptide and a GPI anchor.

FIG. 10 provides data demonstrating cell proliferation and viability in the presence of various concentration of sTNSALP-(DSS)6 RMP005 compound, by analyzing MTT (344,5-dimethyl-2-thiazolyl)-2,5-diphenyl-2H-tetrazolium bromide) incorporation into viable cells. Briefly, plated cells were incubated with about 0.25 mg/ml MTT solution for about 3 h at about 37° C. After incubation, precipitate was dissolved in DMSO. Spectrophotometric measurements were done using 560 nm wavelength in a microplate reader. Data show no toxic effect of RMP005 to the cells even at the highest concentration (10 U/ml).

FIG. 11 provides visualization of mineralization induced by of sTNSALP-(DSS)6 (RMP-005) (right panel). To document a potential mineralization inhibition/rescue a cell culture was initiated in differentiation medium (including ascorbic acid (AA) and a source of phosphate (β-glycerophosphate; βGP). At day 4 various concentrations of sTNSALP-(DSS)6 (RMP-005), 0.1, 0.5, and 1.0 unit/ml were added to the culture medium with or without the mineralization inhibitor sodium pyrophosphate tetrabasic (PPi) at a concentration of about 2.5 μM for the remainder of culture period (10 days). Mineral was visualized by von Kossa staining. Quantification of mineralization is provided in the left panel, wherein insoluble calcium deposited into the extracellular matrix (ECM) was first dissolved with about 0.5 M HCl and spectrophotometrically quantitated in the supernatant (wavelength about 595 nm) using commercially available calcium assay kit. RMP005 compound rescued the PPi induced inhibition of cell mineralization even at the lowest concentration (about 0.1 U/ml). A dose range effect on deposited calcium concentration showing complete rescue of mineralization at about 1.0 U/mL.

FIG. 12 compares in vitro hydroxyapatite (HA) binding for compounds “RMP-002” (SEQ ID NO: 2) and RMP-005 (SEQ ID NO: 5). Untagged sTNSALP (RMP-002) and tagged sTNSALP-(DSS)6 (“RMP005”) were subjected to a pulldown assay (supernatant depletion by mineral (HA)) as follows: Hydroxyapatite crystal solution (Berkeley, 5 μM) (HA) was prepared in 20 mM Tris (pH 7.4); 150 mM NaCl and allowed to equilibrate overnight at room temperature with gentle agitation. The next day, “RMP002” and “RMP005” were incubated with, or without, 0.3 mg HA at different concentration 0.5, 1.0, 5.0, 10.0 and 15.0 μg/ml and allowed to bind for one hour at room temperature on a shaker. Tubes were spun down at about 10,000×g for about 5 min, and the supernatant was used to measure the alkaline phosphatase level for each sample. A standard colorimetric method where alkaline phosphatase is used as a standard and p-nitrophenylphosphate as a phosphatase substrate, turns yellow when dephosphorylated by alkaline phosphatase, and was measured at 405 nm wavelength. Results show strong preferential binding of the tagged “RMP-005” over its non-tagged counterpart (“RMP002”).

FIG. 13 illustrates secreted TNALP activity in supernatant of 293 cells transfected with DNA constructs with wildtype and IgG2G secretion signal peptide. FIG. 13 illustrates that a construct with a secretion signal peptide of IgG2H has greater than 6-fold of secreted TNALP activity as compared with two constructs having the original secretion signal peptide (see SEQ ID NO: 12) including an EF1a promoter and an MND promoter.

FIG. 14 illustrates pharmacokinetic data for all of the test articles expressed in μg/mL (SEQ ID NOS: 1-8).

FIG. 15 provides a comparison of PK curves for RMP-001, RMP-005, RMP-006 and RMP-008 (corresponding to SEQ ID NOS: 1, 5, 6, and 8, respectively), each described herein.

FIG. 16 provides a comparison of PK curves for RMP-001, RMP-004 and RMP-007 (corresponding to SEQ ID NOS: 1, 4, and 7, respectively), each described herein.

FIG. 17 provides Cmax values (Tmax=5 min) for each of the test articles (SEQ ID NOS: 1-8).

FIG. 18 sets forth AUCs extrapolated to infinity for all test articles (SEQ ID NOS: 1-8).

FIG. 19 sets forth the elimination half-lives of the various test articles (SEQ ID NOS: 1-8).

FIG. 20A sets forth a vector map of RMP-100 (FG12w.MND.kz.IgKVIII.TNALPco(mut-miR362a).Fc.(DSS)6.WPRE) (SEQ ID NO: 74). In particular, this vector is comprised of: (1) Lentiviral vector backbone (HIV-1 sequences) without insulator; (2) Promotor/enhancer elements to drive TNALP mRNA expression; (3) 5 UTR sequences and translation initiation sequences to promote mRNA stability and efficient translation; (4) a human Ig kappa light chain V-III region signal peptide and its linker to promote efficient processing, and secretion of TNALP; (5) Codon-optimized TNALP coding sequences for efficient translation, stability, and enzymatic activity; (6) IgG1 Fc fusion partners and its linker for retention of the secreted protein in the plasma; (7) Bone tags for effective targeting of enzymatic activity to the bone; and (8) 3′ UTR sequences for mRNA stability.

FIG. 20B provides a schematic of self-inactivating lentiviral vector constructs LVV-RMP100 (used for lot number v1026). FG12w.MND.kz.IgKVIII-sTNALPco(mut-miR362a).Fc.(DSS)6.WPRE (SEQ ID NO: 74). as determined historically. RRE: Rev response elements; cPPT/CTS: central polypurine trace; hIgKVIII: a human Ig kappa light chain V-III region signal peptide; sTNALPco (mut-miR362a): GPI-anchorless secretory form of tissue-nonspecific alkaline phosphatase contained miR362T mut-A; Fc:) IgG1 Fc fusion partners and its linker for retention of the secreted protein in the plasma; (DSS)6: bone surface binding peptide; WPRE: woodchuck post-transcriptional regulatory element; bGH-poly(A) signal: a bovine growth hormone polyadenylation signal.

FIGS. 21A and 21B illustrate the activity of secreted TNALP from a series of different vectors (namely, EF1a-RMP5 (SEQ ID NO: 19), EF1a-IgG2H-RMP5 (SEQ ID NO: 55), EF1a-SEAP-RMP5 (SEQ ID NO: 65), EF1a-aLA-RMP5 (SEQ ID NO: 64), EF1a-hCD33-RMP5 (SEQ ID NO: 58), EF1a-Secrecon-RMP5 (SEQ ID NO: 60), EF1a-Secrecon-AA-RMP5 (SEQ ID NO: 61), EF1a-mIgKVIII-AA-RMP5 (SEQ ID NO: 62), and EF1a-hIgKVIII-AA-RMP5 (SEQ ID NO: 57)).

FIG. 22A illustrates TNALP activity secreted per vector copy number (VCN) for 293T cells (where the TNALP was secreted from cells transduced with either EF1a-RMP5 (SEQ ID NO: 19) or EF1a-IgG2H-RMP5 (SEQ ID NO: 55)).

FIG. 22B illustrates TNALP activity secreted per VCN for Jurkat cells (where the TNALP was secreted from cells transduced with either EF1a-RMP5 (SEQ ID NO: 19) or EF1a-IgG2H-RMP5 (SEQ ID NO: 55).

FIG. 23A illustrates and an alignment between EF1a-IgG2H-RMP5 (SEQ ID NO: 55), EF1a-IgG2H-RMP5-miR362-A (SEQ ID NO: 81), and EF1a-IgG2H-RMP5-miR362-B (SEQ ID NO: 82).

FIG. 23B illustrates the TNALP activity secreted from 293T cells transfected with vectors having EF1a-IgG2H-RMP5 (SEQ ID NO: 55), EF1a-IgG2H-RMP5-miR362-A (SEQ ID NO: 81) and EF1a-IgG2H-RMP5-miR362-B (SEQ ID NO: 82)

FIG. 23C the TNALP activity secreted from 293T cells transfected with EF1a-IgG2H-RMP5 (SEQ ID NO: 55) or EF1a-IgG2H-RMP5-miR362-A (SEQ ID NO: 81).

FIG. 24 compares TNALP levels secreted from FG12-SD400i-MND-IgKVIII-AA-RMP5-miR362A-Fc-(DSS)6 (SEQ ID NO: 84) with those of FG12-SD400i-MND-Kozak-IgKVIII-AA-RMP5-miR362A-Fc-(DSS)6 (SEQ ID NO: 85).

FIG. 25A compares measured vector titer for a series of vectors, including EF1a-RMP5 (SEQ ID NO: 19), EF1-IgG2H-RMP5 (SEQ ID NO: 55), EF1a-IgG2H-RMP5-miR-A (SEQ ID NO: 79), I400-MND-IgG2H-RMP5 (SEQ ID NO: 86), EF1-IgG2H-RMP5-Fc (SEQ ID NO: 87), and EF1a-IgKVIII-RMP5 (SEQ ID NO: 57).

FIG. 25B compares measured ALP activity per VCN for a series of vectors, including EF1a-RMP5 (SEQ ID NO: 19), EF1-IgG2H-RMP5 (SEQ ID NO: 55), EF1a-IgG2H-RMP5-miR-A (SEQ ID NO: 79), I400-MND-IgG2H-RMP5 (SEQ ID NO: 86), EF1-IgG2H-RMP5-Fc (SEQ ID NO: 87), and EF1a-IgKVIII-RMP5 (SEQ ID NO: 57) in HEK293 cell line.

FIGS. 25C and 25D compare measured TNALP activity per VCN for a series of vectors, including EF1a-RMP5 (SEQ ID NO: 19), EF1-IgG2H-RMP5 (SEQ ID NO: 55), EF1a-IgG2H-RMP5-miR-A (SEQ ID NO: 79), I400-MND-IgG2H-RMP5 (SEQ ID NO: 86), EF1-IgG2H-RMP5-Fc (SEQ ID NO: 87), and EF1a-IgKVIII-RMP5 (SEQ ID NO: 57) in Jurkat and K562 cell lines, respectively.

FIGS. 25A-25D illustrates that (i) IgG2H is better than TNALP wildtype secretion signal in transduced Jurkat and K562 cells; (ii) the MND promoter with 400-bps insulator (1400-MND-IgG2H-RMP5-SEQ ID NO: 86) outperforms the EF1a promoter (EF1-IgG2H-RMP5-SEQ ID NO: 55) in both hematopoietic cell lines shown in FIGS. 25C&D; (iii) the addition of the Fc fusion partner has minimal impact on TNALP secretion in the three different types of cell lines as there is no significant difference between (EF1-IgG2H-RMP5-SEQ ID NO: 55) and (EF1a-IgG2H-RMP5-Fc-SEQ ID NO: 87) for ALP secretion in FIG. 25B; (iv) the miR362-A mutation EF1a-IgG2H-RMP5-miR-A-SEQ ID NO: 79) performs better than the original construct EF1a-RMP5-SEQ ID NO: 19) in both myeloid cell lines shown in FIGS. 25C&D; and (v) TNALP secretion from IgKVIII (EF1a-IgKVIII-RMP5-SEQ ID NO: 57) is superior to IgG2H (EF1-IgG2H-RMP5-SEQ ID NO: 55) in all three different types of cell lines shown in FIGS. 25 B, C & D.

FIG. 26A compares measured titer for a series of vectors, including EF1a-IgG2H-RMP5 (SEQ ID NO: 55), EF1-IgG2H-RMP5-Fc (SEQ ID NO: 87), EF1a-IgG2H-sTNALPwt-DSS6 (SEQ ID NO: 88), EF1a-IgG2H-sTNALPwt-Fc-DSS6 (SEQ ID NO: 89), I400-MND-IgKVIII-RMP5(miR-A)-Fc-DSS6 (SEQ ID NO: 84), and I400-MND-kozak-IgKVIII-RMP5(miR-A)-Fc-DSS6 (SEQ ID NO: 85). The combinatory construct including the 400 bps insulator, the MND promoter, the Kozak element, the IgKVIII secretory signal peptide, the miR362-mutation, and Fc domain (I400-MND-kozak-IgKVIII-RMP5(miR-A)-Fc-DSS6 SEQ ID NO: 85) has comparable titer as rest of vectors (SEQ ID NO:55, 87, 88, 84, 89).

FIG. 26B compares measured ALP per VCN for a series of vectors, including EF1a-IgG2H-RMP5 (SEQ ID NO: 55), EF1-IgG2H-RMP5-Fc (SEQ ID NO: 87), EF1a-IgG2H-sTNALPwt-DSS6 (SEQ ID NO: 88), EF1a-IgG2H-sTNALPwt-Fc-DSS6 (SEQ ID NO: 89), 1400-MND-IgKVIII-RMP5(miR-A)-Fc-DSS6 (SEQ ID NO: 84), and I400-MND-kozak-IgKVIII-RMP5(miR-A)-Fc-DSS6 (SEQ ID NO: 85). The combinatory construct including the 400 bps insulator, the MND promoter, the Kozak element, the IgKVIII secretory signal peptide, the miR362-mutation, and Fc domain (I400-MND-kozak-IgKVIII-RMP5(miR-A)-Fc-DSS6 SEQ ID NO: 85) has highest ALP secretion per VCN among all vectors compared (SEQ ID NO:55, 87, 88, 84, 89) and is almost 30-fold more potent than the vector EF1-IgG2H-RMP5-Fc (SEQ ID NO: 87) based on ALP secretion per VCN.

FIG. 27 illustrates TNALP secretion per VCN. The results indicate that the combinatory construct including the 400 bps insulator, the MND promoter, the Kozak element, the IgKVIII secretory signal peptide, the miR362-mutation, and Fc domain (I400-MND-kozak-IgKVIII-RMP5(miR-A)-Fc-DSS6 SEQ ID NO: 85) had comparatively increased secretion as compared the I400-MND-IgG2H-RMP5-Fc construct (SEQ ID NO: 87) in 293t cells and in adult mobilized peripheral blood CD34+ hematopoietic stem cells.

FIG. 28 compares measured TNALP secretion per VCN for three different vectors, namely, EF1a-IgG2H-RMP5-Fc (SEQ ID NO: 87), EF1a-IgG2H-sTNALPwt-Fc (SEQ ID NO: 88), and i400-MND-kozak-IgKVIII-RMP5(miR-A)-Fc-DSS6 (SEQ ID NO: 85) in three cell lines—Jurkat, HL60 and THP-1.

FIG. 29 compares measured ALP activity per VCN for four different vectors and mock, namely, 1.EF1a-IgG2H-RMP5-Fc (SEQ ID NO: 87), EF1a-IgG2H-sTNALPwt-Fc (SEQ ID NO: 88), and FIG. 28 compares measured TNALP secretion per VCN for three different vectors, namely, EF1a-IgG2H-RMP5-Fc (SEQ ID NO: 87), EF1a-IgG2H-sTNALPwt-Fc (SEQ ID NO: 88), I400-MND-kozak-IgKVIII-RMP5(miR-A)-Fc-DSS6 (SEQ ID NO: 87), and EF1a-kozak-IgKVIII-RMP5(miR-A)-Fc-DSS6 (SEQ ID NO: 90).

FIG. 30A illustrate measured ALP activity per VCN in HEK293 for three different vectors, namely LVV-RMP100 v01 (referred as EF1a-IgG2H-RMP5-Fc (SEQ ID NO: 87), LVV-RMP200 v02 (referred as FG12-SD400i-MND-Kozak-IgKVIII-AA-RMP5-miR362-A-Fc-(DSS)6 (SEQ ID NO: 85), and LVV-RMP100 v03 (referred as EF1a-kozak-IgKVIII-RMP5(miR-A)-Fc-DSS6, SEQ ID NO: 90). Results shows potency of FG12-SD400i-MND-Kozak-IgKVIII-AA-RMP5-miR362-A-Fc-(DSS)6 (SEQ ID NO: 85) is about 27 fold of EF1a-IgG2H-RMP5-Fc (SEQ ID NO: 87 and more than 10 fold of EF1a-kozak-IgKVIII-RMP5(miR-A)-Fc-DSS6, SEQ ID NO: 90) in HEK293, respectively. FIGS. 30B & 30C illustrate measured ALP activity per VCN and VCN in one health donor-derived HSC, respectively for three different vectors, namely EF1a-Kozak-IgKVIII-RMP5-miR362-A-Fc-Dss6 (SEQ ID NO: 90), FG12-SD400i-MND-Kozak-IgKVIII-AA-RMP5-miR362-A-Fc-(DSS)6 (SEQ ID NO: 85), and MND-RMP100 (referred as LVV-RMP100 or MND-kozak-IgKVIII-RMP5(miR-A)-Fc-DSS6, SEQ ID NO: 74).

FIG. 30 B shows potency of FG12-SD400i-MND-Kozak-IgKVIII-AA-RMP5-miR362-A-Fc-(DSS)6 (SEQ ID NO: 85) is about 2 fold of MND-kozak-IgKVIII-RMP5(miR-A)-Fc-DSS6, SEQ ID NO: 74 and about 8 fold of EF1a-kozak-IgKVIII-RMP5(miR-A)-Fc-DSS6, (SEQ ID NO: 90) in HSC, respectively. FIG. 30C showed transduced HSC have similar VCNs between 4 to 10 in this batch of transduction for those 3 vectors.

FIGS. 31A and 31B illustrate colony formation of HSCs transduced with different vectors, namely (i) I400-MND-RMP100 (referred as i400-MND-kozak-IgKVIII-RMP5(miR-A)-Fc-DSS6, SEQ ID NO: 85), (ii) MND-RMP100 (referred as MND-kozak-IgKVIII-RMP5(miR-A)-Fc-DSS6, SEQ ID NO: 74); and (iii) EF1a-RMP100 (referred as EF1a-kozak-IgKVIII-RMP5(miR-A)-Fc-DSS6, SEQ ID NO: 90). CFU data suggested transduced HSC transduced with MND-400 in-RMP100, EF1a-RMP100 and MND-RMP100 at MOIs from 5 to 20 had similar colony-forming capability compared with mock HSC.

FIGS. 32A, 32B, 33C, and 33D illustrate the stability of various cell lines transduced with different vectors, namely (i) I400-MND-RMP100 (referred as i400-MND-kozak-IgKVIII-RMP5(miR-A)-Fc-DSS6, SEQ ID NO: 85), (ii) MND-RMP100 (referred as MND-kozak-IgKVIII-RMP5(miR-A)-Fc-DSS6, SEQ ID NO: 74); and (iii) EF1a-RMP100 (referred as EF1a-kozak-IgKVIII-RMP5(miR-A)-Fc-DSS6, SEQ ID NO: 90). All constructs showed robust stability in the transduced cell lines THP-1, HL-60 and Jurkat.

FIG. 32A illustrates the stability of the THP-1 cell line.

FIG. 32B illustrates the stability of the Jurkat cell line.

FIG. 32C illustrates the stability of the HL-60 cell line.

FIG. 32D illustrates the stability of the Jurkat cell line.

FIG. 33 provides a vector map of FG12-CD11b-Kozak-IgKVIII-RMP5-miR362-A-Fc-Dss6 (SEQ ID NO: 91).

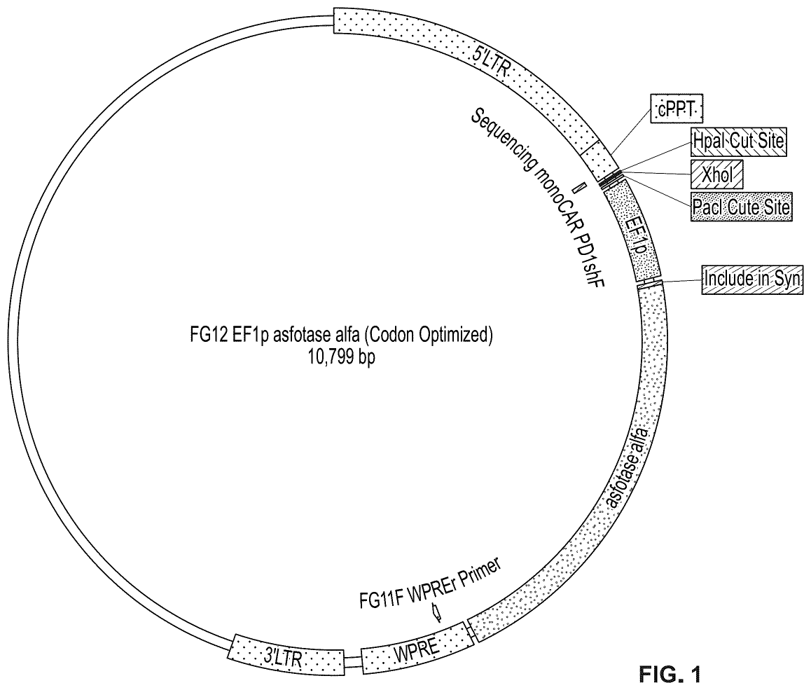

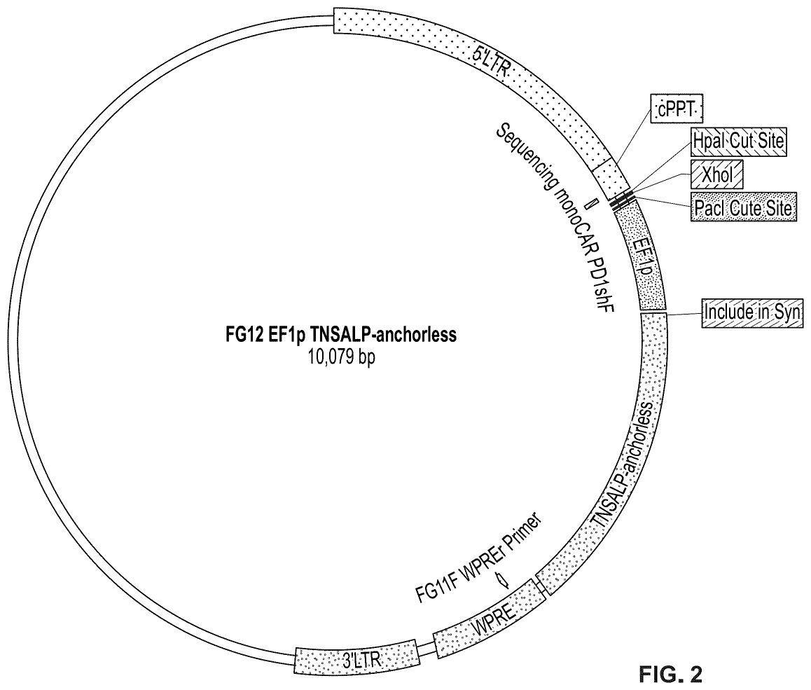

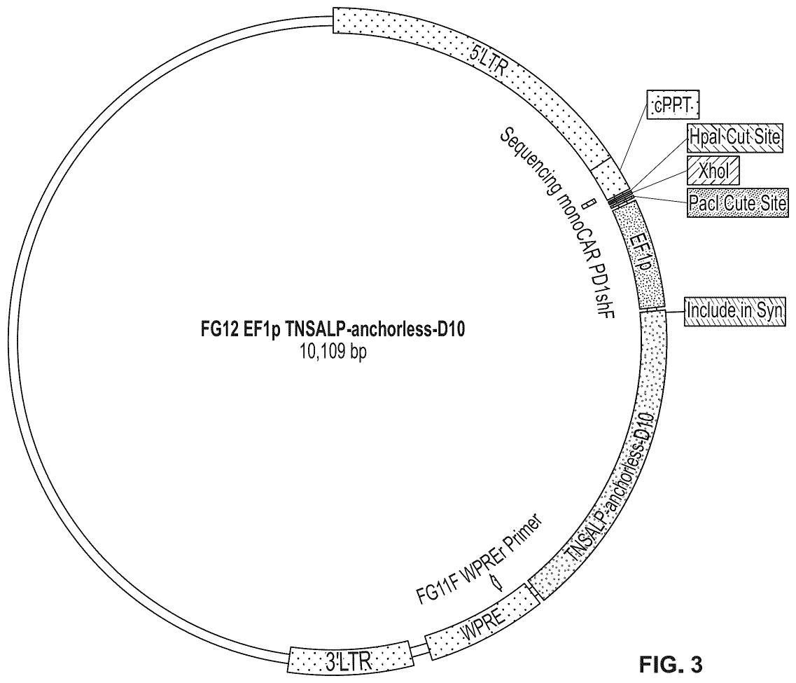

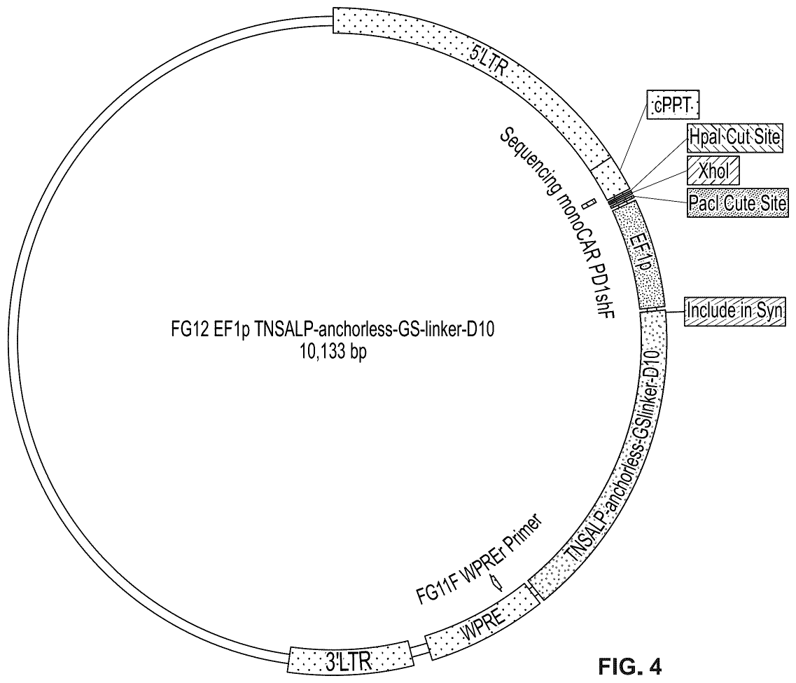

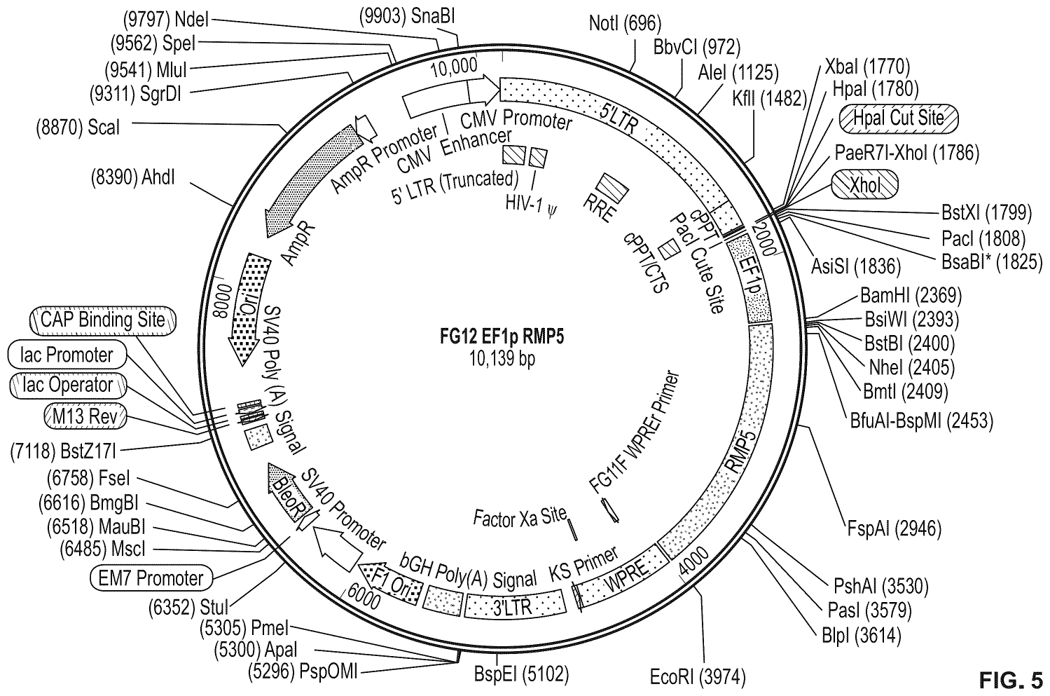

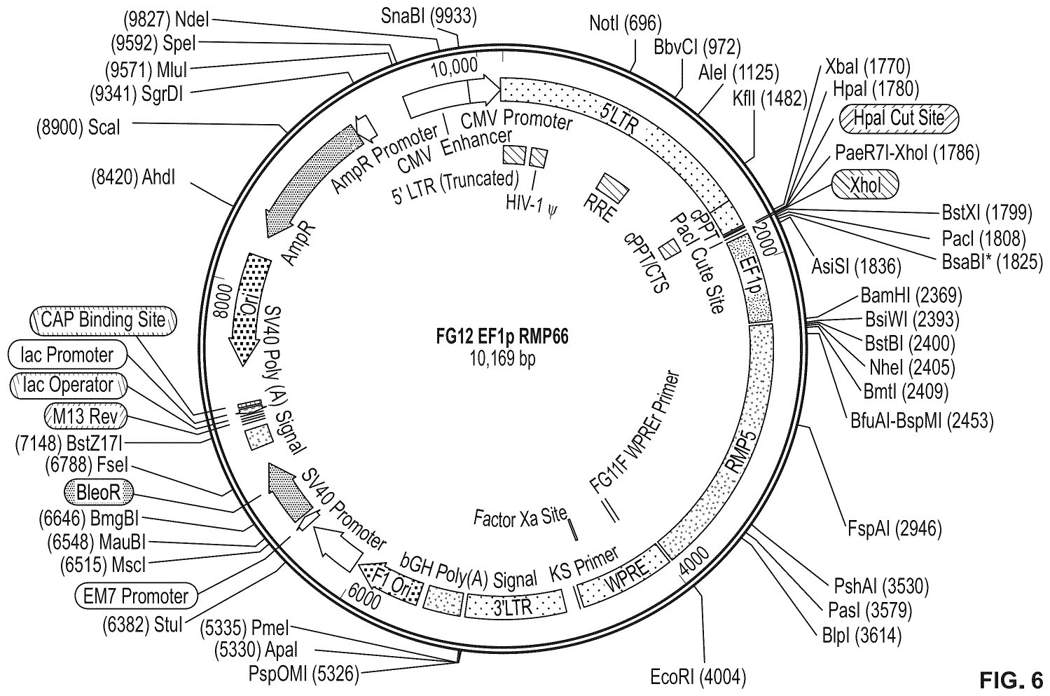

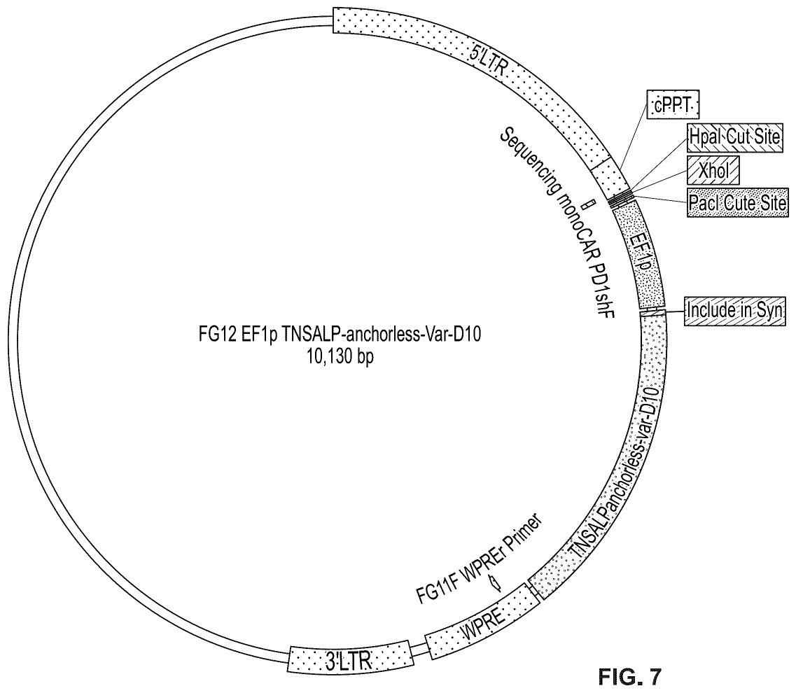

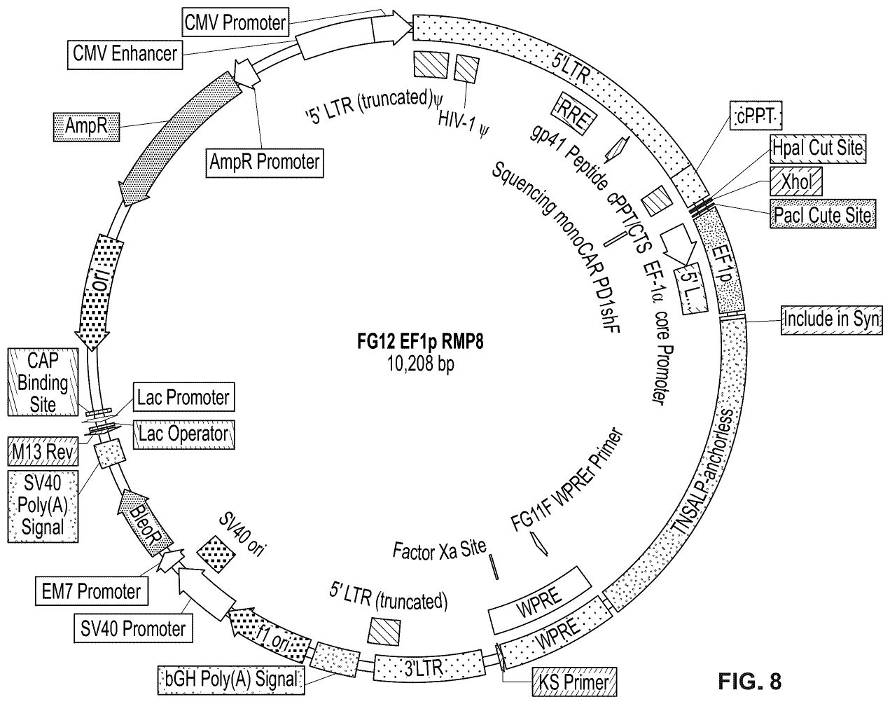

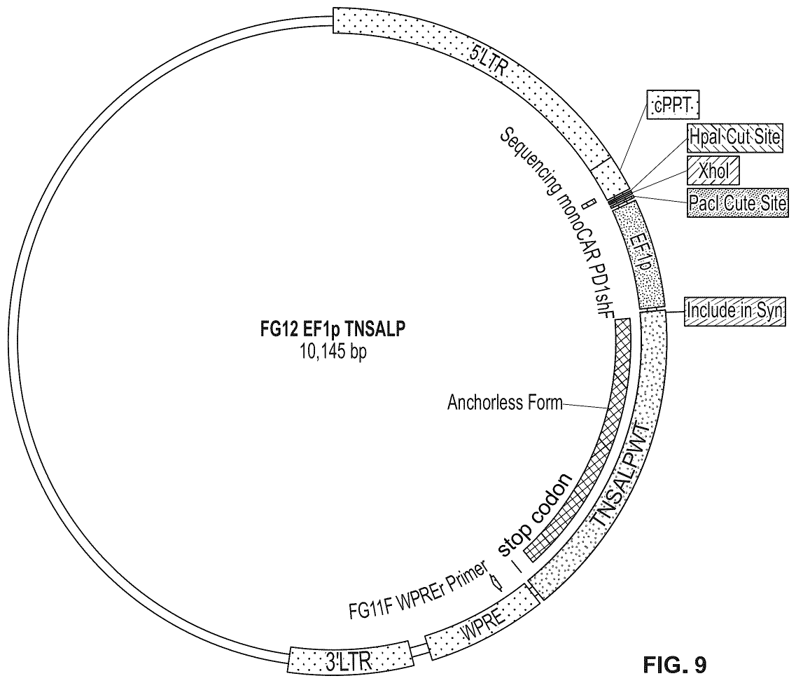

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (e.g. “one or the other but not both”) when preceded by terms of exclusivity, such as “either,” “one of,” “only one of” or “exactly one of.” “Consisting essentially of,” when used in the claims, shall have its ordinary meaning as used in the field of patent law.

As used herein, the terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b, and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “domain” refers to a part of a molecule or structure that shares common physicochemical features, such as, but not limited to, hydrophobic, polar, globular and helical domains or properties. Specific examples of binding domains include, but are not limited to, DNA binding domains and ATP binding domains.

As used herein, the term “Fc” refers to a human IgG Fc domain. Subtypes of IgG such as IgG1, IgG2, IgG3, and IgG4 are all being contemplated for usage as Fc domains. As used herein, the term “fragment,” as applied to a nucleic acid, refers to a subsequence of a larger nucleic acid. A “fragment” of a nucleic acid can be at least about 15 nucleotides in length; for example, at least about SO nucleotides to about 100 nucleotides; at least about 100 to about 500 nucleotides, at least about 500 to about 1000 nucleotides; at least about 1000 nucleotides to about 1500 nucleotides; about 1500 nucleotides to about 2500 nucleotides; or about 2500 nucleotides (and any integer value in between). As used herein, the term “fragment,” as applied to a protein or peptide, refers to a subsequence of a larger protein or peptide. A “fragment” of a protein or peptide can be at least about 20 amino acids in length; for example, at least about 50 amino acids in length; at least about 100 amino acids in length; at least about 200 amino acids in length; at least about 300 amino acids in length; or at least about 400 amino acids in length (and any integer value in between).

As used herein, the terms “hematopoietic cells” or “hematopoietic stem cells” refers to cell types found in the blood and/or lymph. These cell types include the myeloid cells (erythrocytes, thrombocytes, granulocytes (neutrophils, eosinophils, basophils) monocytes and macrophages, mast cells) and the lymphoid cells (B cells, various types of T cells, NK cells). These cells typically arise from hematopoietic stem cells in the bone marrow. It will be appreciated that certain hematopoietic cells, e.g., macrophages, may be present in tissues outside of the vascular or lymphatic systems. White blood cells (e.g., granulocytes (neutrophils, eosinophils, basophils, monocytes, macrophages, mast cells, and lymphoid cells) are a subset of hematopoietic cells.

As used herein, the term “host cell” refers to any cell type that is susceptible to transformation, transfection, transduction, or the like with a nucleic acid construct or expression vector comprising a polynucleotide of the present disclosure. In some embodiments, the term “host cell” encompasses any progeny of a parent cell that is not identical to the parent cell due to mutations that occur during replication. Host cells may include packaging cells, producer cells, and cells infected with viral vectors. In particular embodiments, host cells infected with viral vector of the present disclosure are administered to a subject in need of therapy. In some embodiments, host cells are transduced ex vivo. In other embodiments, host cells are transduced in vivo.

As used herein, the terms “hypophosphatasia” and “HPP” refer to a rare, heritable skeletal disorder caused by, e.g., one or more loss-of-function mutations in the ALPL (alkaline phosphatase, liver/bone/kidney) gene, which encodes tissue-nonspecific alkaline phosphatase (TNSALP). HPP can be further characterized as, e.g., infantile HPP or perinatal HPP (e.g., benign perinatal HPP or lethal perinatal HPP). For instance, “infantile HPP” describes a patient having HPP that is about three years of age or younger, whereas “perinatal HPP” describes a patient having HPP immediately before or after birth (e.g., one to four weeks after birth). The age of onset of HPP, such as when the subject exhibits symptoms of HPP, can also be categorized as, e.g., perinatal-onset HPP and infantile-onset HPP. Patients with HPP can exhibit symptoms of HPP including, but not limited to, skeletal deformity, hypotonia, mobility impairments, gait disturbance, bone deformity, joint pain, bone pain, bone fracture, muscle weakness, muscle pain, rickets (e.g., defects in growth plate cartilage), premature loss of deciduous teeth, incomplete bone mineralization, elevated blood and/or urine levels of phosphoethanolamine (PEA), PPi, pyridoxal 5′-phosphate (PLP), hypomineralization, rachitic ribs, hypercalciuria, short stature, HPP-related seizure, inadequate weight gain, craniosynostosis, and/or calcium pyrophosphate dihydrate crystal deposition (CPPD) in joints leading to, e.g., chondrocalcinosis and premature death. Symptoms of HPP can also include TBM and symptoms of TBM, such as cardio-respiratory arrest, tracheostomy, cardiac arrest, respiratory distress, sputum retention, wheezing, coughing, anoxic spells, cyanosis, bradycardia, tachyarrhythmia, spontaneous hyperextension of the neck, prolonged expiratory breathing phase, failure to thrive, sternal retractions, substernal retractions, intercostal retractions, intermittent or continuous dyspnea, and recurrent bronchitis or pneumonia.

As used therein, the term “lentiviral vector” refers to a non-replicating vector for the transduction of a host cell with a transgene comprising cis-acting lentiviral RNA or DNA sequences, and requiring lentiviral proteins (e.g., Gag, Pol, and/or Env) that are provided in trans. The lentiviral vector lacks expression of functional Gag, Pol, and Env proteins. The lentiviral vector may be present in the form of an RNA or DNA molecule, depending on the stage of production or development of said retroviral vectors.

As used herein, the term “nucleic acid” refers to polynucleotides such as DNA or RNA. Nucleic acids can be single-stranded, partly or completely, double-stranded, and in some cases partly or completely triple-stranded. Nucleic acids include genomic DNA, cDNA, mRNA, etc. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. The term “nucleic acid sequence” as used herein can refer to the nucleic acid material itself and is not restricted to the sequence information (i.e. the succession of letters chosen among the five base letters A, G, C, T, or U) that biochemically characterizes a specific nucleic acid, e.g. a DNA or RNA molecule. A nucleic acid sequence is presented in the 5′ to 3′ direction unless otherwise indicated. The term “nucleic acid segment” is used herein to refer to a nucleic acid sequence that is a portion of a longer nucleic acid sequence.

As used herein, the terms “operably linked” or “operably associated” refer to a functional relationship between two nucleic acids, wherein the expression, activity, localization, etc., of one of the sequences is controlled by, directed by, regulated by, modulated by, etc., the other nucleic acid. The two nucleic acids are said to be operably linked or operably associated or in operable association. “Operably linked” or “operably associated” can also refers to a relationship between two polypeptides wherein the expression of one of the polypeptides is controlled by, directed by, regulated by, modulated by, etc., the other polypeptide. For example, transcription of a nucleic acid is directed by an operably linked promoter; post-transcriptional processing of a nucleic acid is directed by an operably linked processing sequence; translation of a nucleic acid is directed by an operably linked translational regulatory sequence such as a translation initiation sequence; transport, stability, or localization of a nucleic acid or polypeptide is directed by an operably linked transport or localization sequence such as a secretion signal sequence; and post-translational processing of a polypeptide is directed by an operably linked processing sequence. Typically a first nucleic acid sequence that is operably linked to a second nucleic acid sequence, or a first polypeptide that is operatively linked to a second polypeptide, is covalently linked, either directly or indirectly, to such a sequence, although any effective three-dimensional association is acceptable. One of ordinary skill in the art will appreciate that multiple nucleic acids, or multiple polypeptides, may be operably linked or associated with one another.

As used herein, the terms a “packaging signal,” “packaging sequence,” or “psi sequence” refer to any nucleic acid sequence sufficient to direct packaging of a nucleic acid whose sequence comprises the packaging signal into a retroviral particle. The term includes naturally occurring packaging sequences and also engineered variants thereof. Packaging signals of a number of different retroviruses, including lentiviruses, are known in the art.

As used herein, the terms “pharmaceutically acceptable excipient,” “carrier,” or “diluent” refer to pharmaceutical components which do not alter the therapeutic properties of an active agent with which it is administered. One exemplary pharmaceutically acceptable carrier substance is physiological saline. For instance, the pharmaceutically acceptable carrier can include sodium chloride (e.g., 150 mM sodium chloride) and sodium phosphate (e.g., 25 mM sodium phosphate). Other physiologically acceptable excipients, carriers, and diluents, and their formulations, are known to those skilled in the art and described, e.g., in Remington: The Science and Practice of Pharmacy (22nd Ed), Allen (2012). For instance, a pharmaceutically acceptable excipient, carrier, or diluent can include dibasic sodium phosphate, heptahydrate; monobasic sodium phosphate, monohydrate; and sodium chloride at a pH between 7.2 and 7.6.

As used herein, the term “pharmaceutical composition” it is meant a composition containing an active agent as described herein, formulated with at least one pharmaceutically acceptable excipient, carrier, or diluent. The pharmaceutical composition can be manufactured or sold with the approval of a governmental regulatory agency as part of a therapeutic regimen for the treatment or prevention of a disease or event in a patient (e.g., an infant with HPP, such as an infant having perinatal-onset HPP, or an infant having infantile-onset HPP, or juvenile-onset HPP, or a patient having childhood-onset HPP). Pharmaceutical compositions can be formulated, for example, for subcutaneous administration, intravenous administration (e.g., as a sterile solution free of particulate emboli and in a solvent system suitable for intravenous use), for oral administration (e.g., a tablet, capsule, caplet, gelcap, or syrup), or any other formulation described herein, e.g., in unit dosage form.

As used herein, the terms “polynucleotide” or “nucleic acid” refer to messenger RNA (mRNA), RNA, genomic RNA (gRNA), plus strand RNA (RNA(+)), minus strand RNA (RNA(—)), genomic DNA (gDNA), complementary DNA (cDNA) or DNA. Polynucleotides include single and double stranded polynucleotides. In some embodiments, polynucleotides of the present disclosure include polynucleotides or variants having at least about 50%, 55%, 60%, 65%, 70%, 75%, 80%, 85%, 90%, 95%, 96%, 97%, 98%, 99% or 100% sequence identity to any of the reference sequences described herein (see, e.g., Sequence Listing), typically where the variant maintains at least one biological activity of the reference sequence. In various illustrative embodiments, the present disclosure contemplates, in part, viral vector and transfer plasmid polynucleotide sequences and compositions comprising the same. In particular embodiments, the present disclosure provides polynucleotides encoding therapeutic polypeptides.

As used herein, the term “polypeptide,” “polypeptide fragment,” “peptide” and “protein” are used interchangeably, unless specified to the contrary, and according to conventional meaning, i.e., as a sequence of amino acids. Polypeptides are not limited to a specific length, e.g., they may comprise a full-length protein sequence or a fragment of a full length protein, and may include post-translational modifications of the polypeptide, for example, glycosylations, acetylations, phosphorylations and the like, as well as other modifications known in the art, both naturally occurring and non-naturally occurring. In various embodiments, the polypeptides contemplated herein comprise a signal (or leader) sequence at the N-terminal end of the protein, which co-translationally or post-translationally directs transfer of the protein. Polypeptides can be prepared using any of a variety of well-known recombinant and/or synthetic techniques. In some embodiments, the polypeptides contemplated herein encompass alkaline phosphatases, or sequences that have deletions from, additions to, and/or substitutions of one or more amino acid of a CAR as disclosed herein.

Polypeptides include “polypeptide variants.” In some embodiments, polypeptide variants may differ from a naturally occurring polypeptide in one or more substitutions, deletions, additions and/or insertions. Such variants may be naturally occurring or may be synthetically generated, for example, by modifying one or more of the above polypeptide sequences. For example, in particular embodiments, it may be desirable to improve the binding affinity and/or other biological properties of the polypeptides disclosed herein, e.g. the alkaline phosphatase polypeptides, by introducing one or more substitutions, deletions, additions and/or insertions. In some embodiments, polypeptides of the present disclosure include polypeptides having at least about 65%, 70%, 75%, 85%, 90%, 95%, 98%, or 99% amino acid identity thereto.

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

As used herein, the term “promoter,” as used herein, refers to a DNA sequence that determines the site of transcription initiation for an RNA polymerase. Promoter sequences comprise motifs which are recognized and bound by polypeptides, i.e. transcription factors. The said transcription factors shall upon binding recruit RNA polymerases II, preferably, RNA polymerase I, II or III, more preferably, RNA polymerase II or III, and most preferably, RNA polymerase II. Thereby will be initiated the expression of a nucleic acid operatively linked to the transcription control sequence. It is to be understood that dependent on the type of nucleic acid to be expressed, expression as meant herein may comprise transcription of DNA sequences into RNA polynucleotides (as suitable for, e.g., anti-sense approaches, RNAi approaches or ribozyme approaches) or may comprise transcription of DNA sequences into RNA polynucleotides followed by translation of the said RNA polynucleotides into polypeptides (as suitable for, e.g., gene expression and recombinant polypeptide production approaches). In order to govern expression of a nucleic acid sequence, the transcription control sequence may be located immediately adjacent to the nucleic acid to be expressed, i.e. physically linked to the said nucleic acid at its 5′ end. Alternatively, it may be located in physical proximity. In the latter case, however, the sequence must be located so as to allow functional interaction with the nucleic acid to be expressed.

As used herein, the terms “regulatory sequence” or “regulatory element” refer to a nucleic acid sequence that regulates one or more steps in the expression (particularly transcription, but in some cases other events such as splicing or other processing) of nucleic acid sequence(s) with which it is operatively linked. The term includes promoters, enhancers and other transcriptional control elements that direct or enhance transcription of an operatively linked nucleic acid. Regulatory sequences may direct constitutive expression (e.g. expression in most or all cell types under typical physiological conditions in culture or in an organism), cell type specific, lineage specific, or tissue specific expression, and/or regulatable (inducible or repressible) expression. For example, expression may be induced or repressed by the presence or addition of an inducing agent such as a hormone or other small molecule, by an increase in temperature, etc. Non-limiting examples of cell type, lineage, or tissue specific promoters appropriate for use in mammalian cells include lymphoid-specific promoters (see, for example, Calame et al., Adv. Immunol. 43:235, 1988) such as promoters of T cell receptors (see, e.g. Winoto et al., EMBO J. 8:729, 1989) and immunoglobulins (see, for example, Banerji et al., Cell 33:729, 1983; Queen et al., Cell 33:741, 1983), and neuron-specific promoters (e.g., the neurofilament promoter; Byrne et al., Proc. Natl. Acad. Sci. USA 86:5473, 1989). Developmentally-regulated promoters include hox promoters (see, e.g. Kessel et al., Science 249:374, 1990) and the α-fetoprotein promoter (Campes et al., Genes Dev. 3:537, 1989). Some regulatory elements may inhibit or decrease expression of an operatively linked nucleic acid. Such regulatory elements may be referred to as “negative regulatory elements.” A regulatory element whose activity can be induced or repressed by exposure to an inducing or repressing agent and/or by altering environmental conditions is referred to herein as a “regulatable” element.

As used herein, the terms “sALP” and “soluble alkaline phosphatase” refer to a soluble, non-membrane bound ALP or a domain or a biologically active fragment of the soluble, non-membrane bound ALP.

As used herein, the term “signal peptide” refers to a short peptide (about 5 to about 30 amino acids long) at the N-terminus of a polypeptide that directs a polypeptide towards the secretory pathway (e.g., the extracellular space). In some embodiments, the signal peptide is typically cleaved during secretion of the polypeptide. In some embodiments, the signal sequence may direct the polypeptide to an intracellular compartment or organelle. In some embodiments, a signal sequence may be identified by homology, or biological activity, to a peptide with the known function of targeting a polypeptide to a particular region of the cell.

As used herein, the term “subject” refers to any animal subject including laboratory animals (e.g., primates, rats, mice), livestock (e.g., cows, sheep, goats, pigs, turkeys, chickens), household pets (e.g., dogs, cats, rodents, etc.), and humans.

As used herein, the term “therapeutically effective amount” refers to a virus or transduced therapeutic cell may vary according to factors such as the disease state, age, sex, and weight of the individual, and the ability of the stem and progenitor cells to elicit a desired response in the individual. In some embodiments, a therapeutically effective amount is also one in which any toxic or detrimental effects of the virus or transduced therapeutic cells are outweighed by the therapeutically beneficial effects. In some embodiments, the term “therapeutically effective amount” includes an amount that is effective to “treat” a subject (e.g., a patient).

As used herein, the terms “treatment” or “treating” refer to any beneficial or desirable effect on the symptoms or pathology of a disease or pathological condition and may include even minimal reductions in one or more measurable markers of the disease or condition being treated. In some embodiments, the treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, the terms “variants” or “variant” refer to a nucleic acid or polypeptide differing from a reference nucleic acid or polypeptide but retaining essential properties thereof. Generally, variants are overall closely similar, and, in many regions, identical to the reference nucleic acid or polypeptide. Thus, “variant” forms of a transcription factor are overall closely similar, and capable of binding DNA and activate gene transcription.

As used herein, the term “vector” refers to a nucleic acid molecule capable transferring or transporting another nucleic acid molecule. In some embodiments, the transferred nucleic acid is generally linked to, e.g. inserted into, the vector nucleic acid molecule. In some embodiments, a vector may include sequences that direct autonomous replication in a cell or may include sequences sufficient to allow integration into host cell DNA. Useful vectors include, for example, plasmids (typically DNA plasmids, but RNA plasmids are also of use), cosmids, and viral vectors. As will be evident to one of skill in the art, the term “viral vector” is widely used refer either to a nucleic acid molecule (e.g., a plasmid) that includes virus-derived nucleic acid elements that typically facilitate transfer of the nucleic acid molecule or integration into the genome of a cell or to a viral particle that mediates nucleic acid transfer. In some embodiments, the viral particles will typically include various viral components and sometimes also host cell components in addition to nucleic acid(s). In particular, the terms “lentiviral vector,” “lentiviral expression vector,” etc. may be used to refer to lentiviral transfer plasmids and/or lentiviral particles of the present disclosure as described herein.

Polypeptides

In some embodiments, the present disclosure provides for a polypeptide having Formula (I):

[A]ᵥ-[B]-[C]_{w}-[R]_{q}-([D]ₓ-[E]_{y})_{z}, (I)
wherein
A comprises an amino acid sequence encoding a secretion signal peptide;
B comprises an amino acid encoding an alkaline phosphatase;
C comprises an amino acid sequence encoding a GPI anchor;
R is -(Mₒ(Fc)Nₚ)-, where M and N each independently include between 1 and 6 amino acids, where Fc is a Fc domain, and o and p are each independently 0, 1, or 2;
D comprises an amino acid sequence having between 4 and 6 amino acids, or is F(G)ₜF, where each F is the same amino acid, G is an amino acid sequence having 3, 4, or 5 amino acids, and t is an integer ranging from 2-5;
E comprises an amino acid sequence having between 1 and 8 amino acids;
q is 0 or 1;
v is 0 or 1;
w is 0 or 1;
x is 0 or an integer ranging from 1 to 6;
y is 0 or an integer ranging from 1 to 16; and
z is 0 or an integer ranging from 1 to 6.

In some embodiments, the polypeptide of Formula (I) is not conjugated to a dextran.

In some embodiments, the polypeptide of Formula (I) is catalytically competent to allow formation of hydroxyapatite crystals in bone.

In some embodiments, the amino acid encoding the alkaline phosphatase (“[B]”) is a tissue non-specific alkaline phosphatase. In some embodiments, the amino acid encoding the alkaline phosphatase has at least about 85% identity to SEQ ID NO: 11. In other embodiments, the amino acid encoding the alkaline phosphatase has at least about 90% identity to SEQ ID NO: 11. In other embodiments, the amino acid encoding the alkaline phosphatase has at least about 91% identity to SEQ ID NO: 11. In other embodiments, the amino acid encoding the alkaline phosphatase has at least about 92% identity to SEQ ID NO: 11. In other embodiments, the amino acid encoding the alkaline phosphatase has at least about 93% identity to SEQ ID NO: 11. In other embodiments, the amino acid encoding the alkaline phosphatase has at least about 94% identity to SEQ ID NO: 11.

In yet other embodiments, the amino acid encoding the alkaline phosphatase has at least about 95% identity to SEQ ID NO: 11. In further embodiments, the amino acid encoding the alkaline phosphatase has at least about 96% identity to SEQ ID NO: 11. In even further embodiments, the amino acid encoding the alkaline phosphatase has at least about 97% identity to SEQ ID NO: 11. In yet even further embodiments, the amino acid encoding the alkaline phosphatase has at least about 98% identity to SEQ ID NO: 11. In yet even further embodiments, the amino acid encoding the alkaline phosphatase has at least about 99% identity to SEQ ID NO: 11. In yet even further embodiments, the amino acid encoding the alkaline phosphatase has at least about 99% identity to SEQ ID NO: 11 and q is 0. In yet even further embodiments, the amino acid encoding the alkaline phosphatase has at least about 99% identity to SEQ ID NO: 11 and q is 1.

In some embodiments, the amino acid encoding the alkaline phosphatase comprises SEQ ID NO: 11. In some embodiments, the amino acid encoding the alkaline phosphatase comprises SEQ ID NO: 11 and q is 0. In some embodiments, the amino acid encoding the alkaline phosphatase comprises SEQ ID NO: 11 and q is 1.

In some embodiments, the amino acid encoding the alkaline phosphatase comprises a variant of SEQ ID NO: 11, such as a variant comprising one or more amino acid substitutions. In some embodiments, the amino acid encoding the alkaline phosphatase comprises a variant of SEQ ID NO: 11 having a single amino acid substitution. For instance, a variant of SEQ ID NO: 11 may comprises a C102S substitution. By way of another example, a variant of SEQ ID NO: 11 may comprise an E434G substitution. By way of yet another example, a variant of SEQ ID NO: 11 may comprise an A321H substitution. In some embodiments, the amino acid encoding the alkaline phosphatase comprises a variant of SEQ ID NO: 11 having two amino acid substitutions. For instance, a variant of SEQ ID NO: 11 may comprise any two of a C102S substitution, an E434G substitution, or an A321H substitution, e.g. both an A321H substitution and an E434G substitution. In some embodiments, the amino acid encoding the alkaline phosphatase comprises a variant of SEQ ID NO: 11 having three amino acid substitutions. In some embodiments, the amino acid encoding the alkaline phosphatase comprises a variant of SEQ ID NO: 11 having four amino acid substitutions. In some embodiments, the amino acid encoding the alkaline phosphatase comprises a variant of SEQ ID NO: 11 having five amino acid substitutions. In some embodiments, the amino acid encoding the alkaline phosphatase comprises a variant of SEQ ID NO: 11 having six amino acid substitutions.

In some embodiments, the amino acid encoding the secretion signal peptide has at least 90% sequence identity to SEQ ID NO: 12. In other embodiments, the amino acid encoding the secretion signal peptide has at least 95% sequence identity to SEQ ID NO: 12. In yet other embodiments, the amino acid encoding the secretion signal peptide has at least 96% sequence identity to SEQ ID NO: 12. In further embodiments, the amino acid encoding the secretion signal peptide has at least 97% sequence identity to SEQ ID NO: 12. In yet further embodiments, the amino acid encoding the secretion signal peptide has at least 98% sequence identity to SEQ ID NO: 12. In even further embodiments, the amino acid encoding the secretion signal peptide has at least 99% sequence identity to SEQ ID NO: 12. In yet even further embodiments, the amino acid encoding the secretion signal peptide comprises SEQ ID NO: 12.

In some embodiments, the amino acid encoding the secretion signal peptide has at least 90% sequence identity to any one of SEQ ID NOS: 33-43. In other embodiments, the amino acid encoding the secretion signal peptide has at least 95% sequence identity to any one of SEQ ID NOS: 33-43. In yet other embodiments, the amino acid encoding the secretion signal peptide has at least 96% sequence identity to any one of SEQ ID NOS: 33-43. In further embodiments, the amino acid encoding the secretion signal peptide has at least 97% sequence identity to any one of SEQ ID NOS: 33-43. In yet further embodiments, the amino acid encoding the secretion signal peptide has at least 98% sequence identity to any one of SEQ ID NOS: 33-43. In even further embodiments, the amino acid encoding the secretion signal peptide has at least 99% sequence identity to any one of SEQ ID NOS: 33-43. In yet even further embodiments, the amino acid encoding the secretion signal peptide comprises any one of SEQ ID NOS: 33-43.

In some embodiments, the amino acid encoding the GPI anchor has at least 90% sequence identity to SEQ ID NO: 13. In other embodiments, the amino acid encoding the GPI anchor has at least 95% sequence identity to SEQ ID NO: 13. In yet other embodiments, the amino acid encoding the GPI anchor has at least 96% sequence identity to SEQ ID NO: 13. In further embodiments, the amino acid encoding the GPI anchor has at least 97% sequence identity to SEQ ID NO: 13. In yet further embodiments, the amino acid encoding the GPI anchor has at least 98% sequence identity to SEQ ID NO: 13. In even further embodiments, the amino acid encoding the GPI anchor has at least 99% sequence identity to SEQ ID NO: 13. In yet even further embodiments, the amino acid encoding the GPI anchor comprises SEQ ID NO: 13.

In some embodiments, the amino acid encoding the GPI anchor has at least 90% sequence identity to SEQ ID NO: 14. In other embodiments, the amino acid encoding the GPI anchor has at least 95% sequence identity to SEQ ID NO: 14. In yet other embodiments, the amino acid encoding the GPI anchor has at least 96% sequence identity to SEQ ID NO: 14. In further embodiments, the amino acid encoding the GPI anchor has at least 97% sequence identity to SEQ ID NO: 14. In yet further embodiments, the amino acid encoding the GPI anchor has at least 98% sequence identity to SEQ ID NO: 14. In even further embodiments, the amino acid encoding the GPI anchor has at least 99% sequence identity to SEQ ID NO: 14. In yet even further embodiments, the amino acid encoding the GPI anchor comprises SEQ ID NO: 14.

In some embodiments, at least one of v or w is 0 and the amino acid encoding the alkaline phosphatase has at least about 90% identity to SEQ ID NO: 11. In other embodiments, at least one of v or w is 0 and the amino acid encoding the alkaline phosphatase has at least about 95% identity to SEQ ID NO: 11. In further embodiments, at least one of v or w is 0 and the amino acid encoding the alkaline phosphatase has at least about 96% identity to SEQ ID NO: 11. In even further embodiments, at least one of v or w is 0 and the amino acid encoding the alkaline phosphatase has at least about 97% identity to SEQ ID NO: 11. In yet even further embodiments, at least one of v or w is 0 and the amino acid encoding the alkaline phosphatase has at least about 98% identity to SEQ ID NO: 11. In yet even further embodiments, at least one of v or w is 0 and the amino acid encoding the alkaline phosphatase has at least about 99% identity to SEQ ID NO: 11. In other embodiments, at least one of v or w is 0 and the amino acid encoding the alkaline phosphatase comprises SEQ ID NO: 11.

In some embodiments, at least one of v or w is 0, the amino acid encoding the alkaline phosphatase has at least about 90% identity to SEQ ID NO: 11, and x+y=8 to 12. In some embodiments, at least one of v or w is 0, the amino acid encoding the alkaline phosphatase has at least about 95% identity to SEQ ID NO: 11, and x+y=8 to 12. In some embodiments, at least one of v or w is 0, the amino acid encoding the alkaline phosphatase has at least about 96% identity to SEQ ID NO: 11, and x+y=8 to 12. In some embodiments, at least one of v or w is 0, the amino acid encoding the alkaline phosphatase has at least about 97% identity to SEQ ID NO: 11, and x+y=8 to 12. In some embodiments, at least one of v or w is 0, the amino acid encoding the alkaline phosphatase has at least about 98% identity to SEQ ID NO: 11, and x+y=8 to 12. In some embodiments, at least one of v or w is 0, the amino acid encoding the alkaline phosphatase has at least about 99% identity to SEQ ID NO: 11, and x+y=8 to 12. In some embodiments, at least one of v or w is 0, the amino acid encoding the alkaline phosphatase comprises SEQ ID NO: 11, and x+y=8 to 12. In some embodiments, at least one of v or w is 0, the amino acid encoding the alkaline phosphatase comprises SEQ ID NO: 11, and x+y=8 to 12.

In some embodiments, at least one of v or w is 0, the amino acid encoding the alkaline phosphatase comprises SEQ ID NO: 11, and x+y=8 to 12, and q is 1. In some embodiments, at least one of v or w is 0, the amino acid encoding the alkaline phosphatase comprises SEQ ID NO: 11, and x+y=8 to 12, q is 1; and R is (-[LK]-Fc-[DI]-). In some embodiments, at least one of v or w is 0, the amino acid encoding the alkaline phosphatase comprises SEQ ID NO: 11, and x+y=8 to 12, q is 1; and R is (-[LK]-Fc-[DI]-), and [A] comprises an amino acid sequence having at least 95% identity to any one of SEQ ID NOS: 12 and 33-43. In some embodiments, at least one of v or w is 0, the amino acid encoding the alkaline phosphatase comprises SEQ ID NO: 11, and x+y=8 to 12, q is 1; and R is (-[LK]-Fc-[DI]-), and [A] comprises an amino acid sequence having at least 96% identity to any one of SEQ ID NOS: 12 and 33-43. In some embodiments, at least one of v or w is 0, the amino acid encoding the alkaline phosphatase comprises SEQ ID NO: 11, and x+y=8 to 12, q is 1; and R is (-[LK]-Fc-[DI]-), and [A] comprises an amino acid sequence having at least 97% identity to any one of SEQ ID NOS: 12 and 33-43. In some embodiments, at least one of v or w is 0, the amino acid encoding the alkaline phosphatase comprises SEQ ID NO: 11, and x+y=8 to 12, q is 1; and R is (-[LK]-Fc-[DI]-), and [A] comprises an amino acid sequence having at least 98% identity to any one of SEQ ID NOS: 12 and 33-43. In some embodiments, at least one of v or w is 0, the amino acid encoding the alkaline phosphatase comprises SEQ ID NO: 11, and x+y=8 to 12, q is 1; and R is (-[LK]-Fc-[DI]-), and [A] comprises an amino acid sequence having at least 99% identity to any one of SEQ ID NOS: 12 and 33-43. In some embodiments, at least one of v or w is 0, the amino acid encoding the alkaline phosphatase comprises SEQ ID NO: 11, and x+y=8 to 12, q is 1; and R is (-[LK]-Fc-[DI]-), and [A] comprises an amino acid sequence having any one of SEQ ID NOS: 12 and 33-43.

In some embodiments, v is 1 and w is 0, the amino acid encoding the alkaline phosphatase has at least about 90% identity to SEQ ID NO: 11, and x+y=8 to 12. In some embodiments, v is 1 and w is 0, the amino acid encoding the alkaline phosphatase has at least about 95% identity to SEQ ID NO: 11, and x+y=8 to 12. In some embodiments, v is 1 and w is 0, the amino acid encoding the alkaline phosphatase has at least about 96% identity to SEQ ID NO: 11, and x+y=8 to 12. In some embodiments, v is 1 and w is 0, the amino acid encoding the alkaline phosphatase has at least about 97% identity to SEQ ID NO: 11, and x+y=8 to 12. In some embodiments, v is 1 and w is 0, the amino acid encoding the alkaline phosphatase has at least about 98% identity to SEQ ID NO: 11, and x+y=8 to 12. In some embodiments, v is 1 and w is 0, the amino acid encoding the alkaline phosphatase has at least about 99% identity to SEQ ID NO: 11, and x+y=8 to 12. In some embodiments, v is 1 and w is 0, the amino acid encoding the alkaline phosphatase comprises SEQ ID NO: 11, and x+y=8 to 12. In some embodiments, v is 1 and w is 0, the amino acid encoding the alkaline phosphatase comprises SEQ ID NO: 11, and x+y=8 to 12 and q is 1.

In some embodiments, the amino acid encoding the alkaline phosphatase has at least about 95% identity to SEQ ID NO: 11, x+y=8 to 12, and where x and y are both at least 1. In some embodiments, the amino acid encoding the alkaline phosphatase has at least about 97% identity to SEQ ID NO: 11, x+y=8 to 12, and where x and y are both at least 1. In some embodiments, the amino acid encoding the alkaline phosphatase has at least about 99% identity to SEQ ID NO: 11, x+y=8 to 12, and where x and y are both at least 1. In some embodiments, the amino acid encoding the alkaline phosphatase has at least about 99% identity to SEQ ID NO: 11, x+y=8 to 12, where x and y are both at least 1; and q is 0. In some embodiments, the amino acid encoding the alkaline phosphatase has at least about 99% identity to SEQ ID NO: 11, x+y=8 to 12, where x and y are both at least 1; and q is 1. In some embodiments, the amino acid encoding the alkaline phosphatase has at least about 99% identity to SEQ ID NO: 11, and x+y=8 to 12, where x and y are both at least 1; q is 1; and R is (-[LK]-Fc-[DI]-).

In some embodiments, at least one of v or w is 0, the amino acid encoding the alkaline phosphatase has at least about 90% identity to SEQ ID NO: 11, and x+y=4 to 8. In some embodiments, at least one of v or w is 0, the amino acid encoding the alkaline phosphatase has at least about 95% identity to SEQ ID NO: 11, and x+y=4 to 8. In some embodiments, at least one of v or w is 0, the amino acid encoding the alkaline phosphatase has SEQ ID NO: 11, and x+y=4 to 8.

In some embodiments, v is 1, w is 0, the amino acid encoding the alkaline phosphatase has at least about 90% identity to SEQ ID NO: 11, and x+y=4 to 8. In some embodiments, v is 1, w is 0, the amino acid encoding the alkaline phosphatase has at least about 95% identity to SEQ ID NO: 11, and x+y=4 to 8. In some embodiments, v is 1, w is 0, the amino acid encoding the alkaline phosphatase has at least about 96% identity to SEQ ID NO: 11, and wherein x+y=4 to 8. In some embodiments, v is 1, w is 0, the amino acid encoding the alkaline phosphatase has at least about 97% identity to SEQ ID NO: 11, and x+y=4 to 8. In some embodiments, v is 1, w is 0, the amino acid encoding the alkaline phosphatase has at least about 98% identity to SEQ ID NO: 11, and x+y=4 to 8. In some embodiments, v is 1, w is 0, the amino acid encoding the alkaline phosphatase has at least about 99% identity to SEQ ID NO: 11, and x+y=4 to 8. In some embodiments, v is 1, w is 0, the amino acid encoding the alkaline phosphatase comprises SEQ ID NO: 11, and x+y=4 to 8.

In some embodiments, v is 1, w is 0, the amino acid encoding the alkaline phosphatase comprises SEQ ID NO: 11, x+y=4 to 8 and q is 0.

In some embodiments, v is 1, w is 0, the amino acid encoding the alkaline phosphatase comprises SEQ ID NO: 11, x+y=4 to 8 and q is 1. In some embodiments, v is 1, w is 0, the amino acid encoding the alkaline phosphatase comprises SEQ ID NO: 11, x+y=4 to 8, q is 1; and R is (-[LK]-Fc-[DI]-). In some embodiments, v is 1, w is 0, the amino acid encoding the alkaline phosphatase comprises SEQ ID NO: 11, x+y=5 to 7, q is 1; and R is (-[LK]-Fc-[DI]-). In some embodiments, v is 1, w is 0, the amino acid encoding the alkaline phosphatase comprises SEQ ID NO: 11, x+y=6, q is 1; and R is (-[LK]-Fc-[DI]-). In some embodiments, v is 1, w is 0, the amino acid encoding the alkaline phosphatase comprises SEQ ID NO: 11, x+y=6, q is 1; R is (-[LK]-Fc-[DI]-), and [A] comprises an amino acid sequence having at least 95% identity to any one of SEQ ID NOS: 12 and 33-43. In some embodiments, v is 1, w is 0, the amino acid encoding the alkaline phosphatase comprises SEQ ID NO: 11, x+y=6, q is 1; R is (-[LK]-Fc-[DI]-), and [A] comprises an amino acid sequence having at least 96% identity to any one of SEQ ID NOS: 12 and 33-43. In some embodiments, v is 1, w is 0, the amino acid encoding the alkaline phosphatase comprises SEQ ID NO: 11, x+y=6, q is 1; R is (-[LK]-Fc-[DI]-), [A] comprises an amino acid sequence having at least 97% identity to any one of SEQ ID NOS: 12 and 33-43. In some embodiments, v is 1, w is 0, the amino acid encoding the alkaline phosphatase comprises SEQ ID NO: 11, x+y=6 and q is 1; R is (-[LK]-Fc-[DI]-), and [A] comprises an amino acid sequence having at least 98% identity to any one of SEQ ID NOS: 12 and 33-43. In some embodiments, v is 1, w is 0, the amino acid encoding the alkaline phosphatase comprises SEQ ID NO: 11, x+y=6, q is 1; R is (-[LK]-Fc-[DI]-), and [A] comprises an amino acid sequence having at least 99% identity to any one of SEQ ID NOS: 12 and 33-43. In some embodiments, v is 1, w is 0, the amino acid encoding the alkaline phosphatase comprises SEQ ID NO: 11, x+y=6, q is 1; R is (-[LK]-Fc-[DI]-) and [A] comprises any one of SEQ ID NOS: 12 and 33-43.

In some embodiments, at least one of v or w is 0; q is 1; [E]_{y} is [DSS]₆; and the amino acid encoding the alkaline phosphatase comprises SEQ ID NO: 11. In some embodiments, at least one of v or w is 0; q is 1; [E]_{y} is [DSS]₆; the amino acid encoding the alkaline phosphatase comprises SEQ ID NO: 11; and [A] comprises at least 99% identity to any one of SEQ ID NOS: 12 and 33-43. In some embodiments, at least one of v or w is 0; q is 1; [E]_{y} is [DSS]₆; the amino acid encoding the alkaline phosphatase comprises SEQ ID NO: 11; and [A] comprises any one of SEQ ID NOS: 12 and 33-43.

In some embodiments, [A]ᵥ-[B]-[C]_{w} comprises an amino acid sequence having at least 85% sequence identity to SEQ ID NO: 10. In some embodiments, [A]ᵥ-[B]-[C]_{w} comprises an amino acid sequence having at least 90% sequence identity to SEQ ID NO: 10. In some embodiments, [A]ᵥ-[B]-[C]_{w} comprises an amino acid sequence having at least 95% sequence identity to SEQ ID NO: 10. In some embodiments, [A]ᵥ-[B]-[C]_{w} comprises an amino acid sequence having at least 96% sequence identity to SEQ ID NO: 10. In some embodiments, [A]ᵥ-[B]-[C]_{w} comprises an amino acid sequence having at least 97% sequence identity to SEQ ID NO: 10. In some embodiments, [A]ᵥ-[B]-[C]_{w} comprises an amino acid sequence having at least 98% sequence identity to SEQ ID NO: 10. In some embodiments, [A]ᵥ-[B]-[C]_{w} comprises an amino acid sequence having at least 99% sequence identity to SEQ ID NO: 10. In some embodiments, [A]ᵥ-[B]-[C]_{w} comprises an amino acid sequence comprising SEQ ID NO: 10.

In some embodiments, [A]ᵥ-[B]-[C]_{w} comprises an amino acid sequence having at least 85% sequence identity to any one of SEQ ID NOS: 27-32. In some embodiments, [A]ᵥ-[B]-[C]_{w} comprises an amino acid sequence having at least 90% sequence identity to any one of SEQ ID NOS: 27-32. In some embodiments, [A]ᵥ-[B]-[C]_{w} comprises an amino acid sequence having at least 95% sequence identity to any one of SEQ ID NOS: 27-32. In some embodiments, [A]ᵥ-[B]-[C]_{w} comprises an amino acid sequence having at least 96% sequence identity to any one of SEQ ID NOS: 27-32. In some embodiments, [A]ᵥ-[B]-[C]_{w} comprises an amino acid sequence having at least 97% sequence identity to any one of SEQ ID NOS: 27-32. In some embodiments, [A]ᵥ-[B]-[C]_{w} comprises an amino acid sequence having at least 98% sequence identity to any one of SEQ ID NOS: 27-32. In some embodiments, [A]ᵥ-[B]-[C]_{w} comprises an amino acid sequence having at least 99% sequence identity to any one of SEQ ID NOS: 27-32. In some embodiments, [A]ᵥ-[B]-[C]_{w} comprises an amino acid sequence comprising any one of SEQ ID NOS: 27-32.

In some embodiments, the Fc domain has at least 90% sequence identity to that of SEQ ID NO: 130. In some embodiments, the Fc domain has at least 91% sequence identity to that of SEQ ID NO: 130. In some embodiments, the Fc domain has at least 92% sequence identity to that of SEQ ID NO: 130. In some embodiments, the Fc domain has at least 93% sequence identity to that of SEQ ID NO: 130. In some embodiments, the Fc domain has at least 94% sequence identity to that of SEQ ID NO: 130. In some embodiments, the Fc domain has at least 95% sequence identity to that of SEQ ID NO: 130. In some embodiments, the Fc domain has at least 96% sequence identity to that of SEQ ID NO: 130. In some embodiments, the Fc domain has at least 97% sequence identity to that of SEQ ID NO: 130. In some embodiments, the Fc domain has at least 98% sequence identity to that of SEQ ID NO: 130. In some embodiments, the Fc domain has at least 99% sequence identity to that of SEQ ID NO: 130. In some embodiments, the Fc domain comprises SEQ ID NO: 130. In some embodiments, the Fc domain comprises SEQ ID NO: 130 and y ranges from 4 to 6. In some embodiments, the Fc domain comprises SEQ ID NO: 130 and [E]_{y} is [DSS]₆. In some embodiments, the R comprises SEQ ID NO: 9 and [E]_{y} is [DSS]₆.

In some embodiments, M comprises one or more amino acids selected from glycine, serine, threonine, alanine, lysine, and glutamic acid. In some embodiments, M comprises 2 amino acids. For example, M may comprise leucine and lysine. In some embodiments, M is leucine-lysine. By way of another example, M may comprise two alanine amino acids. In other embodiments, M comprises three amino acids.

In some embodiments, N comprises one or more amino acids selected from glycine, serine, threonine, alanine, lysine, and glutamic acid. In some embodiments, N comprises 2 amino acids (i.e., a diamino acid). For example, N may comprise aspartic acid and isoleucine. By way of another example, N may comprise two alanine amino acids. In some embodiments, N is aspartic acid—isoleucine. In other embodiments, N comprises 3 amino acids.

In some embodiments, M is leucine-lysine and N is aspartic acid—isoleucine. In some embodiments, M is leucine-lysine and N is aspartic acid—isoleucine and Fc has at least 90% sequence identity to that of SEQ ID NO: 130. In some embodiments, M is leucine-lysine and N is aspartic acid—isoleucine and Fc has at least 91% sequence identity to that of SEQ ID NO: 130. In some embodiments, M is leucine-lysine and N is aspartic acid—isoleucine and Fc has at least 92% sequence identity to that of SEQ ID NO: 130. In some embodiments, M is leucine-lysine and N is aspartic acid—isoleucine and Fc has at least 93% sequence identity to that of SEQ ID NO: 130. In some embodiments, M is leucine-lysine and N is aspartic acid—isoleucine and Fc has at least 94% sequence identity to that of SEQ ID NO: 130. In some embodiments, M is leucine-lysine and N is aspartic acid—isoleucine and Fc has at least 95% sequence identity to that of SEQ ID NO: 130. In some embodiments, M is leucine-lysine and N is aspartic acid—isoleucine and Fc has at least 96% sequence identity to that of SEQ ID NO: 130. In some embodiments, M is leucine-lysine and N is aspartic acid—isoleucine and Fc has at least 97% sequence identity to that of SEQ ID NO: 130. In some embodiments, M is leucine-lysine and N is aspartic acid—isoleucine and Fc has at least 98% sequence identity to that of SEQ ID NO: 130. In some embodiments, M is leucine-lysine and N is aspartic acid—isoleucine and Fc has at least 99% sequence identity to that of SEQ ID NO: 130. In some embodiments, M is leucine-lysine and N is aspartic acid—isoleucine and Fc comprises SEQ ID NO: 130.

In some embodiments, M is leucine-lysine and N is aspartic acid—isoleucine; and o, p, and q are each 1. In some embodiments, M is leucine-lysine and N is aspartic acid—isoleucine and Fc has at least 90% sequence identity to that of SEQ ID NO: 130; and o, p, and q are each 1. In some embodiments, M is leucine-lysine and N is aspartic acid—isoleucine and Fc has at least 91% sequence identity to that of SEQ ID NO: 130; and o, p, and q are each 1. In some embodiments, M is leucine-lysine and N is aspartic acid—isoleucine and Fc has at least 92% sequence identity to that of SEQ ID NO: 130; and o, p, and q are each 1. In some embodiments, M is leucine-lysine and N is aspartic acid—isoleucine and Fc has at least 93% sequence identity to that of SEQ ID NO: 130; and o, p, and q are each 1. In some embodiments, M is leucine-lysine and N is aspartic acid—isoleucine and Fc has at least 94% sequence identity to that of SEQ ID NO: 130; and o, p, and q are each 1. In some embodiments, M is leucine-lysine and N is aspartic acid—isoleucine and Fc has at least 95% sequence identity to that of SEQ ID NO: 130; and o, p, and q are each 1. In some embodiments, M is leucine-lysine and N is aspartic acid—isoleucine and Fc has at least 96% sequence identity to that of SEQ ID NO: 130; and o, p, and q are each 1. In some embodiments, M is leucine-lysine and N is aspartic acid—isoleucine and Fc has at least 97% sequence identity to that of SEQ ID NO: 130; and o, p, and q are each 1. In some embodiments, M is leucine-lysine and N is aspartic acid—isoleucine and Fc has at least 98% sequence identity to that of SEQ ID NO: 130; and o, p, and q are each 1. In some embodiments, M is leucine-lysine and N is aspartic acid—isoleucine and Fc has at least 99% sequence identity to that of SEQ ID NO: 130; and o, p, and q are each 1. In some embodiments, M is leucine-lysine; N is aspartic acid—isoleucine and Fc comprises SEQ ID NO: 130; and o, p, and q are each 1. In some embodiments, M is leucine-lysine; N is aspartic acid—isoleucine; Fc comprises SEQ ID NO: 130; o, p, and q are each 1; and [A] has at least 99% identity to any one of SEQ ID NOS: 12 and 33-43. In some embodiments, M is leucine-lysine; N is aspartic acid—isoleucine; Fc comprises SEQ ID NO: 130; o, p, and q are each 1; and [A] comprises any one of SEQ ID NOS: 12 and 33-43.

In some embodiments, M is leucine-lysine and N is aspartic acid—isoleucine; o, p, q, and z are each 1; and x is 0. In some embodiments, M is leucine-lysine and N is aspartic acid—isoleucine and Fc has at least 90% sequence identity to that of SEQ ID NO: 130; o, p, q, and z are each 1; and x is 0. In some embodiments, M is leucine-lysine and N is aspartic acid—isoleucine and Fc has at least 91% sequence identity to that of SEQ ID NO: 130; o, p, q, and z are each 1; and x is 0. In some embodiments, M is leucine-lysine and N is aspartic acid—isoleucine and Fc has at least 92% sequence identity to that of SEQ ID NO: 130; o, p, q, and z are each 1; and x is 0. In some embodiments, M is leucine-lysine and N is aspartic acid—isoleucine and Fc has at least 93% sequence identity to that of SEQ ID NO: 130; o, p, q, and z are each 1; and x is 0. In some embodiments, M is leucine-lysine and N is aspartic acid—isoleucine and Fc has at least 94% sequence identity to that of SEQ ID NO: 130; o, p, q, and z are each 1; and x is 0. In some embodiments, M is leucine-lysine and N is aspartic acid—isoleucine and Fc has at least 95% sequence identity to that of SEQ ID NO: 130; o, p, q, and z are each 1; and x is 0. In some embodiments, M is leucine-lysine and N is aspartic acid—isoleucine and Fc has at least 96% sequence identity to that of SEQ ID NO: 130; o, p, q, and z are each 1; and x is 0. In some embodiments, M is leucine-lysine and N is aspartic acid—isoleucine and Fc has at least 97% sequence identity to that of SEQ ID NO: 130; o, p, q, and z are each 1; and x is 0. In some embodiments, M is leucine-lysine and N is aspartic acid—isoleucine and Fc has at least 98% sequence identity to that of SEQ ID NO: 130; o, p, q, and z are each 1; and x is 0. In some embodiments, M is leucine-lysine and N is aspartic acid—isoleucine and Fc has at least 99% sequence identity to that of SEQ ID NO: 130; o, p, q, and z are each 1; and x is 0. In some embodiments, M is leucine-lysine and N is aspartic acid—isoleucine and Fc comprises SEQ ID NO: 130; o, p, q, and z are each 1; and x is 0. In some embodiments, M is leucine-lysine and N is aspartic acid—isoleucine and Fc comprises SEQ ID NO: 130; o, p, q, and z are each 1; x is 0; and [A] has at least 99% identity to any one of SEQ ID NOS: 12 and 33-43. In some embodiments, M is leucine-lysine and N is aspartic acid—isoleucine and Fc comprises SEQ ID NO: 130; o, p, q, and z are each 1; x is 0; and [A] comprises any one of SEQ ID NOS: 12 and 33-43.

In some embodiments, [R] comprises at least 90% identity to SEQ ID NO: 9. In some embodiments, [R] comprises at least 95% identity to SEQ ID NO: 9. In some embodiments, [R] comprises at least 96% identity to SEQ ID NO: 9. In some embodiments, [R] comprises at least 97% identity to SEQ ID NO: 9. In some embodiments, [R] comprises at least 98% identity to SEQ ID NO: 9. In some embodiments, [R] comprises at least 99% identity to SEQ ID NO: 9. In some embodiments, [R] comprises SEQ ID NO: 9.

In some embodiments, D is an amino acid sequence comprising four amino acids. In some embodiments, each of the four amino acids is the same. In other embodiments, three of the four amino acids are the same. In yet other embodiments, two of the four amino acids are the same. In some embodiments, at least two contiguous amino acids of the four amino acids are the same. In other embodiments, at least three contiguous amino acids of the four amino acids are the same. In some embodiments, the four amino acids are selected from glycine, serine, threonine, alanine, lysine, and glutamic acid. In some embodiments, D comprises 4 amino acids, and x ranges from 1 to 6. In some embodiments, D comprises 4 amino acids, and x ranges from 1 to 4. In some embodiments, D comprises 4 amino acids, and x is 1 or 2. In some embodiments, D comprises 4 amino acids, and x is 2. In some embodiments, D comprises 4 amino acids and x is 1. In some embodiments, D comprises 4 amino acids, and x is 1 or 2, and q is 0. In some embodiments, D comprises 4 amino acids, and x is 2 and q is 0. In some embodiments, D comprises 4 amino acids and x is 1 and q is 0. In some embodiments, D comprises 4 amino acids and x is 1 and q is 1.

In some embodiments, D is GGGS. In other embodiments, D is GGSS. In yet other embodiments, D is GSSS. In further embodiments, D is GSGS. In some embodiments, D is GGGS and x is an integer ranging from 1 to 4. In some embodiments, D is GGGS and x is an integer ranging from 1 to 3. In some embodiments, D is GGGS and x is 1 or 2. In some embodiments, D is GGGS, x is an integer ranging from 1 to 4, and y is an integer ranging from 1 to 8. In some embodiments, D is GGGS, x is an integer ranging from 1 to 3, and y is an integer ranging from 1 to 8. In some embodiments, D is GGGS, x is 1 or 2, and y is an integer ranging from 1 to 8. In some embodiments, D is GGGS, x is 1 or 2, and y is an integer ranging from 1 to 8 and q is 0. In some embodiments, D is GGGS, x is 1 or 2, y is an integer ranging from 1 to 8 and q is 1. In some embodiments, D is GGGS, x is 1 or 2, y is an integer ranging from 1 to 8, q is 1, and R is (-[LK]-Fc-[DI]-). In some embodiments, D is GGGS, x is 2, y is an integer ranging from 1 to 8, q is 1, and R is (-[LK]-Fc-[DI]-). In some embodiments, D is GGGS, z is 1, x is 2, y is an integer ranging from 1 to 8, q is 1, and R is (-[LK]-Fc-[DI]-).

In some embodiments, D is an amino acid sequence comprising five amino acids. In some embodiments, each of the five amino acids is the same. In some embodiments, four of the five amino acids are the same. In other embodiments, three of the five amino acids are the same. In other embodiments, two of the five amino acids are the same. In some embodiments, at least two contiguous amino acids of the five amino acids are the same. In other embodiments, at least three contiguous amino acids of the five amino acids are the same. In yet other embodiments, four contiguous amino acids of the five amino acids are the same. In some embodiments, the five amino acids are selected from glycine, serine, threonine, alanine, lysine, and glutamic acid. In some embodiments, D comprises 5 amino acids, and x ranges from 1 to 6. In some embodiments, D comprises 5 amino acids, and x ranges from 1 to 4. In some embodiments, D comprises 5 amino acids, and x is 1 or 2. In some embodiments, D comprises 5 amino acids and x is 1. In some embodiments, D comprises 5 amino acids and x is 1 and q is 0. In some embodiments, D comprises 5 amino acids, x is 1, and q is 1. In some embodiments, D comprises 5 amino acids, x is 1, and q is 1 and R is (-[LK]-Fc-[DI]-).

In some embodiments, D is GGGGS. In other embodiments, D is GGGSS. In yet other embodiments, D is GGSSS. In further embodiments, D is GGSGS. In even further embodiments, D is GGSGS. In some embodiments, D is GGGGS or GGGSS, and wherein x is an integer ranging from 1 to 4. In some embodiments, D is GGGGS or GGGSS, and wherein x is an integer ranging from 1 to 3. In some embodiments, D is GGGGS or GGGSS, and wherein x is 1 or 2. In some embodiments, D is GGGGS or GGGSS, and wherein x is an integer ranging from 1 to 4, and wherein y is an integer ranging from between 1 and 8. In some embodiments, D is GGGGS or GGGSS, and wherein x is an integer ranging from 1 to 3 and wherein y is an integer ranging from between 1 and 8. In some embodiments, D is GGGGS or GGGSS, and wherein x is 1 or 2, and wherein y is an integer ranging from between 1 and 8.

In some embodiments, D is GGGGS or GGGSS, and wherein x is an integer ranging from 1 to 3, E is -D-S-S-, and wherein y is an integer ranging from between 1 and 8. In some embodiments, D is GGGGS or GGGSS, and wherein x is 1 or 2, E is -D-S-S-, and wherein y is an integer ranging from between 1 and 8. In some embodiments, D is GGGGS or GGGSS, and wherein x is an integer ranging from 1 to 3, E is aspartic acid, and wherein y is an integer ranging from between 8 and 12. In some embodiments, D is GGGGS or GGGSS, and wherein x is 1 or 2, E is aspartic acid, and wherein y is 10. In some embodiments, D is GGGGS, and wherein x is 1 or 2, E is aspartic acid, and wherein y ranges from 4 to 16. In some embodiments, D is GGGGS, and wherein x is 1 or 2, E is aspartic acid, and wherein y ranges from 8 to 12. In some embodiments, D is GGGGS, and wherein x is 1 or 2, E is aspartic acid, and wherein y is 10. In some embodiments, D is GGGGS, and wherein x is 1 or 2, E is aspartic acid, and wherein y is 10 and q is 0. In some embodiments, D is GGGGS, and wherein x is 1 or 2, E is aspartic acid, and wherein y is 10 and q is 1. In some embodiments, D is GGGGS, and wherein x is 1 or 2, E is aspartic acid, and wherein y is 10 and q is 1 and R is (-[LK]-Fc-[DI]-).

In some embodiments, D is EAAAK. In some embodiments, D is EAAAK, and wherein x is an integer ranging from 2 to 5. In other embodiments, D is EAAAK, wherein x is an integer ranging from 1 to 4. In yet other embodiments, D is EAAAK, wherein x is an integer ranging from 1 to 3. In further embodiments, D is EAAAK, wherein x is 1 or 2.

In some embodiments, D is F(G)ₜF, where each F is the same amino acid, G is an amino acid sequence having 3, 4, or 5 amino acids, and t is an integer ranging from 2-5. In some embodiments, each F is alanine. In some embodiments, G is EAAAK. In some embodiments, each F is alanine, G is EAAAK, and t is 2 or 3. In some embodiments, each F is alanine, G is EAAAK, t is 2 or 2, and x is 1 or 2. In some embodiments, D is an amino acid sequence comprising six amino acids. In some embodiments, each of the six amino acids is the same. In some embodiments, five of the six amino acids are the same. In some embodiments, four of the six amino acids are the same. In other embodiments, three of the six amino acids are the same. In yet other embodiments, two of the six amino acids are the same. In some embodiments, at least two contiguous amino acids of the six amino acids are the same. In other embodiments, at least three contiguous amino acids of the six amino acids are the same. In yet other embodiments, four contiguous amino acids of the six amino acids are the same. In yet further embodiments, five contiguous amino acids of the six amino acids are the same. In some embodiments, the six amino acids are selected from glycine, serine, threonine, alanine, lysine, and glutamic acid. In some embodiments, D comprises 6 amino acids, and x ranges from 1 to 6. In some embodiments, D comprises 6 amino acids, and x ranges from 1 to 4. In some embodiments, D comprises 6 amino acids, and x is 1 or 2. In some embodiments, D comprises 6 amino acids and x is 1. In some embodiments, D comprises 6 amino acids, and x is 1 or 2 and q is 0. In some embodiments, D comprises 6 amino acids and x is 1 and q is 0. In some embodiments, D comprises 6 amino acids and x is 1 and q is 1. In some embodiments, D comprises 6 amino acids and x is 1 and q is 1 and R is (-[LK]-Fc-[DI]-).

In some embodiments, E comprises 1 amino acid. In some embodiments, the 1 amino acid is selected from aspartic acid, serine, lysine, threonine, tyrosine, alanine, methionine, valine, tryptophan, proline, arginine, glutamine. In other embodiments, the 1 amino acid is selected from aspartic acid and serine. In some embodiments, E comprises 1 amino acid, and y ranges from 1 to 16. In some embodiments, E comprises 1 amino acid, and y ranges from 4 to 16. In some embodiments, E comprises 1 amino acid, and y ranges from 2 to 12. In some embodiments, E comprises 1 amino acid, and y ranges from 2 to 10. In some embodiments, E comprises 1 amino acid, and y ranges from 4 to 12. In some embodiments, E comprises 1 amino acid, and y is 10.

In some embodiments, E is aspartic acid, and y ranges from 1 to 16. In some embodiments, E is aspartic acid, and y ranges from 4 to 12. In some embodiments, E is aspartic acid and y is 10. In some embodiments, E is aspartic acid, v+w is 1, and y ranges from 1 to 16. In some embodiments, E is aspartic acid, v+w is 1, and y ranges from 4 to 12. In some embodiments, E is aspartic acid, v+w is 1, and y is 10. In some embodiments, E is aspartic acid, v+w is 1, x is 0, and y ranges from 1 to 16. In some embodiments, E is aspartic acid, v+w is 1,
x is 0, and y ranges from 4 to 12. In some embodiments, E is aspartic acid, v+w is 1, x is 0, and y is 10. In some embodiments, E is aspartic acid, v+w is 1, x is 0, and y is 10 and q is 1.

In some embodiments, E comprises 2 amino acids. In some embodiments, the 2 amino acids are selected from aspartic acid, serine, lysine, threonine, tyrosine, alanine, methionine, valine, tryptophan, proline, arginine, glutamine. In other embodiments, the 2 amino acid is selected from aspartic acid and serine. In some embodiments, E is -D-S-. In other embodiments, E is -D-D-. In some embodiments, E comprises 2 amino acids, and y ranges from 1 to 16. In some embodiments, E comprises 2 amino acids, and y ranges from 1 to 12. In some embodiments, E comprises 2 amino acids, and y ranges from 1 to 10. In some embodiments, E comprises 2 amino acids, and y ranges from 1 to 8. In some embodiments, E comprises 2 amino acids, and y ranges from 1 to 6. In some embodiments, E comprises 2 amino acids, and y ranges from 1 to 4. In some embodiments, E comprises 2 amino acids, and y is 1 or 2. In some embodiments, E comprises 2 amino acids, and y ranges from 2 to 12. In some embodiments, E comprises 2 amino acids, and y ranges from 2 to 10. In some embodiments, E comprises 2 amino acids, and y ranges from 2 to 8. In some embodiments, E comprises 2 amino acids, and y ranges from 2 to 8 and q is 0.

In some embodiments, E is -D-D- or -D-S-, and y ranges from 1 to 16. In some embodiments, E is -D-D- or -D-S-, and y ranges from 1 to 12. In some embodiments, E is -D-D- or -D-S-, and y ranges from 1 to 10. In some embodiments, E is aspartic acid, and y ranges from 1 to 8. In some embodiments, E is -D-D- or -D-S-, and y is 10. In some embodiments, E is -D-D- or -D-S-, v+w is 1, and y ranges from 1 to 16. In some embodiments, E is -D-D- or -D-S-, y ranges from 1 to 12. In some embodiments, E is -D-D- or -D-S-, v+w is 1, and y ranges from 1 to 10. In some embodiments, E is -D-D- or -D-S-, v+w is 1, and y ranges from 1 to 8. In some embodiments, E is -D-D- or -D-S-, v+w is 1, x is 0, and y ranges from 1 to 16. In some embodiments, E is -D-D- or -D-S-, v+w is 1, x is 0, and y ranges from 1 to 12. In some embodiments, E is -D-D- or -D-S-, v+w is 1, x is 0, and y ranges from 1 to 10. In some embodiments, E is -D-D- or -D-S-, v+w is 1, x is 0, and y ranges from 1 to 8. In some embodiments, E is -D-D- or -D-S-, v+w is 1, x is 0, y ranges from 1 to 8, and z is 1.

In some embodiments, E comprises 3 amino acids. In some embodiments, the 3 amino acids are selected from aspartic acid, serine, lysine, threonine, tyrosine, alanine, methionine, valine, tryptophan, proline, arginine, glutamine. In other embodiments, the 3 amino acid is selected from aspartic acid and serine. In some embodiments, E is -D-S-S-. In other embodiments, E is -D-D-S-. In other embodiments, E is -D-D-D. In other embodiments, E is -D-S-S-.

In some embodiments, E comprises 3 amino acids, and y ranges from 1 to 16. In some embodiments, E comprises 3 amino acids, and y ranges from 1 to 12. In some embodiments, E comprises 3 amino acids, and y ranges from 2 to 12. In some embodiments, E comprises 3 amino acids, and y ranges from 1 to 10. In some embodiments, E comprises 3 amino acids, and y ranges from 2 to 10. In some embodiments, E comprises 3 amino acids, and y ranges from 1 to 8. In some embodiments, E comprises 3 amino acids, and y ranges from 2 to 8. In some embodiments, E comprises 3 amino acids, and y ranges from 1 to 6. In some embodiments, E comprises 3 amino acids, and y ranges from 3 to 6. In some embodiments, E comprises 3 amino acids, and y ranges from 3 to 6 and q is 0.

In some embodiments, E is -D-S-S-, and y ranges from 1 to 16. In some embodiments, E is -D-S-S-, and y ranges from 1 to 12. In some embodiments, E is -D-S-S-, and y ranges from 1 to 10. In some embodiments, E is aspartic acid, and y ranges from 1 to 8. In some embodiments, E is -D-S-S-, and y is 6, i.e. [E]_{y} is [-DSS-]₆. In other embodiments, E is -D-S-S-, y is 6 and z is 1. In some embodiments, E is -D-S-S-, y is 6 and q is 0. In other embodiments, E is -D-S-S-, y is 6, z is 1, and q is 0. In other embodiments, E is -D-S-S-, y is 6, z is 1, q is 0, and x is 0. In other embodiments, E is -D-S-S-, y is 6, z is 1, q is 0, and x is 2.

In some embodiments, E is -D-S-S-, y is 6 and q is 1. In other embodiments, E is -D-S-S-, y is 6, z is 1, and q is 1. In other embodiments, E is -D-S-S-, y is 6, z is 1, x is 0, and q is 1. In other embodiments, E is -D-S-S-, y is 6, z is 1, x is 2, and q is 1.

In some embodiments, E is -D-S-S-, y is 6, q is 1, and R is (-[LK]-Fc-[DI]-). In some embodiments, E is -D-S-S-, y is 6, q is 1, R is (-[LK]-Fc-[DI]-), and [A] has at least 99% identity to any one of SEQ ID NOS: 12 and 33-43. In some embodiments, E is -D-S-S-, y is 6, q is 1, R is (-[LK]-Fc-[DI]-), and [A] has identity to any one of SEQ ID NOS: 12 and 33-43. In some embodiments, E is -D-S-S-, y is 6, q is 1, R is (-[LK]-Fc-[DI]-), [A] has at least 99% identity to any one of SEQ ID NOS: 12 and 33-43; and [B] comprises at least 99% identity to that of SEQ ID NO: 11. In some embodiments, E is -D-S-S-, y is 6, q is 1, R is (-[LK]-Fc-[DI]-), [A] has at least 99% identity to any one of SEQ ID NOS: 12 and 33-43; and [B] comprises SEQ ID NO: 11.

In some embodiments, E comprises 4 amino acids. In some embodiments, the 4 amino acids are selected from aspartic acid, serine, lysine, threonine, tyrosine, alanine, methionine, valine, tryptophan, proline, arginine, glutamine. In other embodiments, the 4 amino acid is selected from aspartic acid and serine. In some embodiments, E is DDSS. In other embodiments, E is -D-D-D-S-. In other embodiments, E is -D-D-D-D-. In other embodiments, E is -D-D-S-S-. In other embodiments, E is -D-S-S-S-. In some embodiments, E comprises 4 amino acids, and y ranges from 1 to 16. In some embodiments, E comprises 4 amino acids, and y ranges from 1 to 12. In some embodiments, E comprises 4 amino acids, and y ranges from 1 to 10. In some embodiments, E comprises 4 amino acids, and y ranges from 1 to 8. In some embodiments, E comprises 4 amino acids, and y ranges from 1 to 6. In some embodiments, E comprises 4 amino acids, and y ranges from 1 to 4. In some embodiments, E comprises 4 amino acids, and y is 1 or 2. In some embodiments, E comprises 4 amino acids, and y ranges from 2 to 12. In some embodiments, E comprises 4 amino acids, and y ranges from 2 to 10. In some embodiments, E comprises 4 amino acids, and y ranges from 2 to 8. In some embodiments, E comprises 4 amino acids, and y ranges from 2 to 8 and q is 0. In some embodiments, E comprises 4 amino acids, and y ranges from 2 to 8. In some embodiments, E comprises 4 amino acids, and y ranges from 2 to 8 and q is 1.

In some embodiments, E comprises 5 or more amino acids. In some embodiments, the 5 or more amino acids are selected from aspartic acid, serine, lysine, threonine, tyrosine, alanine, methionine, valine, tryptophan, proline, arginine, glutamine. In other embodiments, the 5 or more amino acid is selected from aspartic acid and serine. In other embodiments, E comprises 5 amino acids. For example, E may be -D-D-S-S-D- or -D-D-S-S-D-. In other embodiments, E comprises 6 amino acids. For example, E may be -D-D-S-S-D-D-. In other embodiments, E comprises 7 amino acids. For example, E may be -S-D-D-S-S-D-D-. By way of another example, E may be KRRTPVR. By way of yet another example, E may be KNFQSRS. By way of a further example, E may be KTYASMQ. In other embodiments, E comprises 8 amino acids. For example, E may be -S-D-D-S-S-D-D-S-. By way of another example, E may be KRRTPVRE. By way of yet another example, E may be KNFQSRSH. By way of a further example, E may be KTYASMQW.

In some embodiments, q is 0. In some embodiments, q is 0, and x is at least 1. In other embodiments, q is 0 and y is at least 4. In other embodiments, q is 0 and y is 6.

In some embodiments, z is an integer ranging from 1 to 6. In other embodiments, z is an integer ranging from 1 to 4. In yet other embodiments, z is an integer ranging from 1 to 3. In further embodiments, z is 1 or 2. In even further embodiments, z is 1. In even further embodiments, z is 1; and q is 0 and y is 6. In even further embodiments, z is 1; and q is 1 and y is 6.

In some embodiments q is 0. In some embodiments, q is 0 and [E]_{y} is [DSS]₆.

In some embodiments, the present disclosure provides for a polypeptide having Formula (IA):

[A]ᵥ-[B]-[C]_{w}-[R]_{q}-([D]ₓ-[E]_{y})_{z}, (IA)
wherein
A comprises an amino acid sequence encoding a secretion signal peptide;
B comprises an amino acid encoding an alkaline phosphatase;
C comprises an amino acid sequence encoding a GPI anchor;
R is -(Mₒ(Fc)Nₚ)-, where M and N each independently include between 1 and 6 amino acids, where Fc is a Fc domain, and o and p are each independently 0, 1, or 2;
D comprises an amino acid sequence having between 4 and 6 amino acids, or is F(G)ₜF, where each F is the same amino acid, G is an amino acid sequence having 3, 4, or 5 amino acids, and t is an integer ranging from 2-5;
E comprises an amino acid sequence having between 1 and 8 amino acids;
q is 0 or 1;
v is 0 or 1;
w is 0 or 1;
x is 0 or an integer ranging from 1 to 6;
y is 0 or an integer ranging from 1 to 16; and
z is 0 or an integer ranging from 1 to 6;
provided that the polypeptide of Formula (IA) does not have the amino acid sequence of SEQ ID NO: 1 or does not comprise the amino acid sequence encoded by the nucleotide sequence of SEQ ID NO: 107; or provided that the polypeptide of Formula (IA) is not Strensiq® or Asfotase alfa.

For example, in some embodiments, the polypeptide of Formula (IA) has an amino acid sequence having 99% identity or less as compared with SEQ ID NO: 1. By way of another example, in some embodiments, the polypeptide of Formula (IA) has an amino acid sequence having 98% identity or less as compared with SEQ ID NO: 1. By way of yet another example, in some embodiments, the polypeptide of Formula (IA) has an amino acid sequence having 97% identity or less as compared with SEQ ID NO: 1. In some embodiments, the polypeptide of Formula (IA) has an amino acid sequence having 96% identity or less as compared with SEQ ID NO: 1. In other embodiments, the polypeptide of Formula (IA) has an amino acid sequence having 95% identity or less as compared with SEQ ID NO: 1.

In some embodiments, the polypeptide of Formula (IA) is encoded by a nucleotide sequence having less than 99% identity to SEQ ID NO: 107. In some embodiments, the polypeptide of Formula (IA) is encoded by a nucleotide sequence having less than 98% identity to SEQ ID NO: 107. In some embodiments, the polypeptide of Formula (IA) is encoded by a nucleotide sequence having less than 97% identity to SEQ ID NO: 107. In some embodiments, the polypeptide of Formula (IA) is encoded by a nucleotide sequence having less than 96% identity to SEQ ID NO: 107. In some embodiments, the polypeptide of Formula (IA) is encoded by a nucleotide sequence having less than 95% identity to SEQ ID NO: 107.

In some embodiments, when v is 1, w is 0, q is 1, o is 1, p is 1, N is the diamino acid -D-I-, M is the diamino acid -L-K-, [B] comprises SEQ ID NO: 11, Fc comprises SEQ ID NO: 130, and x is 0, then [E]_{y} is not D₁₀-D₁₆. In some embodiments, when v is 1, w is 0, q is 1, o is 1, p is 1, N is the diamino acid -D-I-, M is the diamino acid -L-K-, [B] comprises SEQ ID NO: 11, Fc comprises SEQ ID NO: 130, and x is 0, then [E]_{y} does not comprise ten to sixteen contiguous aspartic acid residues.

In some embodiments, the polypeptide of Formula (IA) is not conjugated to a dextran.

In some embodiments, the polypeptide of Formula (IA) is catalytically competent to allow formation of hydroxyapatite crystals in bone.

In some embodiments, the amino acid encoding the alkaline phosphatase (“[B]”) is a tissue non-specific alkaline phosphatase. In some embodiments, the amino acid encoding the alkaline phosphatase has at least about 85% identity to SEQ ID NO: 11. In other embodiments, the amino acid encoding the alkaline phosphatase has at least about 90% identity to SEQ ID NO: 11. In other embodiments, the amino acid encoding the alkaline phosphatase has at least about 91% identity to SEQ ID NO: 11. In other embodiments, the amino acid encoding the alkaline phosphatase has at least about 92% identity to SEQ ID NO: 11. In other embodiments, the amino acid encoding the alkaline phosphatase has at least about 93% identity to SEQ ID NO: 11. In other embodiments, the amino acid encoding the alkaline phosphatase has at least about 94% identity to SEQ ID NO: 11.

In yet other embodiments, the amino acid encoding the alkaline phosphatase has at least about 95% identity to SEQ ID NO: 11. In further embodiments, the amino acid encoding the alkaline phosphatase has at least about 96% identity to SEQ ID NO: 11. In even further embodiments, the amino acid encoding the alkaline phosphatase has at least about 97% identity to SEQ ID NO: 11. In yet even further embodiments, the amino acid encoding the alkaline phosphatase has at least about 98% identity to SEQ ID NO: 11. In yet even further embodiments, the amino acid encoding the alkaline phosphatase has at least about 99% identity to SEQ ID NO: 11. In yet even further embodiments, the amino acid encoding the alkaline phosphatase has at least about 99% identity to SEQ ID NO: 11 and q is 0. In yet even further embodiments, the amino acid encoding the alkaline phosphatase has at least about 99% identity to SEQ ID NO: 11 and q is 1.

In some embodiments, the amino acid encoding the alkaline phosphatase comprises SEQ ID NO: 11. In some embodiments, the amino acid encoding the alkaline phosphatase comprises SEQ ID NO: 11 and q is 0. In some embodiments, the amino acid encoding the alkaline phosphatase comprises SEQ ID NO: 11 and q is 1.

In some embodiments, the amino acid encoding the alkaline phosphatase comprises a variant of SEQ ID NO: 11, such as a variant comprising one or more amino acid substitutions. In some embodiments, the amino acid encoding the alkaline phosphatase comprises a variant of SEQ ID NO: 11 having a single amino acid substitution. For instance, a variant of SEQ ID NO: 11 may comprises a C102S substitution. By way of another example, a variant of SEQ ID NO: 11 may comprise an E434G substitution. By way of yet another example, a variant of SEQ ID NO: 11 may comprise an A321H substitution. In some embodiments, the amino acid encoding the alkaline phosphatase comprises a variant of SEQ ID NO: 11 having two amino acid substitutions. For instance, a variant of SEQ ID NO: 11 may comprise any two of a C102S substitution, an E434G substitution, or an A321H substitution, e.g. both an A321H substitution and an E434G substitution. In some embodiments, the amino acid encoding the alkaline phosphatase comprises a variant of SEQ ID NO: 11 having three amino acid substitutions. In some embodiments, the amino acid encoding the alkaline phosphatase comprises a variant of SEQ ID NO: 11 having four amino acid substitutions. In some embodiments, the amino acid encoding the alkaline phosphatase comprises a variant of SEQ ID NO: 11 having five amino acid substitutions. In some embodiments, the amino acid encoding the alkaline phosphatase comprises a variant of SEQ ID NO: 11 having six amino acid substitutions.

In some embodiments, the amino acid encoding the secretion signal peptide has at least 90% sequence identity to SEQ ID NO: 12. In other embodiments, the amino acid encoding the secretion signal peptide has at least 95% sequence identity to SEQ ID NO: 12. In yet other embodiments, the amino acid encoding the secretion signal peptide has at least 96% sequence identity to SEQ ID NO: 12. In further embodiments, the amino acid encoding the secretion signal peptide has at least 97% sequence identity to SEQ ID NO: 12. In yet further embodiments, the amino acid encoding the secretion signal peptide has at least 98% sequence identity to SEQ ID NO: 12. In even further embodiments, the amino acid encoding the secretion signal peptide has at least 99% sequence identity to SEQ ID NO: 12. In yet even further embodiments, the amino acid encoding the secretion signal peptide comprises SEQ ID NO: 12. In yet even other embodiments, the amino acid encoding the secretion signal peptide comprises SEQ ID NO: 12, and wherein [D]ₓ is [-GGGGS-]₂. In yet even other embodiments, the amino acid encoding the secretion signal peptide comprises SEQ ID NO: 12, and wherein [E]_{y} is [-DSS-]₆. In yet even other embodiments, the amino acid encoding the secretion signal peptide comprises SEQ ID NO: 12; [E]_{y} is [-DSS-]₆, and q is 0. In yet even other embodiments, the amino acid encoding the secretion signal peptide comprises SEQ ID NO: 12; [E]_{y} is [-DSS-]₆, and q is 1.

In some embodiments, the amino acid encoding the secretion signal peptide has at least 90% sequence identity to any one of SEQ ID NOS: 33-43. In other embodiments, the amino acid encoding the secretion signal peptide has at least 95% sequence identity to any one of SEQ ID NOS: 33-43. In yet other embodiments, the amino acid encoding the secretion signal peptide has at least 96% sequence identity to any one of SEQ ID NOS: 33-43. In further embodiments, the amino acid encoding the secretion signal peptide has at least 97% sequence identity to any one of SEQ ID NOS: 33-43. In yet further embodiments, the amino acid encoding the secretion signal peptide has at least 98% sequence identity to any one of SEQ ID NOS: 33-43. In even further embodiments, the amino acid encoding the secretion signal peptide has at least 99% sequence identity to any one of SEQ ID NOS: 33-43. In yet even further embodiments, the amino acid encoding the secretion signal peptide comprises any one of SEQ ID NOS: 33-43. In yet even other embodiments, the amino acid encoding the secretion signal peptide comprises any one of SEQ ID NOS: 33-43, and wherein [E]_{y} is [-DSS-]₆. In yet even other embodiments, the amino acid encoding the secretion signal peptide comprises any one of SEQ ID NOS: 33-43, [E]_{y} is [-DSS-]₆, and q is 0. In yet even other embodiments, the amino acid encoding the secretion signal peptide comprises any one of SEQ ID NOS: 33-43, [E]_{y} is [-DSS-]₆, and q is 1.

In some embodiments, the amino acid encoding the GPI anchor has at least 90% sequence identity to SEQ ID NO: 13. In other embodiments, the amino acid encoding the GPI anchor has at least 95% sequence identity to SEQ ID NO: 13. In yet other embodiments, the amino acid encoding the GPI anchor has at least 96% sequence identity to SEQ ID NO: 13. In further embodiments, the amino acid encoding the GPI anchor has at least 97% sequence identity to SEQ ID NO: 13. In yet further embodiments, the amino acid encoding the GPI anchor has at least 98% sequence identity to SEQ ID NO: 13. In even further embodiments, the amino acid encoding the GPI anchor has at least 99% sequence identity to SEQ ID NO: 13. In yet even further embodiments, the amino acid encoding the GPI anchor comprises SEQ ID NO: 13. In yet even other embodiments the amino acid encoding the GPI anchor comprises SEQ ID NO: 13, and wherein [E]_{y} is [-DSS-]₆. In yet even other embodiments, the amino acid encoding the GPI anchor comprises SEQ ID NO: 13; [E]_{y} is [-DSS-]₆, and q is 0. In yet even other embodiments, the amino acid encoding the GPI anchor comprises SEQ ID NO: 13; [E]_{y} is [-DSS-]₆, and q is 1.

In some embodiments, the amino acid encoding the GPI anchor has at least 90% sequence identity to SEQ ID NO: 14. In other embodiments, the amino acid encoding the GPI anchor has at least 95% sequence identity to SEQ ID NO: 14. In yet other embodiments, the amino acid encoding the GPI anchor has at least 96% sequence identity to SEQ ID NO: 14. In further embodiments, the amino acid encoding the GPI anchor has at least 97% sequence identity to SEQ ID NO: 14. In yet further embodiments, the amino acid encoding the GPI anchor has at least 98% sequence identity to SEQ ID NO: 14. In even further embodiments, the amino acid encoding the GPI anchor has at least 99% sequence identity to SEQ ID NO: 14. In yet even further embodiments, the amino acid encoding the GPI anchor comprises SEQ ID NO: 14. In yet even other embodiments the amino acid encoding the GPI anchor comprises SEQ ID NO: 14, and wherein [E]_{y} is [-DSS-]₆. In yet even other embodiments, the amino acid encoding the GPI anchor comprises SEQ ID NO: 14; [E]_{y} is [-DSS-]₆, and q is 0. In yet even other embodiments, the amino acid encoding the GPI anchor comprises SEQ ID NO: 14; [E]_{y} is [-DSS-]₆, and q is 1.

In some embodiments, at least one of v or w is 0 and the amino acid encoding the alkaline phosphatase has at least about 90% identity to SEQ ID NO: 11. In other embodiments, at least one of v or w is 0 and the amino acid encoding the alkaline phosphatase has at least about 95% identity to SEQ ID NO: 11. In further embodiments, at least one of v or w is 0 and the amino acid encoding the alkaline phosphatase has at least about 96% identity to SEQ ID NO: 11. In even further embodiments, at least one of v or w is 0 and the amino acid encoding the alkaline phosphatase has at least about 97% identity to SEQ ID NO: 11. In yet even further embodiments, at least one of v or w is 0 and the amino acid encoding the alkaline phosphatase has at least about 98% identity to SEQ ID NO: 11. In yet even further embodiments, at least one of v or w is 0 and the amino acid encoding the alkaline phosphatase has at least about 99% identity to SEQ ID NO: 11. In other embodiments, at least one of v or w is 0 and the amino acid encoding the alkaline phosphatase comprises SEQ ID NO: 11.

In some embodiments, at least one of v or w is 0; q is 0; and the amino acid encoding the alkaline phosphatase comprises SEQ ID NO: 11. In some embodiments, at least one of v or w is 0; q is 0; [E]_{y} is [DSS]₆; and the amino acid encoding the alkaline phosphatase comprises SEQ ID NO: 11.

In some embodiments, at least one of v or w is 0; q is 1; [E]_{y} is [DSS]₆; and the amino acid encoding the alkaline phosphatase comprises SEQ ID NO: 11. In some embodiments, at least one of v or w is 0; q is 1; [E]_{y} is [DSS]₆; the amino acid encoding the alkaline phosphatase comprises SEQ ID NO: 11; and [A] comprises at least 99% identity to any one of SEQ ID NOS: 12 and 33-43. In some embodiments, at least one of v or w is 0; q is 1; [E]_{y} is [DSS]₆; the amino acid encoding the alkaline phosphatase comprises SEQ ID NO: 11; and [A] comprises any one of SEQ ID NOS: 12 and 33-43.

In some embodiments, at least one of v or w is 0, the amino acid encoding the alkaline phosphatase has at least about 90% identity to SEQ ID NO: 11, and x+y=8 to 12. In some embodiments, at least one of v or w is 0, the amino acid encoding the alkaline phosphatase has at least about 95% identity to SEQ ID NO: 11, and x+y=8 to 12. In some embodiments, at least one of v or w is 0, the amino acid encoding the alkaline phosphatase has at least about 96% identity to SEQ ID NO: 11, and x+y=8 to 12. In some embodiments, at least one of v or w is 0, the amino acid encoding the alkaline phosphatase has at least about 97% identity to SEQ ID NO: 11, and x+y=8 to 12. In some embodiments, at least one of v or w is 0, the amino acid encoding the alkaline phosphatase has at least about 98% identity to SEQ ID NO: 11, and x+y=8 to 12. In some embodiments, at least one of v or w is 0, the amino acid encoding the alkaline phosphatase has at least about 99% identity to SEQ ID NO: 11, and x+y=8 to 12. In some embodiments, at least one of v or w is 0, the amino acid encoding the alkaline phosphatase comprises SEQ ID NO: 11, and x+y=8 to 12. In some embodiments, at least one of v or w is 0, the amino acid encoding the alkaline phosphatase comprises SEQ ID NO: 11, and x+y=8 to 12.

In some embodiments, at least one of v or w is 0, the amino acid encoding the alkaline phosphatase comprises SEQ ID NO: 11, and x+y=8 to 12, and q is 0.

In some embodiments, at least one of v or w is 0, the amino acid encoding the alkaline phosphatase comprises SEQ ID NO: 11, and x+y=8 to 12, and q is 1. In some embodiments, at least one of v or w is 0, the amino acid encoding the alkaline phosphatase comprises SEQ ID NO: 11, and x+y=8 to 12, q is 1; and R is (-[LK]-Fc-[DI]-). In some embodiments, at least one of v or w is 0, the amino acid encoding the alkaline phosphatase comprises SEQ ID NO: 11, and x+y=8 to 12, q is 1; and R is (-[LK]-Fc-[DI]-), and [A] comprises an amino acid sequence having at least 95% identity to any one of SEQ ID NOS: 12 and 33-43. In some embodiments, at least one of v or w is 0, the amino acid encoding the alkaline phosphatase comprises SEQ ID NO: 11, and x+y=8 to 12, q is 1; and R is (-[LK]-Fc-[DI]-), and [A] comprises an amino acid sequence having at least 96% identity to any one of SEQ ID NOS: 12 and 33-43. In some embodiments, at least one of v or w is 0, the amino acid encoding the alkaline phosphatase comprises SEQ ID NO: 11, and x+y=8 to 12, q is 1; and R is (-[LK]-Fc-[DI]-), and [A] comprises an amino acid sequence having at least 97% identity to any one of SEQ ID NOS: 12 and 33-43. In some embodiments, at least one of v or w is 0, the amino acid encoding the alkaline phosphatase comprises SEQ ID NO: 11, and x+y=8 to 12, q is 1; and R is (-[LK]-Fc-[DI]-), and [A] comprises an amino acid sequence having at least 98% identity to any one of SEQ ID NOS: 12 and 33-43. In some embodiments, at least one of v or w is 0, the amino acid encoding the alkaline phosphatase comprises SEQ ID NO: 11, and x+y=8 to 12, q is 1; and R is (-[LK]-Fc-[DI]-), and [A] comprises an amino acid sequence having at least 99% identity to any one of SEQ ID NOS: 12 and 33-43. In some embodiments, at least one of v or w is 0, the amino acid encoding the alkaline phosphatase comprises SEQ ID NO: 11, and x+y=8 to 12, q is 1; and R is (-[LK]-Fc-[DI]-), and [A] comprises an amino acid sequence having any one of SEQ ID NOS: 12 and 33-43.

In some embodiments, v is 1 and w is 0, the amino acid encoding the alkaline phosphatase has at least about 90% identity to SEQ ID NO: 11, and x+y=8 to 12. In some embodiments, v is 1 and w is 0, the amino acid encoding the alkaline phosphatase has at least about 95% identity to SEQ ID NO: 11, and x+y=8 to 12. In some embodiments, v is 1 and w is 0, the amino acid encoding the alkaline phosphatase has at least about 96% identity to SEQ ID NO: 11, and x+y=8 to 12. In some embodiments, v is 1 and w is 0, the amino acid encoding the alkaline phosphatase has at least about 97% identity to SEQ ID NO: 11, and x+y=8 to 12. In some embodiments, v is 1 and w is 0, the amino acid encoding the alkaline phosphatase has at least about 98% identity to SEQ ID NO: 11, and x+y=8 to 12. In some embodiments, v is 1 and w is 0, the amino acid encoding the alkaline phosphatase has at least about 99% identity to SEQ ID NO: 11, and x+y=8 to 12. In some embodiments, v is 1 and w is 0, the amino acid encoding the alkaline phosphatase comprises SEQ ID NO: 11, and x+y=8 to 12. In some embodiments, v is 1 and w is 0, the amino acid encoding the alkaline phosphatase comprises SEQ ID NO: 11, and x+y=8 to 12 and q is 1.

In some embodiments, v is 1 and w is 0, the amino acid encoding the alkaline phosphatase comprises SEQ ID NO: 11, and x+y=8 to 12 and q is 0.

In some embodiments, the amino acid encoding the alkaline phosphatase has at least about 95% identity to SEQ ID NO: 11, x+y=8 to 12, and where x and y are both at least 1. In some embodiments, the amino acid encoding the alkaline phosphatase has at least about 97% identity to SEQ ID NO: 11, x+y=8 to 12, and where x and y are both at least 1. In some embodiments, the amino acid encoding the alkaline phosphatase has at least about 99% identity to SEQ ID NO: 11, x+y=8 to 12, and where x and y are both at least 1. In some embodiments, the amino acid encoding the alkaline phosphatase has at least about 99% identity to SEQ ID NO: 11, x+y=8 to 12, where x and y are both at least 1; and q is 0. In some embodiments, the amino acid encoding the alkaline phosphatase has at least about 99% identity to SEQ ID NO: 11, x+y=8 to 12, where x and y are both at least 1; and q is 1. In some embodiments, the amino acid encoding the alkaline phosphatase has at least about 99% identity to SEQ ID NO: 11, and x+y=8 to 12, where x and y are both at least 1; q is 1; and R is (-[LK]-Fc-[DI]-).

In some embodiments, at least one of v or w is 0, the amino acid encoding the alkaline phosphatase has at least about 90% identity to SEQ ID NO: 11, and x+y=4 to 8. In some embodiments, at least one of v or w is 0, the amino acid encoding the alkaline phosphatase has at least about 95% identity to SEQ ID NO: 11, and x+y=4 to 8. In some embodiments, at least one of v or w is 0, the amino acid encoding the alkaline phosphatase has SEQ ID NO: 11, and x+y=4 to 8.

In some embodiments, v is 1, w is 0, the amino acid encoding the alkaline phosphatase has at least about 90% identity to SEQ ID NO: 11, and x+y=4 to 8. In some embodiments, v is 1, w is 0, the amino acid encoding the alkaline phosphatase has at least about 95% identity to SEQ ID NO: 11, and x+y=4 to 8. In some embodiments, v is 1, w is 0, the amino acid encoding the alkaline phosphatase has at least about 96% identity to SEQ ID NO: 11, and wherein x+y=4 to 8. In some embodiments, v is 1, w is 0, the amino acid encoding the alkaline phosphatase has at least about 97% identity to SEQ ID NO: 11, and x+y=4 to 8. In some embodiments, v is 1, w is 0, the amino acid encoding the alkaline phosphatase has at least about 98% identity to SEQ ID NO: 11, and x+y=4 to 8. In some embodiments, v is 1, w is 0, the amino acid encoding the alkaline phosphatase has at least about 99% identity to SEQ ID NO: 11, and x+y=4 to 8. In some embodiments, v is 1, w is 0, the amino acid encoding the alkaline phosphatase comprises SEQ ID NO: 11, and x+y=4 to 8.

In some embodiments, v is 1, w is 0, the amino acid encoding the alkaline phosphatase comprises SEQ ID NO: 11, x+y=4 to 8 and q is 0.

In some embodiments, v is 1, w is 0, the amino acid encoding the alkaline phosphatase comprises SEQ ID NO: 11, x+y=4 to 8 and q is 1. In some embodiments, v is 1, w is 0, the amino acid encoding the alkaline phosphatase comprises SEQ ID NO: 11, x+y=4 to 8, q is 1; and R is (-[LK]-Fc-[DI]-). In some embodiments, v is 1, w is 0, the amino acid encoding the alkaline phosphatase comprises SEQ ID NO: 11, x+y=5 to 7, q is 1; and R is (-[LK]-Fc-[DI]-). In some embodiments, v is 1, w is 0, the amino acid encoding the alkaline phosphatase comprises SEQ ID NO: 11, x+y=6, q is 1; and R is (-[LK]-Fc-[DI]-). In some embodiments, v is 1, w is 0, the amino acid encoding the alkaline phosphatase comprises SEQ ID NO: 11, x+y=6, q is 1; R is (-[LK]-Fc-[DI]-), and [A] comprises an amino acid sequence having at least 95% identity to any one of SEQ ID NOS: 12 and 33-43. In some embodiments, v is 1, w is 0, the amino acid encoding the alkaline phosphatase comprises SEQ ID NO: 11, x+y=6, q is 1; R is (-[LK]-Fc-[DI]-), and [A] comprises an amino acid sequence having at least 96% identity to any one of SEQ ID NOS: 12 and 33-43. In some embodiments, v is 1, w is 0, the amino acid encoding the alkaline phosphatase comprises SEQ ID NO: 11, x+y=6, q is 1; R is (-[LK]-Fc-[DI]-), [A] comprises an amino acid sequence having at least 97% identity to any one of SEQ ID NOS: 12 and 33-43. In some embodiments, v is 1, w is 0, the amino acid encoding the alkaline phosphatase comprises SEQ ID NO: 11, x+y=6 and q is 1; R is (-[LK]-Fc-[DI]-), and [A] comprises an amino acid sequence having at least 98% identity to any one of SEQ ID NOS: 12 and 33-43. In some embodiments, v is 1, w is 0, the amino acid encoding the alkaline phosphatase comprises SEQ ID NO: 11, x+y=6, q is 1; R is (-[LK]-Fc-[DI]-), and [A] comprises an amino acid sequence having at least 99% identity to any one of SEQ ID NOS: 12 and 33-43. In some embodiments, v is 1, w is 0, the amino acid encoding the alkaline phosphatase comprises SEQ ID NO: 11, x+y=6, q is 1; R is (-[LK]-Fc-[DI]-) and [A] comprises any one of SEQ ID NOS: 12 and 33-43.

In some embodiments, [A]ᵥ-[B]-[C]_{w} comprises an amino acid sequence having at least 85% sequence identity to SEQ ID NO: 10. In some embodiments, [A]ᵥ-[B]-[C]_{w} comprises an amino acid sequence having at least 90% sequence identity to SEQ ID NO: 10. In some embodiments, [A]ᵥ-[B]-[C]_{w} comprises an amino acid sequence having at least 95% sequence identity to SEQ ID NO: 10. In some embodiments, [A]ᵥ-[B]-[C]_{w} comprises an amino acid sequence having at least 96% sequence identity to SEQ ID NO: 10. In some embodiments, [A]ᵥ-[B]-[C]_{w} comprises an amino acid sequence having at least 97% sequence identity to SEQ ID NO: 10. In some embodiments, [A]ᵥ-[B]-[C]_{w} comprises an amino acid sequence having at least 98% sequence identity to SEQ ID NO: 10. In some embodiments, [A]ᵥ-[B]-[C]_{w} comprises an amino acid sequence having at least 99% sequence identity to SEQ ID NO: 10. In some embodiments, [A]ᵥ-[B]-[C]_{w} comprises an amino acid sequence comprising SEQ ID NO: 10. In some embodiments, [A]ᵥ-[B]-[C]_{w} comprises an amino acid sequence having at least 97% identity to SEQ ID NO: 10; and wherein [E]_{y} is [-DSS-]₆. In some embodiments, [A]ᵥ-[B]-[C]_{w} comprises an amino acid sequence having at least 98% identity to SEQ ID NO: 10; and wherein [E]_{y} is [-DSS-]₆. In some embodiments, [A]ᵥ-[B]-[C]_{w} comprises an amino acid sequence having at least 99% identity to SEQ ID NO: 10; and wherein [E]_{y} is [-DSS-]₆. In some embodiments, [A]ᵥ-[B]-[C]_{w} comprises an amino acid sequence comprising SEQ ID NO: 10; and wherein [E]_{y} is [-DSS-]₆.

In some embodiments, [A]ᵥ-[B]-[C]_{w} comprises an amino acid sequence having at least 85% sequence identity to any one of SEQ ID NOS: 27-32. In some embodiments, [A]ᵥ-[B]-[C]_{w} comprises an amino acid sequence having at least 90% sequence identity to any one of SEQ ID NOS: 27-32. In some embodiments, [A]ᵥ-[B]-[C]_{w} comprises an amino acid sequence having at least 95% sequence identity to any one of SEQ ID NOS: 27-32. In some embodiments, [A]ᵥ-[B]-[C]_{w} comprises an amino acid sequence having at least 96% sequence identity to any one of SEQ ID NOS: 27-32. In some embodiments, [A]ᵥ-[B]-[C]_{w} comprises an amino acid sequence having at least 97% sequence identity to any one of SEQ ID NOS: 27-32. In some embodiments, [A]ᵥ-[B]-[C]_{w} comprises an amino acid sequence having at least 98% sequence identity to any one of SEQ ID NOS: 27-32. In some embodiments, [A]ᵥ-[B]-[C]_{w} comprises an amino acid sequence having at least 99% sequence identity to any one of SEQ ID NOS: 27-32. In some embodiments, [A]ᵥ-[B]-[C]_{w} comprises an amino acid sequence comprising any one of SEQ ID NOS: 27-32.

In some embodiments, [A]ᵥ-[B]-[C]_{w} comprises an amino acid sequence having at least 90% sequence identity to any one of SEQ ID NOS: 10 and 27-32, and wherein x+y is 4 to 8. In some embodiments, [A]ᵥ-[B]-[C]_{w} comprises an amino acid sequence having at least 95% sequence identity to any one of SEQ ID NOS: 10 and 27-32, and wherein x+y is 4 to 8. In some embodiments, [A]ᵥ-[B]-[C]_{w} comprises an amino acid sequence having at least 96% sequence identity to any one of SEQ ID NOS: 10 and 27-32, and wherein x+y is 4 to 8. In some embodiments, [A]ᵥ-[B]-[C]_{w} comprises an amino acid sequence having at least 97% sequence identity to any one of SEQ ID NOS: 10 and 27-32, and wherein x+y is 4 to 8. In some embodiments, [A]ᵥ-[B]-[C]_{w} comprises an amino acid sequence having at least 98% sequence identity to any one of SEQ ID NOS: 10 and 27-32, and wherein x+y is 4 to 8. In some embodiments, [A]ᵥ-[B]-[C]_{w} comprises an amino acid sequence having at least 99% sequence identity to any one of SEQ ID NOS: 10 and 27-32, and wherein x+y is 4 to 8. In some embodiments, [A]ᵥ-[B]-[C]_{w} comprises an amino acid sequence comprising any one of SEQ ID NOS: 10 and 27-32, and wherein x+y is 4 to 8.

In some embodiments, [A]ᵥ-[B]-[C]_{w} comprises an amino acid sequence comprising any one of SEQ ID NOS: 10 and 27-32; wherein x+y is 4 to 8; and wherein q is 0. In some embodiments, [A]ᵥ-[B]-[C]_{w} comprises an amino acid sequence comprising any one of SEQ ID NOS: 10 and 27-32; wherein x+y is 4 to 8; and wherein q is 0 and z is 1.

In some embodiments, [A]ᵥ-[B]-[C]_{w} comprises an amino acid sequence comprising any one of SEQ ID NOS: 10 and 27-32; wherein x+y is 4 to 8; and wherein q is 1. In some embodiments, [A]ᵥ-[B]-[C]_{w} comprises an amino acid sequence comprising any one of SEQ ID NOS: 10 and 27-32; wherein x+y is 4 to 8; and wherein q is 1 and z is 1.

In some embodiments, [A]ᵥ-[B]-[C]_{w} comprises an amino acid sequence having at least 97% sequence identity to any one of SEQ ID NOS: 27-32; and wherein [E]_{y} is [-DSS-]₆. In some embodiments, [A]ᵥ-[B]-[C]_{w} comprises an amino acid sequence having at least 98% sequence identity to any one of SEQ ID NOS: 27-32; and wherein [E]_{y} is [-DSS-]₆. In some embodiments, [A], —[B]-[C]_{w} comprises an amino acid sequence having at least 99% sequence identity to any one of SEQ ID NOS: 27-32; and wherein [E]_{y} is [-DSS-]₆. In some embodiments, [A]ᵥ-[B]-[C]_{w} comprises an amino acid sequence comprising any one of SEQ ID NOS: 27-32, and wherein [E]_{y} is [-DSS-]₆.

In some embodiments, [A]ᵥ-[B]-[C]_{w} comprises an amino acid sequence having at least 90% sequence identity to any one of SEQ ID NOS: 10 and 27-32, and wherein x+y is 8 to 12. In some embodiments, [A]ᵥ-[B]-[C]_{w} comprises an amino acid sequence having at least 95% sequence identity to any one of SEQ ID NOS: 10 and 27-32, and wherein x+y is 8 to 12. In some embodiments, [A], —[B]-[C]_{w} comprises an amino acid sequence having at least 96% sequence identity to any one of SEQ ID NOS: 10 and 27-32, and wherein x+y is 8 to 12. In some embodiments, [A]ᵥ-[B]-[C]_{w} comprises an amino acid sequence having at least 97% sequence identity to any one of SEQ ID NOS: 10 and 27-32, and wherein x+y is 8 to 12. In some embodiments, [A]ᵥ-[B]-[C]_{w} comprises an amino acid sequence having at least 98% sequence identity to any one of SEQ ID NOS: 10 and 27-32, and wherein x+y is 8 to 12. In some embodiments, [A]ᵥ-[B]-[C]_{w} comprises an amino acid sequence having at least 99% sequence identity to any one of SEQ ID NOS: 10 and 27-32, and wherein x+y is 8 to 12. In some embodiments, [A]ᵥ-[B]-[C]_{w} comprises an amino acid sequence comprising any one of SEQ ID NOS: 10 and 27-32, and wherein x+y is 8 to 12.

In some embodiments, [A]ᵥ-[B]-[C]_{w} comprises an amino acid sequence comprising any one of SEQ ID NOS: 10 and 27-32, wherein x+y is 8 to 12, and wherein q is 0. In some embodiments, [A]ᵥ-[B]-[C]_{w} comprises an amino acid sequence comprising any one of SEQ ID NOS: 10 and 27-32, wherein x+y is 8 to 12, and wherein q is 0 and z is 1. In some embodiments, [A]ᵥ-[B]-[C]_{w} comprises an amino acid sequence comprising any one of SEQ ID NOS: 10 and 27-32, wherein x+y is 8 to 12, and wherein q is 1. In some embodiments, [A], [B]-[C]_{w} comprises an amino acid sequence comprising any one of SEQ ID NOS: 10 and 27-32, wherein x+y is 8 to 12, and wherein q is 1 and z is 1. In some embodiments, [A]ᵥ-[B]-[C]_{w} comprises an amino acid sequence comprising any one of SEQ ID NOS: 10 and 27-32, wherein x+y is 8 to 12, wherein q is 1, z is 1, and [E]_{y} is [-DSS-] 6. In some embodiments, [A]ᵥ-[B]-[C]_{w} comprises an amino acid sequence comprising any one of SEQ ID NOS: 10 and 27-32, wherein x+y is 8 to 12, wherein q is 1, z is 1, and [E]_{y} is [-D-]₁₀.

In some embodiments, the Fc domain has at least 90% sequence identity to that of SEQ ID NO: 130. In some embodiments, the Fc domain has at least 91% sequence identity to that of SEQ ID NO: 130. In some embodiments, the Fc domain has at least 92% sequence identity to that of SEQ ID NO: 130. In some embodiments, the Fc domain has at least 93% sequence identity to that of SEQ ID NO: 130. In some embodiments, the Fc domain has at least 94% sequence identity to that of SEQ ID NO: 130. In some embodiments, the Fc domain has at least 95% sequence identity to that of SEQ ID NO: 130. In some embodiments, the Fc domain has at least 96% sequence identity to that of SEQ ID NO: 130. In some embodiments, the Fc domain has at least 97% sequence identity to that of SEQ ID NO: 130. In some embodiments, the Fc domain has at least 98% sequence identity to that of SEQ ID NO: 130. In some embodiments, the Fc domain has at least 99% sequence identity to that of SEQ ID NO: 130. In some embodiments, the Fc domain comprises SEQ ID NO: 130. In some embodiments, the Fc domain comprises SEQ ID NO: 130 and y ranges from 4 to 6. In some embodiments, the Fc domain comprises SEQ ID NO: 130 and [E]_{y} is [DSS]₆. In some embodiments, the R comprises SEQ ID NO: 9 and [E]_{y} is [DSS]₆.

In some embodiments, M comprises one or more amino acids selected from glycine, serine, threonine, alanine, lysine, and glutamic acid. In some embodiments, M comprises 2 amino acids. For example, M may comprise leucine and lysine. In some embodiments, M is leucine-lysine. By way of another example, M may comprise two alanine amino acids In other embodiments, M comprises three amino acids.

In some embodiments, N comprises one or more amino acids selected from glycine, serine, threonine, alanine, lysine, and glutamic acid. In some embodiments, N comprises 2 amino acids (i.e., a diamino acid). For example, N may comprise aspartic acid and isoleucine. By way of another example, N may comprise two alanine amino acids. In some embodiments, N is aspartic acid—isoleucine. In other embodiments, N comprises 3 amino acids.

In some embodiments, M is leucine-lysine and N is aspartic acid—isoleucine. In some embodiments, M is leucine-lysine and N is aspartic acid—isoleucine and Fc has at least 90% sequence identity to that of SEQ ID NO: 130. In some embodiments, M is leucine-lysine and N is aspartic acid—isoleucine and Fc has at least 91% sequence identity to that of SEQ ID NO: 130. In some embodiments, M is leucine-lysine and N is aspartic acid—isoleucine and Fc has at least 92% sequence identity to that of SEQ ID NO: 130. In some embodiments, M is leucine-lysine and N is aspartic acid—isoleucine and Fc has at least 93% sequence identity to that of SEQ ID NO: 130. In some embodiments, M is leucine-lysine and N is aspartic acid—isoleucine and Fc has at least 94% sequence identity to that of SEQ ID NO: 130. In some embodiments, M is leucine-lysine and N is aspartic acid—isoleucine and Fc has at least 95% sequence identity to that of SEQ ID NO: 130. In some embodiments, M is leucine-lysine and N is aspartic acid—isoleucine and Fc has at least 96% sequence identity to that of SEQ ID NO: 130. In some embodiments, M is leucine-lysine and N is aspartic acid—isoleucine and Fc has at least 97% sequence identity to that of SEQ ID NO: 130. In some embodiments, M is leucine-lysine and N is aspartic acid—isoleucine and Fc has at least 98% sequence identity to that of SEQ ID NO: 130. In some embodiments, M is leucine-lysine and N is aspartic acid—isoleucine and Fc has at least 99% sequence identity to that of SEQ ID NO: 130. In some embodiments, M is leucine-lysine and N is aspartic acid—isoleucine and Fc comprises SEQ ID NO: 130.

In some embodiments, M is leucine-lysine and N is aspartic acid—isoleucine; and o, p, and q are each 1. In some embodiments, M is leucine-lysine and N is aspartic acid—isoleucine and Fc has at least 90% sequence identity to that of SEQ ID NO: 130; and o, p, and q are each 1. In some embodiments, M is leucine-lysine and N is aspartic acid—isoleucine and Fc has at least 91% sequence identity to that of SEQ ID NO: 130; and o, p, and q are each 1. In some embodiments, M is leucine-lysine and N is aspartic acid—isoleucine and Fc has at least 92% sequence identity to that of SEQ ID NO: 130; and o, p, and q are each 1. In some embodiments, M is leucine-lysine and N is aspartic acid—isoleucine and Fc has at least 93% sequence identity to that of SEQ ID NO: 130; and o, p, and q are each 1. In some embodiments, M is leucine-lysine and N is aspartic acid—isoleucine and Fc has at least 94% sequence identity to that of SEQ ID NO: 130; and o, p, and q are each 1. In some embodiments, M is leucine-lysine and N is aspartic acid—isoleucine and Fc has at least 95% sequence identity to that of SEQ ID NO: 130; and o, p, and q are each 1. In some embodiments, M is leucine-lysine and N is aspartic acid—isoleucine and Fc has at least 96% sequence identity to that of SEQ ID NO: 130; and o, p, and q are each 1. In some embodiments, M is leucine-lysine and N is aspartic acid—isoleucine and Fc has at least 97% sequence identity to that of SEQ ID NO: 130; and o, p, and q are each 1. In some embodiments, M is leucine-lysine and N is aspartic acid—isoleucine and Fc has at least 98% sequence identity to that of SEQ ID NO: 130; and o, p, and q are each 1. In some embodiments, M is leucine-lysine and N is aspartic acid—isoleucine and Fc has at least 99% sequence identity to that of SEQ ID NO: 130; and o, p, and q are each 1. In some embodiments, M is leucine-lysine; N is aspartic acid—isoleucine and Fc comprises SEQ ID NO: 130; and o, p, and q are each 1. In some embodiments, M is leucine-lysine; N is aspartic acid—isoleucine; Fc comprises SEQ ID NO: 130; o, p, and q are each 1; and [A] has at least 99% identity to any one of SEQ ID NOS: 12 and 33-43. In some embodiments, M is leucine-lysine; N is aspartic acid—isoleucine; Fc comprises SEQ ID NO: 130; o, p, and q are each 1; and [A] comprises any one of SEQ ID NOS: 12 and 33-43.

In some embodiments, M is leucine-lysine and N is aspartic acid—isoleucine; o, p, q, and z are each 1; and x is 0. In some embodiments, M is leucine-lysine and N is aspartic acid—isoleucine and Fc has at least 90% sequence identity to that of SEQ ID NO: 130; o, p, q, and z are each 1; and x is 0. In some embodiments, M is leucine-lysine and N is aspartic acid—isoleucine and Fc has at least 91% sequence identity to that of SEQ ID NO: 130; o, p, q, and z are each 1; and x is 0. In some embodiments, M is leucine-lysine and N is aspartic acid—isoleucine and Fc has at least 92% sequence identity to that of SEQ ID NO: 130; o, p, q, and z are each 1; and x is 0. In some embodiments, M is leucine-lysine and N is aspartic acid—isoleucine and Fc has at least 93% sequence identity to that of SEQ ID NO: 130; o, p, q, and z are each 1; and x is 0. In some embodiments, M is leucine-lysine and N is aspartic acid—isoleucine and Fc has at least 94% sequence identity to that of SEQ ID NO: 130; o, p, q, and z are each 1; and x is 0. In some embodiments, M is leucine-lysine and N is aspartic acid—isoleucine and Fc has at least 95% sequence identity to that of SEQ ID NO: 130; o, p, q, and z are each 1; and x is 0. In some embodiments, M is leucine-lysine and N is aspartic acid—isoleucine and Fc has at least 96% sequence identity to that of SEQ ID NO: 130; o, p, q, and z are each 1; and x is 0. In some embodiments, M is leucine-lysine and N is aspartic acid—isoleucine and Fc has at least 97% sequence identity to that of SEQ ID NO: 130; o, p, q, and z are each 1; and x is 0. In some embodiments, M is leucine-lysine and N is aspartic acid—isoleucine and Fc has at least 98% sequence identity to that of SEQ ID NO: 130; o, p, q, and z are each 1; and x is 0. In some embodiments, M is leucine-lysine and N is aspartic acid—isoleucine and Fc has at least 99% sequence identity to that of SEQ ID NO: 130; o, p, q, and z are each 1; and x is 0. In some embodiments, M is leucine-lysine and N is aspartic acid—isoleucine and Fc comprises SEQ ID NO: 130; o, p, q, and z are each 1; and x is 0. In some embodiments, M is leucine-lysine and N is aspartic acid—isoleucine and Fc comprises SEQ ID NO: 130; o, p, q, and z are each 1; x is 0; and [A] has at least 99% identity to any one of SEQ ID NOS: 12 and 33-43. In some embodiments, M is leucine-lysine and N is aspartic acid—isoleucine and Fc comprises SEQ ID NO: 130; o, p, q, and z are each 1; x is 0; and [A] comprises any one of SEQ ID NOS: 12 and 33-43.

In some embodiments, M is leucine-lysine and N is aspartic acid—isoleucine; o, p, q, and z are each 1; x is 0; and y is 4-8. In some embodiments, M is leucine-lysine and N is aspartic acid—isoleucine and Fc has at least 90% sequence identity to that of SEQ ID NO: 130; o, p, q, and z are each 1; x is 0; and y is 4-8. In some embodiments, M is leucine-lysine and N is aspartic acid—isoleucine and Fc has at least 91% sequence identity to that of SEQ ID NO: 130; o, p, q, and z are each 1; x is 0; and y is 4-8. In some embodiments, M is leucine-lysine and N is aspartic acid—isoleucine and Fc has at least 92% sequence identity to that of SEQ ID NO: 130; o, p, q, and z are each 1; x is 0; and y is 4-8. In some embodiments, M is leucine-lysine and N is aspartic acid—isoleucine and Fc has at least 93% sequence identity to that of SEQ ID NO: 130; o, p, q, and z are each 1; x is 0; and y is 4-8. In some embodiments, M is leucine-lysine and N is aspartic acid—isoleucine and Fc has at least 94% sequence identity to that of SEQ ID NO: 130; o, p, q, and z are each 1; x is 0; and y is 4-8. In some embodiments, M is leucine-lysine and N is aspartic acid—isoleucine and Fc has at least 95% sequence identity to that of SEQ ID NO: 130; o, p, q, and z are each 1; x is 0; and y is 4-8. In some embodiments, M is leucine-lysine and N is aspartic acid—isoleucine and Fc has at least 96% sequence identity to that of SEQ ID NO: 130; o, p, q, and z are each 1; x is 0; and y is 4-8. In some embodiments, M is leucine-lysine and N is aspartic acid—isoleucine and Fc has at least 97% sequence identity to that of SEQ ID NO: 130; o, p, q, and z are each 1; x is 0; and y is 4-8. In some embodiments, M is leucine-lysine and N is aspartic acid—isoleucine and Fc has at least 98% sequence identity to that of SEQ ID NO: 130; o, p, q, and z are each 1; x is 0; and y is 4-8. In some embodiments, M is leucine-lysine and N is aspartic acid—isoleucine and Fc has at least 99% sequence identity to that of SEQ ID NO: 130; o, p, q, and z are each 1; x is 0; and y is 4-8.

In some embodiments, M is leucine-lysine and N is aspartic acid—isoleucine and Fc comprises SEQ ID NO: 130; o, p, q, and z are each 1; x is 0; and y is 4-8. In some embodiments, M is leucine-lysine and N is aspartic acid—isoleucine and Fc comprises SEQ ID NO: 130; o, p, q, and z are each 1; x is 0; y is 4-8; and at least one of v or w is 0. In some embodiments, M is leucine-lysine and N is aspartic acid—isoleucine and Fc comprises SEQ ID NO: 130; o, p, q, and z are each 1; x is 0; y is 4-8; and at least one of v or w is 0; and [A] comprises at least 99% identity to any one of SEQ ID NOS: 12 and 33-43. In some embodiments, M is leucine-lysine and N is aspartic acid—isoleucine and Fc comprises SEQ ID NO: 130; o, p, q, and z are each 1; x is 0; y is 4-8; and at least one of v or w is 0; and [A] comprises any one of SEQ ID NOS: 12 and 33-43.

In some embodiments, M is leucine-lysine and N is aspartic acid—isoleucine; o, p, q, and z are each 1; x is 0; and y is 6. In some embodiments, M is leucine-lysine and N is aspartic acid—isoleucine and Fc has at least 90% sequence identity to that of SEQ ID NO: 130; o, p, q, and z are each 1; x is 0; and y is 6. In some embodiments, M is leucine-lysine and N is aspartic acid—isoleucine and Fc has at least 91% sequence identity to that of SEQ ID NO: 130; o, p, q, and z are each 1; x is 0; and y is 6. In some embodiments, M is leucine-lysine and N is aspartic acid—isoleucine and Fc has at least 92% sequence identity to that of SEQ ID NO: 130; o, p, q, and z are each 1; x is 0; and y is 6. In some embodiments, M is leucine-lysine and N is aspartic acid—isoleucine and Fc has at least 93% sequence identity to that of SEQ ID NO: 130; o, p, q, and z are each 1; x is 0; and y is 6. In some embodiments, M is leucine-lysine and N is aspartic acid—isoleucine and Fc has at least 94% sequence identity to that of SEQ ID NO: 130; o, p, q, and z are each 1; x is 0; and y is 6. In some embodiments, M is leucine-lysine and N is aspartic acid—isoleucine and Fc has at least 95% sequence identity to that of SEQ ID NO: 130; o, p, q, and z are each 1; x is 0; and y is 6. In some embodiments, M is leucine-lysine and N is aspartic acid—isoleucine and Fc has at least 96% sequence identity to that of SEQ ID NO: 130; o, p, q, and z are each 1; x is 0; and y is 6. In some embodiments, M is leucine-lysine and N is aspartic acid—isoleucine and Fc has at least 97% sequence identity to that of SEQ ID NO: 130; o, p, q, and z are each 1; x is 0; and y is 6. In some embodiments, M is leucine-lysine and N is aspartic acid—isoleucine and Fc has at least 98% sequence identity to that of SEQ ID NO: 130; o, p, q, and z are each 1; x is 0; and y is 6. In some embodiments, M is leucine-lysine and N is aspartic acid—isoleucine and Fc has at least 99% sequence identity to that of SEQ ID NO: 130; o, p, q, and z are each 1; x is 0; and y is 6. In some embodiments, M is leucine-lysine and N is aspartic acid—isoleucine and Fc comprises SEQ ID NO: 130; o, p, q, and z are each 1; x is 0; and y is 6. In some embodiments, M is leucine-lysine and N is aspartic acid—isoleucine and Fc comprises SEQ ID NO: 130; o, p, q, and z are each 1; x is 0; y is 6; and the amino acid encoding the alkaline phosphatase comprises at least 99% identity to SEQ ID NO: 11. In some embodiments, M is leucine-lysine and N is aspartic acid—isoleucine and Fc comprises SEQ ID NO: 130; o, p, q, and z are each 1; x is 0; y is 6; and the amino acid encoding the alkaline phosphatase comprises SEQ ID NO: 11. In some embodiments, M is leucine-lysine and N is aspartic acid—isoleucine and Fc comprises SEQ ID NO: 130; o, p, q, and z are each 1; x is 0; y is 6; and the amino acid encoding the alkaline phosphatase comprises one or two amino acid substitutions as compared with SEQ ID NO: 11.

In some embodiments, D is an amino acid sequence comprising four amino acids. In some embodiments, each of the four amino acids is the same. In other embodiments, three of the four amino acids are the same. In yet other embodiments, two of the four amino acids are the same. In some embodiments, at least two contiguous amino acids of the four amino acids are the same. In other embodiments, at least three contiguous amino acids of the four amino acids are the same. In some embodiments, the four amino acids are selected from glycine, serine, threonine, alanine, lysine, and glutamic acid. In some embodiments, D comprises 4 amino acids, and x ranges from 1 to 6. In some embodiments, D comprises 4 amino acids, and x ranges from 1 to 4. In some embodiments, D comprises 4 amino acids, and x is 1 or 2. In some embodiments, D comprises 4 amino acids, and x is 2. In some embodiments, D comprises 4 amino acids and x is 1. In some embodiments, D comprises 4 amino acids, and x is 1 or 2, and q is 0. In some embodiments, D comprises 4 amino acids, and x is 2 and q is 0. In some embodiments, D comprises 4 amino acids and x is 1 and q is 0. In some embodiments, D comprises 4 amino acids and x is 1 and q is 1.

In some embodiments, D is GGGS. In other embodiments, D is GGSS. In yet other embodiments, D is GSSS. In further embodiments, D is GSGS. In some embodiments, D is GGGS and x is an integer ranging from 1 to 4. In some embodiments, D is GGGS and x is an integer ranging from 1 to 3. In some embodiments, D is GGGS and x is 1 or 2. In some embodiments, D is GGGS, x is an integer ranging from 1 to 4, and y is an integer ranging from 1 to 8. In some embodiments, D is GGGS, x is an integer ranging from 1 to 3, and y is an integer ranging from 1 to 8. In some embodiments, D is GGGS, x is 1 or 2, and y is an integer ranging from 1 to 8. In some embodiments, D is GGGS, x is 1 or 2, and y is an integer ranging from 1 to 8 and q is 0. In some embodiments, D is GGGS, x is 1 or 2, y is an integer ranging from 1 to 8 and q is 1. In some embodiments, D is GGGS, x is 1 or 2, y is an integer ranging from 1 to 8, q is 1, and R is (-[LK]-Fc-[DI]-). In some embodiments, D is GGGS, x is 2, y is an integer ranging from 1 to 8, q is 1, and R is (-[LK]-Fc-[DI]-). In some embodiments, D is GGGS, z is 1, x is 2, y is an integer ranging from 1 to 8, q is 1, and R is (-[LK]-Fc-[DI]-).

In some embodiments, D is GGGS or GGSS, and wherein x is an integer ranging from 1 to 3, E is -D-S-S-, and wherein y is an integer ranging from between 1 and 8. In some embodiments, D is GGGS or GGSS, and wherein x is 1 or 2, E is -D-S-S-, and wherein y is an integer ranging from between 1 and 8. In some embodiments, D is GGGS or GGSS, and wherein x is an integer ranging from 1 to 3, E is aspartic acid, and wherein y is an integer ranging from between 8 and 12. In some embodiments, D is GGGS or GGSS, and wherein x is 1 or 2, E is aspartic acid, and wherein y is 10. In some embodiments, D is GGGS or GGSS, and wherein x is 2, E is aspartic acid, and wherein y is 10. In some embodiments, D is GGGS, and wherein x is 2, E is aspartic acid, and wherein y is an integer ranging from 4 to 16. In some embodiments, D is GGGS, and wherein x is 2, E is aspartic acid, and wherein y is an integer ranging from 4 to 12. In some embodiments, D is GGGS, and wherein x is 2, E is aspartic acid, and wherein y is 10. In some embodiments, D is GGGS, and wherein x is 2, E is aspartic acid, and wherein y is 10 and q is 0. In some embodiments, D is GGGS, and wherein x is 2, E is aspartic acid, wherein y is 10 and q is 1. In some embodiments, D is GGGS, x is 2, E is aspartic acid, wherein y is 10 and q is 1, and R is (-[LK]-Fc-[DI]-).

In some embodiments, D is an amino acid sequence comprising five amino acids. In some embodiments, each of the five amino acids is the same. In some embodiments, four of the five amino acids are the same. In other embodiments, three of the five amino acids are the same. In other embodiments, two of the five amino acids are the same. In some embodiments, at least two contiguous amino acids of the five amino acids are the same. In other embodiments, at least three contiguous amino acids of the five amino acids are the same. In yet other embodiments, four contiguous amino acids of the five amino acids are the same. In some embodiments, the five amino acids are selected from glycine, serine, threonine, alanine, lysine, and glutamic acid. In some embodiments, D comprises 5 amino acids, and x ranges from 1 to 6. In some embodiments, D comprises 5 amino acids, and x ranges from 1 to 4. In some embodiments, D comprises 5 amino acids, and x is 1 or 2. In some embodiments, D comprises 5 amino acids and x is 1. In some embodiments, D comprises 5 amino acids and x is 1 and q is 0. In some embodiments, D comprises 5 amino acids, x is 1, and q is 1. In some embodiments, D comprises 5 amino acids, x is 1, and q is 1 and R is (-[LK]-Fc-[DI]-).

In some embodiments, D is GGGGS. In other embodiments, D is GGGSS. In yet other embodiments, D is GGSSS. In further embodiments, D is GGSGS. In even further embodiments, D is GGSGS. In some embodiments, D is GGGGS or GGGSS, and wherein x is an integer ranging from 1 to 4. In some embodiments, D is GGGGS or GGGSS, and wherein x is an integer ranging from 1 to 3. In some embodiments, D is GGGGS or GGGSS, and wherein x is 1 or 2. In some embodiments, D is GGGGS or GGGSS, and wherein x is an integer ranging from 1 to 4, and wherein y is an integer ranging from between 1 and 8. In some embodiments, D is GGGGS or GGGSS, and wherein x is an integer ranging from 1 to 3 and wherein y is an integer ranging from between 1 and 8. In some embodiments, D is GGGGS or GGGSS, and wherein x is 1 or 2, and wherein y is an integer ranging from between 1 and 8.

In some embodiments, D is GGGGS or GGGSS, and wherein x is an integer ranging from 1 to 3, E is -D-S-S-, and wherein y is an integer ranging from between 1 and 8. In some embodiments, D is GGGGS or GGGSS, and wherein x is 1 or 2, E is -D-S-S-, and wherein y is an integer ranging from between 1 and 8. In some embodiments, D is GGGGS or GGGSS, and wherein x is an integer ranging from 1 to 3, E is aspartic acid, and wherein y is an integer ranging from between 8 and 12. In some embodiments, D is GGGGS or GGGSS, and wherein x is 1 or 2, E is aspartic acid, and wherein y is 10. In some embodiments, D is GGGGS, and wherein x is 1 or 2, E is aspartic acid, and wherein y ranges from 4 to 16. In some embodiments, D is GGGGS, and wherein x is 1 or 2, E is aspartic acid, and wherein y ranges from 8 to 12. In some embodiments, D is GGGGS, and wherein x is 1 or 2, E is aspartic acid, and wherein y is 10. In some embodiments, D is GGGGS, and wherein x is 1 or 2, E is aspartic acid, and wherein y is 10 and q is 0. In some embodiments, D is GGGGS, and wherein x is 1 or 2, E is aspartic acid, and wherein y is 10 and q is 1. In some embodiments, D is GGGGS, and wherein x is 1 or 2, E is aspartic acid, and wherein y is 10 and q is 1 and R is (-[LK]-Fc-[DI]-).

In some embodiments, D is EAAAK. In some embodiments, D is EAAAK, and wherein x is an integer ranging from 2 to 5. In other embodiments, D is EAAAK, wherein x is an integer ranging from 1 to 4. In yet other embodiments, D is EAAAK, wherein x is an integer ranging from 1 to 3. In further embodiments, D is EAAAK, wherein x is 1 or 2.

In some embodiments, D is F(G)ₜF, where each F is the same amino acid, G is an amino acid sequence having 3, 4, or 5 amino acids, and t is an integer ranging from 2-5. In some embodiments, each F is alanine. In some embodiments, G is EAAAK. In some embodiments, each F is alanine, G is EAAAK, and t is 2 or 3. In some embodiments, each F is alanine, G is EAAAK, t is 2 or 2, and x is 1 or 2. In some embodiments, D is an amino acid sequence comprising six amino acids. In some embodiments, each of the six amino acids is the same. In some embodiments, five of the six amino acids are the same. In some embodiments, four of the six amino acids are the same. In other embodiments, three of the six amino acids are the same. In yet other embodiments, two of the six amino acids are the same. In some embodiments, at least two contiguous amino acids of the six amino acids are the same. In other embodiments, at least three contiguous amino acids of the six amino acids are the same. In yet other embodiments, four contiguous amino acids of the six amino acids are the same. In yet further embodiments, five contiguous amino acids of the six amino acids are the same. In some embodiments, the six amino acids are selected from glycine, serine, threonine, alanine, lysine, and glutamic acid. In some embodiments, D comprises 6 amino acids, and x ranges from 1 to 6. In some embodiments, D comprises 6 amino acids, and x ranges from 1 to 4. In some embodiments, D comprises 6 amino acids, and x is 1 or 2. In some embodiments, D comprises 6 amino acids and x is 1. In some embodiments, D comprises 6 amino acids, and x is 1 or 2 and q is 0. In some embodiments, D comprises 6 amino acids and x is 1 and q is 0. In some embodiments, D comprises 6 amino acids and x is 1 and q is 1. In some embodiments, D comprises 6 amino acids and x is 1 and q is 1 and R is (-[LK]-Fc-[DI]-).

In some embodiments, D is GGGGGS. In other embodiments, D is GGGGSS. In yet other embodiments, D is GGGSSS. In further embodiments, D is GGGSGS. In even further embodiments, D is GGSGGS. In yet even further embodiments, D is GGGSGS.

In some embodiments, D is R-GGGGS or R-GGGSS, where R is an amino acid selected from glycine, serine, threonine, alanine, lysine, and glutamic acid and wherein x is an integer ranging from 1 to 4. In some embodiments, D is GGGGS or GGGSS, where R is an amino acid selected from glycine, serine, threonine, alanine, lysine, and glutamic acid, and wherein x is an integer ranging from 1 to 3. In some embodiments, D is GGGGS or GGGSS, where R is an amino acid selected from glycine, serine, threonine, alanine, lysine, and glutamic acid, and wherein x is 1 or 2. In some embodiments, D is GGGGS or GGGSS, where R is an amino acid selected from glycine, serine, threonine, alanine, lysine, and glutamic acid, and wherein x is an integer ranging from 1 to 4, and wherein y is an integer ranging from between 1 and 8. In some embodiments, D is GGGGS or GGGSS, where R is an amino acid selected from glycine, serine, threonine, alanine, lysine, and glutamic acid, and wherein x is an integer ranging from 1 to 3 and wherein y is an integer ranging from between 1 and 8. In some embodiments, D is GGGGS or GGGSS, where R is an amino acid selected from glycine, serine, threonine, alanine, lysine, and glutamic acid, and wherein x is 1 or 2, and wherein y is an integer ranging from between 1 and 8.

In some embodiments, D is R-GGGGS or R-GGGSS, where R is an amino acid selected from glycine, serine, threonine, alanine, lysine, and glutamic acid, and wherein x is an integer ranging from 1 to 3, E is -D-S-S-, and wherein y is an integer ranging from between 1 and 8. In some embodiments, D is GGGGS or GGGSS, where R is an amino acid selected from glycine, serine, threonine, alanine, lysine, and glutamic acid, and wherein x is 1 or 2, E is -D-S-S-, and wherein y is an integer ranging from between 1 and 8. In some embodiments, D is GGGGS or GGGSS, where R is an amino acid selected from glycine, serine, threonine, alanine, lysine, and glutamic acid, and wherein x is an integer ranging from 1 to 3, E is aspartic acid and wherein y is an integer ranging from between 8 and 12. In some embodiments, D is GGGGS or GGGSS, where R is an amino acid selected from glycine, serine, threonine, alanine, lysine, and glutamic acid, and wherein x is 1 or 2, E is aspartic acid, and wherein y is 10.

In some embodiments, E comprises 1 amino acid. In some embodiments, the 1 amino acid is selected from aspartic acid, serine, lysine, threonine, tyrosine, alanine, methionine, valine, tryptophan, proline, arginine, glutamine. In other embodiments, the 1 amino acid is selected from aspartic acid and serine. In some embodiments, E comprises 1 amino acid, and y ranges from 1 to 16. In some embodiments, E comprises 1 amino acid, and y ranges from 4 to 16. In some embodiments, E comprises 1 amino acid, and y ranges from 2 to 12. In some embodiments, E comprises 1 amino acid, and y ranges from 2 to 10. In some embodiments, E comprises 1 amino acid, and y ranges from 4 to 12. In some embodiments, E comprises 1 amino acid, and y is 10.

In some embodiments, E is aspartic acid, and y ranges from 1 to 16. In some embodiments, E is aspartic acid, and y ranges from 4 to 12. In some embodiments, E is aspartic acid and y is 10. In some embodiments, E is aspartic acid, v+w is 1, and y ranges from 1 to 16. In some embodiments, E is aspartic acid, v+w is 1, and y ranges from 4 to 12. In some embodiments, E is aspartic acid, v+w is 1, and y is 10. In some embodiments, E is aspartic acid, v+w is 1, x is 0, and y ranges from 1 to 16. In some embodiments, E is aspartic acid, v+w is 1, x is 0, and y ranges from 4 to 12. In some embodiments, E is aspartic acid, v+w is 1, x is 0, and y is 10. In some embodiments, E is aspartic acid, v+w is 1, x is 0, and y is 10 and q is 1.

In some embodiments, E is aspartic acid, v+w is 1, x is 0, and y is 10 and q is 0. In some embodiments, E is aspartic acid, v+w is 1, x is 0, and y is greater than 16 and q is 1. In some embodiments, E is aspartic acid, v+w is 1, x is 0, and y is less than 10 and q is 1.

In some embodiments, E comprises 2 amino acids. In some embodiments, the 2 amino acids are selected from aspartic acid, serine, lysine, threonine, tyrosine, alanine, methionine, valine, tryptophan, proline, arginine, glutamine. In other embodiments, the 2 amino acid is selected from aspartic acid and serine. In some embodiments, E is -D-S-. In other embodiments, E is -D-D-. In some embodiments, E comprises 2 amino acids, and y ranges from 1 to 16. In some embodiments, E comprises 2 amino acids, and y ranges from 1 to 12. In some embodiments, E comprises 2 amino acids, and y ranges from 1 to 10. In some embodiments, E comprises 2 amino acids, and y ranges from 1 to 8. In some embodiments, E comprises 2 amino acids, and y ranges from 1 to 6. In some embodiments, E comprises 2 amino acids, and y ranges from 1 to 4. In some embodiments, E comprises 2 amino acids, and y is 1 or 2. In some embodiments, E comprises 2 amino acids, and y ranges from 2 to 12. In some embodiments, E comprises 2 amino acids, and y ranges from 2 to 10. In some embodiments, E comprises 2 amino acids, and y ranges from 2 to 8. In some embodiments, E comprises 2 amino acids, and y ranges from 2 to 8 and q is 0.

In some embodiments, E is -D-D- or -D-S-, and y ranges from 1 to 16. In some embodiments, E is -D-D- or -D-S-, and y ranges from 1 to 12. In some embodiments, E is -D-D- or -D-S-, and y ranges from 1 to 10. In some embodiments, E is aspartic acid, and y ranges from 1 to 8. In some embodiments, E is -D-D- or -D-S-, and y is 10. In some embodiments, E is -D-D- or -D-S-, v+w is 1, and y ranges from 1 to 16. In some embodiments, E is -D-D- or -D-S-, y ranges from 1 to 12. In some embodiments, E is -D-D- or -D-S-, v+w is 1, and y ranges from 1 to 10. In some embodiments, E is -D-D- or -D-S-, v+w is 1, and y ranges from 1 to 8. In some embodiments, E is -D-D- or -D-S-, v+w is 1, x is 0, and y ranges from 1 to 16. In some embodiments, E is -D-D- or -D-S-, v+w is 1, x is 0, and y ranges from 1 to 12. In some embodiments, E is -D-D- or -D-S-, v+w is 1, x is 0, and y ranges from 1 to 10. In some embodiments, E is -D-D- or -D-S-, v+w is 1, x is 0, and y ranges from 1 to 8. In some embodiments, E is -D-D- or -D-S-, v+w is 1, x is 0, y ranges from 1 to 8, and z is 1.

In some embodiments, E is -D-D- or -D-S-, v+w is 1, x is 0, y ranges from 1 to 8, and q is 0. In some embodiments, E is -D-D- or -D-S-, v+w is 1, x is 0, y ranges from 1 to 8, q is 0, and z is 1.

In some embodiments, E comprises 3 amino acids. In some embodiments, the 3 amino acids are selected from aspartic acid, serine, lysine, threonine, tyrosine, alanine, methionine, valine, tryptophan, proline, arginine, glutamine. In other embodiments, the 3 amino acid is selected from aspartic acid and serine. In some embodiments, E is -D-S-S-. In other embodiments, E is -D-D-S-. In other embodiments, E is -D-D-D. In other embodiments, E is -D-S-S-.

In some embodiments, E comprises 3 amino acids, and y ranges from 1 to 16. In some embodiments, E comprises 3 amino acids, and y ranges from 1 to 12. In some embodiments, E comprises 3 amino acids, and y ranges from 2 to 12. In some embodiments, E comprises 3 amino acids, and y ranges from 1 to 10. In some embodiments, E comprises 3 amino acids, and y ranges from 2 to 10. In some embodiments, E comprises 3 amino acids, and y ranges from 1 to 8. In some embodiments, E comprises 3 amino acids, and y ranges from 2 to 8. In some embodiments, E comprises 3 amino acids, and y ranges from 1 to 6. In some embodiments, E comprises 3 amino acids, and y ranges from 3 to 6. In some embodiments, E comprises 3 amino acids, and y ranges from 3 to 6 and q is 0.

In some embodiments, E is -D-S-S-, and y ranges from 1 to 16. In some embodiments, E is -D-S-S-, and y ranges from 1 to 12. In some embodiments, E is -D-S-S-, and y ranges from 1 to 10. In some embodiments, E is aspartic acid, and y ranges from 1 to 8. In some embodiments, E is -D-S-S-, and y is 6, i.e. [E]_{y} is [-DSS-]₆. In other embodiments, E is -D-S-S-, y is 6 and z is 1. In some embodiments, E is -D-S-S-, y is 6 and q is 0. In other embodiments, E is -D-S-S-, y is 6, z is 1, and q is 0. In other embodiments, E is -D-S-S-, y is 6, z is 1, q is 0, and x is 0. In other embodiments, E is -D-S-S-, y is 6, z is 1, q is 0, and x is 2.

In some embodiments, E is -D-S-S-, y is 6 and q is 1. In other embodiments, E is -D-S-S-, y is 6, z is 1, and q is 1. In other embodiments, E is -D-S-S-, y is 6, z is 1, x is 0, and q is 1. In other embodiments, E is -D-S-S-, y is 6, z is 1, x is 2, and q is 1.

In some embodiments, E is -D-S-S-, y is 6, q is 1, and R is (-[LK]-Fc-[DI]-). In some embodiments, E is -D-S-S-, y is 6, q is 1, R is (-[LK]-Fc-[DI]-), and [A] has at least 99% identity to any one of SEQ ID NOS: 12 and 33-43. In some embodiments, E is -D-S-S-, y is 6,
q is 1, R is (-[LK]-Fc-[DI]-), and [A] has identity to any one of SEQ ID NOS: 12 and 33-43. In some embodiments, E is -D-S-S-, y is 6, q is 1, R is (-[LK]-Fc-[DI]-), [A] has at least 99% identity to any one of SEQ ID NOS: 12 and 33-43; and [B] comprises at least 99% identity to that of SEQ ID NO: 11. In some embodiments, E is -D-S-S-, y is 6, q is 1, R is (-[LK]-Fc-[DI]-), [A] has at least 99% identity to any one of SEQ ID NOS: 12 and 33-43; and [B] comprises SEQ ID NO: 11.

In some embodiments, E is -D-S-S-, v+w is 1, and y ranges from 1 to 16. In some embodiments, E is -D-S-S-, y ranges from 1 to 12. In some embodiments, E is -D-S-S-, v+w is 1, and y ranges from 1 to 10. In some embodiments, E is -D-S-S-, v+w is 1, and y ranges from 1 to 8. In some embodiments, E is -D-S-S-, v+w is 1, and y is 6. In some embodiments, E is -D-S-S-, v+w is 1, x is 0, and y ranges from 1 to 16. In some embodiments, E is -D-S-S-, v+w is 1, x is 0, and y ranges from 1 to 12. In some embodiments, E is -D-S-S-, v+w is 1, x is 0, and y ranges from 1 to 10. In some embodiments, E is -D-S-S-, v+w is 1, x is 0, and y ranges from 1 to 8. In some embodiments, E is -D-S-S-, v+w is 1, x is 0, and y is 6. In some embodiments, E is -D-S-S-, v+w is 1, x is 0, and y is 6 and q is 0. In some embodiments, E is -D-S-S-, v+w is 1, x is 0, and y is 6. In some embodiments, E is -D-S-S-, v+w is 1, x is 0, and y is 6 and q is 1. In some embodiments, E is -D-S-S-, v+w is 1, x is 0, and y is 6. In some embodiments, E is -D-S-S-, v+w is 1, x is 0, y is 6, q is 1, and R is (-[LK]-Fc-[DI]-). In some embodiments, E is -D-S-S-, v+w is 1, x is 0, y is 6, q is 1, R is (-[LK]-Fc-[DI]-), and [A] has at least 99% identity to any one of SEQ ID NOS: 12 and 33-43. In some embodiments, E is -D-S-S-, v+w is 1, x is 0, y is 6, q is 1, R is (-[LK]-Fc-[DI]-), and [A] comprises any one of SEQ ID NOS: 12 and 33-43. In some embodiments the polypeptide is not conjugated to a dextran.

In some embodiments, E comprises 4 amino acids. In some embodiments, the 4 amino acids are selected from aspartic acid, serine, lysine, threonine, tyrosine, alanine, methionine, valine, tryptophan, proline, arginine, glutamine. In other embodiments, the 4 amino acid is selected from aspartic acid and serine. In some embodiments, E is DDSS. In other embodiments, E is -D-D-D-S-. In other embodiments, E is DDDD. In other embodiments, E is DDSS. In other embodiments, E is -D-S-S-S-. In some embodiments, E comprises 4 amino acids, and y ranges from 1 to 16. In some embodiments, E comprises 4 amino acids, and y ranges from 1 to 12. In some embodiments, E comprises 4 amino acids, and y ranges from 1 to 10. In some embodiments, E comprises 4 amino acids, and y ranges from 1 to 8. In some embodiments, E comprises 4 amino acids, and y ranges from 1 to 6. In some embodiments, E comprises 4 amino acids, and y ranges from 1 to 4. In some embodiments, E comprises 4 amino acids, and y is 1 or 2. In some embodiments, E comprises 4 amino acids, and y ranges from 2 to 12. In some embodiments, E comprises 4 amino acids, and y ranges from 2 to 10. In some embodiments, E comprises 4 amino acids, and y ranges from 2 to 8. In some embodiments, E comprises 4 amino acids, and y ranges from 2 to 8 and q is 0. In some embodiments, E comprises 4 amino acids, and y ranges from 2 to 8. In some embodiments, E comprises 4 amino acids, and y ranges from 2 to 8 and q is 1.

In some embodiments, E is -D-D-S-S-, and y ranges from 1 to 16. In some embodiments, E is -D-D-S-S-, and y ranges from 1 to 12. In some embodiments, E is -D-D-S-S-, and y ranges from 1 to 10. In some embodiments, E is aspartic acid, and y ranges from 1 to 8. In some embodiments, E is -D-D-S-S-, and y is 10. In some embodiments, E is -D-D-S-S-, v+w is 1, and y ranges from 1 to 16. In some embodiments, E is -D-D-S-S-, y ranges from 1 to 12. In some embodiments, E is -D-D-S-S-, v+w is 1, and y ranges from 1 to 10. In some embodiments, E is -D-D-S-S-, v+w is 1, and y ranges from 1 to 8. In some embodiments, E is -D-D-S-S-, v+w is 1, x is 0, and y ranges from 1 to 16. In some embodiments, E is -D-D-S-S-, v+w is 1, x is 0, and y ranges from 1 to 12. In some embodiments, E is -D-D-S-S-, v+w is 1, x is 0, and y ranges from 1 to 10. In some embodiments, E is -D-D-S-S-, v+w is 1, x is 0, and y ranges from 1 to 8. In some embodiments, E is -D-D-S-S-, v+w is 1, x is 0, and y ranges from 1 to 8 and q is 0. In some embodiments, E is -D-D-S-S-, v+w is 1, x is 0, and y ranges from 1 to 8 and q is 1.

In some embodiments, E comprises 5 or more amino acids. In some embodiments, the 5 or more amino acids are selected from aspartic acid, serine, lysine, threonine, tyrosine, alanine, methionine, valine, tryptophan, proline, arginine, glutamine. In other embodiments, the 5 or more amino acid is selected from aspartic acid and serine. In other embodiments, E comprises 5 amino acids. For example, E may be DDS SD or DDS SD. In other embodiments, E comprises 6 amino acids. For example, E may be -D-D-S-S-D-D-. In other embodiments, E comprises 7 amino acids. For example, E may be -S-D-D-S-S-D-D-. By way of another example, E may be KRRTPVR. By way of yet another example, E may be KNFQSRS. By way of a further example, E may be KTYASMQ. In other embodiments, E comprises 8 amino acids.

For example, E may be -S-D-D-S-S-D-D-S-. By way of another example, E may be KRRTPVRE. By way of yet another example, E may be KNFQSRSH. By way of a further example, E may be KTYASMQW.

In some embodiments, q is 0. In some embodiments, q is 0, and x is at least 1. In other embodiments, q is 0 and y is at least 4. In other embodiments, q is 0 and y is 6.

In yet other embodiments, q is 1 and y is at least 4. In yet other embodiments, q is 1 and y is 6. In some embodiments, q is 1, y is 6, and R is (-[LK]-Fc-[DI]-). In some embodiments, q is 1, y is 6, w is 0, and R is (-[LK]-Fc-[DI]-). In some embodiments, q is 1, y is 6, w is 0, x is 0, and R is (-[LK]-Fc-[DI]-). In some embodiments, q is 1, z is 1, y is 6, w is 0, x is 0, and R is (-[LK]-Fc-[DI]-). In some embodiments, q is 1, z is 1, y is 6, w is 0, x is 0, and R is (-[LK]-Fc-[DI]-), where Fc comprises at least 99% identity to that of SEQ ID NO: 11. In some embodiments, q is 1, z is 1, y is 6, w is 0, x is 0, and R is (-[LK]-Fc-[DI]-), where Fc comprises one amino acid substitution compared to that of SEQ ID NO: 11. In some embodiments, q is 1, z is 1, y is 6, w is 0, x is 0, and R is (-[LK]-Fc-[DI]-), where Fc comprises SEQ ID NO: 11. In some embodiments, q is 1, z is 1, y is 6, w is 0, x is 0, and R is (-[LK]-Fc-[DI]-), where Fc comprises SEQ ID NO: 11; and where [A] comprises at least 99% identity to any one of SEQ ID NOS: 12 and 33-43. In some embodiments, q is 1, z is 1, y is 6, w is 0, x is 0, and R is (-[LK]-Fc-[DI]-), where Fc comprises SEQ ID NO: 11; and where [A] comprises any one of SEQ ID NOS: 12 and 33-43. In some embodiments, the polypeptide has less than 99% identity to SEQ ID NO: 1. In some embodiments, the polypeptide has less than 98% identity to SEQ IF NO: 1. In some embodiments, the polypeptide is not conjugated to a dextran.

In some embodiments, z is an integer ranging from 1 to 6. In other embodiments, z is an integer ranging from 1 to 4. In yet other embodiments, z is an integer ranging from 1 to 3. In further embodiments, z is 1 or 2. In even further embodiments, z is 1. In even further embodiments, z is 1; and q is 0 and y is 6. In even further embodiments, z is 1; and q is 1 and y is 6.

In some embodiments, the present disclosure provides for a polypeptide having Formula (IB):

[A]ᵥ-[B]-[C]_{w}-[R]_{q}-([D]ₓ-[E]_{y})_{z}, (IB)
wherein
A comprises an amino acid sequence encoding a secretion signal peptide;
B comprises an amino acid encoding an alkaline phosphatase;
C comprises an amino acid sequence encoding a GPI anchor;
R is -(Mₒ(Fc)Nₚ)-, where M and N each independently include between 1 and 6 amino acids, where Fc is a Fc domain, and o and p are each independently 0, 1, or 2;
D comprises an amino acid sequence having between 4 and 6 amino acids, or is F(G)ₜF, where each F is the same amino acid, G is an amino acid sequence having 3, 4, or 5 amino acids, and t is an integer ranging from 2-5;
E comprises an amino acid sequence having between 1 and 8 amino acids;
q is 0 or 1;
v is 0 or 1;
w is 0 or 1;
x is 0 or an integer ranging from 1 to 6;
y is 0 or an integer ranging from 1 to 16; and
z is 0 or an integer ranging from 1 to 6;
provided that when v is 1, w is 0, q is 1, o is 1, p is 1, N is the diamino acid -D-I-, M is the diamino acid -L-K-, [B] comprises SEQ ID NO: 11, Fc comprises SEQ ID NO: 130, and x is 0, then [E]_{y} does not comprise ten to sixteen contiguous aspartic acid residues.

In some embodiments, the polypeptide of Formula (IB) is not conjugated to a dextran.

In some embodiments, the polypeptide of Formula (IB) is catalytically competent to allow formation of hydroxyapatite crystals in bone.

In some embodiments, the present disclosure provides for a polypeptide having Formula (II):

[A]-[B]-[C]-[R]_{q}-([D]ₓ-[E]_{y}), (II)
wherein
A comprises an amino acid sequence encoding a secretion signal peptide;
B comprises an amino acid encoding an alkaline phosphatase;
C comprises an amino acid sequence encoding a GPI anchor;
R is -(Mₒ(Fc)Nₚ)-, where M and N each independently include between 1 and 6 amino acids, where Fc is a Fc domain, and o and p are each independently 0, 1, or 2;
D comprises an amino acid sequence having between 4 and 6 amino acids;
E comprises an amino acid sequence having between 1 and 8 amino acids;
q is 0 or 1;
x is 0 or an integer ranging from 1 to 6; and
y is 0 or an integer ranging from 1 to 16.

In some embodiments, the polypeptide of Formula (II) is catalytically competent to allow formation of hydroxyapatite crystals in bone.

In some embodiments, E comprises between 2 and 4 amino acids. In some embodiments, E comprises at least two different amino acids. In some embodiments, E comprises at least two different amino acids and wherein y ranges from 4-8.

In some embodiments, q is 0. In some embodiments, q is 1 and x is at least 1. In some embodiments, q is 1 and y is at least 1. In some embodiments, q is 1, and both x and 1 are at least 1.

In some embodiments, the amino acid encoding the alkaline phosphate is a tissue non-specific alkaline phosphatase. In some embodiments, the amino acid encoding the alkaline phosphatase has at least about 85% identity to SEQ ID NO: 11. In other embodiments, the amino acid encoding the alkaline phosphatase has at least about 90% identity to SEQ ID NO: 11. In yet other embodiments, the amino acid encoding the alkaline phosphatase has at least about 95% identity to SEQ ID NO: 11. In further embodiments, the amino acid encoding the alkaline phosphatase has at least about 96% identity to SEQ ID NO: 11. In even further embodiments, the amino acid encoding the alkaline phosphatase has at least about 97% identity to SEQ ID NO: 11. In yet even further embodiments, the amino acid encoding the alkaline phosphatase has at least about 98% identity to SEQ ID NO: 11. In yet even further embodiments, the amino acid encoding the alkaline phosphatase has at least about 99% identity to SEQ ID NO: 11. In some embodiments, the amino acid encoding the alkaline phosphatase comprises SEQ ID NO: 11.

In some embodiments, the amino acid encoding the alkaline phosphatase comprises a variant of SEQ ID NO: 11 comprising one or more amino acid substitutions. In some embodiments, the amino acid encoding the alkaline phosphatase comprises a variant of SEQ ID NO: 11 having a single amino acid substitution. For instance, a variant of SEQ ID NO: 11 may comprises a C102S substitution. By way of another example, a variant of SEQ ID NO: 11 may comprise an E434G substitution. By way of yet another example, a variant of SEQ ID NO: 11 may comprise an A321H substitution. In some embodiments, the amino acid encoding the alkaline phosphatase comprises a variant of SEQ ID NO: 11 having two amino acid substitutions. For instance, a variant of SEQ ID NO: 11 may comprise any two of a C102S substitution, an E434G substitution, or an A321H substitution, e.g. both an A321H substitution and an E434G substitution.

In some embodiments, the amino acid encoding the secretion signal peptide has at least 90% sequence identity to SEQ ID NO: 12. In other embodiments, the amino acid encoding the secretion signal peptide has at least 95% sequence identity to SEQ ID NO: 12. In yet other embodiments, the amino acid encoding the secretion signal peptide has at least 96% sequence identity to SEQ ID NO: 12. In further embodiments, the amino acid encoding the secretion signal peptide has at least 97% sequence identity to SEQ ID NO: 12. In yet further embodiments, the amino acid encoding the secretion signal peptide has at least 98% sequence identity to SEQ ID NO: 12. In even further embodiments, the amino acid encoding the secretion signal peptide has at least 99% sequence identity to SEQ ID NO: 12. In yet even further embodiments, the amino acid encoding the secretion signal peptide comprises SEQ ID NO: 12.

In some embodiments, the amino acid encoding the secretion signal peptide has at least 90% sequence identity to any one of SEQ ID NOS: 33-43. In other embodiments, the amino acid encoding the secretion signal peptide has at least 95% sequence identity to any one of SEQ ID NOS: 33-43. In yet other embodiments, the amino acid encoding the secretion signal peptide has at least 96% sequence identity to any one of SEQ ID NOS: 33-43. In further embodiments, the amino acid encoding the secretion signal peptide has at least 97% sequence identity to any one of SEQ ID NOS: 33-43. In yet further embodiments, the amino acid encoding the secretion signal peptide has at least 98% sequence identity to any one of SEQ ID NOS: 33-43. In even further embodiments, the amino acid encoding the secretion signal peptide has at least 99% sequence identity to any one of SEQ ID NOS: 33-43. In yet even further embodiments, the amino acid encoding the secretion signal peptide comprises any one of SEQ ID NOS: 33-43.

In some embodiments, the amino acid encoding the GPI anchor has at least 90% sequence identity to any one of SEQ ID NOS: 13 and 14. In other embodiments, the amino acid encoding the GPI anchor has at least 95% sequence identity to any one of SEQ ID NOS: 13 and 14. In yet other embodiments, the amino acid encoding the GPI anchor has at least 96% sequence identity to any one of SEQ ID NOS: 13 and 14. In further embodiments, the amino acid encoding the GPI anchor has at least 97% sequence identity to SEQ ID NOS: 13 and 14. In yet further embodiments, the amino acid encoding the GPI anchor has at least 98% sequence identity to any one of SEQ ID NOS: 13 and 14. In even further embodiments, the amino acid encoding the GPI anchor has at least 99% sequence identity to any one of SEQ ID NOS: 13 and 14. In yet even further embodiments, the amino acid encoding the GPI anchor comprises any one of SEQ ID NOS: 13 and 14.

In some embodiments, the amino acid encoding the alkaline phosphatase has at least about 90% identity to SEQ ID NO: 11, and x+y=8 to 12. In some embodiments, the amino acid encoding the alkaline phosphatase has at least about 95% identity to SEQ ID NO: 11, and x+y=8 to 12. In some embodiments, the amino acid encoding the alkaline phosphatase has at least about 96% identity to SEQ ID NO: 11, and x+y=8 to 12. In some embodiments, the amino acid encoding the alkaline phosphatase has at least about 97% identity to SEQ ID NO: 11, and x+y=8 to 12. In some embodiments, the amino acid encoding the alkaline phosphatase has at least about 98% identity to SEQ ID NO: 11, and x+y=8 to 12. In some embodiments, the amino acid encoding the alkaline phosphatase has at least about 99% identity to SEQ ID NO: 11, and x+y=8 to 12. In some embodiments, the amino acid encoding the alkaline phosphatase comprises SEQ ID NO: 11, and x+y=8 to 12. In some embodiments, the amino acid encoding the alkaline phosphatase comprises SEQ ID NO: 11, and x+y=8 to 12.

In some embodiments, the amino acid encoding the alkaline phosphatase comprises SEQ ID NO: 11, and x+y=8 to 12, and q is 1. In some embodiments, the amino acid encoding the alkaline phosphatase comprises SEQ ID NO: 11, and x+y=8 to 12, q is 1; and R is (-[LK]-Fc-[DI]-). In some embodiments, the amino acid encoding the alkaline phosphatase comprises SEQ ID NO: 11, and x+y=8 to 12, q is 1; and R is (-[LK]-Fc-[DI]-), and [A] comprises an amino acid sequence having at least 95% identity to any one of SEQ ID NOS: 12 and 33-43. In some embodiments, the amino acid encoding the alkaline phosphatase comprises SEQ ID NO: 11, and x+y=8 to 12, q is 1; and R is (-[LK]-Fc-[DI]-), and [A] comprises an amino acid sequence having at least 96% identity to any one of SEQ ID NOS: 12 and 33-43. In some embodiments, the amino acid encoding the alkaline phosphatase comprises SEQ ID NO: 11, and x+y=8 to 12, q is 1; and R is (-[LK]-Fc-[DI]-), and [A] comprises an amino acid sequence having at least 97% identity to any one of SEQ ID NOS: 12 and 33-43. In some embodiments, the amino acid encoding the alkaline phosphatase comprises SEQ ID NO: 11, and x+y=8 to 12, q is 1; and R is (-[LK]-Fc-[DI]-), and [A] comprises an amino acid sequence having at least 98% identity to any one of SEQ ID NOS: 12 and 33-43. In some embodiments, the amino acid encoding the alkaline phosphatase comprises SEQ ID NO: 11, and x+y=8 to 12, q is 1; and R is (-[LK]-Fc-[DI]-), and [A] comprises an amino acid sequence having at least 99% identity to any one of SEQ ID NOS: 12 and 33-43. In some embodiments, the amino acid encoding the alkaline phosphatase comprises SEQ ID NO: 11, and x+y=8 to 12, q is 1; and R is (-[LK]-Fc-[DI]-), and [A] comprises an amino acid sequence having any one of SEQ ID NOS: 12 and 33-43.

In some embodiments, the amino acid encoding the alkaline phosphatase has at least about 90% identity to SEQ ID NO: 11, and x+y=4 to 8. In some embodiments, the amino acid encoding the alkaline phosphatase has at least about 95% identity to SEQ ID NO: 11, and x+y=4 to 8. In some embodiments, the amino acid encoding the alkaline phosphatase has at least about 96% identity to SEQ ID NO: 11, and wherein x+y=4 to 8. In some embodiments, the amino acid encoding the alkaline phosphatase has at least about 97% identity to SEQ ID NO: 11, and x+y=4 to 8. In some embodiments, the amino acid encoding the alkaline phosphatase has at least about 98% identity to SEQ ID NO: 11, and x+y=4 to 8. In some embodiments, the amino acid encoding the alkaline phosphatase has at least about 99% identity to SEQ ID NO: 11, and x+y=4 to 8. In some embodiments, the amino acid encoding the alkaline phosphatase comprises SEQ ID NO: 11, and x+y=4 to 8.

In some embodiments, D is GGGGS. In other embodiments, D is GGGSS. In yet other embodiments, D is GGSSS. In further embodiments, D is GGSGS. In even further embodiments, D is GGSGS.

In some embodiments, D is GGGGS or GGGSS, and x is an integer ranging from 1 to 4. In some embodiments, D is GGGGS or GGGSS, and x is an integer ranging from 1 to 3. In some embodiments, D is GGGGS or GGGSS, and x is 1 or 2. In some embodiments, D is GGGGS or GGGSS, and x is 2. In some embodiments, D is GGGGS, and x is 2. In some embodiments, D is GGGGS, and x is 2 and q is 0.

In some embodiments, D is GGGGS or GGGSS, and x is an integer ranging from 1 to 4, and y is an integer ranging from between 4 and 12. In some embodiments, D is GGGGS or GGGSS, x is an integer ranging from 1 to 3 and y is an integer ranging from between 4 and 12. In some embodiments, D is GGGGS or GGGSS, x is 1 or 2, and y is an integer ranging from between 4 and 12. In some embodiments, D is GGGGS or GGGSS, x is 1 or 2, and y is an integer ranging from between 4 and 12 and q is 0.

In some embodiments, E comprises 1 amino acid. In some embodiments, the 1 amino acid is selected from aspartic acid and serine. In some embodiments, E comprises 1 amino acid, and y ranges from 1 to 16. In some embodiments, E comprises 1 amino acid, and y ranges from 4 to 16. In some embodiments, E comprises 1 amino acid, and y ranges from 4 to 12. In some embodiments, E comprises 1 amino acid, and y ranges from 6 to 12. In some embodiments, E comprises 1 amino acid, and y ranges from 8 to 10. In some embodiments, E comprises 1 amino acid, and y is 10. In some embodiments, E comprises 1 amino acid, and y is 10, and wherein q is 0.

In some embodiments, E is aspartic acid, and y ranges from 1 to 16. In some embodiments, E is aspartic acid, and y ranges from 4 to 16. In some embodiments, E is aspartic acid, and y ranges from 4 to 12. In some embodiments, E is aspartic acid, and y ranges from 6 to 12. In some embodiments, E is aspartic acid and y is 10. In some embodiments, E is aspartic acid and y is 10 and q is 0.

In some embodiments, E comprises 3 amino acids. In some embodiments, the 3 amino acids are selected from aspartic acid and serine. In some embodiments, E is -D-S-S-. In other embodiments, E is -D-D-S-. In other embodiments, E is -D-D-D-. In some embodiments, E comprises 3 amino acids, and y ranges from 1 to 16. In some embodiments, E comprises 3 amino acids, and y ranges from 1 to 12. In some embodiments, E comprises 3 amino acids, and y ranges from 1 to 10. In some embodiments, E comprises 3 amino acids, and y ranges from 1 to 8. In some embodiments, E comprises 3 amino acids, and y ranges from 4 to 8. In some embodiments, E comprises 3 amino acids, and y is 6. In some embodiments, E comprises 3 amino acids, and y ranges from 4 to 8. In some embodiments, E comprises 3 amino acids, and y is 6 and q is 0.

In some embodiments, E is -D-S-S-, and y ranges from 1 to 16. In some embodiments, E is -D-S-S-, and y ranges from 1 to 12. In some embodiments, E is -D-S-S-, and y ranges from 1 to 10. In some embodiments, E is -D-S-S-, and y ranges from 4 to 8. In some embodiments, E is aspartic acid, and y ranges from 1 to 8. In some embodiments, E is -D-S-S-, and y is 6. In some embodiments, E is -D-S-S-, and y is 6 and q is 0.

In some embodiments, D is GGGGS or GGGSS, x is an integer ranging from 1 to 3, E is -D-S-S-, and y is an integer ranging from between 1 and 8. In some embodiments, D is GGGGS or GGGSS, x is 1 or 2, E is -D-S-S-, and y is an integer ranging from between 1 and 8. In some embodiments, D is GGGGS or GGGSS, x is 1 or 2, E is -D-S-S-, and y is an integer ranging from between 4 and 8. In some embodiments, D is GGGGS or GGGSS, x is 1 or 2, E is -D-S-S-, and y is 6. In some embodiments, D is GGGGS, x is 1 or 2, E is -D-S-S-, and y is 6. In some embodiments, D is GGGGS, x is 1 or 2, E is -D-S-S-, and y is 6 and q is 0.

In some embodiments, D is GGGGS or GGGSS, x is an integer ranging from 1 to 3, E is aspartic acid, and y is an integer ranging from between 8 and 12. In some embodiments, D is GGGGS or GGGSS, x is 1 or 2, E is aspartic acid, and y is 10. In some embodiments, D is GGGGS or GGGSS, x is 2, E is aspartic acid, and y is 10. In some embodiments, D is GGGGS, x is 2, E is aspartic acid, and y is 10. In some embodiments, D is GGGGS, x is 2, E is aspartic acid, and y is 10 and q is 0.

In some embodiments, E is KRRTPVRE. In other embodiments, E is KNFQSRSH. In yet other embodiments, E is KTYASMQW.

In some embodiments, the present disclosure provides for a polypeptide having Formula (III):

([A]-[B])-([D]ₓ-[E]_{y})_{z}, (III)
wherein
A comprises an amino acid sequence encoding a secretion signal peptide;
B comprises an amino acid encoding an alkaline phosphatase;
D comprises an amino acid sequence having between 4 and 6 amino acids;
E comprises an amino acid sequence having between 1 and 8 amino acids;
x is 0 or an integer ranging from 1 to 6;
y is 0 or an integer ranging from 1 to 16; and
z is an integer ranging from 1 to 6.

In some embodiments, the polypeptide of Formula (III) is catalytically competent to allow formation of hydroxyapatite crystals in bone.

In some embodiments, E comprises between 2 and 4 amino acids. In some embodiments, E comprises at least two different amino acids.

In some embodiments, x+y is at least 2, and where [D] and [E] comprise different amino acid sequences.

In other embodiments, x is 0, and [E] comprises at least three amino acids. In other embodiments, x is 0, and [E] comprises three amino acids. In other embodiments, x is 0, [E] comprises three amino acids, and y ranges from between 4 and 8. In other embodiments, x is 0, [E] comprises three amino acids, and y is 6.

In some embodiments, the amino acid encoding the alkaline phosphatase has at least about 90% identity to SEQ ID NO: 11. In yet other embodiments, the amino acid encoding the alkaline phosphatase has at least about 95% identity to SEQ ID NO: 11. In further embodiments, the amino acid encoding the alkaline phosphatase has at least about 96% identity to SEQ ID NO: 11. In even further embodiments, the amino acid encoding the alkaline phosphatase has at least about 97% identity to SEQ ID NO: 11. In yet even further embodiments, the amino acid encoding the alkaline phosphatase has at least about 98% identity to SEQ ID NO: 11. In yet even further embodiments, the amino acid encoding the alkaline phosphatase has at least about 99% identity to SEQ ID NO: 11. In some embodiments, the amino acid encoding the alkaline phosphatase comprises SEQ ID NO: 11.

In some embodiments, the amino acid encoding the alkaline phosphatase comprises a variant of SEQ ID NO: 11 comprising one or more amino acid substitutions. In some embodiments, the amino acid encoding the alkaline phosphatase comprises a variant of SEQ ID NO: 11 having a single amino acid substitution. For instance, a variant of SEQ ID NO: 11 may comprises a C102S substitution. By way of another example, a variant of SEQ ID NO: 11 may comprise an E434G substitution. By way of yet another example, a variant of SEQ ID NO: 11 may comprise an A321H substitution. In some embodiments, the amino acid encoding the alkaline phosphatase comprises a variant of SEQ ID NO: 11 having two amino acid substitutions. For instance, a variant of SEQ ID NO: 11 may comprise any two of a C102S substitution, an E434G substitution, or an A321H substitution, e.g. both an A321H substitution and an E434G substitution.

In some embodiments, the amino acid encoding the secretion signal peptide has at least 95% sequence identity to SEQ ID NO: 12. In yet other embodiments, the amino acid encoding the secretion signal peptide has at least 96% sequence identity to SEQ ID NO: 12. In further embodiments, the amino acid encoding the secretion signal peptide has at least 97% sequence identity to SEQ ID NO: 12. In yet further embodiments, the amino acid encoding the secretion signal peptide has at least 98% sequence identity to SEQ ID NO: 12. In even further embodiments, the amino acid encoding the secretion signal peptide has at least 99% sequence identity to SEQ ID NO: 12. In yet even further embodiments, the amino acid encoding the secretion signal peptide comprises SEQ ID NO: 12.

In some embodiments, the amino acid encoding the secretion signal peptide has at least 90% sequence identity to any one of SEQ ID NOS: 33-43. In other embodiments, the amino acid encoding the secretion signal peptide has at least 95% sequence identity to any one of SEQ ID NOS: 33-43. In yet other embodiments, the amino acid encoding the secretion signal peptide has at least 96% sequence identity to any one of SEQ ID NOS: 33-43. In further embodiments, the amino acid encoding the secretion signal peptide has at least 97% sequence identity to any one of SEQ ID NOS: 33-43. In yet further embodiments, the amino acid encoding the secretion signal peptide has at least 98% sequence identity to any one of SEQ ID NOS: 33-43. In even further embodiments, the amino acid encoding the secretion signal peptide has at least 99% sequence identity to any one of SEQ ID NOS: 33-43. In yet even further embodiments, the amino acid encoding the secretion signal peptide comprises any one of SEQ ID NOS: 33-43.

In some embodiments, the amino acid encoding the alkaline phosphatase has at least about 90% identity to SEQ ID NO: 11, and x+y=8 to 12. In some embodiments, the amino acid encoding the alkaline phosphatase has at least about 95% identity to SEQ ID NO: 11, and x+y=8 to 12. In some embodiments, the amino acid encoding the alkaline phosphatase has at least about 96% identity to SEQ ID NO: 11, and x+y=8 to 12. In some embodiments, the amino acid encoding the alkaline phosphatase has at least about 97% identity to SEQ ID NO: 11, and x+y=8 to 12. In some embodiments, the amino acid encoding the alkaline phosphatase has at least about 98% identity to SEQ ID NO: 11, and x+y=8 to 12. In some embodiments, the amino acid encoding the alkaline phosphatase has at least about 99% identity to SEQ ID NO: 11, and x+y=8 to 12. In some embodiments, the amino acid encoding the alkaline phosphatase comprises SEQ ID NO: 11, and x+y=8 to 12. In some embodiments, the amino acid encoding the alkaline phosphatase comprises SEQ ID NO: 11, and x+y=8 to 12.

In some embodiments, the amino acid encoding the alkaline phosphatase has at least about 90% identity to SEQ ID NO: 11, and x+y=4 to 8. In some embodiments, the amino acid encoding the alkaline phosphatase has at least about 95% identity to SEQ ID NO: 11, and x+y=4 to 8. In some embodiments, the amino acid encoding the alkaline phosphatase has at least about 96% identity to SEQ ID NO: 11, and wherein x+y=4 to 8. In some embodiments, the amino acid encoding the alkaline phosphatase has at least about 97% identity to SEQ ID NO: 11, and x+y=4 to 8. In some embodiments, the amino acid encoding the alkaline phosphatase has at least about 98% identity to SEQ ID NO: 11, and x+y=4 to 8. In some embodiments, the amino acid encoding the alkaline phosphatase has at least about 99% identity to SEQ ID NO: 11, and x+y=4 to 8. In some embodiments, the amino acid encoding the alkaline phosphatase comprises SEQ ID NO: 11, and x+y=4 to 8.

In some embodiments, D is GGGGS. In other embodiments, D is GGGSS. In yet other embodiments, D is GGSSS. In further embodiments, D is GGSGS. In even further embodiments, D is GGSGS.

In some embodiments, D is GGGGS or GGGSS, and x is an integer ranging from 1 to 4. In some embodiments, D is GGGGS or GGGSS, and x is an integer ranging from 1 to 3. In some embodiments, D is GGGGS or GGGSS, and x is 1 or 2. In some embodiments, D is GGGGS or GGGSS, and x is 2. In some embodiments, D is GGGGS, and x is 2.

In some embodiments, D is GGGGS or GGGSS, and x is an integer ranging from 1 to 4, and y is an integer ranging from between 4 and 12. In some embodiments, D is GGGGS or GGGSS, x is an integer ranging from 1 to 3 and y is an integer ranging from between 4 and 12. In some embodiments, D is GGGGS or GGGSS, x is 1 or 2, and y is an integer ranging from between 4 and 12. In some embodiments, D is GGGGS or GGGSS, x is 1 or 2, and y is 6. In some embodiments, D is GGGGS or GGGSS, x is 1 or 2, and y is 10.

In some embodiments, E comprises 1 amino acid, and y ranges from 4 to 16. In some embodiments, E comprises 1 amino acid, and y ranges from 4 to 12. In some embodiments, E comprises 1 amino acid, and y ranges from 6 to 12. In some embodiments, E comprises 1 amino acid, and y ranges from 8 to 10. In some embodiments, E comprises 1 amino acid, and y is 10.

In some embodiments, E is aspartic acid, and y ranges from 4 to 16. In some embodiments, E is aspartic acid, and y ranges from 4 to 12. In some embodiments, E is aspartic acid, and y ranges from 6 to 12. In some embodiments, E is aspartic acid, and y ranges from 8 to 10. In some embodiments, E is aspartic acid, and y is 10.

In some embodiments, E comprises 3 amino acids. In some embodiments, E comprises 3 amino acids, and y ranges from 1 to 16. In some embodiments, E comprises 3 amino acids, and y ranges from 1 to 12. In some embodiments, E comprises 3 amino acids, and y ranges from 1 to 10. In some embodiments, E comprises 3 amino acids, and y ranges from 1 to 8. In some embodiments, E comprises 3 amino acids, and y ranges from 4 to 8. In some embodiments, E comprises 3 amino acids, and y is 6.

In some embodiments, E is -D-S-S-. In other embodiments, E is -D-D-S-. In yet other embodiments, E is -D-D-D-.

In some embodiments, E is -D-S-S-, and y ranges from 1 to 16. In some embodiments, E is -D-S-S-, and y ranges from 1 to 12. In some embodiments, E is -D-S-S-, and y ranges from 1 to 10. In some embodiments, E is -D-S-S-, and y ranges from 4 to 8. In some embodiments, E is aspartic acid, and y ranges from 1 to 8. In some embodiments, E is -D-S-S-, and y is 6.

In some embodiments, E is KRRTPVRE. In other embodiments, E is KNFQSRSH. In yet other embodiments, E is KTYASMQW.

In some embodiments, D is GGGGS or GGGSS, x is an integer ranging from 1 to 3, E is -D-S-S-, and y is an integer ranging from between 1 and 8. In some embodiments, D is GGGGS or GGGSS, x is 1 or 2, E is -D-S-S-, and y is an integer ranging from between 1 and 8. In some embodiments, D is GGGGS or GGGSS, x is 1 or 2, E is -D-S-S-, and y is an integer ranging from between 4 and 8. In some embodiments, D is GGGGS or GGGSS, x is 1 or 2, E is -D-S-S-, and y is 6. In some embodiments, D is GGGGS, x is 1 or 2, E is -D-S-S-, and y is 6.

In some embodiments, D is GGGGS or GGGSS, x is an integer ranging from 1 to 3, E is aspartic acid, and y is an integer ranging from between 8 and 12. In some embodiments, D is GGGGS or GGGSS, x is 1 or 2, E is aspartic acid, and y is 10. In some embodiments, D is GGGGS or GGGSS, x is 2, E is aspartic acid, and y is 10. In some embodiments, D is GGGGS,
x is 2, E is aspartic acid, and y is 10.

In some embodiments, the present disclosure provides for a polypeptide having Formula (IV):

([A]-[B])-([E]_{y}), (IV)
wherein
A comprises an amino acid sequence encoding a secretion signal peptide;
B comprises an amino acid encoding an alkaline phosphatase;
E comprises an amino acid sequence having between 1 and 8 amino acids; and
y is 0 or an integer ranging from 1 to 16.

In some embodiments, the polypeptide of Formula (IV) is catalytically competent to allow formation of hydroxyapatite crystals in bone.

In some embodiments, E comprises between 2 and 4 amino acids. In some embodiments, E comprises at least two different amino acids.

In some embodiments, the amino acid encoding the alkaline phosphatase has at least about 90% identity to SEQ ID NO: 11. In yet other embodiments, the amino acid encoding the alkaline phosphatase has at least about 95% identity to SEQ ID NO: 11. In further embodiments, the amino acid encoding the alkaline phosphatase has at least about 96% identity to SEQ ID NO: 11. In even further embodiments, the amino acid encoding the alkaline phosphatase has at least about 97% identity to SEQ ID NO: 11. In yet even further embodiments, the amino acid encoding the alkaline phosphatase has at least about 98% identity to SEQ ID NO: 11. In yet even further embodiments, the amino acid encoding the alkaline phosphatase has at least about 99% identity to SEQ ID NO: 11. In some embodiments, the amino acid encoding the alkaline phosphatase comprises SEQ ID NO: 11.

In some embodiments, the amino acid encoding the alkaline phosphatase comprises a variant of SEQ ID NO: 11 comprising one or more amino acid substitutions. In some embodiments, the amino acid encoding the alkaline phosphatase comprises a variant of SEQ ID NO: 11 having a single amino acid substitution. For instance, a variant of SEQ ID NO: 11 may comprises a C102S substitution. By way of another example, a variant of SEQ ID NO: 11 may comprise an E434G substitution. By way of yet another example, a variant of SEQ ID NO: 11 may comprise an A321H substitution. In some embodiments, the amino acid encoding the alkaline phosphatase comprises a variant of SEQ ID NO: 11 having two amino acid substitutions. For instance, a variant of SEQ ID NO: 11 may comprise any two of a C102S substitution, an E434G substitution, or an A321H substitution, e.g. both an A321H substitution and an E434G substitution.

In some embodiments, the amino acid encoding the secretion signal peptide has at least 95% sequence identity to SEQ ID NO: 12. In yet other embodiments, the amino acid encoding the secretion signal peptide has at least 96% sequence identity to SEQ ID NO: 12. In further embodiments, the amino acid encoding the secretion signal peptide has at least 97% sequence identity to SEQ ID NO: 12. In yet further embodiments, the amino acid encoding the secretion signal peptide has at least 98% sequence identity to SEQ ID NO: 12. In even further embodiments, the amino acid encoding the secretion signal peptide has at least 99% sequence identity to SEQ ID NO: 12. In yet even further embodiments, the amino acid encoding the secretion signal peptide comprises SEQ ID NO: 12.

In some embodiments, the amino acid encoding the secretion signal peptide has at least 90% sequence identity to any one of SEQ ID NOS: 33-43. In other embodiments, the amino acid encoding the secretion signal peptide has at least 95% sequence identity to any one of SEQ ID NOS: 33-43. In yet other embodiments, the amino acid encoding the secretion signal peptide has at least 96% sequence identity to any one of SEQ ID NOS: 33-43. In further embodiments, the amino acid encoding the secretion signal peptide has at least 97% sequence identity to any one of SEQ ID NOS: 33-43. In yet further embodiments, the amino acid encoding the secretion signal peptide has at least 98% sequence identity to any one of SEQ ID NOS: 33-43. In even further embodiments, the amino acid encoding the secretion signal peptide has at least 99% sequence identity to any one of SEQ ID NOS: 33-43. In yet even further embodiments, the amino acid encoding the secretion signal peptide comprises any one of SEQ ID NOS: 33-43.

In some embodiments, E comprises 1 amino acid, and y ranges from 4 to 16. In some embodiments, E comprises 1 amino acid, and y ranges from 4 to 12. In some embodiments, E comprises 1 amino acid, and y ranges from 6 to 12. In some embodiments, E comprises 1 amino acid, and y ranges from 8 to 10. In some embodiments, E comprises 1 amino acid, and y is 10.

In some embodiments, E is aspartic acid, and y ranges from 4 to 16. In some embodiments, E is aspartic acid, and y ranges from 4 to 12. In some embodiments, E is aspartic acid, and y ranges from 6 to 12. In some embodiments, E is aspartic acid, and y ranges from 8 to 10. In some embodiments, E is aspartic acid, and y is 10.

In some embodiments, E comprises 3 amino acids. In some embodiments, E comprises 3 amino acids, and y ranges from 1 to 16. In some embodiments, E comprises 3 amino acids, and y ranges from 1 to 12. In some embodiments, E comprises 3 amino acids, and y ranges from 1 to 10. In some embodiments, E comprises 3 amino acids, and y ranges from 1 to 8. In some embodiments, E comprises 3 amino acids, and y ranges from 4 to 8. In some embodiments, E comprises 3 amino acids, and y is 6.

In some embodiments, E is -D-S-S-. In other embodiments, E is -D-D-S-. In other embodiments, E is -D-D-D-.

In some embodiments, E is -D-S-S-, and y ranges from 1 to 16. In some embodiments, E is -D-S-S-, and y ranges from 1 to 12. In some embodiments, E is -D-S-S-, and y ranges from 1 to 10. In some embodiments, E is -D-S-S-, and y ranges from 4 to 8. In some embodiments, E is aspartic acid, and y ranges from 1 to 8. In some embodiments, E is -D-S-S-, and y is 6.

In some embodiments, E comprises KRRTPVRE. In other embodiments, E comprises KNFQSRSH. In yet other embodiments, E comprises KTYASMQW.

In some embodiments, the present disclosure provides for a polypeptide having Formula (VA):

[A]-[B]-[R]_{q}-([E]_{y}) (VA)
wherein
A comprises an amino acid sequence encoding a secretion signal peptide;
B comprises an amino acid encoding an alkaline phosphatase;
R is -(Mₒ(Fc)Nₚ)-, where M and N each independently include between 1 and 6 amino acids, where Fc is a Fc domain, and o and p are each independently 0, 1, or 2;
q is 0 or 1;
E comprises an amino acid sequence having between 2 and 4 amino acids; and
y is integer ranging from 1 to 16.

In some embodiments, the polypeptide of Formula (IV) is catalytically competent to allow formation of hydroxyapatite crystals in bone.

In some embodiments, y is an integer ranging from 4-8. In some embodiments, y is 6. In some embodiments, y is an integer ranging from 4-8 and q is 0. In some embodiments, y is 6 and q is 0. In some embodiments, y is an integer ranging from 4-8 and q is 1. In some embodiments, y is 6 and q is 1.

In some embodiments, the amino acid encoding the alkaline phosphatase (“[B]”) is a tissue non-specific alkaline phosphatase. In some embodiments, the amino acid encoding the alkaline phosphatase has at least about 85% identity to SEQ ID NO: 11. In other embodiments, the amino acid encoding the alkaline phosphatase has at least about 90% identity to SEQ ID NO: 11. In other embodiments, the amino acid encoding the alkaline phosphatase has at least about 91% identity to SEQ ID NO: 11. In other embodiments, the amino acid encoding the alkaline phosphatase has at least about 92% identity to SEQ ID NO: 11. In other embodiments, the amino acid encoding the alkaline phosphatase has at least about 93% identity to SEQ ID NO: 11. In other embodiments, the amino acid encoding the alkaline phosphatase has at least about 94% identity to SEQ ID NO: 11.

In yet other embodiments, the amino acid encoding the alkaline phosphatase has at least about 95% identity to SEQ ID NO: 11. In further embodiments, the amino acid encoding the alkaline phosphatase has at least about 96% identity to SEQ ID NO: 11. In even further embodiments, the amino acid encoding the alkaline phosphatase has at least about 97% identity to SEQ ID NO: 11. In yet even further embodiments, the amino acid encoding the alkaline phosphatase has at least about 98% identity to SEQ ID NO: 11. In yet even further embodiments, the amino acid encoding the alkaline phosphatase has at least about 99% identity to SEQ ID NO: 11. In yet even further embodiments, the amino acid encoding the alkaline phosphatase has at least about 99% identity to SEQ ID NO: 11 and q is 0. In yet even further embodiments, the amino acid encoding the alkaline phosphatase has at least about 99% identity to SEQ ID NO: 11 and q is 1.

In some embodiments, the amino acid encoding the alkaline phosphatase comprises a variant of SEQ ID NO: 11, such as a variant comprising one or more amino acid substitutions. In some embodiments, the amino acid encoding the alkaline phosphatase comprises a variant of SEQ ID NO: 11 having a single amino acid substitution. For instance, a variant of SEQ ID NO: 11 may comprises a C102S substitution. By way of another example, a variant of SEQ ID NO: 11 may comprise an E434G substitution. By way of yet another example, a variant of SEQ ID NO: 11 may comprise an A321H substitution. In some embodiments, the amino acid encoding the alkaline phosphatase comprises a variant of SEQ ID NO: 11 having two amino acid substitutions. For instance, a variant of SEQ ID NO: 11 may comprise any two of a C102S substitution, an E434G substitution, or an A321H substitution, e.g. both an A321H substitution and an E434G substitution. In some embodiments, the amino acid encoding the alkaline phosphatase comprises a variant of SEQ ID NO: 11 having three amino acid substitutions. In some embodiments, the amino acid encoding the alkaline phosphatase comprises a variant of SEQ ID NO: 11 having four amino acid substitutions. In some embodiments, the amino acid encoding the alkaline phosphatase comprises a variant of SEQ ID NO: 11 having five amino acid substitutions. In some embodiments, the amino acid encoding the alkaline phosphatase comprises a variant of SEQ ID NO: 11 having six amino acid substitutions.

In some embodiments, the amino acid encoding the secretion signal peptide has at least 90% sequence identity to SEQ ID NO: 12. In other embodiments, the amino acid encoding the secretion signal peptide has at least 95% sequence identity to SEQ ID NO: 12. In yet other embodiments, the amino acid encoding the secretion signal peptide has at least 96% sequence identity to SEQ ID NO: 12. In further embodiments, the amino acid encoding the secretion signal peptide has at least 97% sequence identity to SEQ ID NO: 12. In yet further embodiments, the amino acid encoding the secretion signal peptide has at least 98% sequence identity to SEQ ID NO: 12. In even further embodiments, the amino acid encoding the secretion signal peptide has at least 99% sequence identity to SEQ ID NO: 12. In yet even further embodiments, the amino acid encoding the secretion signal peptide comprises SEQ ID NO: 12. In yet even other embodiments, the amino acid encoding the secretion signal peptide comprises SEQ ID NO: 12, and wherein [D]ₓ is [-GGGGS-]₂. In yet even other embodiments, the amino acid encoding the secretion signal peptide comprises SEQ ID NO: 12, and wherein [E]_{y} is [-DSS-]₆. In yet even other embodiments, the amino acid encoding the secretion signal peptide comprises SEQ ID NO: 12; [E]_{y} is [-DSS-]₆, and q is 0. In yet even other embodiments, the amino acid encoding the secretion signal peptide comprises SEQ ID NO: 12; [E]_{y} is [-DSS-]₆, and q is 1.

In some embodiments, the amino acid encoding the secretion signal peptide has at least 90% sequence identity to any one of SEQ ID NOS: 33-43. In other embodiments, the amino acid encoding the secretion signal peptide has at least 95% sequence identity to any one of SEQ ID NOS: 33-43. In yet other embodiments, the amino acid encoding the secretion signal peptide has at least 96% sequence identity to any one of SEQ ID NOS: 33-43. In further embodiments, the amino acid encoding the secretion signal peptide has at least 97% sequence identity to any one of SEQ ID NOS: 33-43. In yet further embodiments, the amino acid encoding the secretion signal peptide has at least 98% sequence identity to any one of SEQ ID NOS: 33-43. In even further embodiments, the amino acid encoding the secretion signal peptide has at least 99% sequence identity to any one of SEQ ID NOS: 33-43. In yet even further embodiments, the amino acid encoding the secretion signal peptide comprises any one of SEQ ID NOS: 33-43. In yet even other embodiments, the amino acid encoding the secretion signal peptide comprises any one of SEQ ID NOS: 33-43, and wherein [E]_{y} is [-DSS-]₆. In yet even other embodiments, the amino acid encoding the secretion signal peptide comprises any one of SEQ ID NOS: 33-43, [E]_{y} is [-DSS-]₆, and q is 0. In yet even other embodiments, the amino acid encoding the secretion signal peptide comprises any one of SEQ ID NOS: 33-43, [E]_{y} is [-DSS-]₆, and q is 1.

In some embodiments, the Fc domain has at least 90% sequence identity to that of SEQ ID NO: 130. In some embodiments, the Fc domain has at least 91% sequence identity to that of SEQ ID NO: 130. In some embodiments, the Fc domain has at least 92% sequence identity to that of SEQ ID NO: 130. In some embodiments, the Fc domain has at least 93% sequence identity to that of SEQ ID NO: 130. In some embodiments, the Fc domain has at least 94% sequence identity to that of SEQ ID NO: 130. In some embodiments, the Fc domain has at least 95% sequence identity to that of SEQ ID NO: 130. In some embodiments, the Fc domain has at least 96% sequence identity to that of SEQ ID NO: 130. In some embodiments, the Fc domain has at least 97% sequence identity to that of SEQ ID NO: 130. In some embodiments, the Fc domain has at least 98% sequence identity to that of SEQ ID NO: 130. In some embodiments, the Fc domain has at least 99% sequence identity to that of SEQ ID NO: 130. In some embodiments, the Fc domain comprises SEQ ID NO: 130. In some embodiments, the Fc domain comprises SEQ ID NO: 130 and y ranges from 4 to 6. In some embodiments, the Fc domain comprises SEQ ID NO: 130 and [E]_{y} is [DSS]₆. In some embodiments, the R comprises SEQ ID NO: 9 and [E]_{y} is [DSS]₆.

In some embodiments, M comprises 2 amino acids. For example, M may comprise leucine and lysine. In some embodiments, M is leucine-lysine. By way of another example, M may comprise two alanine amino acids In other embodiments, M comprises three amino acids.

In some embodiments, N comprises 2 amino acids (i.e., a diamino acid). For example, N may comprise aspartic acid and isoleucine. By way of another example, N may comprise two alanine amino acids. In some embodiments, N is aspartic acid—isoleucine. In other embodiments, N comprises 3 amino acids.

In some embodiments, M is leucine-lysine and N is aspartic acid—isoleucine. In some embodiments, M is leucine-lysine and N is aspartic acid—isoleucine and Fc has at least 90% sequence identity to that of SEQ ID NO: 130. In some embodiments, M is leucine-lysine and N is aspartic acid—isoleucine and Fc has at least 91% sequence identity to that of SEQ ID NO: 130. In some embodiments, M is leucine-lysine and N is aspartic acid—isoleucine and Fc has at least 92% sequence identity to that of SEQ ID NO: 130. In some embodiments, M is leucine-lysine and N is aspartic acid—isoleucine and Fc has at least 93% sequence identity to that of SEQ ID NO: 130. In some embodiments, M is leucine-lysine and N is aspartic acid—isoleucine and Fc has at least 94% sequence identity to that of SEQ ID NO: 130. In some embodiments, M is leucine-lysine and N is aspartic acid—isoleucine and Fc has at least 95% sequence identity to that of SEQ ID NO: 130. In some embodiments, M is leucine-lysine and N is aspartic acid—isoleucine and Fc has at least 96% sequence identity to that of SEQ ID NO: 130. In some embodiments, M is leucine-lysine and N is aspartic acid—isoleucine and Fc has at least 97% sequence identity to that of SEQ ID NO: 130. In some embodiments, M is leucine-lysine and N is aspartic acid—isoleucine and Fc has at least 98% sequence identity to that of SEQ ID NO: 130. In some embodiments, M is leucine-lysine and N is aspartic acid—isoleucine and Fc has at least 99% sequence identity to that of SEQ ID NO: 130. In some embodiments, M is leucine-lysine and N is aspartic acid—isoleucine and Fc comprises SEQ ID NO: 130.

In some embodiments, M is leucine-lysine and N is aspartic acid—isoleucine; and o, p, and q are each 1. In some embodiments, M is leucine-lysine and N is aspartic acid—isoleucine and Fc has at least 90% sequence identity to that of SEQ ID NO: 130; and o, p, and q are each 1. In some embodiments, M is leucine-lysine and N is aspartic acid—isoleucine and Fc has at least 91% sequence identity to that of SEQ ID NO: 130; and o, p, and q are each 1. In some embodiments, M is leucine-lysine and N is aspartic acid—isoleucine and Fc has at least 92% sequence identity to that of SEQ ID NO: 130; and o, p, and q are each 1. In some embodiments, M is leucine-lysine and N is aspartic acid—isoleucine and Fc has at least 93% sequence identity to that of SEQ ID NO: 130; and o, p, and q are each 1. In some embodiments, M is leucine-lysine and N is aspartic acid—isoleucine and Fc has at least 94% sequence identity to that of SEQ ID NO: 130; and o, p, and q are each 1. In some embodiments, M is leucine-lysine and N is aspartic acid—isoleucine and Fc has at least 95% sequence identity to that of SEQ ID NO: 130; and o, p, and q are each 1. In some embodiments, M is leucine-lysine and N is aspartic acid—isoleucine and Fc has at least 96% sequence identity to that of SEQ ID NO: 130; and o, p, and q are each 1. In some embodiments, M is leucine-lysine and N is aspartic acid—isoleucine and Fc has at least 97% sequence identity to that of SEQ ID NO: 130; and o, p, and q are each 1. In some embodiments, M is leucine-lysine and N is aspartic acid—isoleucine and Fc has at least 98% sequence identity to that of SEQ ID NO: 130; and o, p, and q are each 1. In some embodiments, M is leucine-lysine and N is aspartic acid—isoleucine and Fc has at least 99% sequence identity to that of SEQ ID NO: 130; and o, p, and q are each 1. In some embodiments, M is leucine-lysine; N is aspartic acid—isoleucine and Fc comprises SEQ ID NO: 130; and o, p, and q are each 1. In some embodiments, M is leucine-lysine; N is aspartic acid—isoleucine; Fc comprises SEQ ID NO: 130; o, p, and q are each 1; and [A] has at least 99% identity to any one of SEQ ID NOS: 12 and 33-43. In some embodiments, M is leucine-lysine; N is aspartic acid—isoleucine; Fc comprises; o, p, and q are each 1; and [A] comprises any one of SEQ ID NOS: 12 and 33-43.

In some embodiments, E comprises 3 amino acids. In some embodiments, E comprises 3 amino acids, and y ranges from 1 to 16. In some embodiments, E comprises 3 amino acids, and y ranges from 1 to 12. In some embodiments, E comprises 3 amino acids, and y ranges from 1 to 10. In some embodiments, E comprises 3 amino acids, and y ranges from 1 to 8. In some embodiments, E comprises 3 amino acids, and y ranges from 4 to 8. In some embodiments, E comprises 3 amino acids, and y is 6.

In some embodiments, E is -D-S-S-. In other embodiments, E is -D-D-S-.

In some embodiments, E is -D-S-S-, and y ranges from 1 to 16. In some embodiments, E is -D-S-S-, and y ranges from 1 to 12. In some embodiments, E is -D-S-S-, and y ranges from 1 to 10. In some embodiments, E is -D-S-S-, and y ranges from 4 to 8. In some embodiments, E is aspartic acid, and y ranges from 1 to 8. In some embodiments, E is -D-S-S-, and y is 6.

In some embodiments, the present disclosure provides for a polypeptide having Formula (VB):

[A]-[B]-[R]_{q}-([E]_{y}) (VB)
wherein
A comprises an amino acid sequence encoding a secretion signal peptide;
B comprises an amino acid encoding an alkaline phosphatase;
R is -(Mₒ(Fc)Nₚ)-, where M and N each independently include between 1 and 6 amino acids, where Fc is a Fc domain, and o and p are each independently 0, 1, or 2;
q is 0 or 1;
E comprises an amino acid sequence having between 2 and 4 amino acids; and
y is integer ranging from 1 to 16,
provided that when q is 1 and when [E] comprises only aspartic acid, then the polypeptide of Formula (V) does not comprise an amino acid sequence having ten to sixteen contiguous aspartic acid residues; or provided that the polypeptide of Formula (V) does not have the amino acid sequence of SEQ ID NO: 1 or does not comprise an amino acid sequence encoded by the nucleotide sequence of SEQ ID NO: 107; or provided that the polypeptide of Formula (V) is not Strensiq® or Asfotase alfa.

In some embodiments, the polypeptide of Formula (IV) is catalytically competent to allow formation of hydroxyapatite crystals in bone.

In some embodiments, y is an integer ranging from 4-8. In some embodiments, y is 6. In some embodiments, y is an integer ranging from 4-8 and q is 0. In some embodiments, y is 6 and q is 0. In some embodiments, y is an integer ranging from 4-8 and q is 1. In some embodiments, y is 6 and q is 1.

In some embodiments, the amino acid encoding the alkaline phosphatase (“[B]”) is a tissue non-specific alkaline phosphatase. In some embodiments, the amino acid encoding the alkaline phosphatase has at least about 85% identity to SEQ ID NO: 11. In other embodiments, the amino acid encoding the alkaline phosphatase has at least about 90% identity to SEQ ID NO: 11. In other embodiments, the amino acid encoding the alkaline phosphatase has at least about 91% identity to SEQ ID NO: 11. In other embodiments, the amino acid encoding the alkaline phosphatase has at least about 92% identity to SEQ ID NO: 11. In other embodiments, the amino acid encoding the alkaline phosphatase has at least about 93% identity to SEQ ID NO: 11. In other embodiments, the amino acid encoding the alkaline phosphatase has at least about 94% identity to SEQ ID NO: 11.

In yet other embodiments, the amino acid encoding the alkaline phosphatase has at least about 95% identity to SEQ ID NO: 11. In further embodiments, the amino acid encoding the alkaline phosphatase has at least about 96% identity to SEQ ID NO: 11. In even further embodiments, the amino acid encoding the alkaline phosphatase has at least about 97% identity to SEQ ID NO: 11. In yet even further embodiments, the amino acid encoding the alkaline phosphatase has at least about 98% identity to SEQ ID NO: 11. In yet even further embodiments, the amino acid encoding the alkaline phosphatase has at least about 99% identity to SEQ ID NO: 11. In yet even further embodiments, the amino acid encoding the alkaline phosphatase has at least about 99% identity to SEQ ID NO: 11 and q is 0. In yet even further embodiments, the amino acid encoding the alkaline phosphatase has at least about 99% identity to SEQ ID NO: 11 and q is 1.

In some embodiments, the amino acid encoding the alkaline phosphatase comprises a variant of SEQ ID NO: 11, such as a variant comprising one or more amino acid substitutions. In some embodiments, the amino acid encoding the alkaline phosphatase comprises a variant of SEQ ID NO: 11 having a single amino acid substitution. For instance, a variant of SEQ ID NO: 11 may comprises a C102S substitution. By way of another example, a variant of SEQ ID NO: 11 may comprise an E434G substitution. By way of yet another example, a variant of SEQ ID NO: 11 may comprise an A321H substitution. In some embodiments, the amino acid encoding the alkaline phosphatase comprises a variant of SEQ ID NO: 11 having two amino acid substitutions. For instance, a variant of SEQ ID NO: 11 may comprise any two of a C102S substitution, an E434G substitution, or an A321H substitution, e.g. both an A321H substitution and an E434G substitution. In some embodiments, the amino acid encoding the alkaline phosphatase comprises a variant of SEQ ID NO: 11 having three amino acid substitutions. In some embodiments, the amino acid encoding the alkaline phosphatase comprises a variant of SEQ ID NO: 11 having four amino acid substitutions. In some embodiments, the amino acid encoding the alkaline phosphatase comprises a variant of SEQ ID NO: 11 having five amino acid substitutions. In some embodiments, the amino acid encoding the alkaline phosphatase comprises a variant of SEQ ID NO: 11 having six amino acid substitutions.

In some embodiments, the amino acid encoding the secretion signal peptide has at least 90% sequence identity to SEQ ID NO: 12. In other embodiments, the amino acid encoding the secretion signal peptide has at least 95% sequence identity to SEQ ID NO: 12. In yet other embodiments, the amino acid encoding the secretion signal peptide has at least 96% sequence identity to SEQ ID NO: 12. In further embodiments, the amino acid encoding the secretion signal peptide has at least 97% sequence identity to SEQ ID NO: 12. In yet further embodiments, the amino acid encoding the secretion signal peptide has at least 98% sequence identity to SEQ ID NO: 12. In even further embodiments, the amino acid encoding the secretion signal peptide has at least 99% sequence identity to SEQ ID NO: 12. In yet even further embodiments, the amino acid encoding the secretion signal peptide comprises SEQ ID NO: 12. In yet even other embodiments, the amino acid encoding the secretion signal peptide comprises SEQ ID NO: 12, and wherein [D]ₓ is [-GGGGS-]₂. In yet even other embodiments, the amino acid encoding the secretion signal peptide comprises SEQ ID NO: 12, and wherein [E]_{y} is [-DSS-]₆. In yet even other embodiments, the amino acid encoding the secretion signal peptide comprises SEQ ID NO: 12; [E]_{y} is [-DSS-]₆, and q is 0. In yet even other embodiments, the amino acid encoding the secretion signal peptide comprises SEQ ID NO: 12; [E]_{y} is [-DSS-]₆, and q is 1.

In some embodiments, the amino acid encoding the secretion signal peptide has at least 90% sequence identity to any one of SEQ ID NOS: 33-43. In other embodiments, the amino acid encoding the secretion signal peptide has at least 95% sequence identity to any one of SEQ ID NOS: 33-43. In yet other embodiments, the amino acid encoding the secretion signal peptide has at least 96% sequence identity to any one of SEQ ID NOS: 33-43. In further embodiments, the amino acid encoding the secretion signal peptide has at least 97% sequence identity to any one of SEQ ID NOS: 33-43. In yet further embodiments, the amino acid encoding the secretion signal peptide has at least 98% sequence identity to any one of SEQ ID NOS: 33-43. In even further embodiments, the amino acid encoding the secretion signal peptide has at least 99% sequence identity to any one of SEQ ID NOS: 33-43. In yet even further embodiments, the amino acid encoding the secretion signal peptide comprises any one of SEQ ID NOS: 33-43. In yet even other embodiments, the amino acid encoding the secretion signal peptide comprises any one of SEQ ID NOS: 33-43, and wherein [E]_{y} is [-DSS-]₆. In yet even other embodiments, the amino acid encoding the secretion signal peptide comprises any one of SEQ ID NOS: 33-43, [E]_{y} is [-DSS-]₆, and q is 0. In yet even other embodiments, the amino acid encoding the secretion signal peptide comprises any one of SEQ ID NOS: 33-43, [E]_{y} is [-DSS-]₆, and q is 1.

In some embodiments, the Fc domain has at least 90% sequence identity to that of SEQ ID NO: 130. In some embodiments, the Fc domain has at least 91% sequence identity to that of SEQ ID NO: 130. In some embodiments, the Fc domain has at least 92% sequence identity to that of SEQ ID NO: 130. In some embodiments, the Fc domain has at least 93% sequence identity to that of SEQ ID NO: 130. In some embodiments, the Fc domain has at least 94% sequence identity to that of SEQ ID NO: 130. In some embodiments, the Fc domain has at least 95% sequence identity to that of SEQ ID NO: 130. In some embodiments, the Fc domain has at least 96% sequence identity to that of SEQ ID NO: 130. In some embodiments, the Fc domain has at least 97% sequence identity to that of SEQ ID NO: 130. In some embodiments, the Fc domain has at least 98% sequence identity to that of SEQ ID NO: 130. In some embodiments, the Fc domain has at least 99% sequence identity to that of SEQ ID NO: 130. In some embodiments, the Fc domain comprises SEQ ID NO: 130. In some embodiments, the Fc domain comprises SEQ ID NO: 130 and y ranges from 4 to 6. In some embodiments, the Fc domain comprises SEQ ID NO: 130 and [E]_{y} is [DSS]₆. In some embodiments, the R comprises SEQ ID NO: 9 and [E]_{y} is [DSS]₆.

In some embodiments, M comprises 2 amino acids. For example, M may comprise leucine and lysine. In some embodiments, M is leucine-lysine. By way of another example, M may comprise two alanine amino acids In other embodiments, M comprises three amino acids.

In some embodiments, N comprises 2 amino acids (i.e., a diamino acid). For example, N may comprise aspartic acid and isoleucine. By way of another example, N may comprise two alanine amino acids. In some embodiments, N is aspartic acid—isoleucine. In other embodiments, N comprises 3 amino acids.

In some embodiments, M is leucine-lysine and N is aspartic acid—isoleucine. In some embodiments, M is leucine-lysine and N is aspartic acid—isoleucine and Fc has at least 90% sequence identity to that of SEQ ID NO: 130. In some embodiments, M is leucine-lysine and N is aspartic acid—isoleucine and Fc has at least 91% sequence identity to that of SEQ ID NO: 130. In some embodiments, M is leucine-lysine and N is aspartic acid—isoleucine and Fc has at least 92% sequence identity to that of SEQ ID NO: 130. In some embodiments, M is leucine-lysine and N is aspartic acid—isoleucine and Fc has at least 93% sequence identity to that of SEQ ID NO: 130. In some embodiments, M is leucine-lysine and N is aspartic acid—isoleucine and Fc has at least 94% sequence identity to that of SEQ ID NO: 130. In some embodiments, M is leucine-lysine and N is aspartic acid—isoleucine and Fc has at least 95% sequence identity to that of SEQ ID NO: 130. In some embodiments, M is leucine-lysine and N is aspartic acid—isoleucine and Fc has at least 96% sequence identity to that of SEQ ID NO: 130. In some embodiments, M is leucine-lysine and N is aspartic acid—isoleucine and Fc has at least 97% sequence identity to that of SEQ ID NO: 130. In some embodiments, M is leucine-lysine and N is aspartic acid—isoleucine and Fc has at least 98% sequence identity to that of SEQ ID NO: 130. In some embodiments, M is leucine-lysine and N is aspartic acid—isoleucine and Fc has at least 99% sequence identity to that of SEQ ID NO: 130. In some embodiments, M is leucine-lysine and N is aspartic acid—isoleucine and Fc comprises SEQ ID NO: 130.

In some embodiments, M is leucine-lysine and N is aspartic acid—isoleucine; and o, p, and q are each 1. In some embodiments, M is leucine-lysine and N is aspartic acid—isoleucine and Fc has at least 90% sequence identity to that of SEQ ID NO: 130; and o, p, and q are each 1. In some embodiments, M is leucine-lysine and N is aspartic acid—isoleucine and Fc has at least 91% sequence identity to that of SEQ ID NO: 130; and o, p, and q are each 1. In some embodiments, M is leucine-lysine and N is aspartic acid—isoleucine and Fc has at least 92% sequence identity to that of SEQ ID NO: 130; and o, p, and q are each 1. In some embodiments, M is leucine-lysine and N is aspartic acid—isoleucine and Fc has at least 93% sequence identity to that of SEQ ID NO: 130; and o, p, and q are each 1. In some embodiments, M is leucine-lysine and N is aspartic acid—isoleucine and Fc has at least 94% sequence identity to that of SEQ ID NO: 130; and o, p, and q are each 1. In some embodiments, M is leucine-lysine and N is aspartic acid—isoleucine and Fc has at least 95% sequence identity to that of SEQ ID NO: 130; and o, p, and q are each 1. In some embodiments, M is leucine-lysine and N is aspartic acid—isoleucine and Fc has at least 96% sequence identity to that of SEQ ID NO: 130; and o, p, and q are each 1. In some embodiments, M is leucine-lysine and N is aspartic acid—isoleucine and Fc has at least 97% sequence identity to that of SEQ ID NO: 130; and o, p, and q are each 1. In some embodiments, M is leucine-lysine and N is aspartic acid—isoleucine and Fc has at least 98% sequence identity to that of SEQ ID NO: 130; and o, p, and q are each 1. In some embodiments, M is leucine-lysine and N is aspartic acid—isoleucine and Fc has at least 99% sequence identity to that of SEQ ID NO: 130; and o, p, and q are each 1. In some embodiments, M is leucine-lysine; N is aspartic acid—isoleucine and Fc comprises SEQ ID NO: 130; and o, p, and q are each 1. In some embodiments, M is leucine-lysine; N is aspartic acid—isoleucine; Fc comprises SEQ ID NO: 130; o, p, and q are each 1; and [A] has at least 99% identity to any one of SEQ ID NOS: 12 and 33-43. In some embodiments, M is leucine-lysine; N is aspartic acid—isoleucine; Fc comprises; o, p, and q are each 1; and [A] comprises any one of SEQ ID NOS: 12 and 33-43.

In some embodiments, E comprises 3 amino acids. In some embodiments, E comprises 3 amino acids, and y ranges from 1 to 16. In some embodiments, E comprises 3 amino acids, and y ranges from 1 to 12. In some embodiments, E comprises 3 amino acids, and y ranges from 1 to 10. In some embodiments, E comprises 3 amino acids, and y ranges from 1 to 8. In some embodiments, E comprises 3 amino acids, and y ranges from 4 to 8. In some embodiments, E comprises 3 amino acids, and y is 6.

In some embodiments, E is -D-S-S-. In other embodiments, E is -D-D-S-.

In some embodiments, E is -D-S-S-, and y ranges from 1 to 16. In some embodiments, E is -D-S-S-, and y ranges from 1 to 12. In some embodiments, E is -D-S-S-, and y ranges from 1 to 10. In some embodiments, E is -D-S-S-, and y ranges from 4 to 8. In some embodiments, E is aspartic acid, and y ranges from 1 to 8. In some embodiments, E is -D-S-S-, and y is 6.

### Examples of Polypeptides Having Any One of Formulas (I), (IA), (IB), (II), (III), (IV), (VA) and (VB) (hereinafter “Formulas (I)-(V)”)

In some embodiments, the polypeptide comprises an amino acid sequence having at least 85% identity to SEQ ID NO: 2. In some embodiments, the polypeptide comprises an amino acid sequence having at least 90% identity to SEQ ID NO: 2. In some embodiments, the polypeptide comprises an amino acid sequence having at least 95% identity to SEQ ID NO: 2. In some embodiments, the polypeptide comprises an amino acid sequence having at least 96% identity to SEQ ID NO: 2. In some embodiments, the polypeptide comprises an amino acid sequence having at least 97% identity to SEQ ID NO: 2. In some embodiments, the polypeptide comprises an amino acid sequence having at least 98% identity to SEQ ID NO: 2. In some embodiments, the polypeptide comprises an amino acid sequence having at least 99% identity to SEQ ID NO: 2. In some embodiments, the polypeptide comprises SEQ ID NO: 2.

In some embodiments, the polypeptide comprises an amino acid sequence having at least 85% identity to SEQ ID NO: 3. In some embodiments, the polypeptide comprises an amino acid sequence having at least 90% identity to SEQ ID NO: 3. In some embodiments, the polypeptide comprises an amino acid sequence having at least 95% identity to SEQ ID NO: 3. In some embodiments, the polypeptide comprises an amino acid sequence having at least 96% identity to SEQ ID NO: 3. In some embodiments, the polypeptide comprises an amino acid sequence having at least 97% identity to SEQ ID NO: 3. In some embodiments, the polypeptide comprises an amino acid sequence having at least 98% identity to SEQ ID NO: 3. In some embodiments, the polypeptide comprises an amino acid sequence having at least 99% identity to SEQ ID NO: 3. In some embodiments, the polypeptide comprises SEQ ID NO: 3.

In some embodiments, the polypeptide comprises an amino acid sequence having at least 85% identity to SEQ ID NO: 4. In some embodiments, the polypeptide comprises an amino acid sequence having at least 90% identity to SEQ ID NO: 4. In some embodiments, the polypeptide comprises an amino acid sequence having at least 95% identity to SEQ ID NO: 4. In some embodiments, the polypeptide comprises an amino acid sequence having at least 96% identity to SEQ ID NO: 4. In some embodiments, the polypeptide comprises an amino acid sequence having at least 97% identity to SEQ ID NO: 4. In some embodiments, the polypeptide comprises an amino acid sequence having at least 98% identity to SEQ ID NO: 4. In some embodiments, the polypeptide comprises an amino acid sequence having at least 99% identity to SEQ ID NO: 4. In some embodiments, the polypeptide comprises SEQ ID NO: 4.

In some embodiments, the polypeptide comprises an amino acid sequence having at least 85% identity to SEQ ID NO: 5. In some embodiments, the polypeptide comprises an amino acid sequence having at least 90% identity to SEQ ID NO: 5. In some embodiments, the polypeptide comprises an amino acid sequence having at least 95% identity to SEQ ID NO: 5. In some embodiments, the polypeptide comprises an amino acid sequence having at least 96% identity to SEQ ID NO: 5. In some embodiments, the polypeptide comprises an amino acid sequence having at least 97% identity to SEQ ID NO: 5. In some embodiments, the polypeptide comprises an amino acid sequence having at least 98% identity to SEQ ID NO: 5. In some embodiments, the polypeptide comprises an amino acid sequence having at least 99% identity to SEQ ID NO: 5. In some embodiments, the polypeptide comprises SEQ ID NO: 5.

In some embodiments, the polypeptide comprises an amino acid sequence having at least 85% identity to SEQ ID NO: 6. In some embodiments, the polypeptide comprises an amino acid sequence having at least 90% identity to SEQ ID NO: 6. In some embodiments, the polypeptide comprises an amino acid sequence having at least 95% identity to SEQ ID NO: 6. In some embodiments, the polypeptide comprises an amino acid sequence having at least 96% identity to SEQ ID NO: 6. In some embodiments, the polypeptide comprises an amino acid sequence having at least 97% identity to SEQ ID NO: 6. In some embodiments, the polypeptide comprises an amino acid sequence having at least 98% identity to SEQ ID NO: 6. In some embodiments, the polypeptide comprises an amino acid sequence having at least 99% identity to SEQ ID NO: 6. In some embodiments, the polypeptide comprises SEQ ID NO: 6.

In some embodiments, the polypeptide comprises an amino acid sequence having at least 85% identity to SEQ ID NO: 7. In some embodiments, the polypeptide comprises an amino acid sequence having at least 90% identity to SEQ ID NO: 7. In some embodiments, the polypeptide comprises an amino acid sequence having at least 95% identity to SEQ ID NO: 7. In some embodiments, the polypeptide comprises an amino acid sequence having at least 96% identity to SEQ ID NO: 7. In some embodiments, the polypeptide comprises an amino acid sequence having at least 97% identity to SEQ ID NO: 7. In some embodiments, the polypeptide comprises an amino acid sequence having at least 98% identity to SEQ ID NO: 7. In some embodiments, the polypeptide comprises an amino acid sequence having at least 99% identity to SEQ ID NO: 7. In some embodiments, the polypeptide comprises SEQ ID NO: 7.

In some embodiments, the polypeptide comprises an amino acid sequence having at least 85% identity to SEQ ID NO: 8. In some embodiments, the polypeptide comprises an amino acid sequence having at least 90% identity to SEQ ID NO: 8. In some embodiments, the polypeptide comprises an amino acid sequence having at least 95% identity to SEQ ID NO: 8. In some embodiments, the polypeptide comprises an amino acid sequence having at least 96% identity to SEQ ID NO: 8. In some embodiments, the polypeptide comprises an amino acid sequence having at least 97% identity to SEQ ID NO: 8. In some embodiments, the polypeptide comprises an amino acid sequence having at least 98% identity to SEQ ID NO: 8. In some embodiments, the polypeptide comprises an amino acid sequence having at least 99% identity to SEQ ID NO: 8. In some embodiments, the polypeptide comprises SEQ ID NO: 8.

In some embodiments, the polypeptide of the present disclosure has an amino acid sequence having at least 85% sequence identity to any one of SEQ ID NOS: 44-54, 68, 75, 105, and 116-125. In some embodiments, the polypeptide of the present disclosure has at least 90% sequence identity to any one of SEQ ID NOS: 44-54, 68, 75, 105, and 116-125. In some embodiments, the polypeptide of the present disclosure has an amino acid sequence having at least 95% sequence identity to any one of SEQ ID NOS: 44-54, 68, 75, 105, and 116-125. In some embodiments, the polypeptide of the present disclosure has an amino acid sequence having at least 96% sequence identity to any one of SEQ ID NOS: 44-54, 68, 75, 105, and 116-125. In some embodiments, the polypeptide of the present disclosure has an amino acid sequence having at least 95% sequence identity to any one of SEQ ID NOS: 44-54, 68, 75, 105, and 116-125. In some embodiments, the polypeptide of the present disclosure has an amino acid sequence having at least 97% sequence identity to any one of SEQ ID NOS: 44-54, 68, 75, 105, and 116-125. In some embodiments, the polypeptide of the present disclosure has an amino acid sequence having at least 98% sequence identity to any one of SEQ ID NOS: 44-54, 68, 75, 105, and 116-125. In some embodiments, the polypeptide of the present disclosure has an amino acid sequence having at least 99% sequence identity to any one of SEQ ID NOS: 44-54, 68, 75, 105, and 116-125. In some embodiments, polypeptide of the present disclosure comprises any one of SEQ ID NOS: 44-54, 68, 75, 105, and 116-125.

In some embodiments, the polypeptide of the present disclosure is encoded by a nucleotide sequence having at least 80% identity to any one of SEQ ID NOS: 106, 108-115, and 131. In some embodiments, the polypeptide of the present disclosure is encoded by a nucleotide sequence having at least 85% identity to any one of SEQ ID NOS: 106, 108-115, and 131. In some embodiments, the polypeptide of the present disclosure is encoded by a nucleotide sequence having at least 90% identity to any one of SEQ ID NOS: 106, 108-115, and 131. In some embodiments, the polypeptide of the present disclosure is encoded by a nucleotide sequence having at least 91% identity to any one of SEQ ID NOS: 106, 108-115, and 131. In some embodiments, the polypeptide of the present disclosure is encoded by a nucleotide sequence having at least 92% identity to any one of SEQ ID NOS: 106, 108-115, and 131. In some embodiments, the polypeptide of the present disclosure is encoded by a nucleotide sequence having at least 93% identity to any one of SEQ ID NOS: 106, 108-115, and 131. In some embodiments, the polypeptide of the present disclosure is encoded by a nucleotide sequence having at least 94% identity to any one of SEQ ID NOS: 106, 108-115, and 131. In some embodiments, the polypeptide of the present disclosure is encoded by a nucleotide sequence having at least 95% identity to any one of SEQ ID NOS: 106, 108-115, and 131. In some embodiments, the polypeptide of the present disclosure is encoded by a nucleotide sequence having at least 96% identity to any one of SEQ ID NOS: 106, 108-115, and 131. In some embodiments, the polypeptide of the present disclosure is encoded by a nucleotide sequence having at least 97% identity to any one of SEQ ID NOS: 106, 108-115, and 131. In some embodiments, the polypeptide of the present disclosure is encoded by a nucleotide sequence having at least 98% identity to any one of SEQ ID NOS: 106, 108-115, and 131. In some embodiments, the polypeptide of the present disclosure is encoded by a nucleotide sequence having at least 99% identity to any one of SEQ ID NOS: 106, 108-115, and 131. In some embodiments, the polypeptide of the present disclosure is encoded by a nucleotide sequence having any one of SEQ ID NOS: 106, 108-115, and 131.

Delivery Platforms

In another aspect of the present disclosure are delivery platforms, e.g. non-viral vectors and viral vectors. In some embodiments, the non-viral vectors include bacterial plasmids, minicircle DNA, minivector DNA, linear DNA, particles, nanoparticles, etc. In some embodiments, non-viral particles include liposomes such as, for example, those disclosed in U.S. Pat. No. 5,422,120, and in PCT Publication Nos. WO 95/13796, WO 94/23697, and WO 91/14445, particularly including heterovesicular liposomal particles, the disclosures of which are hereby incorporated by reference herein in their entireties. In some embodiments, non-viral vectors may be delivered to a cell or a subject according to various methods, which include, but are not limited, to injection, electroporation, gene gun, sonoporation, magnetofection, hydrodynamic delivery, or other physical or chemical methods. In some embodiments, therapeutic genes can be inserted directly into the plasmid, and then this recombinant plasmid can be introduced into cells in a variety of ways. For example, it can be injected directly into targeted tissues as naked-DNA. Other non-limiting examples of non-viral delivery vehicles are described in PCT Publication Nos. WO/2005/123142. WO/2012/017119, WO/1995/021195, WO/2012/017118, WO/2016/016358, WO/2006/122542, and WO/2014/072929, the disclosures of which are hereby incorporated by reference herein in their entireties.

In some embodiments, the delivery platforms are viral expression vectors which encode a polypeptide, such as a polypeptide comprising a nucleic acid sequence encoding an alkaline phosphatase or a variant thereof for expression. In some embodiments, the expression vectors may be retroviral vectors, lentiviral vectors, adenovirus vectors, AAV vectors, etc.

In some embodiments, the expression vectors are lentiviral vectors. Lentiviruses are a subclass of Retroviruses. Lentiviruses resemble 7-retroviruses (γ-RV) in their ability to stably integrate into the target cell genome, resulting in persistent expression of the gene of interest. However, in contrast to γ-retroviruses, lentiviruses also can transduce nondividing cells, which has led to their wide use as gene transfer vectors. In some embodiments, the lentivirus genome is monopartite, linear, dimeric, positive-strand single-stranded RNA (ssRNA(+)″) of 9.75 kb, with a 5′-cap and a 3′poly-A tail. In some embodiments, the lentiviral genome is flanked by the ‘ and 3’ long terminal repeat (LTR) sequences which have promoter/enhancer activity and are essential for the correct expression of the full-length lentiviral vector transcript. In some embodiments, the LTRs also have an important role in reverse transcription and integration of the vector into the target cell genome. In some embodiments, upon viral entry into a cell, the RNA genome is reverse-transcribed into double-stranded DNA, which is then inserted into the genome at a random position by the viral integrase enzyme. In some embodiments, the lentivirus, now called a provirus, remains in the genome and is passed on to the progeny of the cell when it divides.

Examples of species of lentivirus include, for example, human immunodeficiency virus 1 (HIV-1), human immunodeficiency virus 2 (HIV-2), simian immunodeficiency virus (SIV), bovine immunodeficiency virus (BIV), and feline immunodeficiency virus (FIV). In some embodiments, the lentiviral vector of the present disclosure can be based on any lentivirus species. In some embodiments, the lentiviral vector is based on a human immunodeficiency virus (e.g., HIV-1 or HIV-2), most preferably HIV-1.

Lentiviral vectors typically are generated by trans-complementation in packaging cells that are co-transfected with a plasmid containing the vector genome and the packaging constructs that encode only the proteins essential for lentiviral assembly and function. In some embodiments, a self-inactivating (SIN) lentiviral vector can be generated by abolishing the intrinsic promoter/enhancer activity of the HIV-1 LTR, which reduces the likelihood of aberrant expression of cellular coding sequences located adjacent to the vector integration site (see, e.g., Vigna et al., J. Gene Med., 2: 308-316 (2000); Naldini et al., Science, 272: 263-267 (1996); and Mátrai et al., Molecular Therapy, 18(3): 477-490 (2010)). In some embodiments, most common procedure to generate lentiviral vectors is to co-transfect cell lines (e.g., 293T human embryonic kidney cells) with a lentiviral vector plasmid and three packaging constructs encoding the viral Gag-Pol, Rev-Tat, and envelope (Env) proteins.

Methods for generating lentiviral vectors are well-known in the art, and the lentiviral vectors of the present disclosure can be constructed using any suitable such method. In some embodiments, lentiviral vectors typically are produced by co-transfecting 293T human embryonic kidney cells with several different plasmid constructs, which separately contain the lentiviral cis-acting sequences and trans-acting factors that are required for viral particle production, infection, and integration. Lentiviral vector production systems typically include four plasmids. In some embodiments, the transfer vector contains the transgene be delivered in a lentiviral backbone containing all of the cis-acting sequences required for genomic RNA production and packaging. Three additional provide the trans-acting factors required for packaging, namely Gag-Pol, Rev-Tat, and the envelope protein VSVG, respectively. When these four plasmids are transfected into 293T human embryonic kidney cells, viral particles accumulate in the supernatant, and the viral product can be concentrated by ultracentrifugation. Lentiviral production protocols are further described in, for example, Tiscornia et al., Nature Protocols, 1: 241-245 (2006); Stevenson, M., Curr. Top. Microbiol. Immunol., 261: 1-30 (2002); Cronin et al., Curr. Gene Ther., 5: 387-398 (2005); Sandrin et al., Curr. Top. Microbiol. Immunol., 281: 137-178 (2003); Zufferey, R., Curr. Top. Microbiol. Immunol., 261: 107-121 (2002); Sinn et al., Gene Ther., 12: 1089-1098 (2005); and Saenz, D. T. and Poeschla, E. M., J. Gene Med., 6: S95-S104 (2004). Other methods for producing lentiviral vectors are known in the art and described in, for example, U.S. Patent Application Publications 2008/0254008 and 2010/0003746; and Yang et al., Hum Gene Ther. Methods, 23(2): 73-83 (2012).

In some embodiments, the present disclosure provides for a lentiviral vector comprising a nucleotide sequence encoding any one of the polypeptides of Formulas (I), (II), (III), (IV), (V) or a variant or fragment thereof.

In some embodiments, the lentiviral vector includes a nucleic acid sequence encoding a polypeptide, wherein the polypeptide comprises an amino acid sequence having at least 90% identity to any one of SEQ ID NOS: 1-8, 44-54, 105, and 116-125. In some embodiments, the lentiviral vector includes a nucleic acid sequence encoding a polypeptide, wherein the polypeptide comprises an amino acid sequence having at least 91% identity to any one of SEQ ID NOS: 1-8, 44-54, 105, and 116-125. In some embodiments, the lentiviral vector includes a nucleic acid sequence encoding a polypeptide, wherein the polypeptide comprises an amino acid sequence having at least 92% identity to any one of SEQ ID NOS: 1-8, 44-54, 105, and 116-125. In some embodiments, the lentiviral vector includes a nucleic acid sequence encoding a polypeptide, wherein the polypeptide comprises an amino acid sequence having at least 93% identity to any one of SEQ ID NOS: 1-8, 44-54, 105, and 116-125. In some embodiments, the lentiviral vector includes a nucleic acid sequence encoding a polypeptide, wherein the polypeptide comprises an amino acid sequence having at least 94% identity to any one of SEQ ID NOS: 1-8, 44-54, 105, and 116-125. In some embodiments, the lentiviral vector includes a nucleic acid sequence encoding a polypeptide, wherein the polypeptide comprises an amino acid sequence having at least 95% identity to any one of SEQ ID NOS: 1-8, 44-54, 105, and 116-125. In some embodiments, the lentiviral vector includes a nucleic acid sequence encoding a polypeptide, wherein the polypeptide comprises an amino acid sequence having at least 96% identity to any one of SEQ ID NOS: 1-8, 44-54, 105, and 116-125. In some embodiments, the lentiviral vector includes a nucleic acid sequence encoding a polypeptide having at least 97% identity to any one of SEQ ID NOS: 1-8, 44-54, 105, and 116-125. In some embodiments, the lentiviral vector includes a nucleic acid sequence encoding a polypeptide, wherein the polypeptide comprises an amino acid sequence having at least 98% identity to any one of SEQ ID NOS: 1-8, 44-54, 105, and 116-125. In some embodiments, the lentiviral vector includes a nucleic acid sequence encoding a polypeptide, wherein the polypeptide comprises an amino acid sequence having at least 99% identity to any one of SEQ ID NOS: 1-8, 44-54, 105, and 116-125. In some embodiments, the lentiviral vector includes a nucleic acid sequence encoding a polypeptide, wherein the polypeptide comprises an amino acid sequence having any one of SEQ ID NOS: 1-8, 44-54, 105, and 116-125.

In some embodiments, the lentiviral vector includes a nucleic acid sequence encoding a polypeptide, wherein nucleic acid sequence encoding the polypeptide has at least 90% identity to any one of SEQ ID NOS: 106, 108-115, and 131. In some embodiments, the lentiviral vector includes a nucleic acid sequence encoding a polypeptide, wherein nucleic acid sequence encoding the polypeptide has at least 91% identity to any one of SEQ ID NOS: 106, 108-115, and 131. In some embodiments, the lentiviral vector includes a nucleic acid sequence encoding a polypeptide, wherein nucleic acid sequence encoding the polypeptide has at least 92% identity to any one of SEQ ID NOS: 106, 108-115, and 131. In some embodiments, the lentiviral vector includes a nucleic acid sequence encoding a polypeptide, wherein nucleic acid sequence encoding the polypeptide has at least 93% identity to any one of SEQ ID NOS: 106, 108-115, and 131. In some embodiments, the lentiviral vector includes a nucleic acid sequence encoding a polypeptide, wherein nucleic acid sequence encoding the polypeptide has at least 94% identity to any one of SEQ ID NOS: 106, 108-115, and 131. In some embodiments, the lentiviral vector includes a nucleic acid sequence encoding a polypeptide, wherein nucleic acid sequence encoding the polypeptide has at least 95% identity to any one of SEQ ID NOS: 106, 108-115, and 131. In some embodiments, the lentiviral vector includes a nucleic acid sequence encoding a polypeptide, wherein nucleic acid sequence encoding the polypeptide has at least 96% identity to any one of SEQ ID NOS: 106, 108-115, and 131. In some embodiments, the lentiviral vector includes a nucleic acid sequence encoding a polypeptide, wherein nucleic acid sequence encoding the polypeptide has at least 97% identity to any one of SEQ ID NOS: 106, 108-115, and 131. In some embodiments, the lentiviral vector includes a nucleic acid sequence encoding a polypeptide, wherein nucleic acid sequence encoding the polypeptide has at least 98% identity to any one of SEQ ID NOS: 106, 108-115, and 131. In some embodiments, the lentiviral vector includes a nucleic acid sequence encoding a polypeptide, wherein nucleic acid sequence encoding the polypeptide has at least 99% identity to any one of SEQ ID NOS: 106, 108-115, and 131. In some embodiments, the lentiviral vector includes a nucleic acid sequence encoding a polypeptide, wherein nucleic acid sequence encoding the polypeptide comprises any one of SEQ ID NOS: 106, 108-115, and 131.

In some embodiments, the lentiviral expression vector comprises a nucleic acid sequence having at least 80% identity to any one of SEQ ID NOS: 15-26, 55-65, 74, 81, 82, 84-95, 97, and 98. In some embodiments, the lentiviral expression vector comprises a nucleic acid sequence having at least 85% identity to any one of SEQ ID NOS: 15-26, 55-65, 74, 81, 82, 84-95, 97, and 98. In some embodiments, the lentiviral expression vector comprises a nucleic acid sequence having at least 90% identity to any one of SEQ ID NOS: 15-26, 55-65, 74, 81, 82, 84-95, 97, and 98. In some embodiments, the lentiviral expression vector comprises a nucleic acid sequence having at least 91% identity to any one of SEQ ID NOS: 15-26, 55-65, 74, 81, 82, 84-95, 97, and 98. In some embodiments, the lentiviral expression vector comprises a nucleic acid sequence having at least 92% identity to any one of SEQ ID NOS: 15-26, 55-65, 74, 81, 82, 84-95, 97, and 98. In some embodiments, the lentiviral expression vector comprises a nucleic acid sequence having at least 93% identity to any one of SEQ ID NOS: 15-26, 55-65, 74, 81, 82, 84-95, 97, and 98. In some embodiments, the lentiviral expression vector comprises a nucleic acid sequence having at least 94% identity to any one of SEQ ID NOS: 15-26, 55-65, 74, 81, 82, 84-95, 97, and 98. In some embodiments, the lentiviral expression vector comprises a nucleic acid sequence having at least 95% identity to any one of SEQ ID NOS: 15-26, 55-65, 74, 81, 82, 84-95, 97, and 98. In some embodiments, the lentiviral expression vector comprises a nucleic acid sequence having at least 96% identity to any one of SEQ ID NOS: 15-26, 55-65, 74, 81, 82, 84-95, 97, and 98. In some embodiments, the lentiviral expression vector comprises a nucleic acid sequence having at least 97% identity to any one of SEQ ID NOS: 15-26, 55-65, 74, 81, 82, 84-95, 97, and 98. In some embodiments, the lentiviral expression vector comprises a nucleic acid sequence having at least 98% identity to any one of SEQ ID NOS: 15-26, 55-65, 74, 81, 82, 84-95, 97, and 98. In some embodiments, the lentiviral expression vector comprises a nucleic acid sequence having at least 99% identity to any one of SEQ ID NOS: 15-26, 55-65, 74, 81, 82, 84-95, 97, and 98. In some embodiments, the lentiviral expression vector comprises a nucleic acid sequence having any one of SEQ ID NOS: 15-26, 55-65, 74, 81, 82, 84-95, 97, and 98.

Promoters

Any promoter utilized in the art may be utilized to drive expression of one or nucleic acid sequences within the expression vectors described herein, e.g. to drive expression of the nucleic acid sequence encoding the polypeptide. In some embodiments, the promoter is one which is functional in mammalian cells. High-level constitutive promoters are preferred for use in the vectors according to the present disclosure. Examples of such promoters include, without limitation, the retroviral Rous sarcoma virus (RSN) LTR promoter (optionally with the RSV enhancer), the cytomegalovirus (CMV) promoter (optionally with the CMV enhancer) [see, e.g., Boshart et al, Cell, 41:521-530 (1985)], the SN40 promoter, the dihydrofolate reductase promoter, the beta-actin promoter, the beta-active promoter linked to the enhancer derived from the cytomegalovirus (CMN) immediate early (IE) promoter, the phosphoglycerol kinase (PGK) promoter, and the EF1a promoter [Invitrogen]. Inducible promoters are regulated by exogenously supplied compounds, including, the zinc-inducible sheep metallothionine (MT) promoter, the dexamethasone (Dex)-inducible mouse mammary tumor virus (MMTV) promoter, the T7 polymerase promoter system [WO 98/10088]; the ecdysone insect promoter [No et al, Proc. Natl. Acad. Sci. USA, 93:3346-3351 (1996)], the tetracycline-repressible system [Gossen et al, Proc. Natl. Acad. Sci. USA, 89:5547-5551 (1992)], the tetracycline-inducible system [Gossen et al, Science, 268:1766-1769 (1995); see also Harvey et al, Curr. Opin. Chem. Biol, 2:512-518 (1998)], the RU486-inducible system [Wang et al, Nat. Biotech., 15:239-243 (1997) and Wang et al, Gene Ther., 4:432-441 (1997)] and the rapamycin-inducible system [Magari et al, J Clin. Invest., 100:2865-2872 (1997)]. Other types of inducible promoters which may be useful in the present disclosure are those which are regulated by a specific physiological state, e.g., temperature, acute phase, a particular differentiation state of the cell, or in replicating cells only.

Illustrative ubiquitous expression control sequences suitable for use in particular embodiments include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, a short elongation factor 1-alpha (EF1α-short) promoter, a long elongation factor 1-alpha (EF1a-long) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPAS), heat shock protein 90 kDa beta, member 1 (HSP90B1), heat shock protein 70 kDa (HSP70), β-kinesin ((3-KIN), the human ROSA 26 locus Orions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, d1587rev primer-binding site substituted (MND) promoter (Challita et al., J Virol. 69(2):748-55 (1995)).

In some embodiments, the promoter may be selected from a Cytomegalovirus (CMV) minimal promoter and, more preferably, from human CMV (hCMV) such as the hCMV immediate early promoter derived minimal promoter as described in, e.g., Gossen and Bujard (Proc. Natl. Acad. Sci. USA, 1992, 89: 5547-5551). Modified promoters also may be utilized, including insertion and deletion mutation of native promoters and combinations or permutations thereof. One example of a modified promoter is the “minimal CMV promoter” as described by Gossen and Bujard (Proc. Natl. Acad. Sci. USA, 1992, 89: 5547-5551). In any case, any promoter can be tested readily for its effectiveness in the tetracycline-responsive expression system described herein by substitution for the minimal CMV promoter described herein.

In some embodiments, the promoter is an MND promoter. In some embodiments, the MND promoter has at least 90% identity to that of SEQ ID NO: 66. In some embodiments, the MND promoter has at least 95% identity to that of SEQ ID NO: 66. In some embodiments, the MND promoter has at least 96% identity to that of SEQ ID NO: 66. In some embodiments, the MND promoter has at least 97% identity to that of SEQ ID NO: 66. In some embodiments, the MND promoter has at least 98% identity to that of SEQ ID NO: 66. In some embodiments, the MND promoter has at least 99% identity to that of SEQ ID NO: 66. In some embodiments, the MND promoter comprises SEQ ID NO: 66.

In some embodiments, the promoter is an EF1A promoter. In some embodiments, the EF1A promoter has at least 90% identity to any one of SEQ ID NOS: 67 and 100. In some embodiments, the EF1A promoter has at least 95% identity to any one of SEQ ID NOS: 67 and 100. In some embodiments, the EF1A promoter has at least 96% identity to any one of SEQ ID NOS: 67 and 100. In some embodiments, the EF1A promoter has at least 97% identity to any one of SEQ ID NOS: 67 and 100. In some embodiments, the EF1A promoter has at least 98% identity to any one of SEQ ID NOS: 67 and 100. In some embodiments, the EF1A promoter has at least 99% identity to any one of SEQ ID NOS: 67 and 100. In some embodiments, the EF1A promoter comprises SEQ ID NO: 67. In some embodiments, the EF1A promoter comprises SEQ ID NO: 100.

In some embodiments, the promoter is a CD11b promoter. In some embodiments, the CD11b promoter has at least 90% identity to SEQ ID NO: 96. In some embodiments, the CD11b promoter has at least 95% identity to SEQ ID NO: 96. In some embodiments, the CD11b promoter has at least 96% identity to SEQ ID NO: 96. In some embodiments, the CD11b promoter has at least 97% identity to SEQ ID NO: 96. In some embodiments, the CD11b promoter has at least 98% identity to SEQ ID NO: 96. In some embodiments, the CD11b promoter has at least 99% identity to SEQ ID NO: 96. In some embodiments, the CD11b promoter has SEQ ID NO: 96.

In some embodiments, the promoter is a EFS promoter. In some embodiments, the EFS promoter has at least 90% identity to SEQ ID NO: 99. In some embodiments, the EFS promoter has at least 95% identity to SEQ ID NO: 99. In some embodiments, the EFS promoter has at least 96% identity to SEQ ID NO: 99. In some embodiments, the EFS promoter has at least 97% identity to SEQ ID NO: 99. In some embodiments, the EFS promoter has at least 98% identity to SEQ ID NO: 99. In some embodiments, the EFS promoter has at least 99% identity to SEQ ID NO: 99. In some embodiments, the EFS promoter has SEQ ID NO: 99.

In some embodiments, the promoter is a Ubc promoter. In some embodiments, the Ubc promoter has at least 90% identity to SEQ ID NO: 101. In some embodiments, the Ubc promoter has at least 95% identity to SEQ ID NO: 101. In some embodiments, the Ubc promoter has at least 96% identity to SEQ ID NO: 101. In some embodiments, the Ubc promoter has at least 97% identity to SEQ ID NO: 101. In some embodiments, the Ubc promoter has at least 98% identity to SEQ ID NO: 101. In some embodiments, the Ubc promoter has at least 99% identity to SEQ ID NO: 101. In some embodiments, the Ubc promoter has SEQ ID NO: 101.

In some embodiments, the promoter is a CD68LPp promoter. In some embodiments, the CD68LPp promoter has at least 90% identity to SEQ ID NO: 126. In some embodiments, the CD68LPp promoter has at least 95% identity to SEQ ID NO: 126. In some embodiments, the CD68LPp promoter has at least 96% identity to SEQ ID NO: 126. In some embodiments, the CD68LPp promoter has at least 97% identity to SEQ ID NO: 126. In some embodiments, the CD68LPp promoter has at least 98% identity to SEQ ID NO: 126. In some embodiments, the CD68LPp promoter has at least 99% identity to SEQ ID NO: 126. In some embodiments, the CD68LPp promoter has SEQ ID NO: 99.

In some embodiments, the promoter is a tissue-specific promoter, where the tissue-specific promoter is used to achieve cell type specific, lineage specific, or tissue-specific expression of a desired polynucleotide sequence (e.g., to express a particular nucleic acid encoding a polypeptide in only a subset of cell types or tissues or during specific stages of development). Illustrative examples of tissue specific promoters include, but are not limited to: an B29 promoter (B cell expression), a runt transcription factor (CBFa2) promoter (stem cell specific expression), an CD14 promoter (monocytic cell expression), an CD43 promoter (leukocyte and platelet expression), an CD45 promoter (hematopoietic cell expression), an CD68 promoter (macrophage expression), a CYP450 3A4 promoter (hepatocyte expression), an desmin promoter (muscle expression), an elastase 1 promoter (pancreatic acinar cell expression, an endoglin promoter (endothelial cell expression), a fibroblast specific protein 1 promoter (FSP1) promoter (fibroblast cell expression), a fibronectin promoter (fibroblast cell expression), a fms-related tyrosine kinase 1 (FLT1) promoter (endothelial cell expression), a glial fibrillary acidic protein (GFAP) promoter (astrocyte expression), an insulin promoter (pancreatic beta cell expression), an integrin, alpha 2b (ITGA2B) promoter (megakaryocytes), an intracellular adhesion molecule 2 (ICAM-2) promoter (endothelial cells), an interferon beta (IFN-β) promoter (hematopoietic cells), a keratin 5 promoter (keratinocyte expression), a myoglobin (MB) promoter (muscle expression), a myogenic differentiation 1 (MYOD1) promoter (muscle expression), a nephrin promoter (podocyte expression), a bone gamma-carboxyglutamate protein 2 (OG-2) promoter (osteoblast expression), an 3-oxoacid CoA transferase 2B (Oxct2B) promoter, (haploid-spermatid expression), a surfactant protein B (SP-B) promoter (lung expression), a synapsin promoter (neuron expression), a Wiskott-Aldrich syndrome protein (WASP) promoter (hematopoietic cell expression).

In some embodiments, the native promoter for the transgene is utilized. In some embodiments, the native promoter may be preferred when it is desired that expression of the gene should mimic the native expression. In some embodiments, the native promoter may be used when expression of the gene must be regulated temporally or developmentally, or in a tissue-specific manner, or in response to specific transcriptional stimuli. In some embodiments, other native expression control elements, such as enhancer elements, polyadenylation sites or Kozak consensus sequences may also be used to mimic the native expression. In some embodiments, the transgene product or other desirable product to be expressed is operably linked to a tissue-specific promoter. For instance, if expression in skeletal muscle is desired, a promoter active in muscle should be used. These include the promoters from genes encoding skeletal α-actin, myosin light chain 2A, dystrophin, muscle creatine kinase, as well as synthetic muscle promoters with activities higher than naturally-occurring promoters [see Li et al., Nat. Biotech, 17:241-245 (1999)]. Examples of promoters that are tissue-specific are known for liver [albumin, Miyatake et al. J Virol, 71:5124-32 (1997); hepatitis B virus core promoter, Sandig et al, Gene Ther., 3:1002-9 (1996); and alpha-fetoprotein (AFP), Arbuthnot et al, Hum. Gene Ther, 7:1503-14 (1996)], bone [osteocalcin, Stein et al, Mol. Biol. Rep., 24:185-96 (1997); and bone sialoprotein, Chen et al, J Bone Miner. Res., 11:654-64 (1996)], lymphocytes [CD2, Hansal et al., J Immunol, 161:1063-8 (1998); immunoglobulin heavy chain; T cell receptor a chain], neuronal [neuron-specific enolase (NSE) promoter, Andersen et al. Cell. Mol. Neurobiol, 13:503-15 (1993); neurofilament light-chain gene, Piccioli et al., 1991, Proc. Natl. Acad. Sci. USA, 88:5611-5 (1991); and the neuron-specific vgf gene, Piccioli et al, Neuron 15:373-84 (1995)]; among others.

In some embodiments, the lentiviral vector includes a first nucleic acid sequence operably linked to an EF1A promoter, wherein the first nucleic acid sequence encodes for a polypeptide having any one of Formulas (I)-(V).

In some embodiments, the lentiviral vector includes a first nucleic acid sequence operably linked to an EF1A promoter, wherein the first nucleic acid sequence encodes a polypeptide having at least 90% identity to any one of SEQ ID NOS: 1-8, 44-54, 68, 75, 105, and 116-125. In some embodiments, the lentiviral vector includes a first nucleic acid sequence operably linked to an EF1A promoter, wherein the first nucleic acid sequence encodes a polypeptide having at least 91% identity to any one of SEQ ID NOS: 1-8, 44-54, 68, 75, 105, and 116-125. In some embodiments, the lentiviral vector includes a first nucleic acid sequence operably linked to an EF1A promoter, wherein the first nucleic acid sequence encodes a polypeptide having at least 92% identity to any one of SEQ ID NOS: 1-8, 44-54, 68, 75, 105, and 116-125. In some embodiments, the lentiviral vector includes a first nucleic acid sequence operably linked to an EF1A promoter, wherein the first nucleic acid sequence encodes a polypeptide having at least 93% identity to any one of SEQ ID NOS: 1-8, 44-54, 68, 75, 105, and 116-125. In some embodiments, the lentiviral vector includes a first nucleic acid sequence operably linked to an EF1A promoter, wherein the first nucleic acid sequence encodes a polypeptide having at least 94% identity to any one of SEQ ID NOS: 1-8, 44-54, 68, 75, 105, and 116-125. In some embodiments, the lentiviral vector includes a first nucleic acid sequence operably linked to an EF1A promoter, wherein the first nucleic acid sequence encodes a polypeptide having at least 95% identity to any one of SEQ ID NOS: 1-8, 44-54, 68, 75, 105, and 116-125. In some embodiments, the lentiviral vector includes a first nucleic acid sequence operably linked to an EF1A promoter, wherein the first nucleic acid sequence encodes a polypeptide having at least 96% identity to any one of SEQ ID NOS: 1-8, 44-54, 68, 75, 105, and 116-125. In some embodiments, the lentiviral vector includes a first nucleic acid sequence operably linked to an EF1A promoter, wherein the first nucleic acid sequence encodes a polypeptide having at least 97% identity to any one of SEQ ID NOS: 1-8, 44-54, 68, 75, 105, and 116-125. In some embodiments, the lentiviral vector includes a first nucleic acid sequence operably linked to an EF1A promoter, wherein the first nucleic acid sequence encodes a polypeptide having at least 98% identity to any one of SEQ ID NOS: 1-8, 44-54, 68, 75, 105, and 116-125. In some embodiments, the lentiviral vector includes a first nucleic acid sequence operably linked to an EF1A promoter, wherein the first nucleic acid sequence encodes a polypeptide having at least 99% identity to any one of SEQ ID NOS: 1-8, 44-54, 68, 75, 105, AND 116-125. In some embodiments, the lentiviral vector includes a first nucleic acid sequence operably linked to an EF1A promoter, wherein the first nucleic acid sequence encodes a polypeptide having any one of SEQ ID NOS: 1-8, 44-54, 68, 75, 105, and 116-125.

In some embodiments, the lentiviral vector includes a first nucleic acid sequence operably linked to an MND promoter, wherein the first nucleic acid sequence encodes for a polypeptide having any one of Formulas (I)-(V).

In some embodiments, the lentiviral vector includes a first nucleic acid sequence operably linked to an MND promoter, wherein the first nucleic acid sequence encodes for a polypeptide having at least 90% identity to any one of SEQ ID NOS: 1-8, 44-54, 68, 75, 105, and 116-125. In some embodiments, the lentiviral vector includes a first nucleic acid sequence operably linked to an MND promoter, wherein the first nucleic acid sequence encodes a polypeptide having at least 91% identity to any one of SEQ ID NOS: 1-8, 44-54, 68, 75, 105, and 116-125. In some embodiments, the lentiviral vector includes a first nucleic acid sequence operably linked to an MND promoter, wherein the first nucleic acid sequence encodes a polypeptide having at least 92% identity to any one of SEQ ID NOS: 1-8, 44-54, 68, 75, 105, and 116-125. In some embodiments, the lentiviral vector includes a first nucleic acid sequence operably linked to an MND promoter, wherein the first nucleic acid sequence encodes a polypeptide having at least 93% identity to any one of SEQ ID NOS: 1-8, 44-54, 68, 75, 105, and 116-125. In some embodiments, the lentiviral vector includes a first nucleic acid sequence operably linked to an MND promoter, wherein the first nucleic acid sequence encodes a polypeptide having at least 94% identity to any one of SEQ ID NOS: 1-8, 44-54, 68, 75, 105, and 116-125. In some embodiments, the lentiviral vector includes a first nucleic acid sequence operably linked to an MND promoter, wherein the first nucleic acid sequence encodes a polypeptide having at least 95% identity to any one of SEQ ID NOS: 1-8, 44-54, 68, 75, 105, and 116-125. In some embodiments, the lentiviral vector includes a first nucleic acid sequence operably linked to an MND promoter, wherein the first nucleic acid sequence encodes a polypeptide having at least 96% identity to any one of SEQ ID NOS: 1-8, 44-54, 68, 75, 105, and 116-125. In some embodiments, the lentiviral vector includes a first nucleic acid sequence operably linked to an MND promoter, wherein the first nucleic acid sequence encodes a polypeptide having at least 97% identity to any one of SEQ ID NOS: 1-8, 44-54, 68, 75, 105, and 116-125. In some embodiments, the lentiviral vector includes a first nucleic acid sequence operably linked to an MND promoter, wherein the first nucleic acid sequence encodes a polypeptide having at least 98% identity to any one of SEQ ID NOS: 1-8, 44-54, 68, 75, 105, and 116-125. In some embodiments, the lentiviral vector includes a first nucleic acid sequence operably linked to an MND promoter, wherein the first nucleic acid sequence encodes a polypeptide having at least 99% identity to any one of SEQ ID NOS: 1-8, 44-54, 68, 75, 105, and 116-125. In some embodiments, the lentiviral vector includes a first nucleic acid sequence operably linked to an MND promoter, wherein the first nucleic acid sequence encodes a polypeptide having any one of SEQ ID NOS: 1-8, 44-54, 68, 75, 105, and 116-125.

In some embodiments, the lentiviral vector includes a first nucleic acid sequence operably linked to an CD11b promoter, wherein the first nucleic acid sequence encodes for a polypeptide having any one of Formulas (I)-(V).

In some embodiments, the lentiviral vector includes a first nucleic acid sequence operably linked to an CD11b promoter, wherein the first nucleic acid sequence encodes for a polypeptide having at least 90% identity to any one of SEQ ID NOS: 1-8, 44-54, 68, 75, 105, and 116-125. In some embodiments, the lentiviral vector includes a first nucleic acid sequence operably linked to an CD11b promoter, wherein the first nucleic acid sequence encodes a polypeptide having at least 91% identity to any one of SEQ ID NOS: 1-8, 44-54, 68, 75, 105, and 116-125. In some embodiments, the lentiviral vector includes a first nucleic acid sequence operably linked to an CD11b promoter, wherein the first nucleic acid sequence encodes a polypeptide having at least 92% identity to any one of SEQ ID NOS: 1-8, 44-54, 68, 75, 105, and 116-125. In some embodiments, the lentiviral vector includes a first nucleic acid sequence operably linked to an CD11b promoter, wherein the first nucleic acid sequence encodes a polypeptide having at least 93% identity to any one of SEQ ID NOS: 1-8, 44-54, 68, 75, 105, and 116-125. In some embodiments, the lentiviral vector includes a first nucleic acid sequence operably linked to an CD11b promoter, wherein the first nucleic acid sequence encodes a polypeptide having at least 94% identity to any one of SEQ ID NOS: 1-8, 44-54, 68, 75, 105, and 116-125. In some embodiments, the lentiviral vector includes a first nucleic acid sequence operably linked to an CD11b promoter, wherein the first nucleic acid sequence encodes a polypeptide having at least 95% identity to any one of SEQ ID NOS: 1-8, 44-54, 68, 75, 105, and 116-125. In some embodiments, the lentiviral vector includes a first nucleic acid sequence operably linked to an CD11b promoter, wherein the first nucleic acid sequence encodes a polypeptide having at least 96% identity to any one of SEQ ID NOS: 1-8, 44-54, 68, 75, 105, and 116-125. In some embodiments, the lentiviral vector includes a first nucleic acid sequence operably linked to an CD11b promoter, wherein the first nucleic acid sequence encodes a polypeptide having at least 97% identity to any one of SEQ ID NOS: 1-8, 44-54, 68, 75, 105, and 116-125. In some embodiments, the lentiviral vector includes a first nucleic acid sequence operably linked to an CD11b promoter, wherein the first nucleic acid sequence encodes a polypeptide having at least 98% identity to any one of SEQ ID NOS: 1-8, 44-54, 68, 75, 105, and 116-125. In some embodiments, the lentiviral vector includes a first nucleic acid sequence operably linked to an CD11b promoter, wherein the first nucleic acid sequence encodes a polypeptide having at least 99% identity to any one of SEQ ID NOS: 1-8, 44-54, 68, 75, 105, and 116-125. In some embodiments, the lentiviral vector includes a first nucleic acid sequence operably linked to an CD11b promoter, wherein the first nucleic acid sequence encodes a polypeptide having any one of SEQ ID NOS: 1-8, 44-54, 68, 75, 105, and 116-125.

In some embodiments, the lentiviral vector includes a first nucleic acid sequence operably linked to an EFS promoter, wherein the first nucleic acid sequence encodes for a polypeptide having any one of Formulas (I)-(V).

In some embodiments, the lentiviral vector includes a first nucleic acid sequence operably linked to an EFS promoter, wherein the first nucleic acid sequence encodes for a polypeptide having at least 90% identity to any one of SEQ ID NOS: 1-8, 44-54, 68, 75, 105, and 116-125. In some embodiments, the lentiviral vector includes a first nucleic acid sequence operably linked to an EFS promoter, wherein the first nucleic acid sequence encodes a polypeptide having at least 91% identity to any one of SEQ ID NOS: 1-8, 44-54, 68, 75, 105, and 116-125. In some embodiments, the lentiviral vector includes a first nucleic acid sequence operably linked to an EFS promoter, wherein the first nucleic acid sequence encodes a polypeptide having at least 92% identity to any one of SEQ ID NOS: 1-8, 44-54, 68, 75, 105, and 116-125. In some embodiments, the lentiviral vector includes a first nucleic acid sequence operably linked to an EFS promoter, wherein the first nucleic acid sequence encodes a polypeptide having at least 93% identity to any one of SEQ ID NOS: 1-8, 44-54, 68, 75, 105, and 116-125. In some embodiments, the lentiviral vector includes a first nucleic acid sequence operably linked to an EFS promoter, wherein the first nucleic acid sequence encodes a polypeptide having at least 94% identity to any one of SEQ ID NOS: 1-8, 44-54, 68, 75, 105, and 116-125. In some embodiments, the lentiviral vector includes a first nucleic acid sequence operably linked to an EFS promoter, wherein the first nucleic acid sequence encodes a polypeptide having at least 95% identity to any one of SEQ ID NOS: 1-8, 44-54, 68, 75, 105, and 116-125. In some embodiments, the lentiviral vector includes a first nucleic acid sequence operably linked to an EFS promoter, wherein the first nucleic acid sequence encodes a polypeptide having at least 96% identity to any one of SEQ ID NOS: 1-8, 44-54, 68, 75, 105, and 116-125. In some embodiments, the lentiviral vector includes a first nucleic acid sequence operably linked to an EFS promoter, wherein the first nucleic acid sequence encodes a polypeptide having at least 97% identity to any one of SEQ ID NOS: 1-8, 44-54, 68, 75, 105, and 116-125. In some embodiments, the lentiviral vector includes a first nucleic acid sequence operably linked to an EFS promoter, wherein the first nucleic acid sequence encodes a polypeptide having at least 98% identity to any one of SEQ ID NOS: 1-8, 44-54, 68, 75, 105, and 116-125. In some embodiments, the lentiviral vector includes a first nucleic acid sequence operably linked to an EFS promoter, wherein the first nucleic acid sequence encodes a polypeptide having at least 99% identity to any one of SEQ ID NOS: 1-8, 44-54, 68, 75, 105, and 116-125. In some embodiments, the lentiviral vector includes a first nucleic acid sequence operably linked to an EFS promoter, wherein the first nucleic acid sequence encodes a polypeptide having any one of SEQ ID NOS: 1-8, 44-54, 68, 75, 105, and 116-125.

In some embodiments, the lentiviral vector includes a first nucleic acid sequence operably linked to an Ubc promoter, wherein the first nucleic acid sequence encodes for a polypeptide having any one of Formulas (I)-(V).

In some embodiments, the lentiviral vector includes a first nucleic acid sequence operably linked to an Ubc promoter, wherein the first nucleic acid sequence encodes for a polypeptide having at least 90% identity to any one of SEQ ID NOS: 1-8, 44-54, 68, 75, 105, and 116-125. In some embodiments, the lentiviral vector includes a first nucleic acid sequence operably linked to an Ubc promoter, wherein the first nucleic acid sequence encodes a polypeptide having at least 91% identity to any one of SEQ ID NOS: 1-8, 44-54, 68, 75, 105, and 116-125. In some embodiments, the lentiviral vector includes a first nucleic acid sequence operably linked to an Ubc promoter, wherein the first nucleic acid sequence encodes a polypeptide having at least 92% identity to any one of SEQ ID NOS: 1-8, 44-54, 68, 75, 105, and 116-125. In some embodiments, the lentiviral vector includes a first nucleic acid sequence operably linked to an Ubc promoter, wherein the first nucleic acid sequence encodes a polypeptide having at least 93% identity to any one of SEQ ID NOS: 1-8, 44-54, 68, 75, 105, and 116-125. In some embodiments, the lentiviral vector includes a first nucleic acid sequence operably linked to an Ubc promoter, wherein the first nucleic acid sequence encodes a polypeptide having at least 94% identity to any one of SEQ ID NOS: 1-8, 44-54, 68, 75, 105, and 116-125. In some embodiments, the lentiviral vector includes a first nucleic acid sequence operably linked to an Ubc promoter, wherein the first nucleic acid sequence encodes a polypeptide having at least 95% identity to any one of SEQ ID NOS: 1-8, 44-54, 68, 75, 105, and 116-125. In some embodiments, the lentiviral vector includes a first nucleic acid sequence operably linked to an Ubc promoter, wherein the first nucleic acid sequence encodes a polypeptide having at least 96% identity to any one of SEQ ID NOS: 1-8, 44-54, 68, 75, 105, and 116-125. In some embodiments, the lentiviral vector includes a first nucleic acid sequence operably linked to an Ubc promoter, wherein the first nucleic acid sequence encodes a polypeptide having at least 97% identity to any one of SEQ ID NOS: 1-8, 44-54, 68, 75, 105, and 116-125. In some embodiments, the lentiviral vector includes a first nucleic acid sequence operably linked to an Ubc promoter, wherein the first nucleic acid sequence encodes a polypeptide having at least 98% identity to any one of SEQ ID NOS: 1-8, 44-54, 68, 75, 105, and 116-125. In some embodiments, the lentiviral vector includes a first nucleic acid sequence operably linked to an Ubc promoter, wherein the first nucleic acid sequence encodes a polypeptide having at least 99% identity to any one of SEQ ID NOS: 1-8, 44-54, 68, 75, 105, and 116-125. In some embodiments, the lentiviral vector includes a first nucleic acid sequence operably linked to an Ubc promoter, wherein the first nucleic acid sequence encodes a polypeptide having any one of SEQ ID NOS: 1-8, 44-54, 68, 75, 105, and 116-125.

In some embodiments, the lentiviral vector includes a first nucleic acid sequence operably linked to an CD68LPp promoter, wherein the first nucleic acid sequence encodes for a polypeptide having any one of Formulas (I)-(V).

In some embodiments, the lentiviral vector includes a first nucleic acid sequence operably linked to an CD68LPp promoter, wherein the first nucleic acid sequence encodes for a polypeptide having at least 90% identity to any one of SEQ ID NOS: 1-8, 44-54, 68, 75, 105, and 116-125. In some embodiments, the lentiviral vector includes a first nucleic acid sequence operably linked to an CD68LPp promoter, wherein the first nucleic acid sequence encodes a polypeptide having at least 91% identity to any one of SEQ ID NOS: 1-8, 44-54, 68, 75, 105, and 116-125. In some embodiments, the lentiviral vector includes a first nucleic acid sequence operably linked to an CD68LPp promoter, wherein the first nucleic acid sequence encodes a polypeptide having at least 92% identity to any one of SEQ ID NOS: 1-8, 44-54, 68, 75, 105, and 116-125. In some embodiments, the lentiviral vector includes a first nucleic acid sequence operably linked to an CD68LPp promoter, wherein the first nucleic acid sequence encodes a polypeptide having at least 93% identity to any one of SEQ ID NOS: 1-8, 44-54, 68, 75, 105, and 116-125. In some embodiments, the lentiviral vector includes a first nucleic acid sequence operably linked to an CD68LPp promoter, wherein the first nucleic acid sequence encodes a polypeptide having at least 94% identity to any one of SEQ ID NOS: 1-8, 44-54, 68, 75, 105, and 116-125. In some embodiments, the lentiviral vector includes a first nucleic acid sequence operably linked to an CD68LPp promoter, wherein the first nucleic acid sequence encodes a polypeptide having at least 95% identity to any one of SEQ ID NOS: 1-8, 44-54, 68, 75, 105, and 116-125. In some embodiments, the lentiviral vector includes a first nucleic acid sequence operably linked to an CD68LPp promoter, wherein the first nucleic acid sequence encodes a polypeptide having at least 96% identity to any one of SEQ ID NOS: 1-8, 44-54, 68, 75, 105, and 116-125. In some embodiments, the lentiviral vector includes a first nucleic acid sequence operably linked to an CD68LPp promoter, wherein the first nucleic acid sequence encodes a polypeptide having at least 97% identity to any one of SEQ ID NOS: 1-8, 44-54, 68, 75, 105, and 116-125. In some embodiments, the lentiviral vector includes a first nucleic acid sequence operably linked to an CD68LPp promoter, wherein the first nucleic acid sequence encodes a polypeptide having at least 98% identity to any one of SEQ ID NOS: 1-8, 44-54, 68, 75, 105, and 116-125. In some embodiments, the lentiviral vector includes a first nucleic acid sequence operably linked to an CD68LPp promoter, wherein the first nucleic acid sequence encodes a polypeptide having at least 99% identity to any one of SEQ ID NOS: 1-8, 44-54, 68, 75, 105, and 116-125. In some embodiments, the lentiviral vector includes a first nucleic acid sequence operably linked to an CD68LPp promoter, wherein the first nucleic acid sequence encodes a polypeptide having any one of SEQ ID NOS: 1-8, 44-54, 68, 75, 105, and 116-125.

Transcription may be increased by inserting an enhancer sequence into the vector(s). Enhancers are typically cis-acting elements of DNA, usually about 10 to 300 bp in length, that act on a promoter to increase its transcription. Many enhancer sequences are now known from mammalian genes (globin, elastase, albumin, alpha-fetoprotein, and insulin) and from eukaryotic cell viruses. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the antigen-specific polynucleotide sequence but is preferably located at a site 5′ from the promoter.

In some embodiments, the vectors of the present disclosure include an insulator element, e.g., a cHS insulator. In some embodiments, the insulator has a nucleotide sequencing having at least 95% identity to any one of SEQ ID NOS: 127-129. In some embodiments, the insulator has a nucleotide sequencing having at least 97% identity to any one of SEQ ID NOS: 127-129. In some embodiments, the insulator has a nucleotide sequencing having at least 99% identity to any one of SEQ ID NOS: 127-129. In some embodiments, the insulator has a nucleotide sequencing having any one of SEQ ID NOS: 127-129.

Production of Viral Particles

Any of a variety of methods already known in the art may be used to produce infectious lentiviral particles whose genome comprises an RNA copy of the viral vector genome. In one method, the viral vector genome is introduced into a packaging cell line that contains all the components necessary to package viral genomic RNA, transcribed from the viral vector genome, into viral particles. Alternatively, the viral vector genome may comprise one or more genes encoding viral components in addition to the one or more sequences of interest. In order to prevent replication of the genome in the target cell, however, endogenous viral genes required for replication will usually be removed and provided separately in the packaging cell line.

In general, the lentiviral vector particles are produced by a cell line that is transfected with one or more plasmid vectors containing the components necessary to generate the particles. These lentiviral vector particles are typically not replication-competent, i.e., they are only capable of a single round of infection. Most often, multiple plasmid vectors are utilized to separate the various genetic components that generate the lentiviral vector particles, mainly to reduce the chance of recombination events that might otherwise generate replication competent viruses. A single plasmid vector having all of the lentiviral components can be used if desired, however. As one example of a system that employs multiple plasmid vectors, a cell line is transfected with at least one plasmid containing the viral vector genome (i.e., the vector genome plasmid), including the LTRs, the cis-acting packaging sequence, and the sequence(s) of interest, which are often operably linked to a heterologous promoter, at least one plasmid encoding the virus enzymatic and structural components (i.e., the packaging plasmid that encodes components such as Gag and Pol), and at least one envelope plasmid encoding an Arbovirus envelope glycoprotein. Additional plasmids can be used to enhance retrovirus particle production, e.g., Rev-expression plasmids, as described herein and known in the art. Viral particles bud through the cell membrane and comprise a core that includes a genome containing the sequence of interest and an Arbovirus envelope glycoprotein that targets dendritic cells. When the Arbovirus glycoprotein is Sindbis virus E2 glycoprotein, the glycoprotein is engineered to have reduced binding to heparan sulfate compared to the reference strain HR.

Transfection of packaging cells with plasmid vectors can be accomplished by well-known methods, and the method to be used is not limited in any way. A number of non-viral delivery systems are known in the art, including for example, electroporation, lipid-based delivery systems including liposomes, delivery of “naked” DNA, and delivery using polycyclodextrin compounds, such as those described in Schatzlein A. G. (2001, Non-Viral Vectors in Cancer Gene Therapy: Principles and Progresses. Anticancer Drugs, which is incorporated herein by reference in its entirety). Cationic lipid or salt treatment methods are typically employed, see, for example. Graham et al. (1973, Virol. 52:456; Wigler et al.; 1979, Proc. Natl. Acad. Sci. USA 76:1373-76), each of the foregoing which is incorporated herein by reference in its entirety. The calcium phosphate precipitation method is most often used. However, other methods for introducing the vector into cells may also be used, including nuclear microinjection and bacterial protoplast fusion.

The packaging cell line provides the components, including viral regulatory and structural proteins, that are required in trans for the packaging of the viral genomic RNA into lentiviral vector particles. The packaging cell line may be any cell line that is capable of expressing lentiviral proteins and producing functional lentiviral vector particles. Some suitable packaging cell lines include 293 (ATCC CCL X), 293T, HeLa (ATCC CCL 2), D17 (ATCC CCL 183), MDCK (ATCC CCL 34), BHK (ATCC CCL-10) and Cf2Th (ATCC CRL 1430) cells. The packaging cell line may stably express the necessary viral proteins. Such a packaging cell line is described, for example, in U.S. Pat. No. 6,218,181, which is incorporated herein by reference in its entirety. Alternatively a packaging cell line may be transiently transfected with nucleic acid molecules encoding one or more necessary viral proteins along with the viral vector genome. The resulting viral particles are collected and used to infect a target cell. The gene(s) encoding envelope glycoprotein(s) is usually cloned into an expression vector, such as pcDNA3 (Invitrogen, CA USA). Eukaryotic cell expression vectors are well known in the art and are available from a number of commercial sources. Packaging cells, such as 293T cells are then co-transfected with the viral vector genome encoding a sequence of interest (typically encoding an antigen), at least one plasmid encoding virus packing components, and a vector for expression of the targeting molecule. The envelope is expressed on the membrane of the packaging cell and incorporated into the viral vector.

Compositions Comprising a Polynucleotide

Another aspect of the present disclosure is directed to compositions comprising one or more of the polynucleotides having any one of Formulas (I), (II), (III), (IV), (V) or a variant or fragment thereof. For example, the composition may comprise a polypeptide having an amino acid sequence having at least 90% identity (e.g., 91% identity, 92% identity, 93% identity, 94% identity, 95% identity, 96% identity, 97% identity, 98% identity, 99% identity) to any one of SEQ ID NOS: 2-8, 44-54, 68, 75, 105, and 116-125. In some embodiments, the composition may comprise a polypeptide having an amino acid sequence having any one of SEQ ID NOS: 2-8, 44-54, 68, 75, 105, and 116-125.

In some embodiments, the composition further comprises a physiologically acceptable carrier, excipient, or stabilizer. See, e.g., Remington's Pharmaceutical Sciences (1990) Mack Publishing Co., Easton, Pa. Acceptable carriers, excipients, or stabilizers can include those that are nontoxic to a subject. In some embodiments, the composition or one or more components of the composition are sterile. A sterile component can be prepared, for example, by filtration (e.g., by a sterile filtration membrane) or by irradiation (e.g., by gamma irradiation).

Suitable compositions include aqueous and non-aqueous isotonic sterile solutions, which can contain anti-oxidants, buffers, and bacteriostats, and aqueous and non-aqueous sterile suspensions that can include suspending agents, solubilizers, thickening agents, stabilizers, and preservatives. The composition can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the carrier is a buffered saline solution. In some embodiments, the lentiviral vector is part of a composition formulated to protect the lentiviral vector from damage prior to administration. For example, the composition can be formulated to reduce loss of the lentiviral vector on devices used to prepare, store, or administer lentiviral vector, such as glassware, syringes, or needles. In some embodiments, the composition can be formulated to decrease the light sensitivity and/or temperature sensitivity of lentiviral vector. To this end, the composition preferably comprises a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the lentiviral vector and facilitate its administration. Formulations for lentiviral-containing compositions are further described in, for example, Ausubel et al., Bioprocess Int., 10(2): 32-43 (2012), U.S. Pat. No. 7,575,924, and International Patent Application Publication WO 2013/139300.

The compositions comprising the one or more polypeptides of any one of Formulas (I), (II), (III), (IV), and (V) may be administered to humans or other animals on whose tissues they are effective in various manners such as topically, orally, intravenously, intramuscularly, intraperitoneally, intranasally, intradermally, intrathecally, subcutaneously, intraocularly, via inhalation, or via suppository. In one example, the compounds are administered to the subject subcutaneously. In another example, the compounds are administered to the subject intravenously.

Compositions Comprising an Expression Vector

Another aspect of the present disclosure is directed to compositions comprising one or more expression vectors, e.g., an expression vector having at least 95%, 96%, 97%, 98%, or 99% identity to any one of SEQ ID NOS: 15-26, 55-65, 74, 81, 82, 84-95, 97, and 98. In some embodiments, the present disclosure provides a composition comprising one or more of the expression vectors described herein and a carrier therefor (e.g., a pharmaceutically acceptable carrier). The composition desirably is a physiologically acceptable (e.g., pharmaceutically acceptable) composition, which comprises a carrier, e.g. a physiologically (e.g., pharmaceutically) acceptable carrier, and the lentiviral vector. Any suitable carrier can be used within the context of the present disclosure, and such carriers are well known in the art, including any of those described above.

Host Cells

The present disclosure is also directed to a method of producing a recombinant polypeptide, comprising: culturing an isolated host cell as described herein and isolating the recombinant polypeptide from the host cell. Techniques for isolating polypeptides from cultured host cells can be any technique known to be used or routinely modified when isolating polypeptides from the host cell selected for expression and will be apparent to the ordinarily skilled artisan. Suitable host cells are those cell types that can be transformed or transfected with exogenous DNA and grown in culture, and include bacteria, fungal cells, and cultured higher eukaryotic cells (including cultured cells of multicellular organisms), particularly cultured mammalian cells. Techniques for manipulating cloned DNA molecules and introducing exogenous DNA into a variety of host cells are disclosed by Sambrook and Russell, Molecular Cloning: A Laboratory Manual (3rd ed., Cold Spring Harbor Laboratory Press, Cold Spring Harbor, N.Y., 2001), and Ausubel et al., Short Protocols in Molecular Biology (4th ed., John Wiley & Sons, 1999). For example, the recombinant polypeptides of the present disclosure may be expressed from bacterial Escherichia coli cells.

Cultured mammalian cells can be suitable hosts for production of recombinant polypeptides for use within the present disclosure. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-845, 1982), DEAE-dextran mediated transfection (Ausubel et al., supra), and liposome-mediated transfection (Hawley-Nelson et al., Focus 15:73, 1993; Ciccarone et al., Focus 15:80, 1993). The production of recombinant polypeptides in cultured mammalian cells is disclosed by, for example, Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-Kl; ATCC CCL61; CHO DG44; CHO DXB11 (Hyclone, Logan, Utah); see also, e.g., Chasin et al., Som. Cell. Molec. Genet. 12:555, 1986)), rat pituitary cells (GH1; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650) and murine embryonic cells (NIH-3T3; ATCC CRL 1658). Additional suitable cell lines are known in the art and available from public depositories such as the American Type Culture Collection, Manassas, Va. Strong transcription promoters can be used, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

The present disclosure also provides a host cell (or a population of host cells) transduced with any one of the expression vectors (e.g., lentiviral expression vectors) or composition comprising any one of the expression vectors described herein. For instance, the host cells may be transduced with an expression vector according to any of the embodiments described herein, e.g., an expression vector having at least 95%, 96%, 97%, 98%, or 99% identity to any one of SEQ ID NOS: 15-26, 55-65, 74, 81, 82, 84-95, 97, and 98. In some embodiments, the host cells are transduced with an expression vector to express any one of the polypeptides described herein, e.g., a polypeptide having an amino acid sequence having at least 90% identity (e.g., 91% identity, 92% identity, 93% identity, 94% identity, 95% identity, 96% identity, 97% identity, 98% identity, 99% identity) to any one of SEQ IF NOS: 2-8, 44-54, 68, 75, 105, and 116-125.

In some embodiments, host cells are those that can be easily and reliably grown, have reasonably fast growth rates, have well characterized expression systems, and can be transformed, transfected, or transduced easily and efficiently with the lentiviral vectors of the present disclosure. The host cell can be any suitable eukaryotic cell known in the art including, for example, yeast cells, insect cells, and mammalian cells. In some embodiments, mammalian cells are utilized in the present disclosure. In one embodiment, the host cells are packaging cells used for producing lentiviral vector particles, including, for example, 293T cells (ATCC No. CRL-3216) and HT1080 cells (ATCC No. CCL-121).

In some embodiments, the host cell is a hematopoietic stem cell or a progenitor cell produced or derived therefrom (including but not limited to cells in the blood such as granulocytes/neutrophils, lymphocytes, erythrocytes, megakaryocytes/platelets, and monocytes/macrophages; HSPC-derived tissue-resident cells such as dendritic cells, Kupffer cells, microglia, and osteoclasts). Hematopoietic stem cells (HSCs) are multipotent, self-renewing progenitor cells that develop from mesodermal hemangioblast cells. All differentiated blood cells (i.e., myelocytes, lymphocytes, erythrocytes, and platelets) arise from HSCs. HSCs can be found in adult bone marrow, peripheral blood, and umbilical cord blood. In another embodiment, the host cell is a cell that expresses the CD34 protein, which is also referred to as a “CD34+” cell. CD34 is a cell surface glycoprotein that functions as a cell-cell adhesion factor and may also mediate the attachment of stem cells to bone marrow extracellular matrix or directly to stromal cells. CD34 is a marker for primitive blood- and bone marrow-derived progenitor cells, especially for HSCs.

In some embodiments, the host cell is a mesenchymal stem cell. Mesenchymal stem cells are multipotent adult stem cells that are present in multiple tissues, including umbilical cord, bone marrow and fat tissue. Mesenchymal stem cells can self-renew by dividing and can differentiate into multiple tissues including bone, cartilage, muscle and fat cells, and connective tissue.

In some embodiments, the host cells are bone marrow cells. In some embodiments, the host cells are hepatocytes. In some embodiments, the host cells are endothelial cells.

The methods of the present disclosure may be used in gene therapy approaches, in treatments for either genetic or acquired diseases. The general approach of gene therapy involves the introduction of nucleic acid into cells such that one or more gene products encoded by the introduced genetic material are produced in the cells to restore or enhance a functional activity. For reviews on gene therapy approaches see Anderson, W. F. (1992) Science 256:808-813; Miller, A. D. (1992) Nature 357:455-460; Friedmann, T. (1989) Science 244:1275-1281; and Cournoyer, D., et al. (1990) Curr. Opin. Biotech. 1:196-208). Cells types which can be modified for gene therapy purposes include hematopoietic stem cells, myoblasts, hepatocytes, lymphocytes, skin epithelium and airway epithelium. For further descriptions of cell types, genes and methods for gene therapy see e.g., Wilson, J. M et al. (1988) Proc. Natl Acad. Sci. USA 85:3014-3018; Armentano, D. et al. (1990) Proc. Natl Acad. Sci. USA 87:6141-6145; Wolff, J. A. et al. (1990) Science 247:1465-1468; Chowdhury, J. R. et al. (1991) Science 254:1802-1805; Ferry, N. et al (1991) Proc. Natl Acad. Sci. USA 88:8377-8381; Wilson, J. M. et al. (1992) J. Biol Chem. 267:963-967; Quantin, B. et al (1992) Proc. Natl. Acad. Sci. USA 89:2581-2584; Dai, Y. et al. (1992) Proc. Natl Acad. Sci. USA 89:10892-10895; van Beusechem, V. W. et al. (1992) Proc. Natl. Acad. Sci. USA 89:7640-7644; Rosenfeld, M. A. et al. (1992) Cell 68:143-155; Kay, M. A. et al. (1992) Human Gene Therapy 3:641-647; Cristiano, R. J. et al. (1993) Proc. Natl. Acad. Sci. USA 90:2122-2126; Hwu, P. et al. (1993) J. Immunol. 150:4104-4115; and Herz, J. and Gerard, R. D. (1993) Proc. Natl. Acad. Sci. USA 90:2812-2816.

Methods of Treatment

Another aspect of the present disclosure is directed to a method of treating hypophosphatasia in a mammal, e.g. a human, in need thereof. Another aspect of the present disclosure is directed to a method of treating, mitigating, or preventing a symptom of hypophosphatasia in a mammal, e.g. a human. In some embodiments, the present disclosure is directed to treating a condition or disease related to a bone defect characterized by a lack of or an insufficient amount of functional alkaline phosphatase.

In some embodiments, the method comprises (a) harvesting hematopoietic stem cells from the mammal, (b) transducing the hematopoietic stein cells with an expression vector (e.g., a lentiviral expression vector) or a composition comprising an expression vector, and (c) transplanting the transduced hematopoietic stem cells into in the mammal. In some embodiments, the expression vector comprises at least 95%, 96%, 97%, 98%, or 99% identity to any one of SEQ ID NOS: 15-26, 55-65, 74, 81, 82, 84-95, 97, and 98. In some embodiments, the lentiviral vector comprises a nucleic acid sequence encoding a polypeptide, such as any of the polypeptides having Formulas (I), (II), (III), (IV), and (V), e.g., a polypeptide having an amino acid sequence having at least 90% identity (e.g., 91% identity, 92% identity, 93% identity, 94% identity, 95% identity, 96% identity, 97% identity, 98% identity, 99% identity) to any one of SEQ IDS NOS: 1-8, 44-54, 68, 75, 105, and 116-125.

Hematopoietic stem cells can be harvested from bone marrow, peripheral blood, or umbilical cord blood of the mammal (e.g., a human) using methods known in the art, such as those described in, for example, Wognum et al., Arch Med Res., 34(6): 461-75 (2003); Ng et al., Methods Mol. Biol., 506: 13-21 (2009); Weissman and Shizuru, Blood, 112(9): 3543-3553 (2008); Frisch and Calvi, Skeletal Development and Repair Methods in Molecular Biology, 1130: 315-324 (2014); and U.S. Pat. No. 8,383,404. For example, HSCs can be harvested from the pelvis, at the iliac crest, using a needle and syringe. Alternatively, HSCs can be isolated from circulating peripheral blood by injecting the mammal (or allogeneic donor) with a cytokine, such as granulocyte-colony stimulating factor (G-CSF), that induce cells to leave the bone marrow and circulate in the blood vessels.

In some embodiments, the harvested HSCs can be “autologous” or “allogeneic.” In some embodiments, autologous HSCs are removed from a mammal, stored (and optionally modified), and returned back to the same mammal. In some embodiments, allogeneic HSCs are removed from a mammal, stored (and optionally modified), and transplanted into a genetically similar, but not identical, recipient. In some embodiments, the cells are autologous to the mammal.

The expression vector or composition comprising the expression vector of the present disclosure may be introduced into a hematopoietic cell by “transfection,” “transformation,” or “transduction.” The terms “transfection,” “transformation,” or “transduction,” as used herein, refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)). Lentiviral vectors typically are introduced into host cells after growth of infectious particles in suitable packaging cells.

The HSCs may be transduced with the expression vector ex vivo, in vivo or in vitro, depending on the ultimate application. In some embodiments, the HSCs are transduced in vitro with the expression vector or composition comprising the expression vector followed by infusion of the transduced stem cells into the mammal. In this embodiment, the human stem cell can be removed from a human patient using methods well known to in the art and transduced as described above. In some embodiments, the transduced HSCs are then reintroduced into the same (autologous) or different mammal (allogeneic). In some embodiments, the HSCs are transduced ex vivo.

In some embodiments, the methods of the present disclosure comprise transplanting the HSCs into a mammal in need thereof. Hematopoietic stem cell transplantation (HSCT) has become the standard of care for many patients with certain congenital or acquired disorders of the hematopoietic system or with chemosensitive, radiosensitive, or immunosensitive malignancies (see, e.g., Gratwohl et al., JAMA, 303(16): 1617-1624 (2010); and Copelan, E. A., NEJM, 354: 1813-1826 (2006)). Methods of isolating stem cells from a subject, transducing them with a therapeutic gene (e.g., an anti-sickling human (3-globin gene), and returning the modified stem cells to the subject are well known in the art (see, e.g., Pawliuk et al., Science, 294(5550): 2368-2371 (2001); Tyndall et al., Bone Marrow Transplant, 24 (7): 729-34 (1999); and Burt et al., JAMA, 299 (8): 925-36 (2008)). Other methods for transplanting HSCs into a subject in need thereof that can be used in the method described herein include those used, for example, for bone marrow transplantation or peripheral blood stem cell transplantation.

In some embodiments, the hematopoietic stem cells can be transduced with the expression vector in vivo by directly injecting into a mammal in need thereof the aforementioned composition comprising the lentiviral vector and a carrier therefor (e.g., a pharmaceutically acceptable carrier). In some embodiments, the composition comprising the expression vector can be administered to a mammal using standard administration techniques (e.g. parenteral administration). The term “parenteral,” as used herein, includes intravenous, intramuscular, subcutaneous, rectal, vaginal, and intraperitoneal administration. In some embodiments, the composition is administered to a mammal using peripheral systemic delivery by intravenous, intraperitoneal, or subcutaneous injection.

In some embodiments, the methods of the present disclosure provide for the treatment of hypophosphatasia in the mammal. As used herein, the terms “treatment,” “treating,” and the like refer to obtaining a desired pharmacologic and/or physiologic effect. In some embodiments, the effect is therapeutic, i.e., the effect partially or completely cures a disease and/or adverse symptom attributable to the disease. In some embodiments, a “therapeutically effective amount” of the transduced HSCs or the composition comprising the expression vector are administered to the subject tin need of treatment thereof. A “therapeutically effective amount” refers to an amount effective, at dosages and for periods of time necessary, to achieve a desired therapeutic result.

In some embodiments, the therapeutically effective amount may vary according to factors such as the disease state, age, sex, and weight of the individual, and the ability of the transduced HSC's and encoded protein or variant thereof to elicit a desired response in the individual. For example, in some embodiments a therapeutically effective amount of transduced HSCs of the present disclosure is an amount which results in expression of a polypeptide having any one of Formulas (I), (II), (III), (IV), or (V) or a variant or fragment thereof at levels that ameliorates or reverses hypophosphatasia in a human.

The dose of lentiviral vector delivered to hematopoietic stem cells, either by in vitro or in vivo methods, typically can be, for example, a multiplicity of infection (MOI) in the range of 1 to 100 (e.g., 1, 5, 10, 15, 20, 25, 30, 35, 40, 45, 50, 55, 60, 65, 70, 75, 80, 85, 90, 95, 100 MOI, or a range defined by any two of the forgoing values); however, doses below or above this exemplary range are within the scope of the present disclosure.

In some embodiments, the lentiviral vector particles are in a dose of 1×106 TU, 2×106 TU, 3×106 TU, 4×106 TU, 5×106 TU, 6×106 TU, 7×106 TU, 8×106 TU, 9×106, 1×107 TU, 2×107 TU, 3×107 TU, 4×107 TU, 5×107 TU, 6×107 TU, 7′107 TU, 8×107 TU, 9×107 TU, 1×108 TU, 2×108 TU, 3×108 TU, 4×108 TU, 5×108 TU, 6×108 TU, 7×108 TU, 8×108 TU, 9×108 TU, or 1×109 TU. In some embodiments, the lentiviral vector particles are in a dose of from 1×106 TU to 5×106 TU, from 5×106 TU to 1×107 TU, from 1×107 TU to 5×107 TU, from 5×107 TU to 1×108 TU, from 1×108 TU to 5×108 TU, or from 5×108 TU to 1×109 TU.

In some embodiments, the pharmacologic and/or physiologic effect may be prophylactic, i.e., the effect completely or partially prevents a disease or symptom thereof. In this respect, the present disclosures provides a method of treatment which comprises administering a “prophylactically effective amount” of the transduced HSCs or the composition comprising one or more of the expression vectors described herein. A “prophylactically effective amount” refers to an amount effective, at dosages and for periods of time necessary, to achieve a desired prophylactic result (e.g., prevention of disease onset).

Therapeutic or prophylactic efficacy can be monitored by periodic assessment of treated patients. For repeated administrations over several days or longer, depending on the condition, the treatment can be repeated until a desired suppression of disease symptoms occurs. In some embodiments, the desired dosage can be delivered by a single bolus administration of the composition or by multiple bolus administrations of the composition.

In some embodiments, transduced HSCs or the composition comprising one or more of the expression vectors described herein may be provided to a mammal alone or in combination with other drugs (e.g., as an adjuvant). For example, the transduced HSCs or the composition comprising the expression vector can be administered in combination with other agents for the treatment or prevention of hypophosphatasia. For example, transduced HSCs may be provided to a subject in need of treatment thereof, and wherein the subject is concurrently being treated with a recombinant alkaline phosphatase, such as Asfotase alfa, available from Alexion Pharmaceuticals, Inc. Alternatively, the transduced HSCs may be provided to a subject in need of treatment thereof, and wherein the subject was previously treated with a recombinant alkaline phosphatase or will be treated in the future with an alkaline phosphatase, such as Asfotase alfa, available from Alexion Pharmaceuticals, Inc.

Alternatively, the transduced HSCs can be administered in combination with other agents that reduce or prevent one or more complications associated with HSC transplantation. Such complications include, for example, infections, sepsis, mucositis, and graft-versus-host-disease (GVHD). In this respect, the transduced HSCs can be used in combination with antiviral agents, anticoagulants (e.g., defibrotide), ursodeoxycholic acid, and/or corticosteroids (e.g., prednisone). some

In particular embodiments, patients receive a dose of transduced hematopoietic stem cells of about 1×10⁵ cells/kg, about 5×10⁵ cells/kg, about 1×10⁶ cells/kg, about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 5×10⁷ cells/kg, about 1×10⁸ cells/kg, or more in one single intravenous dose. In a certain embodiment, patients receive a dose of transduced hematopoietic stem cells of about 1×10⁵ cells/kg to about 1×10⁸ cells/kg, about 1×10⁶ cells/kg to about 1×10⁸ cells/kg, about 1×10⁶ cells/kg to about 9×10⁶ cells/kg, about 2×10⁶ cells/kg to about 8×10⁶ cells/kg, about 2×10⁶ cells/kg to about 8×10⁶ cells/kg, about 2×10⁶ cells/kg to about 5×10⁶ cells/kg, about 3×10⁶ cells/kg to about 5×10⁶ cells/kg, about 3×10⁶ cells/kg to about 4×10⁸ cells/kg, or any intervening dose of cells/kg.

Transduced cells can be stimulated with cytokines for expansion using existing methods in the art. In various embodiments, subjects are administered 1, 2, 3, 4, 5, or more doses over days, months, or years, as needed to maintain or increase the therapy.

### Examples

### Example 1—Cell Mineralization Inhibition/Rescue Experiment for the Polypeptide Having SEQ ID NO: 5

Cell culture—Mouse calvarial pre-osteoblasts MC3T3-E1 cells (subclone 14) were used for these experiments, since they show robust mineralization associated with extracellular matrix production and assembly, similar to bone in vivo. Many other osteoblast cell culture systems mineralize dystrophically, without properly elaborating and assembling an extracellular matrix.

Cultures were maintained and cultured in minimum essential medium (MEM) (Gibco) supplemented with 10% fetal bovine serum (FBS) (Hyclone), 1% penicillin-streptomycin and 2 mM L-Glutamine (Gibco) at 37° C. in a humidified atmosphere of 5% CO₂. Cells were plated at 50,000 cells per cm 2 and differentiation into mature osteoblasts was induced after 24 h of adhesion (day 0) by adding 50 μg/ml ascorbic acid (AA) (Sigma) to facilitate collagen secretion and assembly, and 10 mM β-glycerophosphate ((3GP) (Sigma) to facilitate mineralization in providing an organic phosphate source. The differentiation medium was changed every 48 h.

Cell proliferation (MTT)—Cell proliferation and viability was tested in the presence of various concentrations of the polypeptide having SEQ ID NO: 5, by analyzing MTT (344,5-dimethyl-2-thiazolyl)-2,5-diphenyl-2H-tetrazolium bromide) (Sigma) incorporation into viable cells. Briefly, plated cells in 96 well plate were incubated with 0.25 mg/ml MTT solution for 3 h at 37° C. After incubation, precipitate was dissolved in DMSO. Spectrophotometric measurements were done using 562 nm wavelength in a microplate reader.

As measured by the MTT assay, there was no toxic effect of the polypeptide having SEQ ID NO: 5 to the cells even at the highest concentration (10 U/ml).

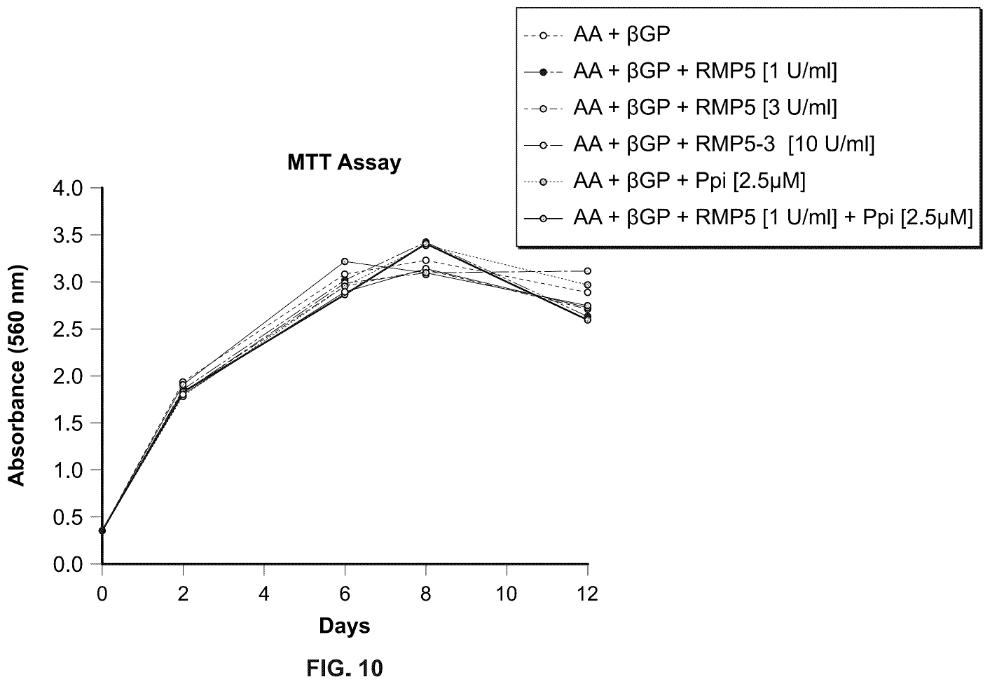

To document a potential mineralization inhibition/rescue a cell culture was initiated in differentiation medium (including AA and (3GP). At day 4, various concentrations of the polypeptide having SEQ ID NO: 5 were tested, namely 0.1, 0.3, and 1.0 unit/ml to the culture medium with or without mineralization inhibitor sodium pyrophosphate tetrabasic (PPi) at a concentration of 2.5 μM for the remainder of culture period (10 days) (see FIG. 10)

Mineral was visualized by von Kossa staining at day 10. Cells were fixed with 95% ethanol for 10 min at 37° C. After hydrated the cells with d.H2O, cells were incubated with 5% silver nitrate solution (Sigma) for an hour at 37° C. Cells were washed with d.H2O an exposed to bright light for an hour at room temperature.

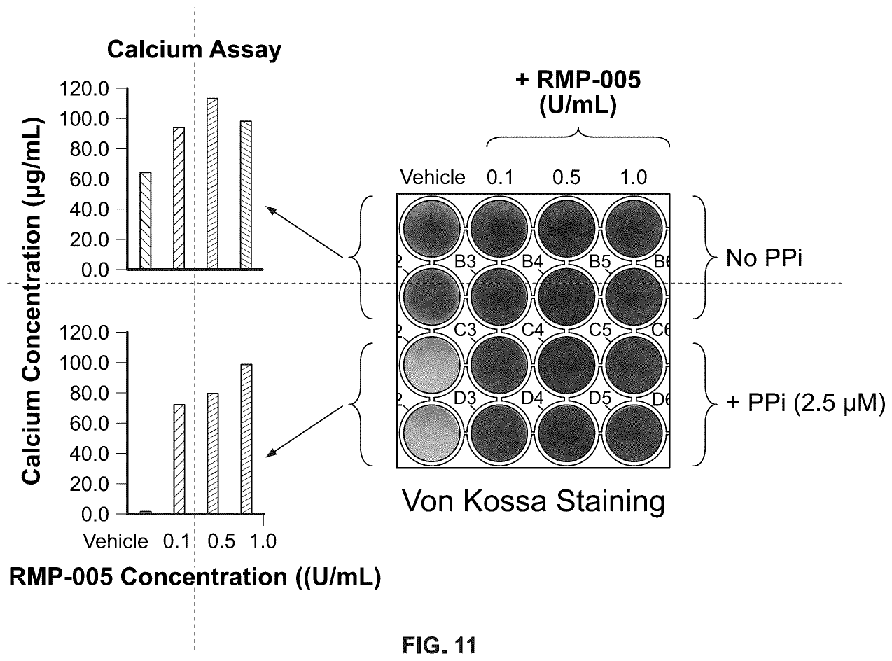

Insoluble calcium deposited into the extracellular matrix (ECM) was first dissolved in 0.5 M HCl for 1 h at 4° C. with gentle shaking. The calcium concentration in the supernatant was determined colorimetrically (wavelength 595 nm) using a calcium assay kit (Sekisui Diagnostics) at day 10 (see FIG. 11).

The polypeptide having SEQ ID NO: 5 rescued the PPi induced inhibition of cell mineralization even at the lowest concentration (0.1 U/ml). A dose range effect on deposited calcium concentration showing maximum effect at 1.0 U/mL.

Collagen matrix deposition was quantified by Picrosirius Red staining (Direct Red 80, Sigma), followed by dissolving the stain using 0.1 N NaOH (Fisher). Collagen type 1 from rat tail (Sigma) was used as a standard to generate standard curve. Spectrophotometric measurement was done using 562 nm wavelength.

### Example 2—In Vitro Hydroxyapatite Binding Assay for the Polypeptides Having SEQ ID NOS: 2 and 5

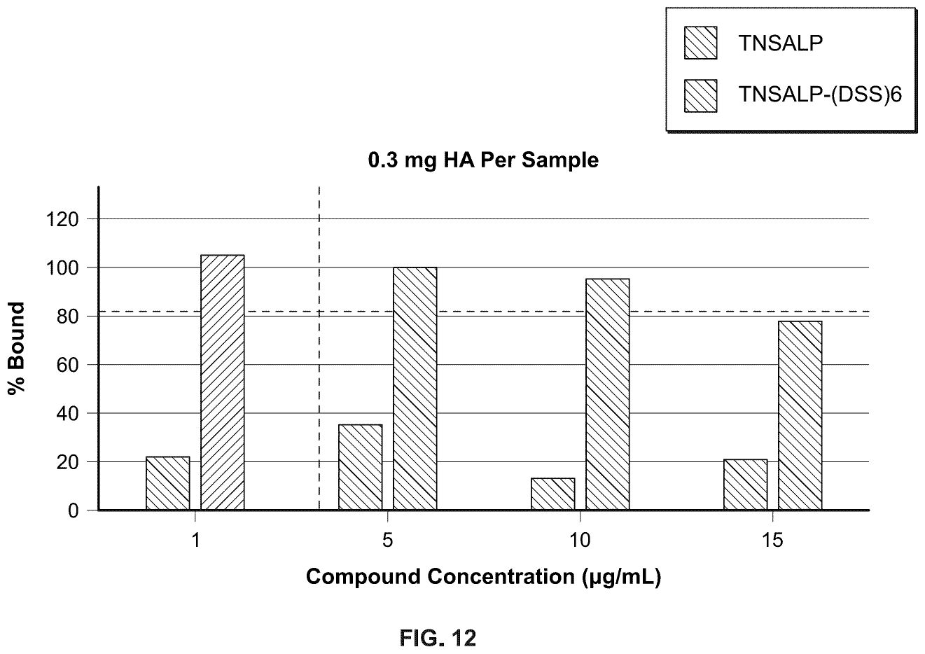

Untagged polypeptide having SEQ ID NO: 2 and tagged polypeptide having SEQ ID NO: 5 were subjected to pulldown assay (supernatant depletion by mineral) as follows: Hydroxyapatite crystal solution (Berkeley, 5 μM) (HA) was prepared in 20 mM Tris (pH 7.4); 150 mM NaCl and allowed to equilibrate overnight at room temperature with gentle agitation. The next day, untagged polypeptide having SEQ ID NO: 2 and tagged polypeptide having SEQ ID NO: 5 were incubated with, or without, 0.3 mg HA at different concentration 0.5, 1.0, 5.0, 10.0 and 15.0 μg/ml and allowed to bind for one hour at room temperature on a shaker. Tubes were spun down at 10,000×g for 5 min, and the supernatant was used to measure the alkaline phosphatase level for each sample. A standard colorimetric method where alkaline phosphatase is used as a standard (Sigma) and p-nitrophenylphosphate (Sigma) as a phosphatase substrate, turns yellow when dephosphorylated by alkaline phosphatase, and was measured at 405 nm wavelength. Results in FIG. 12 show strong preferential binding of the tagged polypeptide having SEQ ID NO: 5 over its non-tagged counterpart.

### Example 3—Secreted TNALP Activity for DNA Constructs Having SEQ ID Nos: 19 and 55)

Cell culture—293T cells were maintained and cultured in Dublbeco's minimum essential medium (DMEM) (Gibco) supplemented with 10% fetal bovine serum (FBS) (Gibco), 1% penicillin-streptomycin and 2 mM L-Glutamine (Gibco) at 37° C. in a humidified atmosphere of 5% CO₂. 4e6 of 293T cells were plated in 50 mm cell culture dish the day before transfection.

Transfection with calcium phosphate—Calcium phosphate kit (Clonetech) was used to introduce DNA constructs having SEQ ID Nos: 19, 25 and 55 into cultured 293 cells. Mixed 0.5 μg of plasmid DNAs from different constructs with 2M calcium solution; then vortexed it with the same volume of HEPES-Buffered Saline (HBS). Incubated 10 minutes before applying transfection solution to cell culture.

Secreted TNALP activity assay—1-10 uL of supernatant from transfected 293 cells were taken out and put into 96-well plate. A standard colorimetric method where alkaline phosphatase is used as a standard (Abcam) and p-nitrophenylphosphate (pNPP) (Abcam) as a phosphatase substrate, turns yellow when dephosphorylated by alkaline phosphatase, and was measured at 405 nm wavelength for 20 minutes at 37° C. in a microplate reader. TNALP activity (U/L) in the test samples was calculated based on the hydrolysis of one micromole of pNPP per minute at pH 9.6 and 25° C. (glycine buffer).

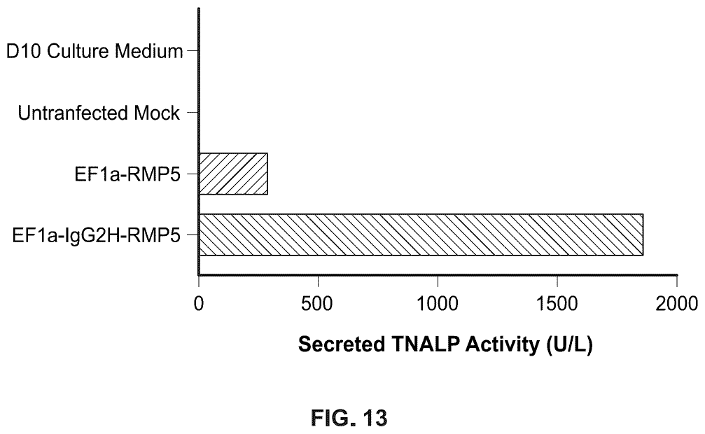

EF1a-promoter-driven TNALP DNA construct having SEQ ID No: 19 and EF1a promoter-driven TNALP DNA construct with IgG2H secretion signal peptide having SEQ ID No: 55 were transfected into 293t cells then the supernatant of cell culture was subjected to secreted TNALP assay. Results in FIG. 13 show the construct with secretion signal peptide of IgG2H has more than 6-fold of secreted TNALP activity compared with two constructs having the original secretion signal peptide with EF1a promoter and MND promoter.

### Example 4—Pharmacokinetics Study

Introduction

The objective of the study was to determine and compare the profile of 8 soluble recombinant forms (or constructs) of the tissue-nonspecific alkaline phosphatase (RMP-001 to RMP-008, corresponding to SEQ ID NOS: 1-8), following a single intravenous injection to the CD 1 mouse. The constructs are meant to be secreted by transduced cells after injection of recombinant lentiviral vectors into hypophosphatasia (HPP) patients as a potential novel gene therapy. To fulfil their therapeutic activity, the constructs have to be secreted from transduced cells and distribute into bones and various other organs at levels compatible with target tissue efficacy, while maintaining an adequate systemic safety profile. It has been hypothesized that systemic exposure and bone distribution should be similar to those found after repeated sub-cutaneous injections of Asfotase alfa, an enzyme replacement therapy currently approved for the treatment of perinatal/infantile and juvenile-onset HPP. Critical parameters to be established in this pharmacokinetic study are thus the systemic exposure (measured by the area under the curve) and half-life of each of the construct, both parameters to be compared to those Asfotase alfa. Any construct that would be cleared to rapidly would be disqualified from future development plan. This pharmacokinetic study would thus serve as a first screening step, which should be followed by a biodistribution study where the efficacy for each of the compound to home-in to bonne tissue would be assessed.

Study Plan

The test and control items were planned to be administered to groups of mice on a single occasion by slow bolus intravenous injection (over 30 to 60 seconds) as described in the table below.

TABLE 1
Study design.

| | | Dose | Dose | Dose | Number |
|---|---|---|---|---|---|
| Group | Group | Level | Concentration | Volume | of Male |
| Number | Designation | (mg/kg) | (mg/mL) | (ml/kg) | Animals |
| 1 | Control* | 0 | 0 | 2.5 | 3 |
| 2 | RMP-001 | 5 | 2 | 2.5 | 24 |
| | (SEQ ID | | | | |
| | NO: 1) | | | | |
| 3 | RMP-002 | 5 | 2 | 2.5 | 24 |
| | (SEQ ID | | | | |
| | NO: 2) | | | | |
| 4 | RMP-003 | 5 | 2 | 2.5 | 24 |
| | (SEQ ID | | | | |
| | NO: 3) | | | | |
| 5 | RMP-004 | 5 | 2 | 2.5 | 24 |
| | (SEQ ID | | | | |
| | NO: 4) | | | | |
| 6 | RMP-005 | 5 | 2 | 2.5 | 24 |
| | (SEQ ID | | | | |
| | NO: 5) | | | | |
| 7 | RMP-006 | 5 | 2 | 2.5 | 24 |
| | (SEQ ID | | | | |
| | NO: 6) | | | | |
| 8 | RMP-007 | 5 | 2 | 2.5 | 24 |
| | (SEQ ID | | | | |
| | NO: 7) | | | | |
| 9 | RMP-008 | 5 | 2 | 2.5 | 24 |
| | (SEQ ID | | | | |
| | NO: 8) | | | | |

*Control animals were administered with Phosphate Buffered Saline (PBS) alone.

A series of blood samples were collected at the following timepoints relative to dosing: 5, 15, and 30 minutes, and 2, 12, 24, 48 and 72 hours (post dose). Serum was collected and analyzed for ALP concentrations and the subsequent calculation of pharmacokinetic parameters. Following collection of the blood sample, each animal was euthanized and discarded without further examination.

Dose level was selected as it was close to the daily dose previously shown as well tolerated and fully efficacious for preventing bone defect in a hypophosphatasia mouse model (Millan et al. J. Bone Mineral Res. 2008, 23: 777-787.).

In a previous pharmacokinetic study, single intravenous administration of RMP-001 at a dose of 10 mg/kg in male CD-1 mice was well tolerated and did not result to signs of overt toxicity. In this study, the highest serum Alkaline Phosphatase (ALP) concentrations would be generally noted at 5 minutes post dosing.

Test Item Information
Characterization of Test and Control Items

| | | |
|---|---|---|
| Test item 1 | Identity: | RMP-001 in PBS 100 mM |
| | | L-arginine pH 7.2 |
| | Alternative Name: | Asfotase alfa |
| Test item 2 | Identity: | RMP-002 in 50 mM Tris pH 8, |
| | | 300 mM NaCl, 300 mM Imidazole |
| | Alternative Name: | sTNSALP |
| Test item 3 | Identity: | RMP-003 in 50 mM Tris pH 8, |
| | | 300 mM NaCl, 300 mM Imidazole |
| | Alternative Name: | sTNSALP-D10 |
| Test item 4 | Identity: | RMP-004 in 50 mM Tris pH 8, |
| | | 300 mM NaCl, 300 mM Imidazole |
| | Alternative Name: | sTNSALP-GSlinker-D10 |
| Test item 5 | Identity: | RMP-005 in 50 mM Tris pH 8, |
| | | 300 mM NaCl, 300 mM Imidazole |
| | Alternative Name: | sTNSALP-(DSS)6 |
| Test item 6 | Identity: | RMP-006 in 50 mM Tris pH 8, |
| | | 300 mM NaCl, 300 mM Imidazole |
| | Alternative Name: | sTNSALP-GSlinker-(DSS)6 |
| Test item 7 | Identity: | RMP-007 in 50 mM Tris pH 8, |
| | | 300 mM NaCl, 300 mM Imidazole |
| | Alternative Name: | STNSALP-VAR-D10 |
| Test item 8 | Identity: | RMP-008 in 50 mM Tris pH 8, |
| | | 300 mM NaCl, 300 mM Imidazole |
| | Alternative Name: | sTNSALP-VAR-(DSS)6 |
| Control item: | Identity: | Phosphate Buffered Saline (PBS) |

The test items forms were provided as pre-formulated solutions that were ready for use or for dilution and were received in 2 batches.

TABLE 2
Group dosing

| 1^{st} Batch | | | 2^{nd} Batch | | |
|---|---|---|---|---|---|
| Group | Test | Storage | Group | Test | Storage |
| dosed | Item | condition | dosed | Item | condition |
| 2 | RMP-001 | ≤−60° C. | 2 | RMP-001 | 2 to 8° C. |
| | | | 3 | RMP-002 | |
| | | | 4 | RMP-003 | |
| 5 | RMP-004 | | 5 | RMP-004 | |
| | | | 6 | RMP-005 | |
| | | | 7 | RMP-006 | |
| | | | 8 | RMP-007 | |
| | | | 9 | RMP-008 | |

On the day of dosing, the 1st batch of test items was removed from the freezer and was thawed at room temperature; the second batch of test items was removed from storage (2 to 8° C.) for dilution or use. The control item was removed from storage (2 to 8° C.) and was used for the dilution of the appropriate test items.

Following complete thawing or removal from storage:

Test Item 3 was used as it was received at a concentration of 2 mg/mL for dosing of animals in Group 4.

Test Items 1, 2, 4, 5, 6, 7, and 8 were diluted by adding the appropriate volume of the control item (PBS), to reach a final concentration of 2 mg/mL for each dose group.

Remaining test items not used were kept refrigerated at 2 to 8° C.

Dosing formulations intended for Groups 2 to 9 were administered within 3 to 4 hours following complete thawing and/or dilution.

Analysis of Test Item Formulations

Analysis of Achieved Concentration

In order to verify the concentration of the test item in the formulations (diluted to 2 mg/mL), representative samples (0.5 mL in duplicate were taken from the middle of each concentration (except Group 3 formulations, since there was no dilution) intended for dosing on Day 1.

Due to insufficient volumes, the second batch of undiluted test item received (RMP-001, RMP-002, RMP-003, RMP-004, RMP-005, RMP-006, RMP-007, RMP-008) (corresponding to SEQ ID NOS: 1-8, respectively) were not analyzed.

Samples were stored frozen (≤−60° C.) until transfer to ITR Immunology Department. Analysis of formulation samples were performed by ITR using an analytical UV A280 method.

As shown in the Table below, the concentrations of the original RMP-002, RMP-003, RMP-004, RMP-005, RMP-006, RMP-007 and RMP-008 measured by the Bradford method were on the average roughly 2 times higher than the concentrations measured by UV absorbance.

TABLE 3
Concentration values in formulation and stock solutions.

| | Nominal | Measured | Expected | | Corrected |
|---|---|---|---|---|---|
| | concentration | concentration | concentration | | concentration |
| Sample | (mg/mL) in | (mg/mL) in | (mg/mL) in | % | (mg/mL) in |
| ID | stock solution | formulation | formulation | Accuracy | stock solution |
| RMP-001 | 4.1 | 1.874 | 2 | 93.7 | 3.84 |
| RMP-002 | 2.5 | 1.017 | 2 | 50.9 | 1.27 |
| RMP-003 | 2 | * | * | * | * |
| RMP-004 | 2.5 | 1.037 | 2 | 51.9 | 1.30 |
| RMP-005 | 2.8 | 1.195 | 2 | 59.8 | 1.67 |
| RMP-006 | 3.5 | 1.068 | 2 | 53.4 | 1.87 |
| RMP-007 | 3.8 | 1.136 | 2 | 56.8 | 2.16 |
| RMP-008 | 2.8 | 0.886 | 2 | 44.3 | 1.24 |

Notes:
% Accuracy = (Conc. measured/Expected Conc) × 100.
* sample not collected for dose formulation analysis due to insufficient volume

The enzymatic activities of all the test articles were measured in a Hitachi Automatic Analyzer c311 using the same conditions as the ones used for clinical samples. Activities are measured in International Units (IU). Based on the concentrations obtained by measuring the UV absorbance, the specific activities in IU/mg were then computed. Results are presented in Table 4 below.

TABLE 4
Specific activities expressed in IU measured at ITR. RMP-001
(ref.) is the first batch received on Nov. 14, 2019 and kept
frozen until the analysis on Mar. 20, 2020. RMP-001* is the
batch received on Nov. 14, 2019 thawed, kept at 4-8° C.
until the analysis and used as a reference standard for all the
enzymatic assays during the PK study. RMP-001 is the batch
received on Mar. 2, 2020 and used for the formulation of the
test item used for dosing the animals during the PK study. No
value is available for RMP-003 as there was not enough
material left for the analysis. All the concentration values
used in computing the specific activities were obtained by
UV absorbance reading at 280 nm.

| | RMP- | RMP- | | | | | | | |
|---|---|---|---|---|---|---|---|---|---|
| | 001 | 001 | Dose | Dose | Dose | Dose | Dose | Dose | Dose |
| | received | received | formu- | formu- | formu- | formu- | formu- | formu- | formu- |
| | on | on | lation | lation | lation | lation | lation | lation | lation |
| | 14-11- | 20-02- | RMP- | RMP- | RMP- | RMP- | RMP- | RMP- | RMP- |
| | 19* | 20 | 001 | 002 | 004 | 005 | 006 | 007 | 008 |
| Sample | 12928.0 | 12419.0 | 5156.0 | 1018.0 | 4571.0 | 8562.0 | 8942.0 | 10174.0 | 5959.0 |
| activity | | | | | | | | | |
| measured | | | | | | | | | |
| (U/L) | | | | | | | | | |
| Dilution factor | 100.0 | 100.0 | 100.0 | 100.0 | 100.0 | 100.0 | 100.0 | 100.0 | 100.0 |
| in PBS 1X | | | | | | | | | |
| Activity × | 1292800.0 | 1241900.0 | 515600.0 | 101800.0 | 457100.0 | 856200.0 | 894200.0 | 1017400.0 | 595900.0 |
| Dilution (U/L) | | | | | | | | | |
| Activity | 1292.8 | 1241.9 | 515.6 | 101.8 | 457.1 | 856.2 | 894.2 | 1017.4 | 595.9 |
| (U/mL) | | | | | | | | | |
| Nominal | 4.1 | 4.1 | 2.0 | 2.0 | 2.0 | 2.0 | 2.0 | 2.0 | 2.0 |
| sample conc. | | | | | | | | | |
| (mg/mL) | | | | | | | | | |
| Measured | 4.1 | 4.1 | 1.9 | 1.0 | 1.0 | 1.2 | 1.1 | 1.1 | 0.9 |
| conc (UV | | | | | | | | | |
| A280). | | | | | | | | | |
| Sample spec. | 315.3 | 302.9 | 275.1 | 100.1 | 440.8 | 716.5 | 837.3 | 895.6 | 672.6 |
| activity | | | | | | | | | |
| (U/mg) | | | | | | | | | |
| Sp act. ratio | 1.000 | 1.041 | 1.146 | 3.150 | 0.715 | 0.440 | 0.377 | 0.352 | 0.469 |
| (RMP1*/ | | | | | | | | | |
| RMPX) | | | | | | | | | |

Analysis of Homogeneity

The two RMP-001 samples received had been purified from crude CHO supernatant cultures by protein-A capture followed by preparative Size Exclusion Chromatography (SEC) and analyzed by SEC-HPLC in native conditions. They were shown to be more than 98% pure with no contamination from aggregates. RMP-002 to RMP-008 test items contained a His-tag extension at the C-terminus that allowed purification on a Ni-Sepharose column by the UCLA core lab. They were analyzed by SDS-PAGE conditions in reducing and non-reducing conditions but were not submitted to SEC-HPLC analysis. As conditions for SDS-PAGE might have resulted in solubilizing any aggregates present in the crude spent culture supernatant, it is currently unknown whether test items received from the UCLA core lab might be contaminated by aggregates. It is indeed known that recombinant soluble tissue-nonspecific ALP has a tendency to form aggregates.

Analysis of Stability

The stability of formulations under the conditions of use and/or storage on the study including sufficient room temperature stability to cover the period of dosing and the concentrations used on the study were estimated from previous experience with this kind of enzyme. A more rigorous approach to stability testing should have been performed by periodically measuring the specific activity of all test items and the presence of aggregates or fragments in native conditions using techniques such as SEC-HPLC or nephelometry.

Administration of the Test and Control items

The test and control items were administered once by slow bolus intravenous injection (over 30 to 60 seconds) into the tail vein with a hypodermic needle attached to a syringe. The dose volume for each dose group was 2.5 mL/kg, including controls. The actual volume administered to each mouse was calculated and adjusted based on the most recent practical body weight of each animal.

The dosing formulations for Groups 2 to 9 were administered to all respective animals within 3 to 4 hours following complete thawing and/or dilution.

Remaining test and control item formulations following each dosing were discarded.

In-Life Observations

Clinical Observations

For all animals, cage-side clinical signs (ill health, behavioral changes, etc.) were recorded at least once daily during all phases of the study, except on detailed clinical examination (DCE) days, where the cage-side clinical signs were replaced by a DCE.

A DCE of each mouse was performed once at arrival as part of the health status evaluation and on Day −1.

Body Weights

Body weights were recorded for all animals as per health status section and then at least once prior to group assignment. Body weights were also recorded for all animals on Day −1.

Pharmacokinetics

Sample Collection, Processing and Bioanalysis

A series of blood samples (approximately 0.5 mL) were collected from 3 mice/timepoint (except for Group 1, where blood samples were collected at one time point) on Day 1 post dose as indicated in Table 5 below.

TABLE 5
Design of the pharmacokinetic post dose sample collection

| | Number | Pharmacokinetic time point Post Dose | | | | | | | |
|---|---|---|---|---|---|---|---|---|---|
| Group | of | 5 | 15 | 30 | 2 | 12 | 24 | 48 | 72 |
| number | animals | min | min | min | hr | hr | hr | hr | hr |
| 1 | 3 | | | √ | | | | | |
| 2 to 9 | 3* | √ | | | | | | | |
| | 3* | | √ | | | | | | |
| | 3* | | | √ | | | | | |
| | 3* | | | | √ | | | | |
| | 3* | | | | | √ | | | |
| | 3* | | | | | | √ | | |
| | 3* | | | | | | | √ | |
| | 3* | | | | | | | | √ |

For this purpose, each animal was anesthetized with Isoflurane to allow blood sample collection and were bled via cardiac puncture.

Following its last blood sampling, each animal was euthanized by exsanguination and discarded without further examination.

Following collection, blood samples for serum were allowed to stand at room temperature for approximately 30 minutes to clot and then the samples were centrifuged (2500 rpm for 10 minutes at approximately 4° C.) and the resulting serum was recovered and stored frozen (≤−60° C.) in appropriately labeled tubes. The serum samples were analyzed at ITR using an Alkaline Phosphatase (ALP) Enzymatic Assay qualified under ITR Study No. 52086.

There were no pharmacokinetic time point deviations noted in this study. The location of blood withdrawal was noted in the raw data.

The stability of the test item in the biological matrix at concentrations suitable for the calibration range should be confirmed in the Bioanalytical Report. This should include the duration from sample collection until completion of sample analysis and at the storage conditions used on the study.

Non-Compartmental Analysis

The pharmacokinetic parameters were calculated at ITR.

Non-compartmental analysis of ALP concentrations in serum data set obtained on Day 1 was performed by using the Phoenix® WinNonlin® software.

The following configuration was used for the analysis by default.

Sampling Method: Sparse

AUC Calculation Method: Linear Trapezoidal with Linear Interpolation.

Lambda Z (λz) Method: Best fit for λz, Log regression

Weighting (λz calculation): Uniform

Pharmacokinetic parameters (including abbreviation and description for each parameter) are described in the following table:

| Parameters | Abbreviation |
|---|---|
| Area under the serum drug concentration-time curve | AUC_{0-Tlast} |
| from the time of dosing to the last quantifiable | |
| concentration | |
| Area under the serum drug concentration-time curve | AUC_{INF} |
| from the time of dosing extrapolated to infinity | |
| Terminal elimination half-life | t_{1/2} |
| The maximum serum concentration | Cₘₐₓ |
| Time to maximum serum concentration | Tₘₐₓ |
| Total body clearance per kg body weight | Cl |
| Volume of distribution per kg body weight | V_{z} |

Results

Dose Formulation Analysis

No ALP was detected in any of the samples collected from the control (Group 1) formulations.

No dose formulation sample was collected from Group 4-RMP-003 due to insufficient volume.

The analysis of the dose formulation samples collected from Group 2-RMP-001 (2 mg/mL) determined that the ALP concentration was 93.7% of the nominal concentration and were therefore considered to be acceptable for use on the study (within the accepted criteria of 90 to 110% of the nominal concentration).

The analysis of the dose formulation samples collected from Groups 3 to 7—RMP-002 RMP-004, RMP-005, RMP-006, RMP-007, and RMP-008—(all at 2 mg/mL), determined that the ALP concentrations ranged from 44.3 to 59.8% of the nominal concentrations and were unexpectedly below the accepted criteria of 90 to 110% of the nominal concentration).

Review of related raw data showed that these unexpected results and serum ALP concentrations are due to discrepancies between the initial estimation of the concentration of RMP-002 to RMP-008 at the UCLA core lab and the concentration of the test articles measured by UV absorbance reading at ITR. Animals have thus been administered with various doses of the test items as summarized in Table 6 below. Actual dose for RMP-003 could not be established as there was not enough material left for UV absorbance measurement.

TABLE 6
Estimation of the actual dose received by each
group of mice at the initiation of the study.

| | Nominal Dose | Coeff | Actual dose |
|---|---|---|---|
| Analyte | mg/kg | correct. | (mg/kg) |
| RMP-001 | 5 | 0.937 | 4.69 |
| RMP-002 | 5 | 0.509 | 2.54 |
| RMP-003 | 5 | | |
| RMP-004 | 5 | 0.519 | 2.59 |
| RMP-005 | 5 | 0.598 | 2.99 |
| RMP-006 | 5 | 0.534 | 2.67 |
| RMP-007 | 5 | 0.568 | 2.84 |
| RMP-008 | 5 | 0.443 | 2.22 |

Mortality

There was no mortality during the course this study.

Clinical Signs

There were no clinical signs that could be attributed to the intravenous administration of the 8 forms of the test items at a nominal dose level of 5 mg/kg.

Body Weight

Body weights were recorded during the pre-treatment period for general health status evaluation and on Day −1 for the purpose of dose volumes calculation. Body weights recorded during the pre-treatment period were within the normal biological range and suggested that the animals were suitable for use throughout the study.

PK Curves

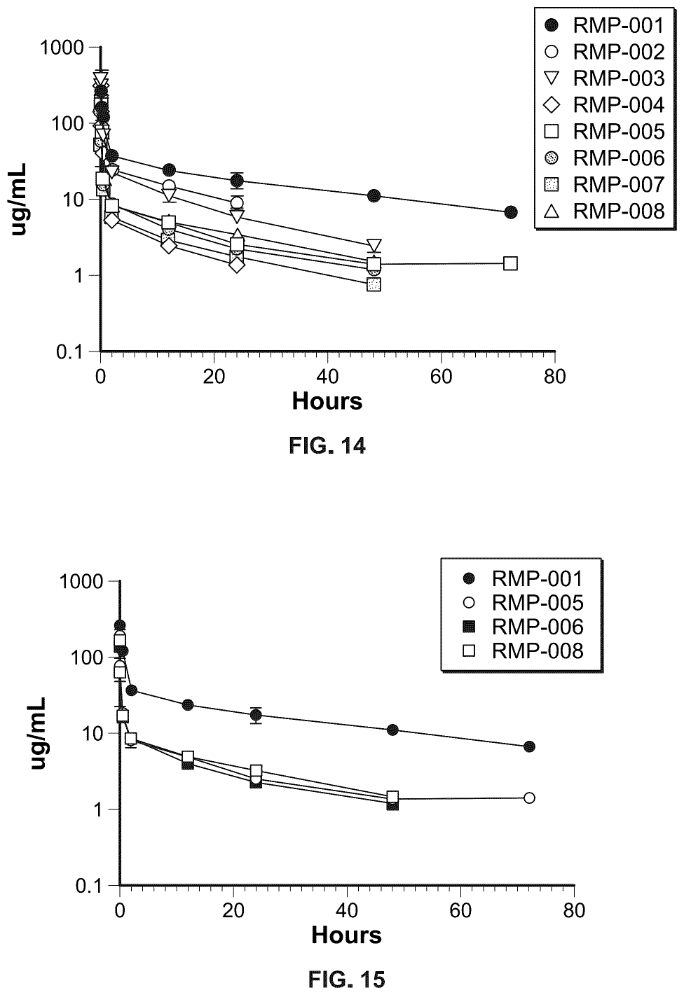

The curves related to variation of the concentrations of all the 8 test items over the 72-h period post dosing are shown in FIG. 14.

The values for each time-point and each test item represent the actual concentrations corrected for the specific activity of each test item. They were computed as follow. Raw data (expressed as Vmax) generated by the determination of the activity in each blood samples were first transformed in concentrations (μg/mL) using the calibration curve (Vmax vs concentration) obtained for the reference standard RMP-001* for which the specific activity is known (315.3 IU/mg). Then each concentration value was corrected for specific activity using the ratio RMP-001*/RMP-00X for each test item (see Table 4). Results show that most of the PK curves (RMP-004 to RMP-008) are very close to each other and located significantly below the curve for RMP-001. This was expected as RMP-001 is an Fc-fusion construct, which are characterized by a higher systemic exposure due to a lower clearance rate. Of note, RMP-002 and RMP-003 are clearly outliers in the series of data. RMP-002 is a compound that was shared by two different laboratories and probably submitted to suboptimal storage and shipping conditions, which might explain its low specific activity. Quantities of RMP-003 were low and insufficient for allowing the determination of its true concentration by measuring the UV A280. Thus no correction was done for this compound. If the concentration of RMP-003 was also overestimated by a factor of 2, as it was for all the other test items, the PK curve retatedhttps://www.nytimes.com/2020/08/20/health/covid-oleandrin-trump-mypillow.html to RMP-003 would also be similar to the main group of compounds without an Fc domain.

As seen in FIG. 15, all the PK curves related to (DSS)6-tagged compounds (RMP-005, RMP-006 and RMP-008) are very similar. Moreover the introduction of a (GGGGS)2 linker to RMP-005 (as done in RMP-006) has little impact on the PK profile.

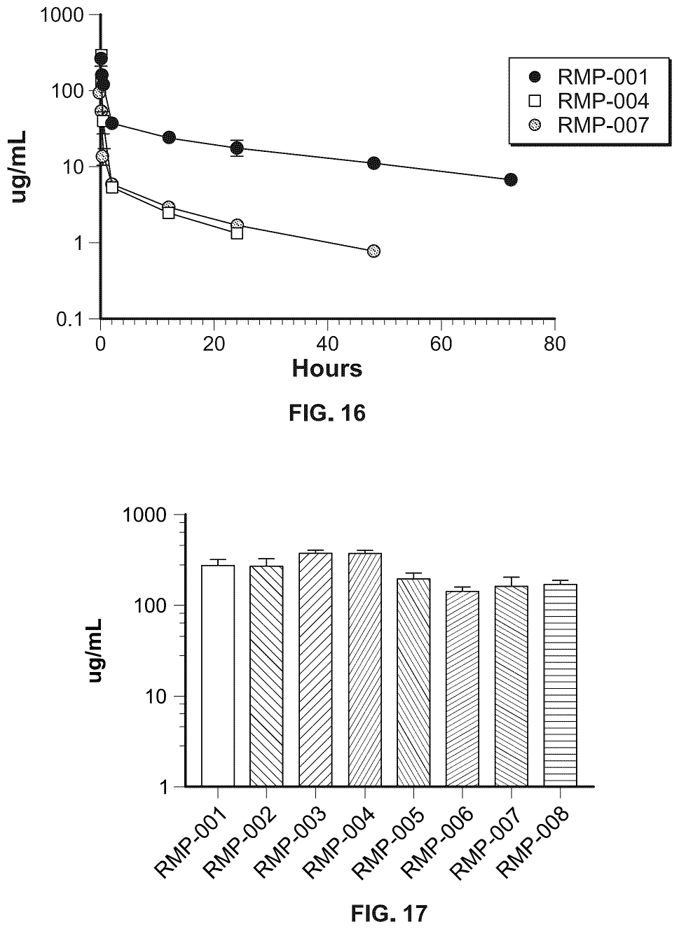

The same conclusion can be made for RMP-004 and RMP-007, which both carry a D10 tag (see FIG. 16).

Cmax/Tmax

Cmax values can also be extracted easily from the above PK data and Tmax coincides with the earliest blood sample time (5 min post dosing). There is considerable variations in those values between the same group as withdrawal has to be made 5 min only after dosing when injected compounds are rapidly removed from the central compartment (blood) through their distribution into other body compartments. For a systemically injected drug, those values are not very meaningful. Nonetheless they are shown in FIG. 17.

Area Under the Curve (Extrapolated to Infinity)

The area under the plasma drug concentration-time curve (AUC) reflects the actual body exposure to drug after administration of a dose of the drug and is expressed in mg*h/L. This area under the curve is dependent on the rate of elimination of the drug from the body and the dose administered. The total amount of drug eliminated by the body is assessed by adding up or integrating the amounts eliminated in each time interval, from time zero (time of the administration of the drug) to infinite time. This total amount corresponds to the fraction of the dose administered that reaches the systemic circulation. The AUC is inversely proportional to the clearance of the drug.

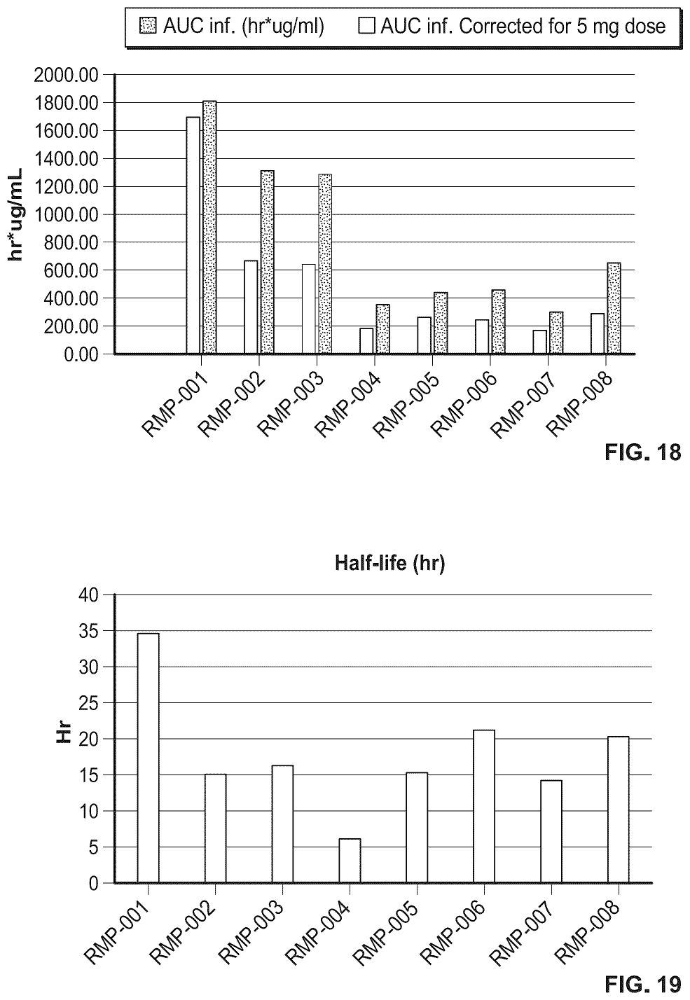

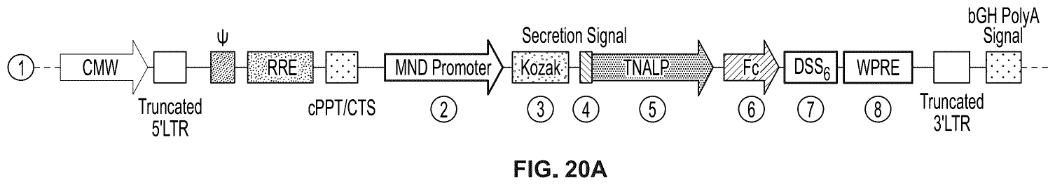

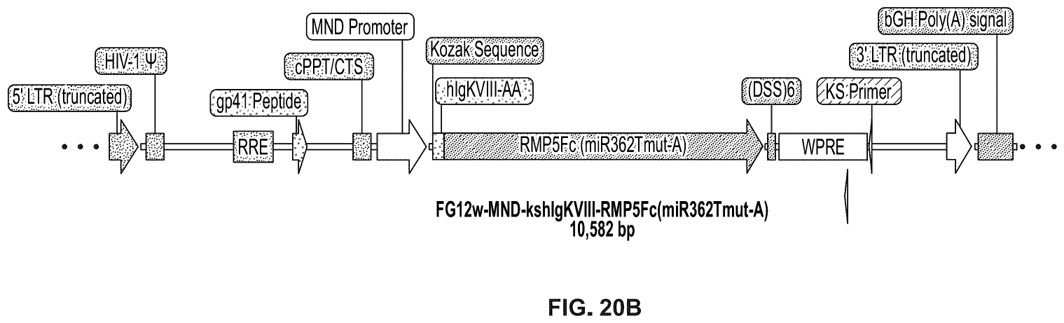

The original AUCinf values computed by ITR were corrected to take into account the specific activity of each test items and are presented in FIG. 18 (blue bars). Moreover another corrective step was introduced to normalize all AUC data for a 5 mg/Kg dosage (green bars). This correction was needed as the AUC is generally proportional to the dose. It should minimize any discrepancies due to the original erroneous estimation of the doses administered to each group. There is a single value for each test item AUC.

No SEs or confidence intervals could be calculated because a sparse sampling method was used for data collection.

Data related to groups that had received RMP-002 and RMP-003 have to be interpreted with cautions as these compounds behaved as outliers in the overall PK study for the reasons explained before. Interestingly, test items RMP-004 to RMP-008 have all AUC values that are roughly 20% to 40% that of RMP-001.

Test Item Clearance

After Tmax, test item serum concentrations declined at an estimated t½ ranging from 6.11 hours (RMP-004) to 34.6 hours (RMP-001), see data in FIG. 19. Most of the test items (RMP-002 to RMP-008) are in the 15 to 20 hour range. The reason why RMP-004 has such a low half-life value is not clear.

The test item was cleared (Cl) at a mean rate of 3.37 mL/hr/kg (RMP-001) up to 23.6 mL/hr/kg (RMP-002). The mean volume of distribution (Vz) ranged from 168 mL/kg (RMP-001) to 515 mL/kg (RMP-002), suggesting that all test items were minimally distributed among tissues. There was no correction needed for all the above clearance parameters as their assessment does not rely on concentration values.

### CONCLUSION

The aim of the study was to compare the PK parameters of various forms of bone-targeted TNSALP to those of Asfotase alfa (RMP-001). There is a good convergence of PK data showing that non-Fc versions of the test items are cleared from the bloodstream faster than Asfotase alfa, as expected. However half-lives and systemic exposure remain in a range compatible with an efficacious bone-targeted drug delivery strategy. In this strategy smaller molecules should have an increased access to the mineralized portion of the bone compared to larger Fc-fusions as they are expected to extravasate more easily from the capillary network into the mineralized surface of bone. This however remains to be verified experimentally in biodistribution studies.

### Example 5—Lead Candidate Selection

The selection of the lead candidate “FG12w.MND.kz.IgKVIII.TNALPco(mut-miR362a).Fc.(DSS)6.WPRE” (SEQ ID NO:74) is described herein.

Screening of Secretion Signal Peptides

Initially, various vectors were screened based on secreted TNALP activity. The vectors screen included: EF1a-RMP5, EF1a-IgG2H-RMP5 (SEQ ID NO: 55), EF1a-SEAP-RMP5 (SEQ ID NO: 65), EF1a-aLA-RMP5 (SEQ ID NO: 64), EF1a-hCD33-RMP5 (SEQ ID NO: 58), EF1a-Secrecon-RMP5 (SEQ ID NO: 60), EF1a-Secrecon-AA-RMP5 (SEQ ID NO: 61), EF1a-mIgKVIII-AA-RMP5 (SEQ ID NO: 62), and EF1a-hIgKVIII-AA-RMP5 (SEQ ID NO: 57).

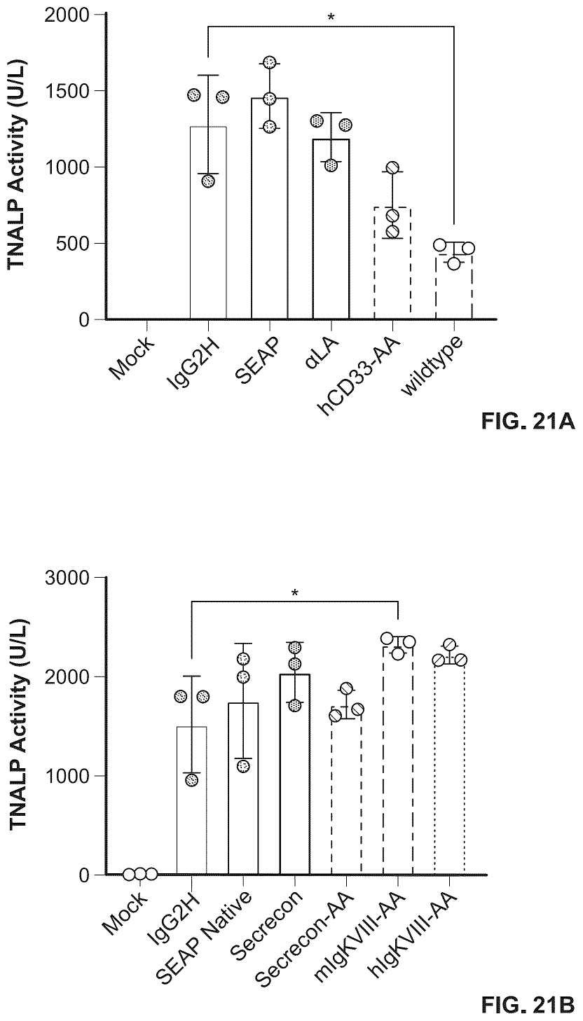

1e5 293T cells per well were transfected with constructs (SEQ ID NOS: 19, 55, 65, 64 and 66) using calcium phosphate method at d0. ALP activity was measured at d2. Results showed IgG2H (MGWSCIILFLVATATGVHS) (SEQ ID NO: 33) stood out by mediating >2X secreted ALP activity compared to endogenous TNALP signal peptide in 293 transfection. In a second-round screening, 1e5 293T cells per well were transfected with constructs (SEQ ID NOS: 19, 55, 65, 60, 61, 62 and 57) using calcium phosphate method at d0. ALP activity was measured at day 2. hIgKVIII (MDMRVPAQLLGLLLLWLRGARCAA) (SWEQ ID NO: 35) was chosen as lead secretion signal candidate due to an about 46% increase of ALP secretion over IgG2H and its comparatively low immunogenicity risk compared with mIgKVIII. Results demonstrated engineering signal peptide could greatly improve the secretion of TNALP of the disclosed lentivector constructs. The results are set forth in FIGS. 21A and 21B.

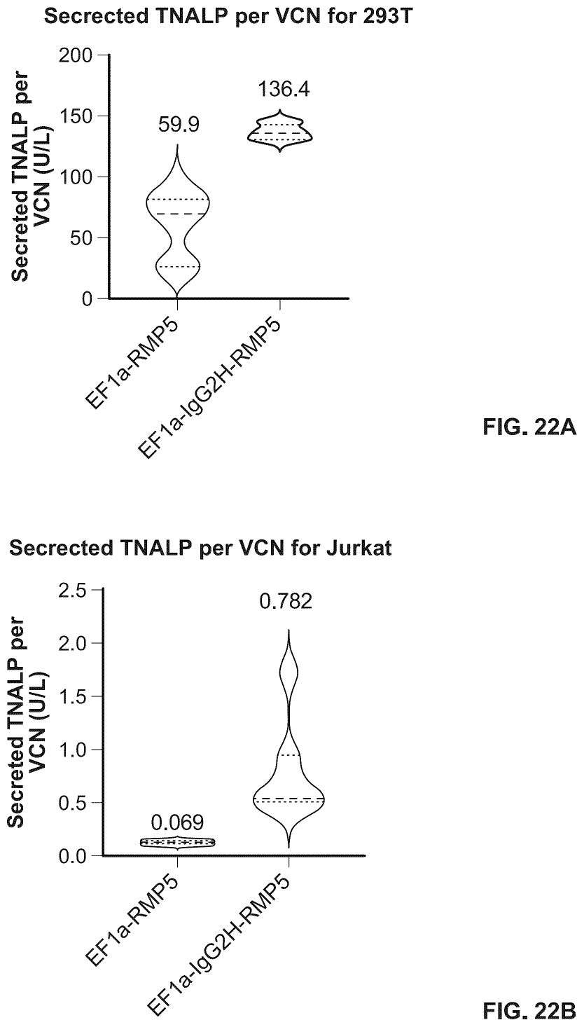

Subsequently, EF1a-promoter-driven TNALP DNA constructs having SEQ ID NO: 19 and EF1a promoter-driven TNALP DNA construct with IgG2H secretion signal peptide having SEQ ID No: 55 were used to generate VSVG-pseudotyped lentivectors by co-transfecting 293T human embryonic kidney cells with other 3 plasmids. Lentivirus supernatants were harvested to transduce 293 and Jurkat cells at dilution factors 4, 16 and 64, respectively. The supernatant was harvested for ALP activity assay and cell pellets were harvested for VCN analysis on ddPCR at day 14. Results showed the IgG2H signal peptide mediated a modest 2-fold increase in ALP secretion from 59.9 U/L to 136.4 U/L in 293T cells (FIG. 22A), but a significantly increased secretion over 10-fold in Jurkat cells from 0.069 U/L to 0.782 U/L (FIG. 22B).

These results demonstrated that replacement of wildtype TNALP signal peptide with engineering signal peptides IgG2H could increase the secretion of TNALP in not just human embryonic kidney cell but also in a hematopoietic T cell line which is a representative of an HSC-derived lineage.

Removal of Susceptible Myeloid-Lineage miRNA Binding Sites

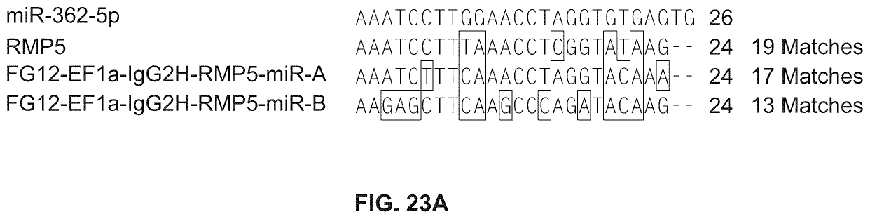

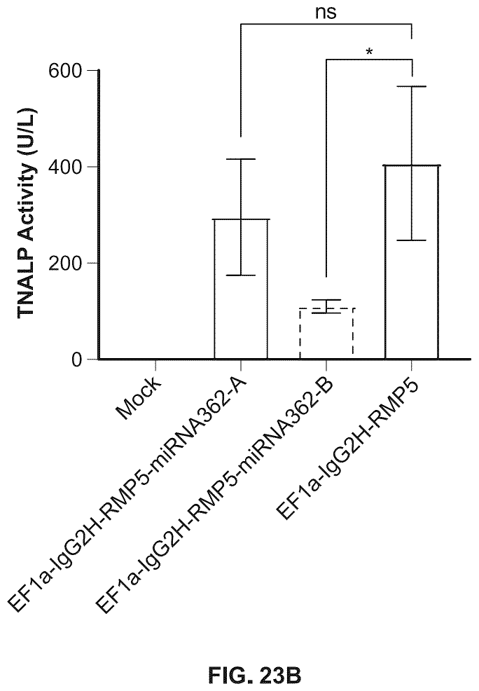

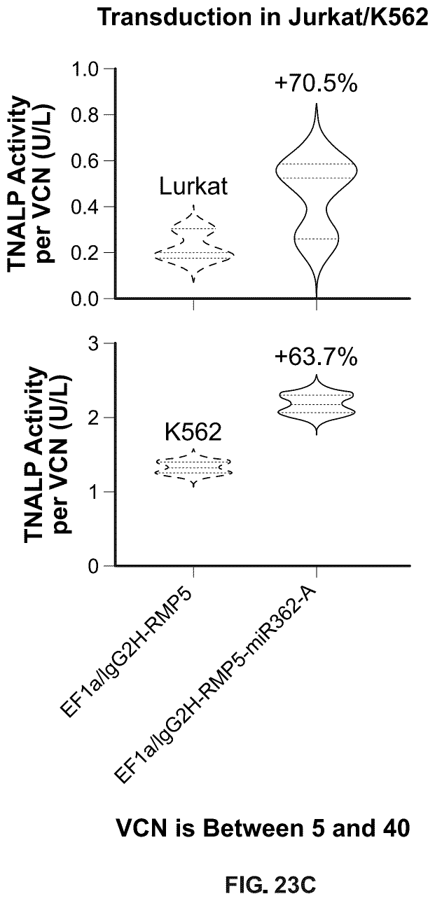

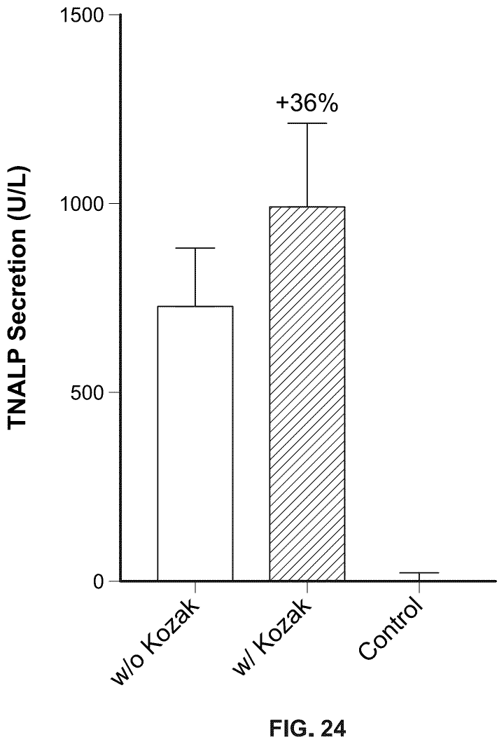

Next, susceptible myeloid-lineage miRNA binding sites from RMP5 were removed. The sequence of RMP5 (described herein) between 762-792 bp was believed to be susceptible to being targeted by miRNA362-5p, while Yang et al indicated MiR-362-5p is highly expressed in hemopoietic lymphoid and myeloid lineages (Yang, P., Ni, F., Deng, Rq. et al. MiR-362-5p promotes the malignancy of chronic myelocytic leukemia via down-regulation of GADD45α. Mol Cancer 14, 190 (2015)). To eliminate potential miR-362-5p silencing in hemopoietic lymphoid and myeloid lineages, two mutations were created (miRNA362-A and miRNA362-B) through switching codon usage in RMP5 between 769 and 792 bps. Calcium phosphate was used to transfect 293T cells with 3 plasmids (SEQ ID NOS: 19, 81 and 82, respectively). Supernatant was harvested for ALP activity assay at day 3 after transfection (see FIG. 23A). Mutant A shows comparable TNALP secretion as parental construct in 293 cell transfection while miR362-B was discarded due to dramatically reduced TNALP secretion (FIG. 23B). The construct (SEQ ID NO:81) was further used to generate lentivector.

Lentivirus supernatants were harvested to transduce Jurkat and K562 cells at dilution factors 4, 16 and 64, respectively. Supernatant was harvested for ALP activity assay and cell pellets were harvested for VCN analysis on ddPCR at day 14. The construct (SEQ ID NO:81) outperformed the original construct (SEQ ID NO:19) in two myeloid cell lines with high expression of miR-362-5p by more than about 60%. These results reflect efforts to improve the potency of the disclosed constructs through the removal of one or more susceptible myeloid-lineage-miRNA binding sites.

Addition of Translation Initiation Sequences to Promote Efficient Translation of RMP5 mRNA

Next, 1e5 293T cells per well were transfected with the constructs (SEQ ID NO:84 and 85) using calcium phosphate method at d0 and ALP activity is measured at d2. Results showed addition of Kozak sequence increased TNALP secretion by about 36%. This suggested that the addition of translation initiation sequences could promote efficient translation of RMP5 mRNA

Lead Candidate Selection and Qualification

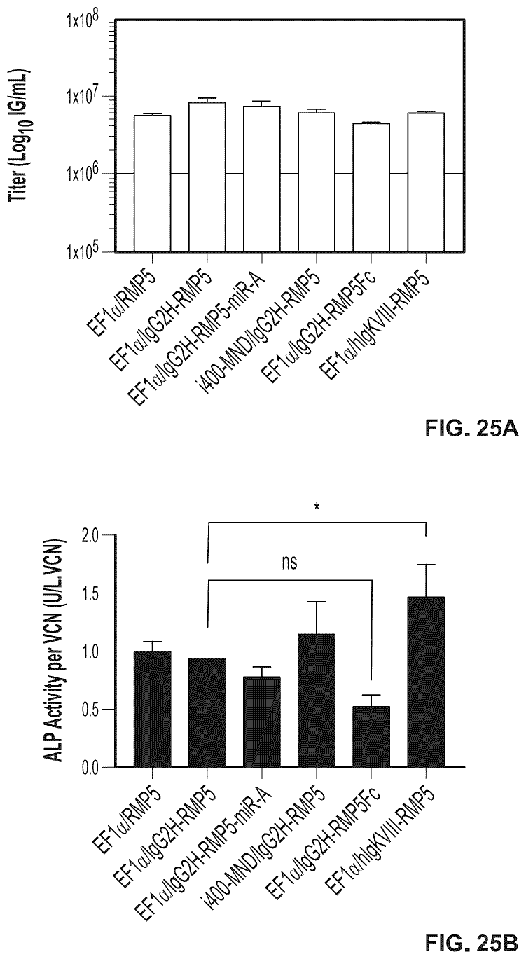

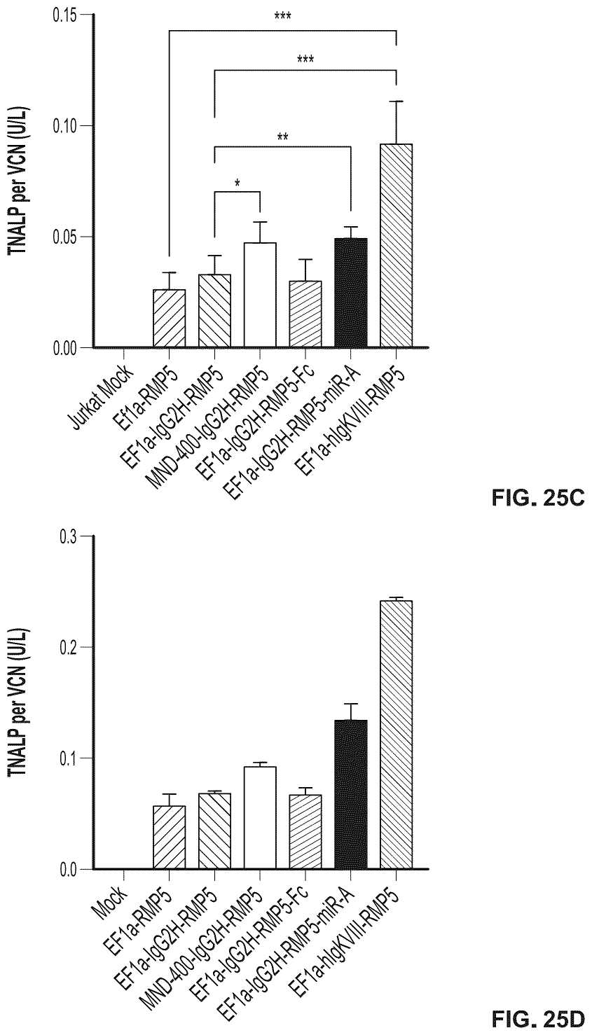

Lead candidates were then selected based on the optimized vector designs. Six TNALP LVV constructs with different promoters MND/EF1a and signal peptides wildtype, Gigha and IgKVIII (SEQ ID No: 19, 55, 57, 79, 86 and 87) were used to generate VSVG-pseudotyped lentivectors by co-transfecting 293T human embryonic kidney cells with other 3 plasmids. Lentivirus supernatants were harvested to transduce 293, K562 and Jurkat cells at dilution factors 4, 16 and 64, respectively. Supernatant was then harvested for ALP activity assay and cell pellets were harvested for VCN analysis on ddPCR at day 14. Those lentivectors had similar titer between 5e+6 and 1e+7 IU/mL (FIG. 25A). Data (FIGS. 25B-25D) indicates that IgG2H was better than TNALP wildtype secretion signal in transduced Jurkat and K562; and that MND promoter with 400-bps insulator outperformed the EF1a promoter in both hematopoietic cell lines. The addition of an Fc fusion partner was believed to have minimal impact on TNALP secretion in three cell lines. It was also found that the miR362-A mutation performed better than the original construct in both myeloid cell lines; and that the TNALP secretion from IgKVIII was superior to IgG2H in all three cell lines. From this experiment, a combinatory construct of MND promoter, IgKVIII, miR362-mutation, and Fc was suggested to be the lead construct.

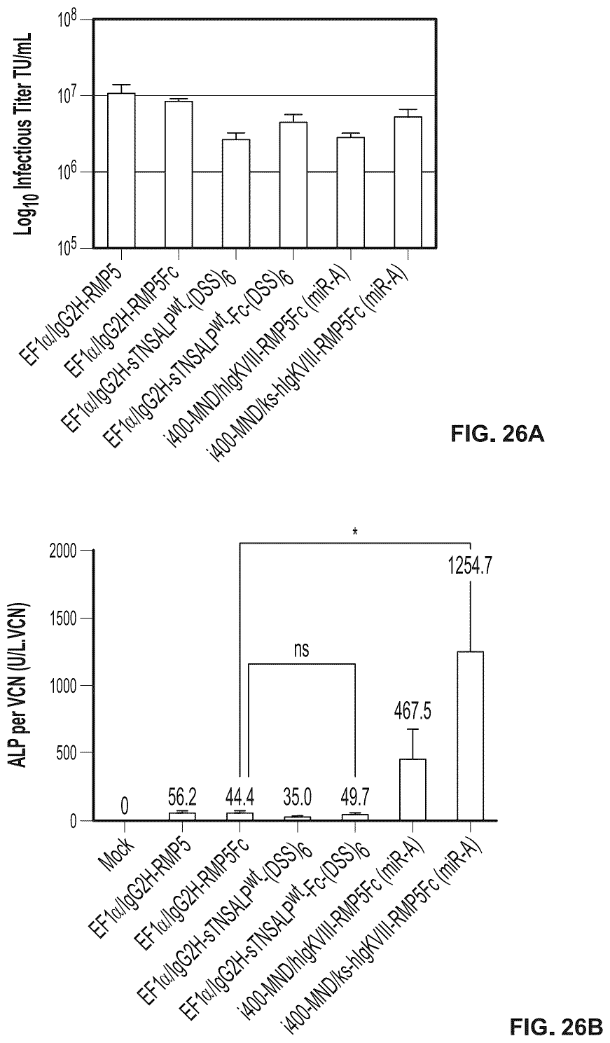

Six TNALP LVV constructs with two different promoters (MND or EF1a), two different signal peptides (IgGH or IgKVIII), non-Fc-fused or Fc-fused RMP5 constructs (SEQ ID No: 55, 84, 85, 87, 88 and 89) were used to generate VSVG-pseudotyped lentivectors by co-transfecting 293T human embryonic kidney cells with other 3 plasmids. Lentivirus supernatants were harvested to transduce 293 cells at dilution factors of 4, 16 and 64, respectively. Supernatant was then harvested for ALP activity assay and cell pellets were harvested for VCN analysis on ddPCR at day 14. Those lentivectors had similar titer between 5e+6 and 1e+7 IU/mL (FIG. 26A). FIG. 26B indicated that the potency (ALP per VCN) of Fc-fused construct—EF1-IgG2H-RMP5-Fc (SEQ ID: 87) is only about 20% lower than that of non-Fc-fused construct—EF1a-IgG2H-RMP5 (SEQ ID: 55). Considering that no toxicity was observed in the mouse model for the Fc-fused version and further considering that the circulation time of the Fc-fused version is more than double of that of non-Fc-fused version (6.1 vs 15-20 hrs.), it was believed worthy to include Fc into the lead candidate. Data suggested that the potency of the codon-optimized non-Fc-fused/Fc-fused constructs (SEQ ID NOS: 55 and 87)) was similar, if not better than that of cDNA non-Fc-fused/Fc-fused constructs (SEQ ID NOS: 88 and 89). Based on data, it was believed that the addition of Kozak increased the potency about 2-fold. Overall, the ultimate combinatory construct of 400 bps insulator, MND promoter, Kozak element, IgKVIII, miR362-mutation and Fc (1400-MND-kozak-IgKVIII-RMP5(miR-A)-Fc-DSS6 (SEQ ID: 85) demonstrated about 30 fold higher potency as compared with the candidate construct EF1-IgG2H-RMP5-Fc (SEQ ID: 87) in 293T cells. This suggested that it is necessary to combine multiple engineering approaches to lead to achieve superior potency.

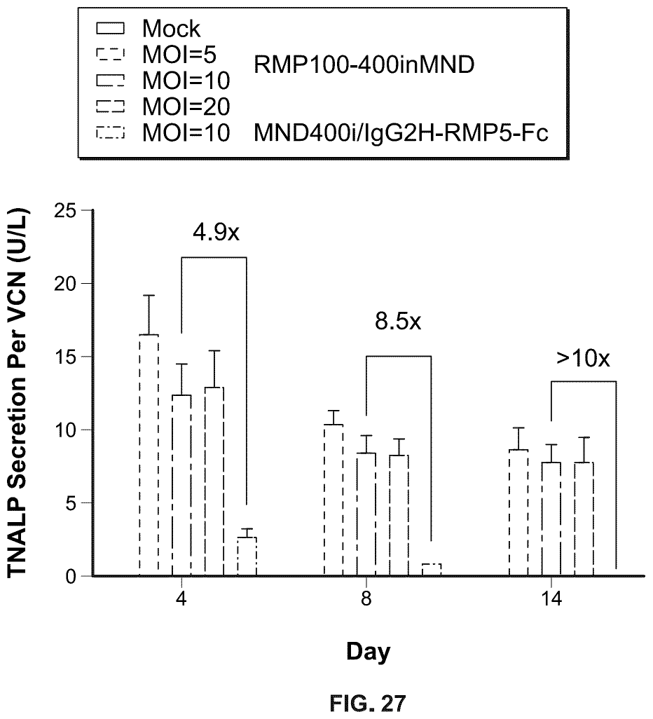

Two TNALP LVV constructs (I400-MND-kozak-IgKVIII-RMP5(miR-A)-Fc-DSS6 SEQ ID: 85 and EF1a-IgG2H-RMP5-Fc SEQ ID: 87) used to generate VSVG-pseudotyped lentivectors by co-transfecting 293T human embryonic kidney cells with other 3 plasmids. Lentivirus supernatants were harvested and concentrated through TFF. Human adult mobilized peripheral blood CD34+ hematopoietic stem cells were thawed and pre-stimulated with SCF, 113, TPO and Flt3 overnight. Transduction was then carried out by adding vectors into the cells in the presence of the transduction enhancers dmPGE2 and F108. The media of the transduced cells was refreshed to SFEMII supplemented with IL3 and G-CSF for 7 days and changed to G-CSF-contained SFEM II from day 8 to day 14. The supernatant was subsequently taken out for ALP analysis at day 4, 8 and 14. VCN analysis was carried out on cell pellets on day 14. Results show that the potency of 400inMND-RMP100 is 4.9 fold greater day 4, 8.5 fold greater at day 8 and more than 10 fold greater at day 14 as compared with I400-MND-IgG2H-RMP5-Fc (see FIG. 27). The results confirmed the finding in 293T and the combinatory construct of 400 bps insulator, MND promoter, Kozak element, IgKVIII, miR362-mutation and Fc (I400-MND-kozak-IgKVIII-RMP5(miR-A)-Fc-DSS6 SEQ ID: 85) had significantly increased ALP secretion not just in 293t but more importantly in our target cells—adult mobilized peripheral blood CD34+ hematopoietic stem cells.

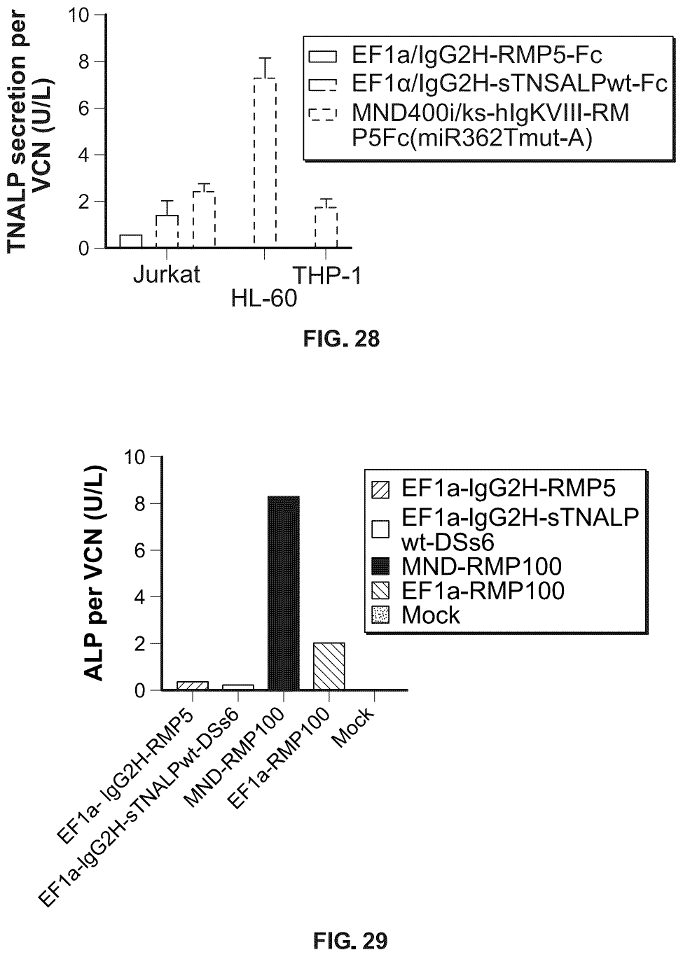

Five TNALP LVV constructs with two different promoters (MND or EF1a), two different signal peptides (IgGH or IgKVIII) (SEQ ID NOS: 55, 88, 85, 74 and 89) were used to generate VSVG-pseudotyped lentivectors by co-transfecting 293T human embryonic kidney cells with other 3 plasmids. Virus supernatants were used to transduce Jurkat, HL-60 and THP-1 cells at dilution factors of about 4, about 16, and about 64. Supernatant was harvested for ALP activity assay and cell pellets were harvested for VCN analysis on ddPCR at day 14. Results (FIGS. 28 and 29) illustrated that the MND promoter provided for increased expression of TNFAMP activity (per VCN) as compared with the EF1a promoter in Jurkat and HL-60 cells.

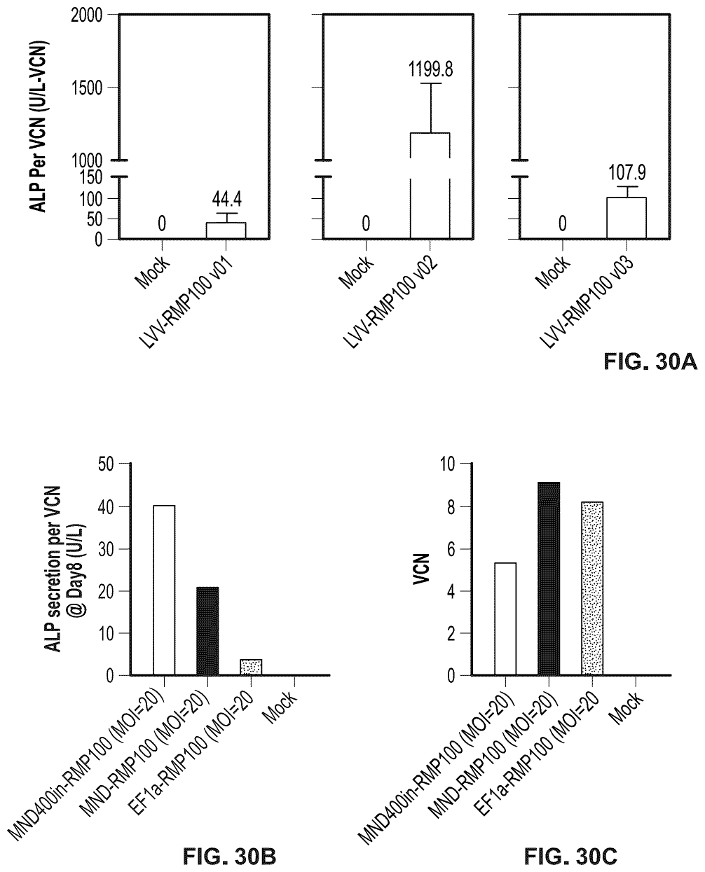

Four TNALP LVV constructs (SEQ ID NOS: 55, 85, 74 and 90) were used to generate VSVG-pseudotyped lentivectors by co-transfecting 293T human embryonic kidney cells with three other plasmids. Lentivirus supernatants were harvested and concentrated through TFF [0301]. Virus supernatants were used to transduce 293T cells at MOI=0.1, 0.2, 0.5, 1, 2, 5 & 10 and human adult CD34+ hematopoietic stem cells at MOI=20. Human adult mobilized peripheral blood CD34+ hematopoietic stem cells were thawed and pre-stimulated with SGSM serum-free media supplemented with SCF, 113, TPO, Flt3, UM171&SR1 overnight. Then transduction was carried out at different MOIs by adding vectors into the cells in the presence of transduction enhancers dmPGE2 and F108. The media of transduced cells were refreshed to SFEMII supplemented with SCF, 113, TPO, Flt3, UM171&SR1 for 14 days. The supernatant was removed for ALP analysis at day 8 and VCN analysis was carried out on cell pellets on day 14. Results shows potency of 400inMND-RMP100 was about 2-fold of MND-RMP100 and about 8 fold of EF1a-RMP100 in HSC, respectively (see FIGS. 30A-30C).

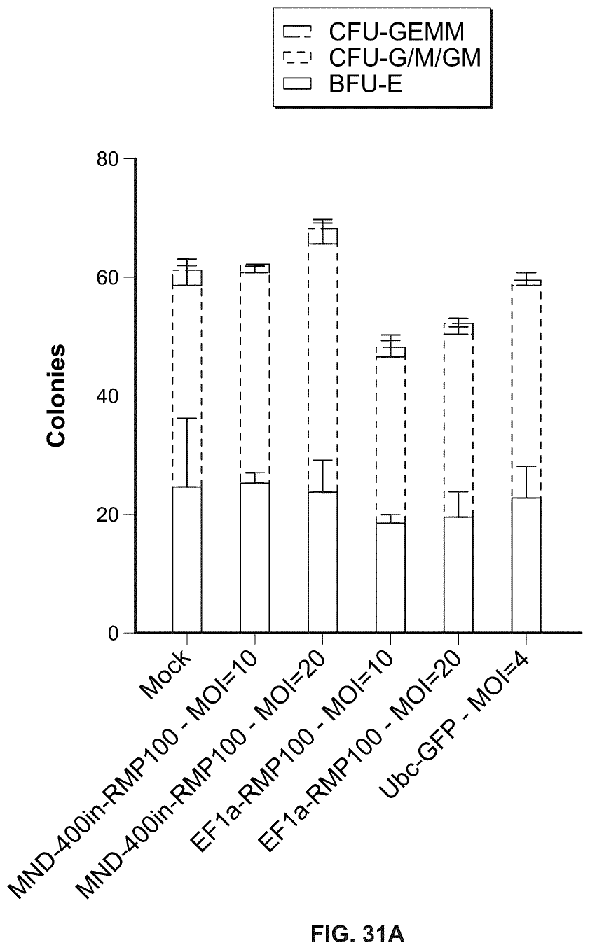

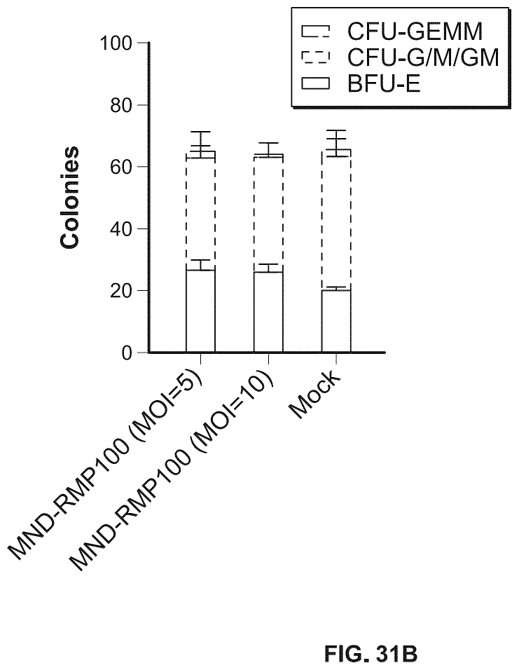

Three TNALP LVV constructs (SEQ ID NOS: 85, 74 and 90) were used to generate VSVG-pseudotyped lentivectors by co-transfecting 293T human embryonic kidney cells with three other plasmids. Lentivirus supernatants were harvested and concentrated through TFF. Human adult mobilized peripheral blood CD34+ hematopoietic stem cells were thawed and pre-stimulated with SCF, 113, TPO and Flt3 overnight. Then transduction was carried out at different MOIs by adding vectors into the cells in the presence of transduction enhancers dmPGE2 and F108. The transduced HSC was seeded in Methocult cellulose for 14 days before readout of colonies. CFU data suggested transduced HSC transduced with MND-400 in-RMP100, EF1a-RMP100 and MND-RMP100 at MOIs from 5 to 20 had similar colony-forming capability compared with mock HSC. No oblivious toxicity were observed for all constructs in HSC (see FIGS. 31A and 31B).

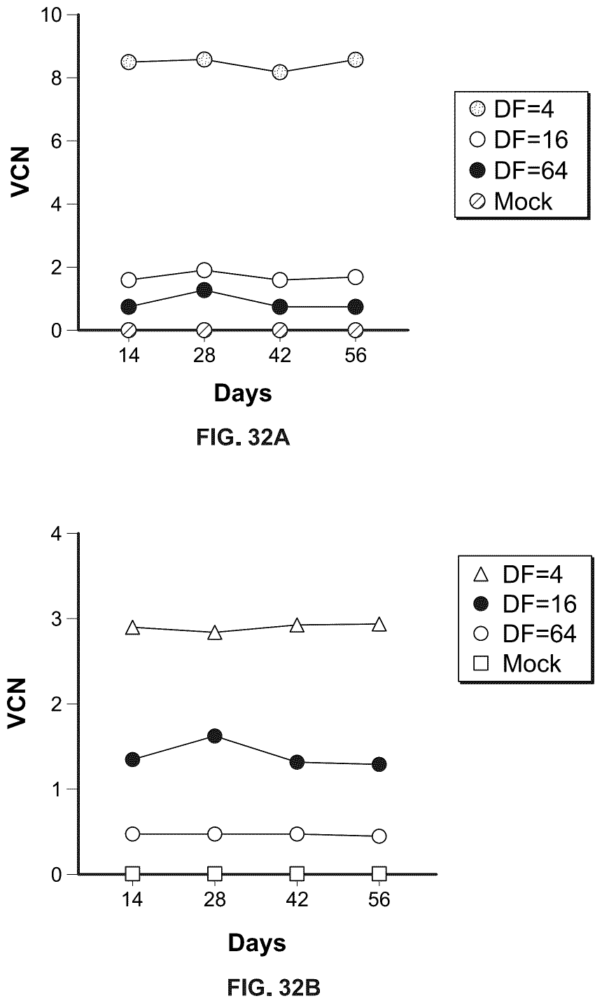

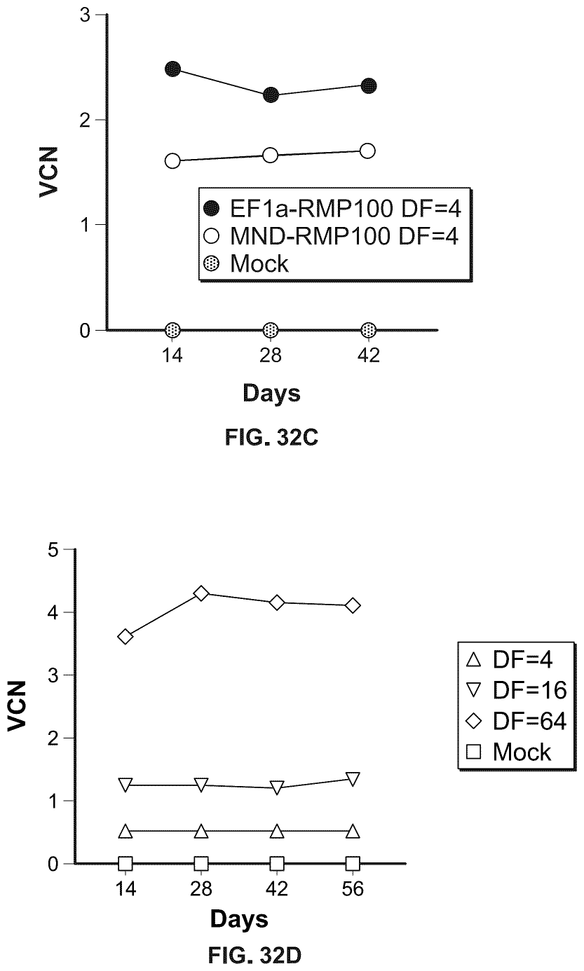

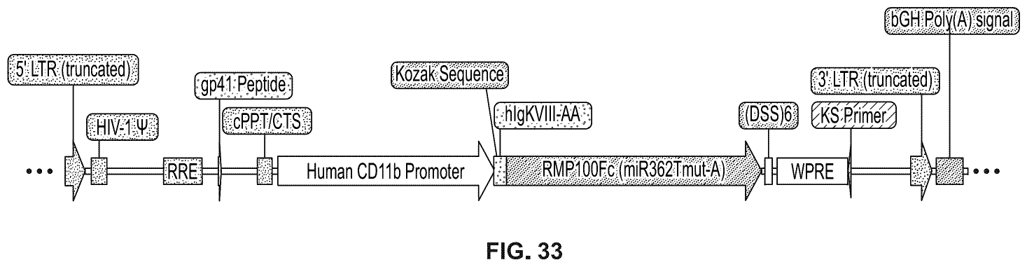

Three TNALP LVV constructs (SEQ ID NOS: 85, 74 and 90) were used to generate VSVG-pseudotyped lentivectors by co-transfecting 293T human embryonic kidney cells with three other plasmids. Lentivirus supernatants were harvested and used to transduce THP-1, Jurkat & HLP-60 at dilution factor 4, 16 and 64. After transduction, the transduced cells were cultured for up-to 56 days and cell pellets were collected for VCN analysis at day 14, 28, 42 and 56, respectively. All constructs showed robust stability in transduced cell lines THP-1, HL_60 & Jurkat (see FIGS. 32A-32D).

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications, and publications to provide yet further embodiments.

Disclosed herein are additional embodiments of the present disclosure, including alternatives of certain additional embodiments. Where an additional embodiment depends from a referenced additional embodiment that includes one or more alternatives, that dependent additional embodiment hereby refers back to the referenced additional embodiment and any of its described alternatives.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.
Additional Embodiment 1. A Polypeptide Comprising Formula (I):

[A]ᵥ-[B]-[C]_{w}-[R]_{q}-([D]ₓ[E]_{y})_{z}, (I)
wherein
   A comprises an amino acid sequence encoding a secretion signal peptide;
   B comprises an amino acid sequence having at least 90% sequence identity to SEQ ID NO: 11 (e.g., at least about 95% sequence identity to SEQ ID NO: 11, at least about 96% sequence identity to SEQ ID NO: 11, at least about 97% sequence identity to SEQ ID NO: 11, at least about 98% sequence identity to SEQ ID NO: 11, at least about 99% sequence identity to SEQ ID NO: 11, or 100% sequence identity to SEQ ID NO: 11);
   C comprises an amino acid sequence encoding a GPI anchor;
   R is -(Mₒ(Fc)Nₚ)-, where M and N each independently include between 1 and 6 amino acids, where Fc is a Fc domain, and o and p are each independently 0, 1, or 2;
   D comprises an amino acid sequence having between 4 and 6 amino acids, or is F(G)ₜF, where each F is the same amino acid, G is an amino acid sequence having 3, 4, or 5 amino acids, and t is an integer ranging from 2-5;
   E comprises an amino acid sequence having between 1 and 8 amino acids;
   q is 0 or 1;
   v is 0 or 1;
   w is 0 or 1;
   x is 0 or an integer ranging from 1 to 6;
   y is 0 or an integer ranging from 1 to 16; and
   z is 0 or an integer ranging from 1 to 6.
Additional Embodiment 2. The polypeptide of additional embodiment 1, wherein [A]ᵥ-[B]-[C]_{w} comprises an amino acid sequence having at least 90% sequence identity to SEQ ID NO: 10.
Additional Embodiment 3. The polypeptide of additional embodiment 1, wherein [A]ᵥ-[B]-[C]_{w} comprises an amino acid sequence having SEQ ID NO: 10.
Additional Embodiment 4. The polypeptide of additional embodiment 1, wherein A comprises an amino acid sequence having at least 95% identity to any one of SEQ ID NOS: 12 and 33-43.
Additional Embodiment 5. The polypeptide of additional embodiment 1, wherein A comprises an amino acid sequence having any one of SEQ ID NOS: 12 and 33-43.
Additional Embodiment 6. The polypeptide of any additional embodiment 1, wherein v is 1 and w is 0. Alternatively, the polypeptide of any additional embodiment 1, wherein v is 1, w is 0, and q is 0. Alternatively, the polypeptide of any additional embodiment 1, wherein v is 1, w is 0, q is 0, and z is 1. Alternatively, the polypeptide of any additional embodiment 1, wherein v is 1, w is 0, and q is 1. Alternatively, the polypeptide of any additional embodiment 1, wherein v is 1, w is 0, q is 1, and z is 1.
Additional Embodiment 7. The polypeptide of any one of additional embodiments 1 and 4 to 5, wherein z is 0.
Additional Embodiment 8. The polypeptide of any one of additional embodiments 1 and 4 to 6, wherein x is 0.
Additional Embodiment 9. The polypeptide of additional embodiment 1, wherein the polypeptide has at least 95% sequence identity to SEQ ID NO: 2.
Additional Embodiment 10. The polypeptide of additional embodiment 1, wherein the polypeptide comprises SEQ ID NO: 2.
Additional Embodiment 11. The polypeptide of any one of additional embodiments 1, 4, and 5 wherein E comprises at most two amino acids.
Additional Embodiment 12. The polypeptide of any one of additional embodiments 1, 4, and 5, wherein E comprises 1 amino acid.
Additional Embodiment 13. The polypeptide of any one of additional embodiments 11 and 12, where E comprises aspartic acid.
Additional Embodiment 14. The polypeptide of any one of additional embodiments 11 to 13, wherein y ranges from between 8 and 12. Alternatively, the polypeptide of any one of additional embodiments 11 to 13, wherein y ranges from between 8 and 12, and q is 0. Alternatively, the polypeptide of any one of additional embodiments 11 to 13, wherein y ranges from between 8 and 12, q is 0, and z is 1. Alternatively, the polypeptide of any one of additional embodiments 11 to 13, wherein y ranges from between 8 and 12, and q is 1. Alternatively, the polypeptide of any one of additional embodiments 11 to 13, wherein y ranges from between 8 and 12, q is 1, and z is 1.
Additional Embodiment 15. The polypeptide of any one of additional embodiments 11 to 14, wherein v is 1 and w is 0. Alternatively, the polypeptide of any one of additional embodiments 11 to 14, wherein v is 1, w is 0, and q is 0. Alternatively, the polypeptide of any one of additional embodiments 11 to 14, wherein v is 1, w is 0, q is 0, and z is 1. Alternatively, the polypeptide of any one of additional embodiments 11 to 14, wherein v is 1 and w is 0, and q is 1. Alternatively, the polypeptide of any one of additional embodiments 11 to 14, wherein v is 1 and w is 0, q is 1, and z is 1.
Additional Embodiment 16. The polypeptide of any one of additional embodiments 11 to 15, wherein x is 0.
Additional Embodiment 17. The polypeptide of any one of additional embodiments 11 to 16, where y is 10. Alternatively, the polypeptide of any one of additional embodiments 11 to 16, where y is 10 and q is 0. Alternatively, the polypeptide of any one of additional embodiments 11 to 16, where y is 10, q is 0, and z is 1. Alternatively, the polypeptide of any one of additional embodiments 11 to 16, where y is 10 and q is 1. Alternatively, the polypeptide of any one of additional embodiments 11 to 16, where y is 10, q is 1, and z is 1.
Additional Embodiment 18. The polypeptide of additional embodiment 1, wherein the polypeptide has at least 95% sequence identity to SEQ ID NO: 3.
Additional Embodiment 19. The polypeptide of additional embodiment 1, wherein the polypeptide comprises SEQ ID NO: 3.
Additional Embodiment 20. The polypeptide of any one of additional embodiments 1, 4, and 5, wherein D comprises at most 5 amino acids.
Additional Embodiment 21. The polypeptide of additional embodiment 20, wherein at least three contiguous amino acids of the at most 5 amino acids are the same. Alternatively, the polypeptide of additional embodiment 20, wherein at least four contiguous amino acids of the at most 5 amino acids are the same.
Additional Embodiment 22. The polypeptide of additional embodiment 21, wherein the at least three contiguous amino acids are each glycine. Alternatively, the polypeptide of additional embodiment 21, wherein the at least three contiguous amino acids are each glycine and where q is 0. Alternatively, the polypeptide of additional embodiment 21, wherein the at least three contiguous amino acids are each glycine and where q is 1.
Additional Embodiment 23. The polypeptide of any one of additional embodiments 20 to 22, wherein x is an integer ranging from 1 to 3.
Additional Embodiment 24. The polypeptide of any one of additional embodiments 20 to 22, wherein x is 2.
Additional Embodiment 25. The polypeptide of any one of additional embodiments 20-24, wherein y is an integer ranging from between 4 and 8.
Additional Embodiment 26. The polypeptide of additional embodiment 25, wherein y is between 5 and 7.
Additional Embodiment 27. The polypeptide of any one of additional embodiments 25-26, wherein y is 6.
Additional Embodiment 28. The polypeptide of any one of additional embodiments 20-27, wherein E comprises 3 amino acids.
Additional Embodiment 29. The polypeptide of additional embodiment 28, wherein at least 2 contiguous amino acids of the three amino acids are the same.
Additional Embodiment 30. The polypeptide of any one of additional embodiments 20-29, wherein E comprises -asp-ser-ser-.
Additional Embodiment 31. The polypeptide of additional embodiment 1, wherein the polypeptide has at least 95% sequence identity to SEQ ID NO: 6.
Additional Embodiment 32. The polypeptide of additional embodiment 1, wherein the polypeptide comprises SEQ ID NO: 6.
Additional Embodiment 33. The polypeptide of any one of additional embodiments 20-24, wherein y is between 8 and 12.
Additional Embodiment 34. The polypeptide of any one of additional embodiments 20-24, wherein y is 10.
Additional Embodiment 35. The polypeptide of any one of additional embodiments 20-24 and 33-34, wherein E comprises 1 or 2 amino acids.
Additional Embodiment 36. The polypeptide of any one of additional embodiments 20-24 and 33-35, wherein E comprises 1 amino acid.
Additional Embodiment 37. The polypeptide of any one of additional embodiments 20-24 and 33-36, where E comprises only aspartic acid amino acid.
Additional Embodiment 38. The polypeptide of additional embodiment 1, wherein the polypeptide has at least 95% sequence identity to SEQ ID NO: 4.
Additional Embodiment 39. The polypeptide of additional embodiment 1, wherein the polypeptide comprises SEQ ID NO: 4.
Additional Embodiment 40. The polypeptide of any one of additional embodiments 1, 4, and 5, where E comprises between 2 and 4 amino acids. Alternatively, the polypeptide of any one of additional embodiments 1, 4, and 5, where E comprises between 2 and 4 amino acids and where q is 0. Alternatively, the polypeptide of any one of additional embodiments 1, 4, and 5, where E comprises between 2 and 4 amino acids, q is 0, and z is 1. Alternatively, the polypeptide of any one of additional embodiments 1, 4, and 5, where E comprises between 2 and 4 amino acids and where q is 1. Alternatively, the polypeptide of any one of additional embodiments 1, 4, and 5, where E comprises between 2 and 4 amino acids, q is 1, and z is 1.
Additional Embodiment 41. The polypeptide of additional embodiment 40, wherein E comprises 3 amino acids.
Additional Embodiment 42. The polypeptide of any one of additional embodiments 40-41, wherein at least 2 contiguous amino acids of the 3 amino acids are the same.
Additional Embodiment 43. The polypeptide of any one of additional embodiments 40-42, wherein y ranges from 3 to 8, preferably wherein y ranges from 4 to 8.
Additional Embodiment 44. The polypeptide of any one of additional embodiments 40-42, wherein y ranges from 5 to 7.
Additional Embodiment 45. The polypeptide of any one of additional embodiments 40-42, wherein y is 6.
Additional Embodiment 46. The polypeptide of any one of additional embodiments 40-45, wherein E comprises -asp-ser-ser-.
Additional Embodiment 47. The polypeptide of any one of additional embodiments 40-46, wherein v is 1 and w is 0.
Additional Embodiment 48. The polypeptide of additional embodiment 1, wherein the polypeptide has at least 95% sequence identity to any one of SEQ ID NOS: 5 and 44-54.
Additional Embodiment 49. The polypeptide of additional embodiment 1, wherein the polypeptide comprises any one of SEQ ID NOS: 5 and 44-54.
Additional Embodiment 50. The polypeptide of any one of additional embodiments 40-47, wherein the amino acid sequence encoding the GPI anchor has at least 95% sequence identity to SEQ ID NO: 13 (e.g., at least 96% sequence identity to SEQ ID NO: 13, at least 97% sequence identity to SEQ ID NO: 13, at least 98% sequence identity to SEQ ID NO: 13, at least 99% sequence identity to SEQ ID NO: 13, or where the GPI anchor comprises SEQ ID NO: 13).
Additional Embodiment 51. The polypeptide of any one of additional embodiments 40-47, wherein the amino acid sequence encoding the GPI anchor has SEQ ID NO: 14 (e.g., at least 96% sequence identity to SEQ ID NO: 14, at least 97% sequence identity to SEQ ID NO: 14, at least 98% sequence identity to SEQ ID NO: 14, at least 99% sequence identity to SEQ ID NO: 14, or where the GPI anchor comprises SEQ ID NO: 14).
Additional Embodiment 52. The polypeptide of additional embodiment 1, wherein the polypeptide has at least 95% sequence identity to SEQ ID NO: 8.
Additional Embodiment 53. The polypeptide of additional embodiment 1, wherein the polypeptide comprises SEQ ID NO: 8.
Additional Embodiment 54. The polypeptide of any one of additional embodiments 11-17, wherein v is 1 and w is 1.
Additional Embodiment 55. The polypeptide of additional embodiment 54, wherein the amino acid sequence encoding the GPI anchor has at least 95% sequence identity to SEQ ID NO: 13 (e.g., at least 96% sequence identity to SEQ ID NO: 13, at least 97% sequence identity to SEQ ID NO: 13, at least 98% sequence identity to SEQ ID NO: 13, at least 99% sequence identity to SEQ ID NO: 13, or where the GPI anchor comprises SEQ ID NO: 13).
Additional Embodiment 56. The polypeptide of additional embodiment 54, the amino acid sequence encoding the GPI anchor has SEQ ID NO: 14 (e.g., at least 96% sequence identity to SEQ ID NO: 14, at least 97% sequence identity to SEQ ID NO: 14, at least 98% sequence identity to SEQ ID NO: 14, at least 99% sequence identity to SEQ ID NO: 14, or where the GPI anchor comprises SEQ ID NO: 14).
Additional Embodiment 57. The polypeptide of additional embodiment 1, wherein the polypeptide has at least 95% sequence identity to SEQ ID NO: 7.
Additional Embodiment 58. The polypeptide of additional embodiment 1, wherein the polypeptide comprises SEQ ID NO: 7.
Additional Embodiment 59. A lentiviral vector including a nucleic acid sequence encoding the polypeptide of any one of additional embodiments 1 to 58.
Additional Embodiment 60. The lentiviral vector of additional embodiment 59, wherein the nucleic acid sequence encoding the polypeptide is operably linked to a promoter.
Additional Embodiment 61. The lentiviral vector of any one of additional embodiments 59 to 60, wherein the promoter is selected from the group consisting of EF1A, MND, CD11b, CD68LPp, EF1a 1, EFS, and UbC. In some embodiments, the promoter has at least 95% identity to any one of SEQ ID NOS: 66, 67, 96, 99, 100, 101, and 126. In some embodiments, the promoter has at least 96% identity to any one of SEQ ID NOS: 66, 67, 96, 99, 100, 101, and 126. In some embodiments, the promoter has at least 97% identity to any one of SEQ ID NOS: 66, 67, 96, 99, 100, 101, and 126. In some embodiments, the promoter has at least 95% identity to any one of SEQ ID NOS: 66, 67, 96, 99, 100, 101, and 126. In some embodiments, the promoter has at least 98% identity to any one of SEQ ID NOS: 66, 67, 96, 99, 100, 101, and 126. In some embodiments, the promoter has at least 99% identity to any one of SEQ ID NOS: 66, 67, 96, 99, 100, 101, and 126. In some embodiments, the promoter comprises any one of SEQ ID NOS: 66, 67, 96, 99, 100, 101, and 126.
Additional Embodiment 62. The lentiviral vector of any one of additional embodiments 59-61, further comprising a UCOE promoter element.
Additional Embodiment 63. The lentiviral vector of any one of additional embodiments 59-62, further comprising an insulator is selected from the group consisting of cHS 400 bp insulator, engineered mutant cHS 400 bp insulators—m3SD400 in & m6SD400 in.+−
Additional Embodiment 64. The lentiviral vector of any one of additional embodiments 59-63, further comprising one or more Scaffold/Matrix Attachment Regions.
Additional Embodiment 65. The lentiviral vector of any one of additional embodiments 59-64, further comprising a WPRE element. Alternatively, the lentiviral vector of any one of additional embodiments 59-64, further comprising a WPRE element and further including RRE, cPPT/CTS, and bGH-poly(A) signal.
Additional Embodiment 66. The lentiviral vector of any one of additional embodiments 59-64, wherein the lentiviral vector does not include a WPRE element. Alternatively, the lentiviral vector of any one of additional embodiments 59-64, wherein the lentiviral vector does not include a WPRE element, but does include RRE, cPPT/CTS, and bGH-poly(A) signal.
Additional Embodiment 67. The lentiviral vector of additional embodiment 59, wherein the lentiviral vector comprises a nucleotide sequence having at least 90% sequence identity to any one of SEQ ID NOS: 16-26, 55-65, 74, 81, 82, 84-95, 97, and 98.
Additional Embodiment 68. The lentiviral vector of additional embodiment 59, wherein the lentiviral vector comprises a nucleotide sequence having at least 95% sequence identity to any one of SEQ ID NOS: 16-26, 55-65, 74, 81, 82, 84-95, 97, and 98 (e.g., at least 96% sequence identity to any one of SEQ ID NOS: 16-26, 55-65, 74, 81, 82, 84-95, 97, and 98; 97% sequence identity to any one of SEQ ID NOS: 16-26, 55-65, 74, 81, 82, 84-95, 97).
Additional Embodiment 69. The lentiviral vector of additional embodiment 59, wherein the lentiviral vector comprises a nucleotide sequence having at least 98% sequence identity to any one of SEQ ID NOS: 16-26, 55-65, 74, 81, 82, 84-95, 97, and 98.
Additional Embodiment 70. The lentiviral vector of additional embodiment 59, wherein the lentiviral vector comprises a nucleotide sequence having at least 99% sequence identity to any one of SEQ ID NOS: 16-26, 55-65, 74, 81, 82, 84-95, 97, and 98.
Additional Embodiment 71. The lentiviral vector of additional embodiment 59, wherein the lentiviral viral vector comprises a nucleotide sequence having any one of SEQ ID NOS: 16-26, 55-65, 74, 81, 82, 84-95, 97, and 98.
Additional Embodiment 72. A lentiviral vector comprising a nucleotide sequence having at least 90% sequence identity to any one of SEQ ID NOS: 15-26, 55-65, 74, 81, 82, 84 95, 97, and 98.
Additional Embodiment 73. The lentiviral vector of additional embodiment 72, wherein the nucleotide sequence has at least 95% sequence identity to any one of SEQ ID NOS: 15 26, 55-65, 74, 81, 82, 84-95, 97, and 98.
Additional Embodiment 74. The lentiviral vector of additional embodiment 72, wherein the nucleotide sequence has at least 96% sequence identity to any one of SEQ ID NOS: 15 26, 55-65, 74, 81, 82, 84-95, 97, and 98.
Additional Embodiment 75. The lentiviral vector of additional embodiment 72, wherein the nucleotide sequence has at least 97% sequence identity to any one of SEQ ID NOS: 15 26, 55-65, 74, 81, 82, 84-95, 97, and 98.
Additional Embodiment 76. The lentiviral vector of additional embodiment 72, wherein the nucleotide sequence has at least 98% sequence identity to any one of SEQ ID NOS: 15 26, 55-65, 74, 81, 82, 84-95, 97, and 98.
Additional Embodiment 77. The lentiviral vector of additional embodiment 72, wherein the nucleotide sequence has at least 99% sequence identity to any one of SEQ ID NOS: 15 26, 55-65, 74, 81, 82, 84-95, 97, and 98.
Additional Embodiment 78. The lentiviral vector of additional embodiment 72, wherein the nucleotide sequence comprises any one of SEQ ID NOS: 15-26, 55-65, 74, 81, 82, 84-95, 97, and 98.
Additional Embodiment 79. A population of host cells transduced with any the lentiviral vector of any one of additional embodiments 59-78.
Additional Embodiment 80. A method of transducing a population of host cells comprising: obtaining a population of host cells and contacting the obtained population of host cells with the lentiviral vector of any one of additional embodiments 59-78.
Additional Embodiment 81. The method of additional embodiment 80, wherein the transduction occurs ex vivo.
Additional Embodiment 82. A pharmaceutical composition comprising the transduced host cells of additional embodiment 79 and a pharmaceutically acceptable excipient or carrier.
Additional Embodiment 83. A method of treating a mammalian subject comprising administering a therapeutically effective amount of the transduced host cells of additional embodiment 79 or the pharmaceutical composition of additional embodiment 82 to the mammalian subject.
Additional Embodiment 84. The polypeptide of any one of additional embodiments 1-83, wherein the polypeptide of Formula (I) does not have the amino acid sequence of SEQ ID NO: 1 or the amino acid sequence encoded by the nucleotide sequence of SEQ ID NO: 107; or provided that the polypeptide of Formula (I) is not Strensiq® or Asfotase alfa.
Additional Embodiment 85. The polypeptide of any one of additional embodiments 1-83, wherein when v is 1, w is 0, q is 1, o is 1, p is 1, N is the diamino acid -D-I-, M is the diamino acid -L-K-, [B] comprises SEQ ID NO: 11, Fc comprises, and x is 0, then [E]_{y} is not D₁₀-D₁₆.
Additional Embodiment 86. A polypeptide comprising Formula (I):

[A]ᵥ-[B]-[C]_{w}-[R]_{q}-([D]ₓ-[E]_{y})_{z}, (I)
wherein
   A comprises an amino acid sequence encoding a secretion signal peptide;
   B comprises an amino acid sequence having at least 90% sequence identity to SEQ ID NO: 11 (e.g., at least about 95% sequence identity to SEQ ID NO: 11, at least about 96% sequence identity to SEQ ID NO: 11, at least about 97% sequence identity to SEQ ID NO: 11, at least about 98% sequence identity to SEQ ID NO: 11, at least about 99% sequence identity to SEQ ID NO: 11, or 100% sequence identity to SEQ ID NO: 11);
   C comprises an amino acid sequence encoding a GPI anchor;
   R is -(Mₒ(Fc)Nₚ)-, where M and N each independently include between 1 and 6 amino acids, where Fc is a Fc domain, and o and p are each independently 0, 1, or 2;
   D comprises an amino acid sequence having between 4 and 6 amino acids, or is F(G)ₜF, where each F is the same amino acid, G is an amino acid sequence having 3, 4, or 5 amino acids, and t is an integer ranging from 2-5;
   E comprises an amino acid sequence having between 1 and 8 amino acids;
   q is 0 or 1;
   v is 0 or 1;
   w is 0 or 1;
   x is 0 or an integer ranging from 1 to 6;
   y is 0 or an integer ranging from 1 to 16; and
   z is 0 or an integer ranging from 1 to 6;
      provided that the polypeptide of Formula (I) is not conjugated to a sugar molecule, such as dextran.
Additional Embodiment 87. A polypeptide comprising Formula (I):

[A]ᵥ-[B]-[C]_{w}-[R]_{q}-([D]ₓ-[E]_{y})_{z}, (I)
wherein
A comprises an amino acid sequence encoding a secretion signal peptide;
B comprises an amino acid sequence having at least 90% sequence identity to SEQ ID NO: 11 (e.g., at least about 95% sequence identity to SEQ ID NO: 11, at least about 96% sequence identity to SEQ ID NO: 11, at least about 97% sequence identity to SEQ ID NO: 11, at least about 98% sequence identity to SEQ ID NO: 11, at least about 99% sequence identity to SEQ ID NO: 11, or 100% sequence identity to SEQ ID NO: 11);
C comprises an amino acid sequence encoding a GPI anchor;
R is -(Mₒ(Fc)Nₚ)-, where M and N each independently include between 1 and 6 amino acids, where Fc is a Fc domain, and o and p are each independently 0, 1, or 2;
D comprises an amino acid sequence having between 4 and 6 amino acids, or is F(G)ₜF, where each F is the same amino acid, G is an amino acid sequence having 3, 4, or 5 amino acids, and t is an integer ranging from 2-5;
E comprises an amino acid sequence having between 1 and 8 amino acids;
q is 0 or 1;
v is 0 or 1;
w is 0 or 1;
x is 0 or an integer ranging from 1 to 6;
y is 0 or an integer ranging from 1 to 16; and
z is 0 or an integer ranging from 1 to 6,
provided that the polypeptide having Formula (I) does not comprise SEQ ID NO: 1, or where the polypeptide having Formula (I) has less than 100% sequence identity to that of SEQ ID NO: 1, preferably less than 99% sequence identity to that of SEQ ID NO: 1, more preferably less than 98% sequence identity to that of SEQ ID NO: 1.
Additional Embodiment 88. A lentiviral vector including a nucleic acid sequence encoding the polypeptide of any one of additional embodiments 84-87.
Additional Embodiment 89. The lentiviral vector of additional embodiment 88, wherein the nucleic acid sequence encoding the polypeptide is operably linked to a promoter.
Additional Embodiment 90. The lentiviral vector of any one of additional embodiments 88 to 89, wherein the promoter is selected from the group consisting of EF1A, MND, CD11b, CD68LP, EF1a1, EFS, and UbC. In some embodiments, the promoter has at least 95% identity to any one of SEQ ID NOS: 66, 67, 96, 99, 100, 101, and 126. In some embodiments, the promoter has at least 96% identity to any one of SEQ ID NOS: 66, 67, 96, 99, 100, 101, and 126. In some embodiments, the promoter has at least 97% identity to any one of SEQ ID NOS: 66, 67, 96, 99, 100, 101, and 126. In some embodiments, the promoter has at least 95% identity to any one of SEQ ID NOS: 66, 67, 96, 99, 100, 101, and 126. In some embodiments, the promoter has at least 98% identity to any one of SEQ ID NOS: 66, 67, 96, 99, 100, 101, and 126. In some embodiments, the promoter has at least 99% identity to any one of SEQ ID NOS: 66, 67, 96, 99, 100, 101, and 126. In some embodiments, the promoter comprises any one of SEQ ID NOS: 66, 67, 96, 99, 100, 101, and 126.
Additional Embodiment 91. The lentiviral vector of any one of additional embodiments 88-90, further comprising a UCOE promoter element.
Additional Embodiment 92. The lentiviral vector of any one of additional embodiments 88-91, further comprising an insulator.
Additional Embodiment 93. The lentiviral vector of any one of additional embodiments 88-92, further comprising one or more Scaffold/Matrix Attachment Regions.
Additional Embodiment 94. The lentiviral vector of any one of additional embodiments 88-93, further comprising a WPRE element. Alternatively, the lentiviral vector of any one of additional embodiments 59-64, further comprising a WPRE element and further including RRE, cPPT/CTS, and bGH-poly(A) signal.
Additional Embodiment 95. The lentiviral vector of any one of additional embodiments 88-94, wherein the lentiviral vector does not include a WPRE element. Alternatively, the lentiviral vector of any one of additional embodiments 88-94, wherein the lentiviral vector does not include a WPRE element, but does include RRE, cPPT/CTS, and bGH-poly(A) signal.
Additional Embodiment 96. A population of host cells transduced with any the lentiviral vector of any one of additional embodiments 88-95.
Additional Embodiment 97. A method of transducing a population of host cells comprising: obtaining a population of host cells and contacting the obtained population of host cells with the lentiviral vector of any one of additional embodiments 88-94.
Additional Embodiment 98. The method of additional embodiment 97, wherein the transduction occurs ex vivo.
Additional Embodiment 99. A pharmaceutical composition comprising the transduced host cells of additional embodiment 97 and a pharmaceutically acceptable excipient or carrier.
Additional Embodiment 100. A method of treating a mammalian subject comprising administering a therapeutically effective amount of the transduced host cells of additional embodiment 97 or the pharmaceutical composition of additional embodiment 99 to the mammalian subject.
Additional Embodiment 101. A polypeptide comprising Formula (I):

[A]ᵥ-[B]-[C]_{w}-[R]_{q}-([D]ₓ-[E]_{y})_{z}, (I)
wherein
   A comprises an amino acid sequence encoding a secretion signal peptide;
   B comprises an amino acid sequence having at least 90% sequence identity to a SEQ ID NO: 11;
   C comprises an amino acid sequence encoding a GPI anchor;
   R is -(Mₒ(Fc)Nₚ)-, where M and N each independently include between 1 and 6 amino acids, where Fc is a Fc domain, and o and p are each independently 0, 1, or 2;
   D comprises an amino acid sequence having between 4 and 6 amino acids, or is F(G)ₜF, where each F is the same amino acid, G is an amino acid sequence having 3, 4, or 5 amino acids, and t is an integer ranging from 2-5;
   E comprises an amino acid sequence having between 1 and 8 amino acids;
   q is 0 or 1;
   v is 0 or 1;
   w is 0 or 1;
   x is 0 or an integer ranging from 1 to 6;
   y is 0 or an integer ranging from 1 to 16; and
   z is 0 or an integer ranging from 1 to 6.
Additional Embodiment 102. The polypeptide of additional embodiment 101, wherein [A]ᵥ-[B]-[C]_{w} comprises an amino acid sequence having at least 90% sequence identity to SEQ ID NO: 10.
Additional Embodiment 103. The polypeptide of additional embodiment 101, wherein [A]ᵥ-[B]-[C]_{w} comprises an amino acid sequence having SEQ ID NO: 10.
Additional Embodiment 104. The polypeptide of additional embodiment 101, wherein A comprises an amino acid sequence having at least 95% identity to any one of SEQ ID NOS: 12 and 33-43.
Additional Embodiment 105. The polypeptide of additional embodiment 101, wherein A comprises an amino acid sequence having any one of SEQ ID NOS: 12 and 33-43.
Additional Embodiment 106. The polypeptide of any one of additional embodiments 104 and 105, wherein v is 1 and w is 0.
Additional Embodiment 107. The polypeptide of any one of additional embodiments 104 to 106, wherein z is 0.
Additional Embodiment 108. The polypeptide of any one of additional embodiments 104 to 106, wherein x is 0.
Additional Embodiment 109. The polypeptide of additional embodiment 101, wherein the polypeptide has at least 95% sequence identity to SEQ ID NO: 2.
Additional Embodiment 110. The polypeptide of additional embodiment 101, wherein the polypeptide comprises SEQ ID NO: 2.
Additional Embodiment 111. The polypeptide of additional embodiment 101, wherein E comprises at most two amino acids.
Additional Embodiment 112. The polypeptide of additional embodiment 101, wherein E comprises 1 amino acid.
Additional Embodiment 113. The polypeptide of any one of additional embodiments 111 and 112, where E comprises aspartic acid.
Additional Embodiment 114. The polypeptide of any one of additional embodiments 111 to 113, wherein y ranges from between 8 and 12.
Additional Embodiment 115. The polypeptide of any one of additional embodiments 111 to 113, wherein v is 1 and w is 0.
Additional Embodiment 116. The polypeptide of any one of additional embodiments 111 to 115, wherein x is 0.
Additional Embodiment 117. The polypeptide of any one of additional embodiments 111 to 113, where y is 10.
Additional Embodiment 118. The polypeptide of additional embodiment 101, wherein the polypeptide has at least 95% sequence identity to SEQ ID NO: 3.
Additional Embodiment 119. The polypeptide of additional embodiment 101, wherein the polypeptide comprises SEQ ID NO: 3.
Additional Embodiment 120. The polypeptide of additional embodiment 101, D comprises at most 5 amino acids.
Additional Embodiment 121. The polypeptide of additional embodiment 120, wherein at least three contiguous amino acids of the at most 5 amino acids are the same.
Additional Embodiment 122. The polypeptide of additional embodiment 121, wherein the at least three contiguous amino acids are each glycine.
Additional Embodiment 123. The polypeptide of any one of additional embodiments 120 to 122, wherein x is an integer ranging from 1 to 3.
Additional Embodiment 124. The polypeptide of any one of additional embodiments 120 to 122, wherein x is 2.
Additional Embodiment 125. The polypeptide of any one of additional embodiments 120 124, wherein y is an integer ranging from between 4 and 8.
Additional Embodiment 126. The polypeptide of any one of additional embodiments 120 124, wherein y is between 5 and 7.
Additional Embodiment 127. The polypeptide of any one of additional embodiments 121 124, wherein y is 6.
Additional Embodiment 128. The polypeptide of any one of additional embodiments 120 127, wherein E comprises 3 amino acids.
Additional Embodiment 129. The polypeptide of additional embodiment 128, wherein at least 2 contiguous amino acids of the three amino acids are the same.
Additional Embodiment 130. The polypeptide of any one of additional embodiments 120 129, wherein E comprises -asp-ser-ser-.
Additional Embodiment 131. The polypeptide of additional embodiment 101, wherein the polypeptide has at least 95% sequence identity to SEQ ID NO: 6.
Additional Embodiment 132. The polypeptide of additional embodiment 101, wherein the polypeptide comprises SEQ ID NO: 6.
Additional Embodiment 133. The polypeptide of any one of additional embodiments 120 124, wherein y is between 8 and 12.
Additional Embodiment 134. The polypeptide of any one of additional embodiments 120 124, wherein y is 10.
Additional Embodiment 135. The polypeptide of any one of additional embodiments 120-124 and 133-134, wherein E comprises 1 or 2 amino acids.
Additional Embodiment 136. The polypeptide of any one of additional embodiments 120-124 and 133-135, wherein E comprises 1 amino acid.
Additional Embodiment 137. The polypeptide of any one of additional embodiments 120-124 and 133-136, where E comprises a single aspartic acid amino acid.
Additional Embodiment 138. The polypeptide of additional embodiment 101, wherein the polypeptide has at least 95% sequence identity to SEQ ID NO: 4.
Additional Embodiment 139. The polypeptide of additional embodiment 101, wherein the polypeptide comprises SEQ ID NO: 4.
Additional Embodiment 140. The polypeptide of additional embodiment 101, where E comprises between 2 and 4 amino acids.
Additional Embodiment 141. The polypeptide of additional embodiment 140, wherein E comprises 3 amino acids.
Additional Embodiment 142. The polypeptide of any one of additional embodiments 140-141, wherein at least 2 contiguous amino acids of the 3 amino acids are the same.
Additional Embodiment 143. The polypeptide of any one of additional embodiments 140-142, wherein y ranges from 4 to 8.
Additional Embodiment 144. The polypeptide of any one of additional embodiments 140-143, wherein y ranges from 5 to 7.
Additional Embodiment 145. The polypeptide of any one of additional embodiments 140-144, wherein y is 6.
Additional Embodiment 146. The polypeptide of any one of additional embodiments 140-145, wherein E comprises -asp-ser-ser-.
Additional Embodiment 147. The polypeptide of any one of additional embodiments 140-146, wherein v is 1 and w is 0.
Additional Embodiment 148. The polypeptide of additional embodiment 101, wherein the polypeptide has at least 95% sequence identity to any one of SEQ ID NOS: 5 and 44-54.
Additional Embodiment 149. The polypeptide of additional embodiment 101, wherein the polypeptide comprises any one of SEQ ID NOS: 5 and 44-54.
Additional Embodiment 150. The polypeptide of any one of additional embodiments 140-147, wherein the amino acid sequence encoding the GPI anchor has at least 95% sequence identity to SEQ ID NO: 13.
Additional Embodiment 151. The polypeptide of any one of additional embodiments 140-147, wherein the amino acid sequence encoding the GPI anchor has SEQ ID NO: 14.
Additional Embodiment 152. The polypeptide of additional embodiment 101, wherein the polypeptide has at least 95% sequence identity to SEQ ID NO: 8.
Additional Embodiment 153. The polypeptide of additional embodiment 101, wherein the polypeptide comprises SEQ ID NO: 8.
Additional Embodiment 154. The polypeptide of any one of additional embodiments 111-114, wherein v is 1 and w is 1.
Additional Embodiment 155. The polypeptide of additional embodiment 154, wherein the amino acid sequence encoding the GPI anchor has at least 95% sequence identity to SEQ ID NO: 13.
Additional Embodiment 156. The polypeptide of additional embodiment 154, the amino acid sequence encoding the GPI anchor has SEQ ID NO: 14.
Additional Embodiment 157. The polypeptide of additional embodiment 101, wherein the polypeptide has at least 95% sequence identity to SEQ ID NO: 7.
Additional Embodiment 158. The polypeptide of additional embodiment 101, wherein the polypeptide comprises SEQ ID NO: 7.
Additional Embodiment 159. The polypeptide of additional embodiment 101, wherein R comprises at least 99% identity to that of SEQ ID NO: 9.
Additional Embodiment 160. The polypeptide of additional embodiment 101, wherein R comprises SEQ ID NO: 9.
Additional Embodiment 161. The polypeptide of additional embodiment 101, wherein o, p, and q are each 1.
Additional Embodiment 162. The polypeptide of additional embodiment 161, wherein M comprises 2 amino acids; and wherein N comprises two amino acids; and wherein M and N are different.
Additional Embodiment 163. The polypeptide of additional embodiment 162, wherein M is -L-K-.
Additional Embodiment 164. The polypeptide of additional embodiment 162, wherein N is -D-I-.
Additional Embodiment 165. The polypeptide of additional embodiment 101, wherein [R] is -[L-K]-Fc-[D-I]-, and wherein Fc comprises at least 99% identity to SEQ ID NO: 130.
Additional Embodiment 166. The polypeptide of additional embodiment 101, wherein the polypeptide does not comprise a terminal poly-Asp.
Additional Embodiment 167. The polypeptide of additional embodiment 101, wherein the polypeptide does not comprise ten to sixteen contiguous aspartic acid residues.
Additional Embodiment 168. The polypeptide of additional embodiment 101, wherein the polypeptide does not comprise a terminal poly-Asp having ten to sixteen contiguous aspartic acid residues.
Additional Embodiment 169. The polypeptide of additional embodiment 101, wherein the polypeptide does not a GPI signal peptide.
Additional Embodiment 170. The polypeptide of additional embodiment 101, wherein the polypeptide comprises seventeen or more contiguous aspartic acid residues.
Additional Embodiment 171. The polypeptide of additional embodiment 101, wherein the polypeptide comprises a terminal poly-Asp having seventeen or more contiguous aspartic acid residues, and optionally an Fc domain.
Additional Embodiment 172. The polypeptide of additional embodiment 101, wherein the polypeptide comprises ten to sixteen contiguous negatively charged amino acids, wherein the negatively charged amino acids are other than aspartic acid.
Additional Embodiment 173. The polypeptide of additional embodiment 101, wherein the polypeptide comprises ten to sixteen contiguous glutamic acid residues.
Additional Embodiment 174. The polypeptide of additional embodiment 101, wherein [E] comprises at least two different amino acids.
Additional Embodiment 175. An expression vector comprising a nucleotide sequence encoding the polypeptide of any one of additional embodiments 101 to 175.

1. A polypeptide comprising Formula (IB):
[A]ᵥ-[B]-[C]_{w}-[R]_{q}-([D]ₓ-[E]_{y})_{z}, (IB)
wherein
A comprises an amino acid sequence encoding a secretion signal peptide;
B comprises an amino acid sequence having at least 90% sequence identity to a SEQ ID NO: 11;
C comprises an amino acid sequence encoding a GPI anchor;
R is -(Mₒ(Fc)Nₚ)-, where M and N each independently include between 1 and 6 amino acids, where Fc is a Fc domain, and o and p are each independently 0, 1, or 2;
D comprises an amino acid sequence having between 4 and 6 amino acids, or is F(G) t F, where each F is the same amino acid, G is an amino acid sequence having 3, 4, or 5 amino acids, and t is an integer ranging from 2-5;
E comprises an amino acid sequence having between 1 and 8 amino acids;
q is 0 or 1;
v is 0 or 1;
w is 0 or 1;
x is 0 or an integer ranging from 1 to 6;
y is 0 or an integer ranging from 1 to 16; and
z is 0 or an integer ranging from 1 to 6;
wherein E comprises -D-S-S-.

2. The polypeptide of claim 1, wherein q is 1.

3. The polypeptide of claim 2, wherein v is 1, w is 1, x is 0, and z is 1.

4. The polypeptide of claim 3, wherein y is an integer ranging from 4-8.

5. The polypeptide of claim 4, wherein y is 6.

6. The polypeptide of claim 3, wherein R comprises SEQ ID NO: 9.

7. The polypeptide of claim 3, wherein [R] is -[L-K]-Fc-[D-I]-, and wherein Fc comprises at least 97% identity to SEQ ID NO: 130.

8. The polypeptide of claim 1, wherein x is 0 and [E]_{y} is [-D-S-S-]₆.

9. The polypeptide of claim 1, wherein [D]ₓ is [-G-G-G-G-S-]₁ or [-G-G-G-G-S-]₂.

10. The polypeptide of claim 1, wherein q is 0 and [E]_{y} is [-D-S-S-]₆.

11. The polypeptide of claim 1, wherein the polypeptide has an amino acid sequence having at least 97% identity to any one of SEQ ID NOS: 5, 44-54, 105, and 116-125.

12. The polypeptide of claim 1, wherein the polypeptide has an amino acid sequence having at least 98% identity to any one of SEQ ID NOS: 5, 44-54, 105, and 116-125.

13. The polypeptide of claim 1, wherein the polypeptide has an amino acid sequence having at least 99% identity to any one of SEQ ID NOS: 5, 44-54, 105, and 116-125.

14. The polypeptide of claim 1, wherein the polypeptide has any one of SEQ ID NOS: 5, 44-54, 105, and 116-125.

15. The polypeptide of claim 1, wherein the polypeptide is encoded by a nucleotide sequence having at least 95% identity to any one of SEQ ID NOS: 111, 115, and 131.

16. The polypeptide of claim 1, wherein the polypeptide is encoded by a nucleotide sequence having at least 98% identity to any one of SEQ ID NOS: 111, 115, and 131.

17. The polypeptide of claim 1, wherein the signal secretion peptide has at least 95% identity to any one of SEQ ID NOS: 33-43.

18. The polypeptide of claim 1, wherein the GPI anchor has at least 95% identity to any one of SEQ ID NOS: 13 and 14.

19. The polypeptide of claim 1, wherein o is 0 or 1; p is 0 or 1; x is 0 or 1; and z is 0 or 1.

20. A lentiviral vector which expresses the polypeptide of claim 1.
